(19) United States
(12) Patent Application Publication
(10) Pub. No.: US 2024/0114778 A1
(43) Pub. Date: Apr. 4, 2024

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young JUNG, Gyeonggi-do (KR); Hyo-Nim SHIN, Gyeonggi-do (KR); Seung-Hyun YOON, Gyeonggi-do (KR); Hyun-Ju KANG, Gyeonggi-do (KR); Ye-Jin JEON, Gyeonggi-do (KR); Tae-Jun HAN, Gyeonggi-do (KR); Mi-Ja LEE, Gyeonggi-do (KR); Dong-Gil KIM, Gyeonggi-do (KR); Sang-Hee CHO, Gyeonggi-do (KR)

(21) Appl. No.: 18/449,386

(22) Filed: Aug. 14, 2023

(30) Foreign Application Priority Data
Aug. 30, 2022 (KR) 10-2022-0109049
Jul. 7, 2023 (KR) 10-2023-0088225

(51) Int. Cl. H10K 85/60; C07D 263/52; C07D 307/77; C09K 11/06

(52) U.S. Cl. CPC H10K 85/636; C07D 263/52; C07D 307/77; C09K 11/06; H10K 50/11

(57) ABSTRACT

The present disclosure relates to an organic electroluminescent compound, a plurality of host materials, and an organic electroluminescent device comprising the same. By comprising the compound according to the present disclosure or by comprising a specific combination of compounds according to the present disclosure as a plurality of host materials, it is possible to produce an organic electroluminescent device having improved driving voltage, luminous efficiency, and/or lifetime properties compared to the conventional organic electroluminescent devices.

### TECHNICAL FIELD

The present disclosure relates to an organic electroluminescent compound, a plurality of host materials, and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. However, in many applications such as TVs and lightings, the lifetime of OLEDs is insufficient and higher efficiency of OLEDs is still required. Typically, the higher the luminance of an OLED, the shorter the lifetime that the OLED has. Therefore, an OLED having high luminous efficiency and/or long lifetime characteristics is required for long time use and high resolution of a display.

In order to enhance luminous efficiency, driving voltage and/or lifetime, various materials or concepts for an organic layer of an OLED have been proposed. However, they were not satisfied in practical use. In addition, there has been a need to develop an organic electroluminescent material having more improved performances, for example, improved driving voltage, luminous efficiency, power efficiency, and/or lifetime properties compared to a combination of specific compounds previously disclosed.

Meanwhile, Korean Patent Application Laying-Open No. 2018-0011429 and Chinese Patent Application Laying-Open No. 113372337 disclose an organic electroluminescent compound comprising three amine groups, and Korean Patent Application Laying-Open No. 2019-0038108 discloses an organic optoelectronic device including a compound having a structure in which three amine groups are substituted on a benzene ring. However, the aforementioned references do not specifically disclose an organic electroluminescent compound claimed herein and a plurality of host materials comprising a specific combination of compounds claimed herein.

Thus, it is continuously required to develop a light-emitting material having more improved performances, for example, improved driving voltage, luminous efficiency and/or lifetime properties, as compared with the previously disclosed combination of specific compounds.

### DISCLOSURE OF INVENTION

### Technical Problem

The objective of the present disclosure is to provide an organic electroluminescent compound having a new structure suitable for applying to an organic electroluminescent device. Another objective of the present disclosure is to provide a plurality of host materials capable of providing an organic electroluminescent device having improved driving voltage, luminous efficiency and/or lifetime properties. Still another objective of the present disclosure is to provide an organic electroluminescent device having improved driving voltage, luminous efficiency and/or lifetime properties by comprising a compound or a specific combination of compounds of the present disclosure.

### Solution to Problem

As a result of intensive studies to solve the technical problems, the present inventors found that the above objective can be achieved by a compound represented by the following formula 1; or a plurality of host materials comprising a first host material comprising an organic electroluminescent compound represented by the following formula 1 and a second host material comprising an organic electroluminescent compound represented by the following formula 2.

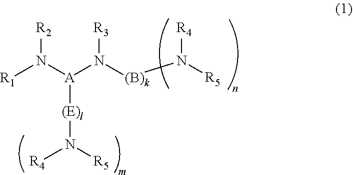

In formula 1,
R₁ to R₅, each independently, represent a substituted or unsubstituted (C6-C30)aryl, or a substituted or unsubstituted (3- to 30-membered)heteroaryl; with the proviso that at least one of R 1 to R5 represents -L-Har;
L, each independently, represents a single bond, a substituted or unsubstituted (C6-C30)arylene, or a substituted or unsubstituted (3- to 30-membered)heteroarylene;
Har, each independently, represents a substituted or unsubstituted phenanthrooxazolyl, a substituted or unsubstituted phenanthrothiazolyl, a substituted or unsubstituted phenanthrobenzofuranyl, or a substituted or unsubstituted benzophenanthrothiophenyl;
A represents a substituted or unsubstituted (C6-C30)arentriyl, or a substituted or unsubstituted (3- to 30-membered) heteroarentriyl;
B and E, each independently, represent a substituted or unsubstituted (C6-C30)arylene, or a substituted or unsubstituted (3- to 30-membered)heteroarylene; and
k represents an integer of 1 or 2, l to n, each independently, represent an integer of 0 or 1; in which if k represents an integer of 2, each of B may be the same or different;
provided that m+n=1; when n represents an integer of 0, B has the same definition as R₁, and when m represents an integer of 0, l represents an integer of 0, and A has the same definition as E.

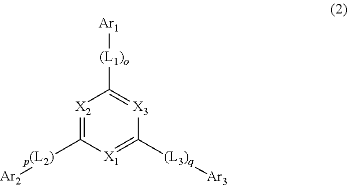

In formula 2,
X₁ to X₃, each independently, represent CR′ or N; with the proviso that at least two of X₁ to X₃ represent N;
R′ represents hydrogen or deuterium;
L₁ to L₃, each independently, represent a single bond, a substituted or unsubstituted (C6-C30)arylene, or a substituted or unsubstituted (3- to 30-membered)heteroarylene;
Ar₁ to Ar₃, each independently, represent a substituted or unsubstituted (C6-C30)aryl, or a substituted or unsubstituted (3- to 30-membered)heteroaryl; and
o to q, each independently, represent an integer of 1 or 2; in which if o to q represent an integer of 2 or more, each of L₁ to each of L₃ may be the same or different.

### Advantageous Effects of Invention

An organic electroluminescent device having low driving voltage, high luminous efficiency and/or excellent lifetime properties compared to conventional organic electroluminescent devices is provided by comprising a compound according to the present disclosure, or by comprising a specific combination of compounds according to the present disclosure as a plurality of host materials, and it is possible to produce a display system or a lighting system using the same.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may respectively be comprised in different light-emitting layers. For example, the at least two host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of 0, S, and N. The above heterocycloalkyl may include tetrahydrofuran, pyrrolidine, thiolan, tetrahydropyran, etc. The term “(C6-C30)aryl”, “(C6-C30)arylene”, and “(C6-C30)arentriyl” are meant to be a monocyclic or fused ring radical derived from an aromatic hydrocarbon having 6 to 30 ring backbone carbon atoms. The above aryl, arylene, and arentriyl may be partially saturated, and may comprise a spiro structure. The above aryl may include phenyl, biphenyl, terphenyl, quinquephenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentene-fluoren]yl, spiro[dihydroindene-fluoren]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11, 11-diphenyl-9-benzo[b]fluorenyl, 11, 11-di phenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl”, “(3- to 30-membered)heteroarylene”, and “(3- to 30-membered)heteroarentriyl” are meant to be an aryl group having 3 to 30 ring backbone atoms, and including at least one, preferably 1 to 4 heteroatoms selected from the group consisting of B, N, O, S, Si, P, Se, Te, and Ge. The above heteroaryl may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, di benzothiophenyl, dibenzoselenophenyl, naphthobenzofuranyl, naphthobenzothiophenyl, naphthooxazolyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, phenanthrooxazolyl, phenanthrothiazolyl, phenanthrobenzofuranyl, benzophenanthrothiophenyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, di benzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, di methyl benzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tent-butyl-1-indolyl, 2-tert-butyl-3-indolyl, 4-tent-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-13]-benzofuranyl, 2-naphtho-[1,2-13]-benzofuranyl, 3-naphtho-[1,2-13]-benzofuranyl, 4-naphtho-[1,2-13]-benzofuranyl, 5-naphtho-[1,2-13]-benzofuranyl, 6-naphtho-[1,2-13]-benzofuranyl, 7-naphtho-[1,2-13]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl , 1-naphtho-[2,1 -b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2 or positions 2 and 3, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as one heteroaryl substituent, or as substituents in which two heteroaryl substituents are linked. Herein, the substituent(s) of the substituted aryl, the substituted arylene, the substituted heteroaryl, the substituted heteroarylene, the substituted arentriyl and the substituted heteroarentriyl, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with deuterium; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C2-C30)alkenylamino; a mono- or di- (C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (06-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and (C1-C30)alkyl(C6-C30)aryl. According to one embodiment of the present disclosure, the substituent(s), each independently, are at least one selected from the group consisting of deuterium, a (6- to 20-membered)heteroaryl unsubstituted or substituted with deuterium, a (C6-C18)aryl unsubstituted or substituted with deuterium, and a (C6-C18)aryl(C1-C10)alkyl unsubstituted or substituted with deuterium. According to another embodiment of the present disclosure, the substituent(s), each independently, are at least one selected from the group consisting of deuterium, a (6- to 15-membered)heteroaryl unsubstituted or substituted with deuterium, a (C6-C15)aryl unsubstituted or substituted with deuterium, and a (C6-C10)aryl(C1-C6)alkyl unsubstituted or substituted with deuterium. For example, the substituent(s) may be deuterium, a methyl, a phenyl, a biphenyl, a naphthyl, a carbazolyl, or an isopropylphenyl, in which the substituents may be further substituted with deuterium.

In the formulas of the present disclosure, when a ring is formed by a linkage of adjacent substituents, the ring may be a substituted or unsubstituted, mono- or polycyclic, (3- to 30-membered) alicyclic or aromatic ring, or the combination thereof, which is formed by linkage of at least two adjacent substituents. In addition, the formed ring may contain at least one heteroatom selected from B, N, O, S, Si, and P, preferably at least one heteroatom selected from N, O, and S. According to one embodiment of the present disclosure, the number of the ring backbone atoms is 5 to 20, and according to another embodiment of the present disclosure, the number of the ring backbone atoms is 5 to 15.

The present disclosure provides the organic electroluminescent compound represented by formula 1.

Hereinafter, the compound represented by formula 1 will be described in more detail.

In formula 1, R₁ to R₅, each independently, represent a substituted or unsubstituted (C6-C30)aryl, or a substituted or unsubstituted (3- to 30-membered)heteroaryl; with the proviso that at least one of R₁ to R₅ represents -L-Har. According to one embodiment of the present disclosure, R₁ to R₅, each independently, represent a (C6-C25)aryl unsubstituted or substituted with at least one of deuterium, a (C1-C10)alkyl(s), and a (C6-C18)aryl(C1-C10)alkyl(s), or a (6- to 18-membered)heteroaryl unsubstituted or substituted with a (C6-C18)aryl(s); with the proviso that any one of R₁ to R₅ may be -L-Har. According to another embodiment of the present disclosure, R₁ to R₅, each independently, represent a (C6-C20)aryl unsubstituted or substituted with at least one of deuterium, a (C1-C6)alkyl(s), and a (C6-C10)aryl(C1-C6)alkyl(s), or a (6- to 15-membered)heteroaryl unsubstituted or substituted with a (C6-C10)aryl(s); with the proviso that any one of R₁ to R₅ may be -L-Har. For example, R₁ to R₅, each independently, may be a phenyl unsubstituted or substituted with at least one of deuterium and an isopropylphenyl(s), a biphenyl, a terphenyl, a naphthyl, a dimethylfluorenyl, a pyridyl, a dibenzofuranyl, a dibenzothiophenyl, or a carbazolyl unsubstituted or substituted with a phenyl(s); with the proviso that any one of R₁ to R₅ may be -L-Har.

In formula 1, L, each independently, represents a single bond, a substituted or unsubstituted (C6-C30)arylene, or a substituted or unsubstituted (3- to 30-membered)heteroarylene. According to one embodiment of the present disclosure, L, each independently, represents a single bond, a (C6-C18)arylene unsubstituted or substituted with deuterium, or a (5- to 25-membered)heteroaryl unsubstituted or substituted with deuterium. According to another embodiment of the present disclosure, L, each independently, represents a single bond, a (C6-C10)arylene unsubstituted or substituted with deuterium, or an unsubstituted (6- to 15-membered)heteroarylene. For example, L may be a single bond, a phenylene unsubstituted or substituted with deuterium, or an unsubstituted pyridylene.

In formula 1, Har, each independently, represents a substituted or unsubstituted phenanthrooxazolyl, a substituted or unsubstituted phenanthrothiazolyl, a substituted or unsubstituted phenanthrobenzofuranyl, or a substituted or unsubstituted benzophenanthrothiophenyl. According to one embodiment of the present disclosure, the substituent(s) of the substituted phenanthrooxazolyl, the substituted phenanthrothiazolyl, the substituted phenanthrobenzofuranyl, or the substituted benzophenanthrothiophenyl, each independently, are at least one of deuterium, a (C6-C30)aryl unsubstituted or substituted with deuterium, and a (3- to 30-membered)heteroaryl unsubstituted or substituted with deuterium. For example, Har, each independently, may be a substituted or unsubstituted phenanthrooxazolyl, a substituted or unsubstituted phenanthrothiazolyl, a substituted or unsubstituted phenanthrobenzofuranyl, or a substituted or unsubstituted benzophenanthrothiophenyl, of which the substituents, each independently, may be deuterium; or at least one of a phenyl, a biphenyl and a dibenzofuranyl, in which the substituents may be further substituted with deuterium.

In formula 1, A represents a substituted or unsubstituted (C6-C30)arentriyl, or a substituted or unsubstituted (3- to 30-membered)heteroarentriyl. According to one embodiment of the present disclosure, A represents a (C6-C18)arentriyl unsubstituted or substituted with deuterium, or a (6- to 20-membered)heteroarentriyl unsubstituted or substituted with deuterium. According to another embodiment of the present disclosure, A represents an unsubstituted (C6-C15)arentriyl, or an unsubstituted (6- to 15-membered)heteroarentriyl. For example, A may be a phenyltriyl, a biphenyltriyl, a pyridinetriyl, or a carbazolytriyl.

In formula 1, B and E, each independently, represent a substituted or unsubstituted (C6-C30)arylene, or a substituted or unsubstituted (3- to 30-membered)heteroarylene. According to one embodiment of the present disclosure, B and E, each independently, represent a (C6-C20)arylene unsubstituted or substituted with at least one of deuterium, a (C1-C10)alkyl(s), a (C6-C18)aryl(s), and a (6- to 20-membered)heteroaryl(s), or a (6- to 20-membered)heteroarylene unsubstituted or substituted with at least one of deuterium and a (C6-C18)aryl(s). According to another embodiment of the present disclosure, B and E, each independently, represent a (C6-C15)arylene unsubstituted or substituted with at least one of deuterium, a (C1-C6)alkyl(s), a (C6-C10)aryl(s), and a (6- to 15-membered)heteroaryl(s), or a (6- to 15-membered)heteroarylene unsubstituted or substituted with a (C6-C10)aryl(s). For example, B and E, each independently, may be a phenylene unsubstituted or substituted with at least one of deuterium, a phenyl(s), and a carbazolyl(s); a biphenylene; a dimethylfluorenylene; a pyridylene; a dibenzofuranylene; a dibenzothiophenylene; or a carbazolylene unsubstituted or substituted with a phenyl(s).

In formula 1, k represents an integer of 1 or 2, l to n, each independently, represent an integer of 0 or 1; in which if k represents an integer of 2, each of B may be the same or different.

In formula 1, m+n=1.

In formula 1, when n represents an integer of 0, B has the same definition and specific embodiments as R₁.

In formula 1, when m represents an integer of 0, l represents an integer of 0, and A has the same definition and specific embodiments as E.

According to one embodiment of the present disclosure, in formula 1, Har, each independently, may be represented by the following formula a.

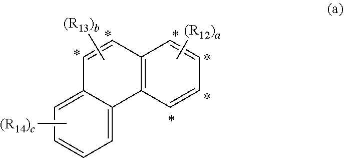

In formula a, adjacent * is connected to the following formula a-1 or a-2.

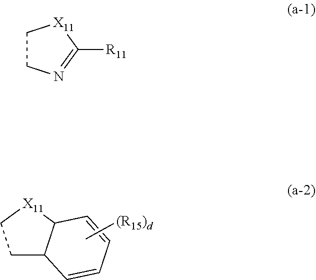

In formulas a-1 and a-2, X₁₁, each independently, represents O or S.

In formulas a, a-1 and a-2, any one of R₁₁ to R₁₅ is connected to L.

In formulas a, a-1 and a-2, if R₁₁ is not connected to L, R₁₁ represents a substituted or unsubstituted (C6-C30)aryl, or a substituted or unsubstituted (3- to 30-membered)heteroaryl; and if R₁₂ to R₁₅ are not connected to L, R₁₂ to R₁₅, each independently, represent hydrogen, deuterium, a substituted or unsubstituted (C6-C30)aryl, or a substituted or unsubstituted (3- to 30-membered)heteroaryl.

According to one embodiment of the present disclosure, R₁₁ represent a (C6-C18)aryl unsubstituted or substituted with deuterium, or an unsubstituted (6- to 20-membered)heteroaryl; and R₁₂ to R₁₅, each independently, represent hydrogen, deuterium, a substituted or unsubstituted (C6-C18)aryl, or a substituted or unsubstituted (6- to 20-membered)heteroaryl. According to another embodiment of the present disclosure, R₁₁ represent a (C6-C15)aryl unsubstituted or substituted with deuterium, or an unsubstituted (6- to 15-membered)heteroaryl. For example, R₁₁ may be a phenyl unsubstituted or substituted with deuterium, a biphenyl, or a dibenzofuranyl, and R₁₂ to R₁₅, each independently, may be hydrogen or deuterium.

In formulas a, a-1 and a-2, a, c and d, each independently, represent an integer of 1 to 4, b, each independently, represents an integer of 0 to 2; in which if a to d represent an integer of 2 or more, each of R₁₂ to each of R₁₅, may be the same or different.

According to one embodiment of the present disclosure, in formula 1, Har, each independently, may be represented by the following formula 1-1 or 1-2.

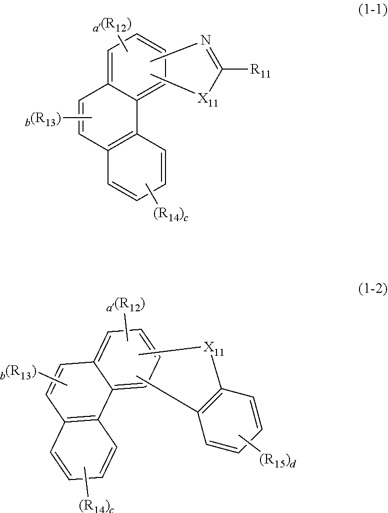

In formulas 1-1 and 1-2, a′, each independently, represents an integer of 1 or 2, in which if a′ represents an integer of 2, each of R₁₂ may be the same or different.

In formulas 1-1 and 1-2, X₁₁, R₁₁ to R₁₅, and b to d are as defined in formulas a, a-1, a-2.

According to another embodiment of the present disclosure, in formula 1, Har, each independently, may be represented by any one of the following formulas 1-11 to 1-15.

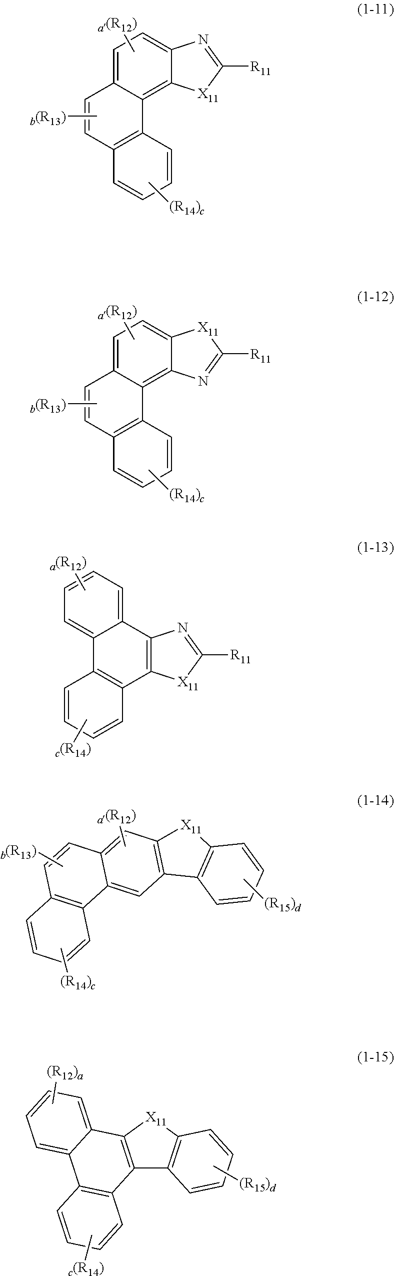

In formulas 1-11, 1-12, and 1-14, a′ represents an integer of 1 or 2, in which if a′ represents an integer of 2 or more, each of R₁₂ may be the same or different.

In formulas 1-11 to 1-15, X₁₁, R₁₁ to R₁₅, and a to d are as defined in formulas a, a-1, a-2.
According to one embodiment of the present disclosure, in formula 1, when n represents an integer of 1 and m represents an integer of 0, at least one of A and B may be represented by any one of the following formulas.

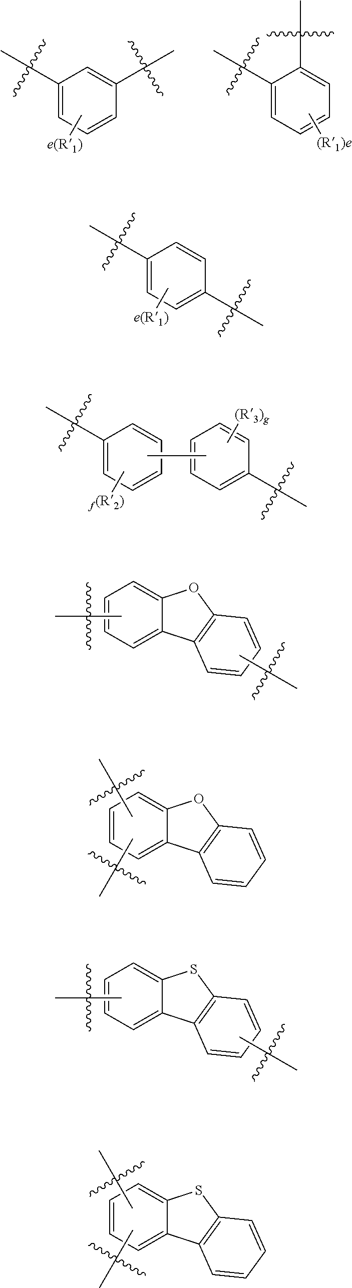

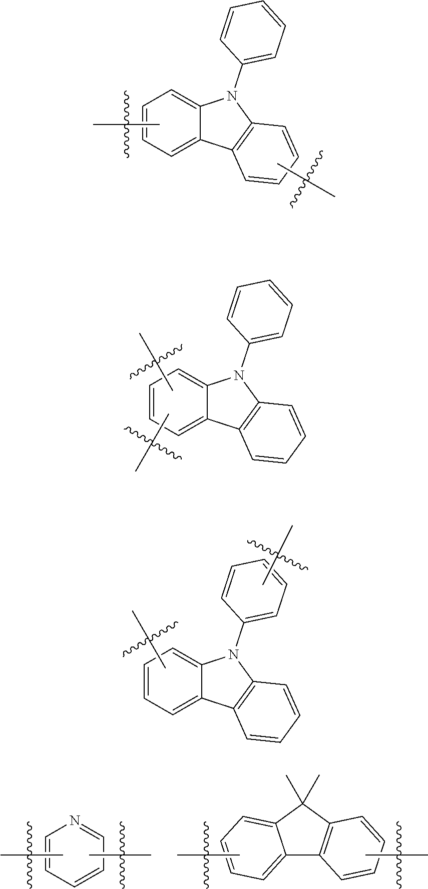

In the formulas, hydrogen may be replaced by deuterium. In the formulas, R′₁ to R′₃, each independently, represent hydrogen, deuterium, a substituted or unsubstituted (C6-C30)aryl, or a substituted or unsubstituted (3- to 30-membered)heteroaryl. According to one embodiment of the present disclosure, R′₁ to R′₃, each independently, represent hydrogen, deuterium, an unsubstituted (C6-C18)aryl, or an unsubstituted (6- to 20-membered)heteroaryl. According to another embodiment of the present disclosure, R′₁ to R′₃, each independently, represent hydrogen, deuterium, an unsubstituted (C6-C10)aryl, or an unsubstituted (6- to 15-membered)heteroaryl. For example, R′₁ to R′₃, each independently, may be hydrogen, deuterium, a phenyl or a carbazolyl, which may be further substituted with deuterium.

In the formulas, e to g, each independently, represent an integer of 1 to 4, in which if e to g represent an integer of 2 or more, each of R′₁ to each of R′₃ may be the same or different.

According to one embodiment of the present disclosure, in formula 1, when n represents an integer of 0 and m represents an integer of 1, A is represented by any one of the following formulas.

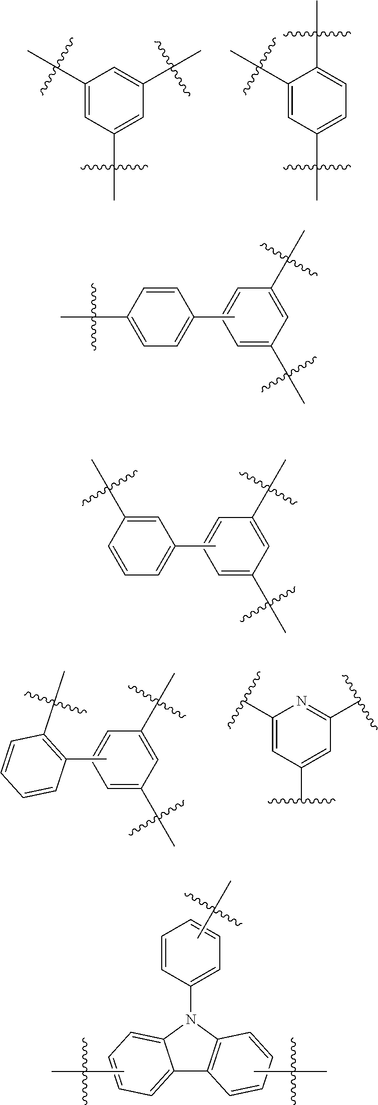

In the formulas, hydrogen may be replaced by deuterium.

The compound represented by formula 1 may be selected from the group consisting of the following compounds, but is not limited thereto.

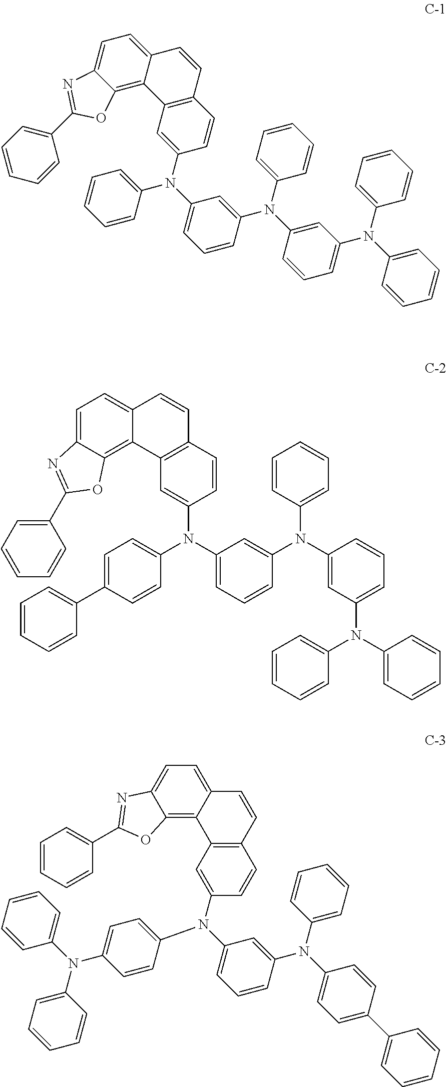 [SMILES: c1ccc(-c2nc3ccc4ccc5ccc(N(c6ccccc6)c6cccc(N(c7ccccc7)c7cccc(N(c8ccccc8)c8ccccc8)c7)c6)cc5c4c3o2)cc1 ; c1ccc(-c2ccc(N(c3cccc(N(c4ccccc4)c4cccc(N(c5ccccc5)c5ccccc5)c4)c3)c3ccc4ccc5ccc6nc(-c7ccccc7)oc6c5c4c3)cc2)cc1 ; c1ccc(-c2ccc(N(c3ccccc3)c3cccc(N(c4ccc(N(c5ccccc5)c5ccccc5)cc4)c4ccc5ccc6ccc7nc(-c8ccccc8)oc7c6c5c4)c3)cc2)cc1]

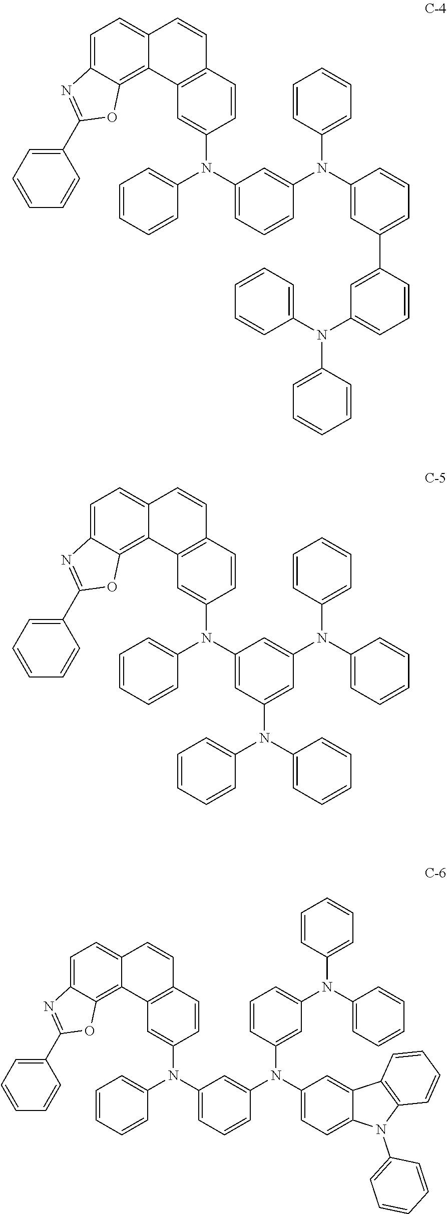 [SMILES: c1ccc(-c2nc3ccc4ccc5ccc(N(c6ccccc6)c6cccc(N(c7ccccc7)c7cccc(-c8cccc(N(c9ccccc9)c9ccccc9)c8)c7)c6)cc5c4c3o2)cc1 ; c1ccc(-c2nc3ccc4ccc5ccc(N(c6ccccc6)c6cc(N(c7ccccc7)c7ccccc7)cc(N(c7ccccc7)c7ccccc7)c6)cc5c4c3o2)cc1 ; c1ccc(-c2nc3ccc4ccc5ccc(N(c6ccccc6)c6cccc(N(c7cccc(N(c8ccccc8)c8ccccc8)c7)c7ccc8c(c7)c7ccccc7n8-c7ccccc7)c6)cc5c4c3o2)cc1]

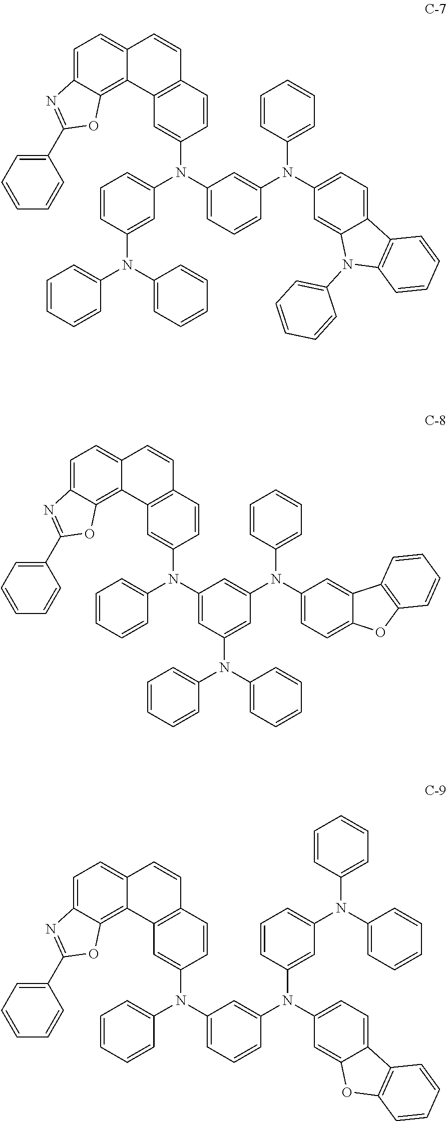 [SMILES: c1ccc(-c2nc3ccc4ccc5ccc(N(c6cccc(N(c7ccccc7)c7ccccc7)c6)c6cccc(N(c7ccccc7)c7ccc8c9ccccc9n(-c9ccccc9)c8c7)c6)cc5c4c3o2)cc1 ; c1ccc(-c2nc3ccc4ccc5ccc(N(c6ccccc6)c6cc(N(c7ccccc7)c7ccccc7)cc(N(c7ccccc7)c7ccc8oc9ccccc9c8c7)c6)cc5c4c3o2)cc1 ; c1ccc(-c2nc3ccc4ccc5ccc(N(c6ccccc6)c6cccc(N(c7cccc(N(c8ccccc8)c8ccccc8)c7)c7ccc8c(c7)oc7ccccc78)c6)cc5c4c3o2)cc1]

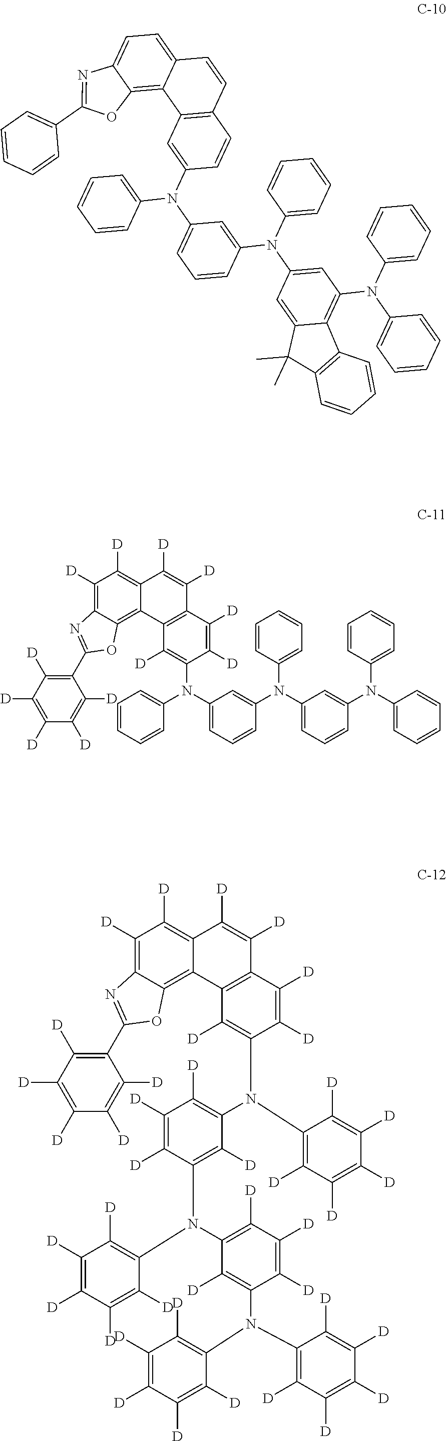

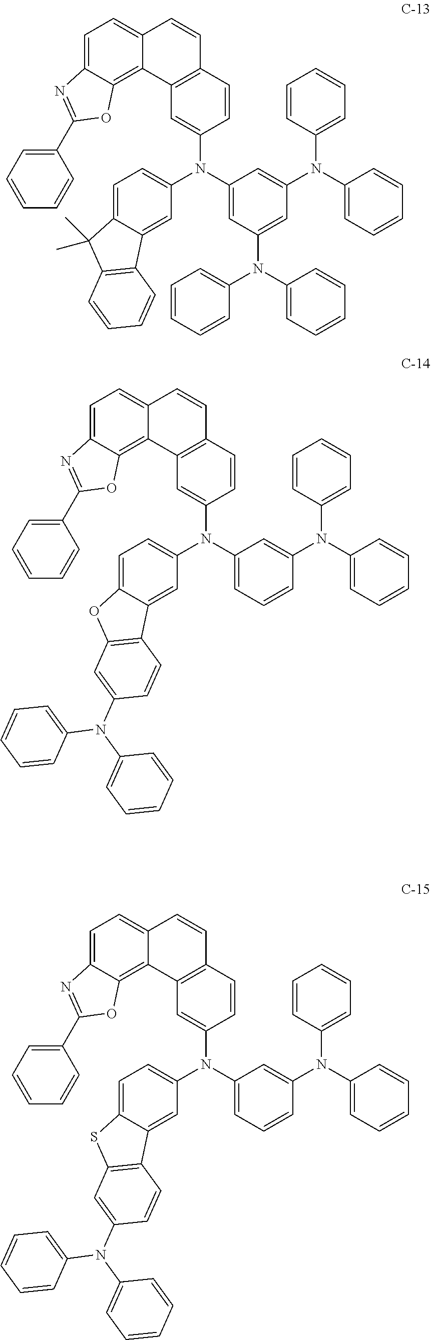 [SMILES: CC1(C)c2ccccc2-c2cc(N(c3cc(N(c4ccccc4)c4ccccc4)cc(N(c4ccccc4)c4ccccc4)c3)c3ccc4ccc5ccc6nc(-c7ccccc7)oc6c5c4c3)ccc21 ; c1ccc(-c2nc3ccc4ccc5ccc(N(c6cccc(N(c7ccccc7)c7ccccc7)c6)c6ccc7oc8cc(N(c9ccccc9)c9ccccc9)ccc8c7c6)cc5c4c3o2)cc1 ; c1ccc(-c2nc3ccc4ccc5ccc(N(c6cccc(N(c7ccccc7)c7ccccc7)c6)c6ccc7sc8cc(N(c9ccccc9)c9ccccc9)ccc8c7c6)cc5c4c3o2)cc1]

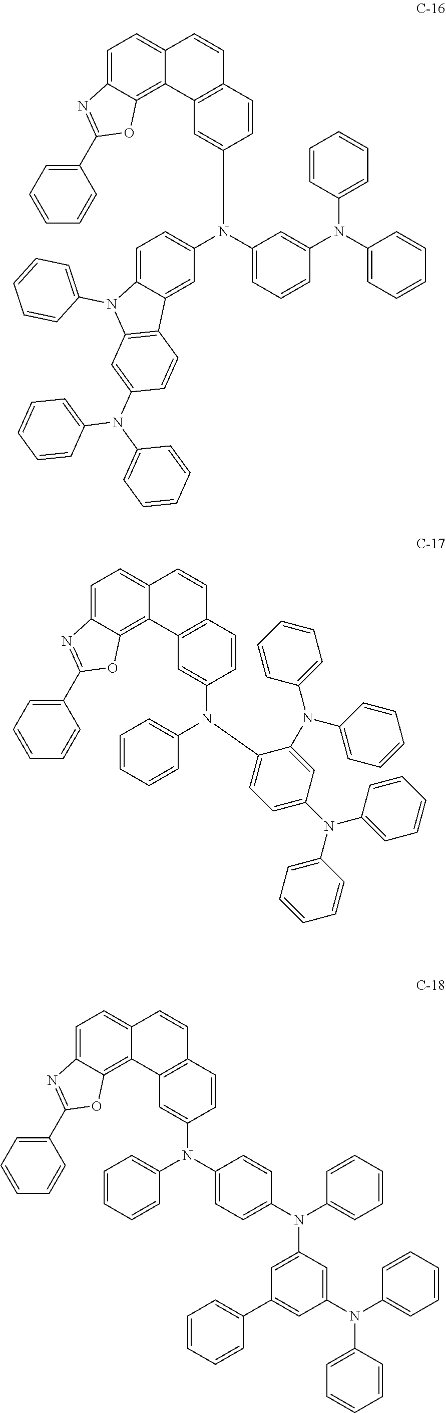 [SMILES: c1ccc(-c2nc3ccc4ccc5ccc(N(c6cccc(N(c7ccccc7)c7ccccc7)c6)c6ccc7c(c6)c6ccc(N(c8ccccc8)c8ccccc8)cc6n7-c6ccccc6)cc5c4c3o2)cc1 ; c1ccc(-c2nc3ccc4ccc5ccc(N(c6ccccc6)c6ccc(N(c7ccccc7)c7ccccc7)cc6N(c6ccccc6)c6ccccc6)cc5c4c3o2)cc1 ; c1ccc(-c2cc(N(c3ccccc3)c3ccccc3)cc(N(c3ccccc3)c3ccc(N(c4ccccc4)c4ccc5ccc6ccc7nc(-c8ccccc8)oc7c6c5c4)cc3)c2)cc1]

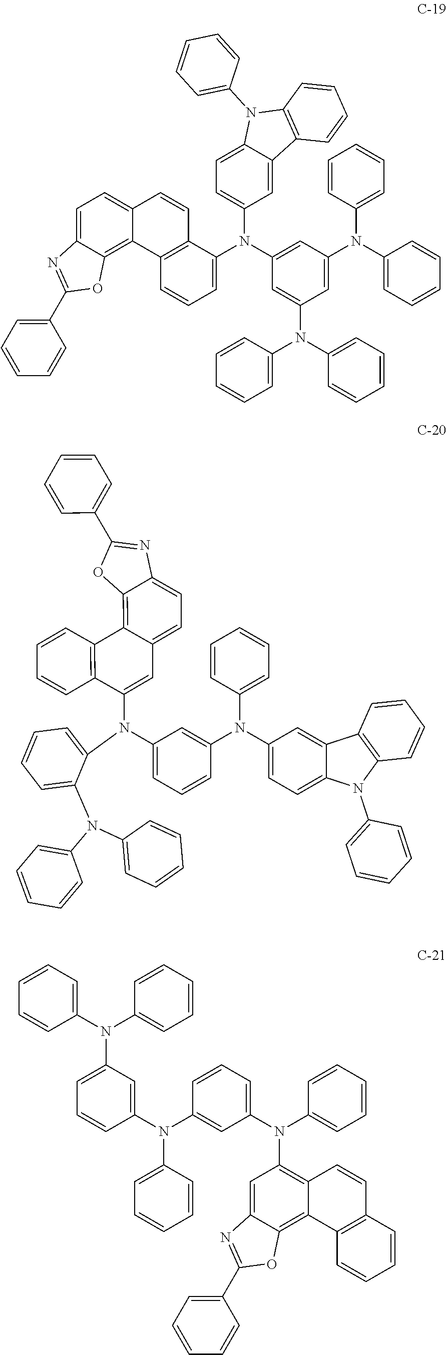 [SMILES: c1ccc(-c2nc3ccc4ccc5c(N(c6cc(N(c7ccccc7)c7ccccc7)cc(N(c7ccccc7)c7ccccc7)c6)c6ccc7c(c6)c6ccccc6n7-c6ccccc6)cccc5c4c3o2)cc1 ; c1ccc(-c2nc3ccc4cc(N(c5cccc(N(c6ccccc6)c6ccc7c(c6)c6ccccc6n7-c6ccccc6)c5)c5ccccc5N(c5ccccc5)c5ccccc5)c5ccccc5c4c3o2)cc1 ; c1ccc(-c2nc3cc(N(c4ccccc4)c4cccc(N(c5ccccc5)c5cccc(N(c6ccccc6)c6ccccc6)c5)c4)c4ccc5ccccc5c4c3o2)cc1]

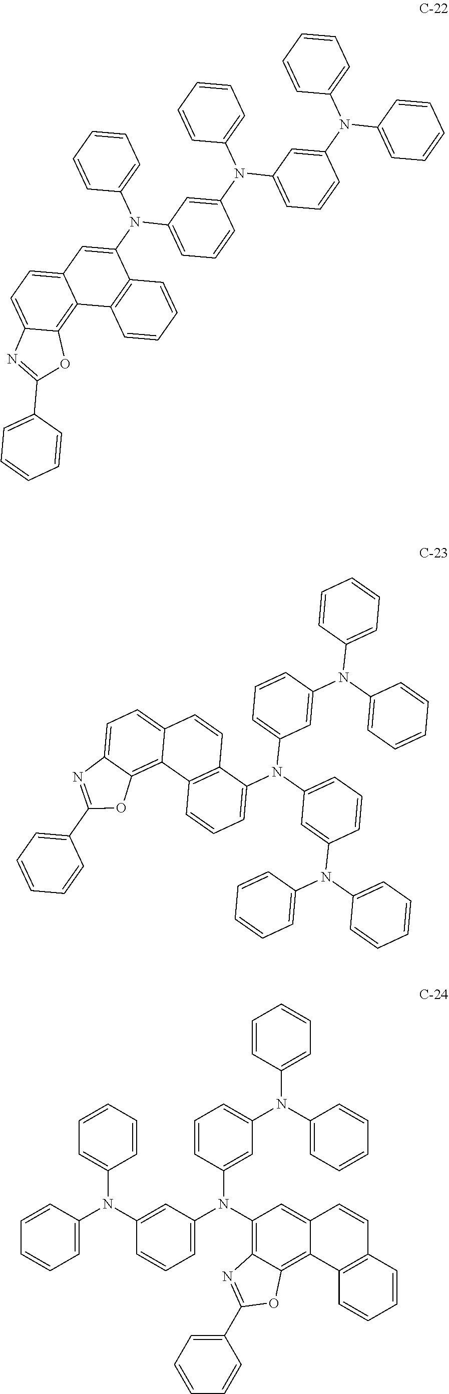 [SMILES: c1ccc(-c2nc3ccc4cc(N(c5ccccc5)c5cccc(N(c6ccccc6)c6cccc(N(c7ccccc7)c7ccccc7)c6)c5)c5ccccc5c4c3o2)cc1 ; c1ccc(-c2nc3ccc4ccc5c(N(c6cccc(N(c7ccccc7)c7ccccc7)c6)c6cccc(N(c7ccccc7)c7ccccc7)c6)cccc5c4c3o2)cc1 ; c1ccc(-c2nc3c(N(c4cccc(N(c5ccccc5)c5ccccc5)c4)c4cccc(N(c5ccccc5)c5ccccc5)c4)cc4ccc5ccccc5c4c3o2)cc1]

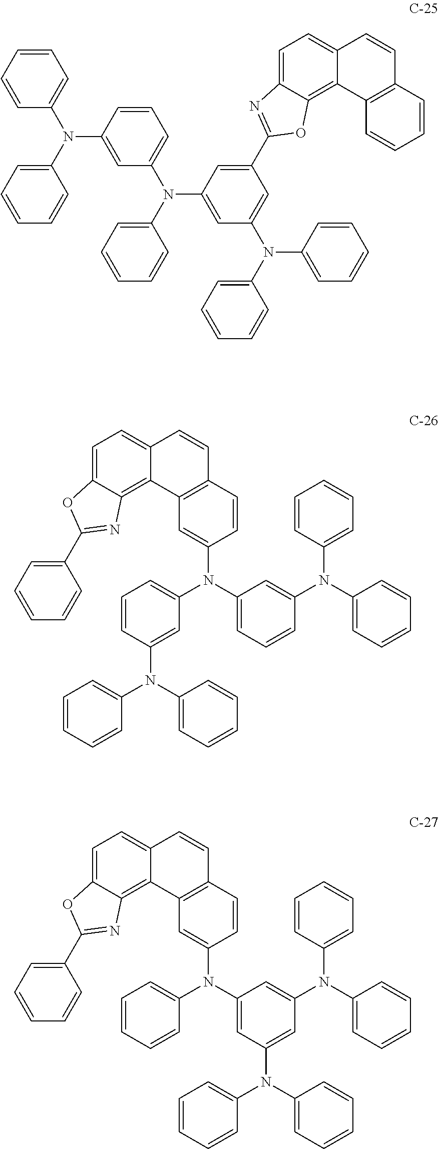 [SMILES: c1ccc(N(c2ccccc2)c2cccc(N(c3ccccc3)c3cc(-c4nc5ccc6ccc7ccccc7c6c5o4)cc(N(c4ccccc4)c4ccccc4)c3)c2)cc1 ; c1ccc(-c2nc3c(ccc4ccc5ccc(N(c6cccc(N(c7ccccc7)c7ccccc7)c6)c6cccc(N(c7ccccc7)c7ccccc7)c6)cc5c43)o2)cc1 ; c1ccc(-c2nc3c(ccc4ccc5ccc(N(c6ccccc6)c6cc(N(c7ccccc7)c7ccccc7)cc(N(c7ccccc7)c7ccccc7)c6)cc5c43)o2)cc1]

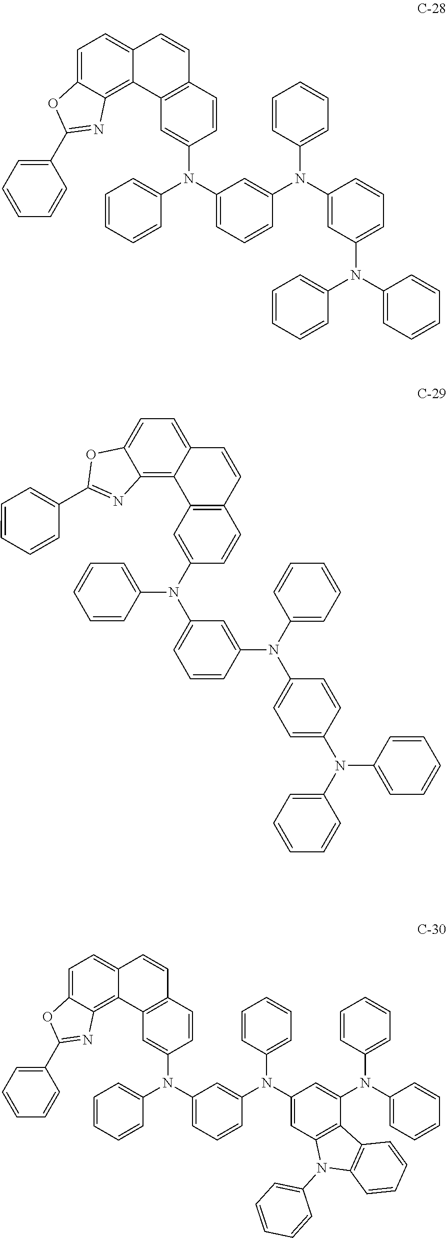 [SMILES: c1ccc(-c2nc3c(ccc4ccc5ccc(N(c6ccccc6)c6cccc(N(c7ccccc7)c7cccc(N(c8ccccc8)c8ccccc8)c7)c6)cc5c43)o2)cc1 ; c1ccc(-c2nc3c(ccc4ccc5ccc(N(c6ccccc6)c6cccc(N(c7ccccc7)c7ccc(N(c8ccccc8)c8ccccc8)cc7)c6)cc5c43)o2)cc1 ; c1ccc(-c2nc3c(ccc4ccc5ccc(N(c6ccccc6)c6cccc(N(c7ccccc7)c7cc(N(c8ccccc8)c8ccccc8)c8c9ccccc9n(-c9ccccc9)c8c7)c6)cc5c43)o2)cc1]

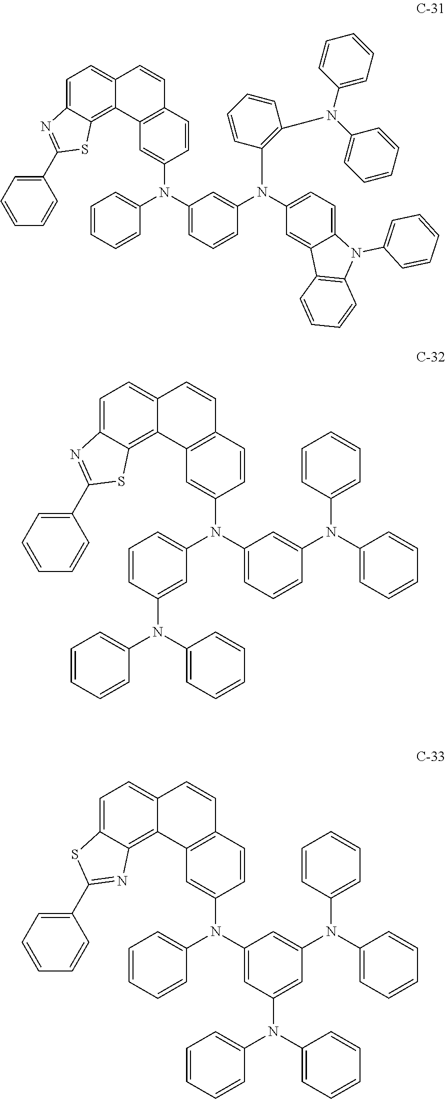 [SMILES: c1ccc(-c2nc3ccc4ccc5ccc(N(c6ccccc6)c6cccc(N(c7ccc8c(c7)c7ccccc7n8-c7ccccc7)c7ccccc7N(c7ccccc7)c7ccccc7)c6)cc5c4c3s2)cc1 ; c1ccc(-c2nc3ccc4ccc5ccc(N(c6cccc(N(c7ccccc7)c7ccccc7)c6)c6cccc(N(c7ccccc7)c7ccccc7)c6)cc5c4c3s2)cc1 ; c1ccc(-c2nc3c(ccc4ccc5ccc(N(c6ccccc6)c6cc(N(c7ccccc7)c7ccccc7)cc(N(c7ccccc7)c7ccccc7)c6)cc5c43)s2)cc1]

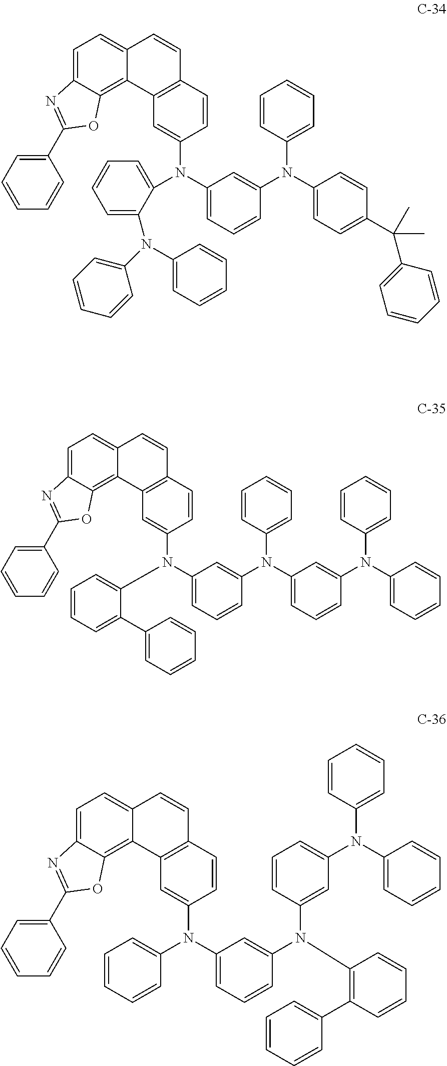 [SMILES: CC(C)(c1ccccc1)c1ccc(N(c2ccccc2)c2cccc(N(c3ccc4ccc5ccc6nc(-c7ccccc7)oc6c5c4c3)c3ccccc3N(c3ccccc3)c3ccccc3)c2)cc1 ; c1ccc(-c2nc3ccc4ccc5ccc(N(c6cccc(N(c7ccccc7)c7cccc(N(c8ccccc8)c8ccccc8)c7)c6)c6ccccc6-c6ccccc6)cc5c4c3o2)cc1 ; c1ccc(-c2nc3ccc4ccc5ccc(N(c6ccccc6)c6cccc(N(c7cccc(N(c8ccccc8)c8ccccc8)c7)c7ccccc7-c7ccccc7)c6)cc5c4c3o2)cc1]

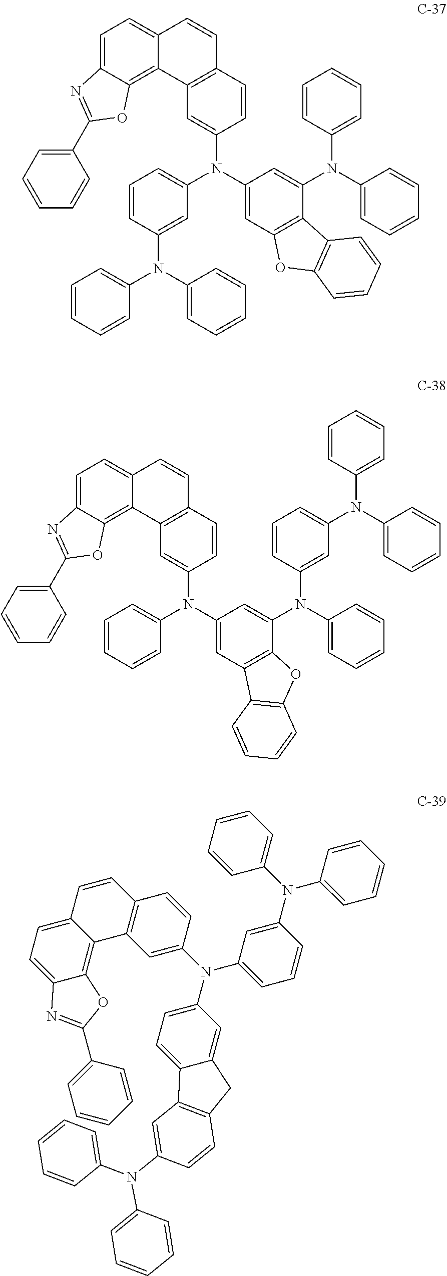 [SMILES: c1ccc(-c2nc3ccc4ccc5ccc(N(c6cccc(N(c7ccccc7)c7ccccc7)c6)c6cc(N(c7ccccc7)c7ccccc7)c7c(c6)oc6ccccc67)cc5c4c3o2)cc1 ; c1ccc(-c2nc3ccc4ccc5ccc(N(c6ccccc6)c6cc(N(c7ccccc7)c7cccc(N(c8ccccc8)c8ccccc8)c7)c7oc8ccccc8c7c6)cc5c4c3o2)cc1 ; c1ccc(-c2nc3ccc4ccc5ccc(N(c6cccc(N(c7ccccc7)c7ccccc7)c6)c6ccc7c(c6)Cc6ccc(N(c8ccccc8)c8ccccc8)cc6-7)cc5c4c3o2)cc1]

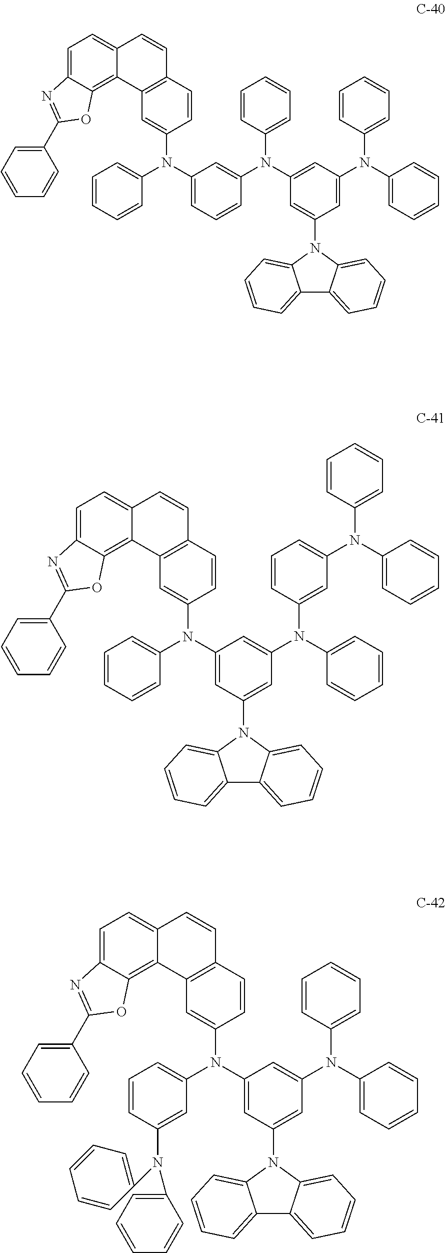 [SMILES: c1ccc(-c2nc3ccc4ccc5ccc(N(c6ccccc6)c6cccc(N(c7ccccc7)c7cc(N(c8ccccc8)c8ccccc8)cc(-n8c9ccccc9c9ccccc98)c7)c6)cc5c4c3o2)cc1 ; c1ccc(-c2nc3ccc4ccc5ccc(N(c6ccccc6)c6cc(N(c7ccccc7)c7cccc(N(c8ccccc8)c8ccccc8)c7)cc(-n7c8ccccc8c8ccccc87)c6)cc5c4c3o2)cc1 ; c1ccc(-c2nc3ccc4ccc5ccc(N(c6cccc(N(c7ccccc7)c7ccccc7)c6)c6cc(N(c7ccccc7)c7ccccc7)cc(-n7c8ccccc8c8ccccc87)c6)cc5c4c3o2)cc1]

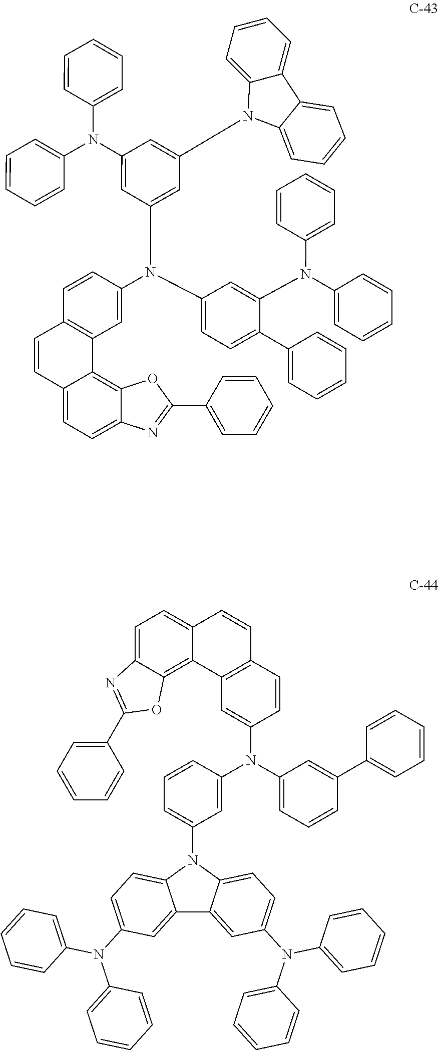 [SMILES: c1ccc(-c2nc3ccc4ccc5ccc(N(c6cc(N(c7ccccc7)c7ccccc7)cc(-n7c8ccccc8c8ccccc87)c6)c6ccc(-c7ccccc7)c(N(c7ccccc7)c7ccccc7)c6)cc5c4c3o2)cc1 ; c1ccc(-c2cccc(N(c3cccc(-n4c5ccc(N(c6ccccc6)c6ccccc6)cc5c5cc(N(c6ccccc6)c6ccccc6)ccc54)c3)c3ccc4ccc5ccc6nc(-c7ccccc7)oc6c5c4c3)c2)cc1]

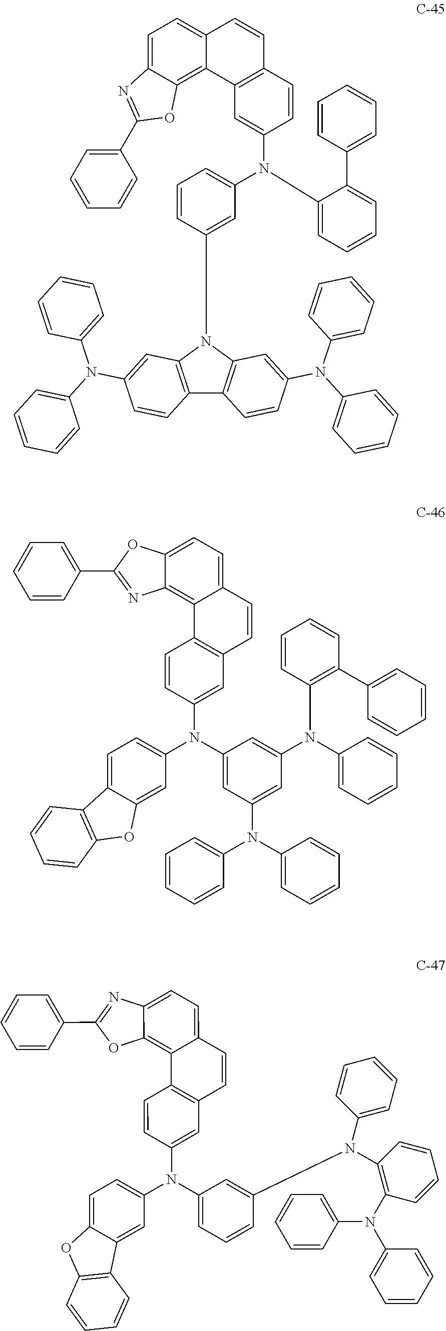 [SMILES: c1ccc(-c2nc3ccc4ccc5ccc(N(c6cccc(-n7c8cc(N(c9ccccc9)c9ccccc9)ccc8c8ccc(N(c9ccccc9)c9ccccc9)cc87)c6)c6ccccc6-c6ccccc6)cc5c4c3o2)cc1 ; c1ccc(-c2nc3c(ccc4ccc5cc(N(c6cc(N(c7ccccc7)c7ccccc7)cc(N(c7ccccc7)c7ccccc7-c7ccccc7)c6)c6ccc7c(c6)oc6ccccc67)ccc5c43)o2)cc1 ; c1ccc(-c2nc3ccc4ccc5cc(N(c6cccc(N(c7ccccc7)c7ccccc7N(c7ccccc7)c7ccccc7)c6)c6ccc7oc8ccccc8c7c6)ccc5c4c3o2)cc1]

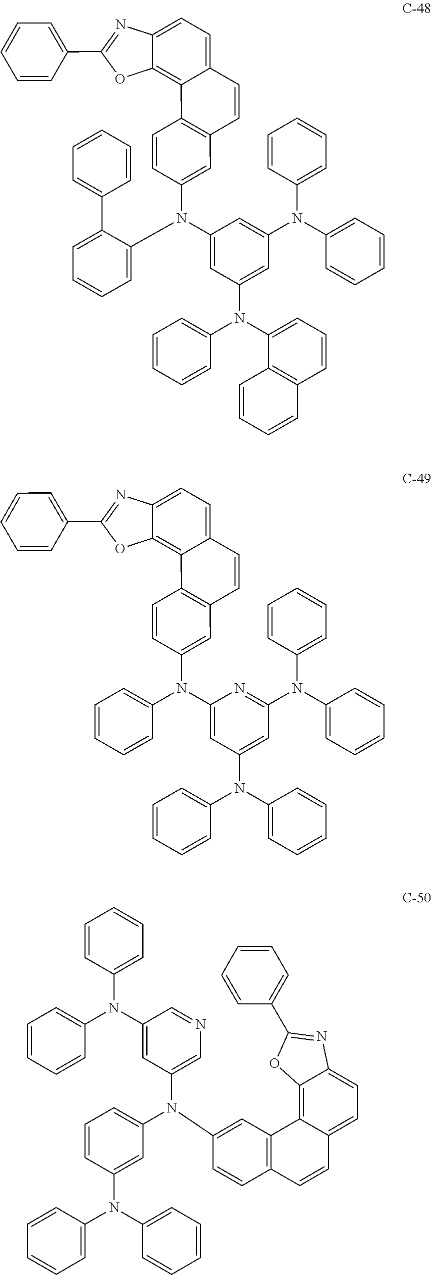 [SMILES: c1ccc(-c2nc3ccc4ccc5cc(N(c6cc(N(c7ccccc7)c7ccccc7)cc(N(c7ccccc7)c7cccc8ccccc78)c6)c6ccccc6-c6ccccc6)ccc5c4c3o2)cc1 ; c1ccc(-c2nc3ccc4ccc5cc(N(c6ccccc6)c6cc(N(c7ccccc7)c7ccccc7)cc(N(c7ccccc7)c7ccccc7)n6)ccc5c4c3o2)cc1 ; c1ccc(-c2nc3ccc4ccc5ccc(N(c6cccc(N(c7ccccc7)c7ccccc7)c6)c6cncc(N(c7ccccc7)c7ccccc7)c6)cc5c4c3o2)cc1]

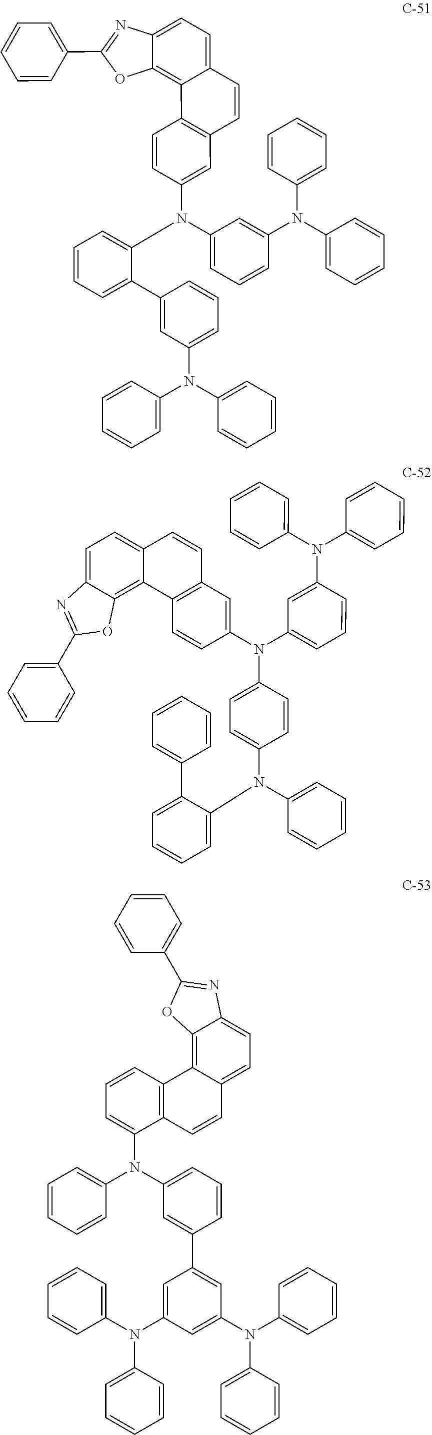

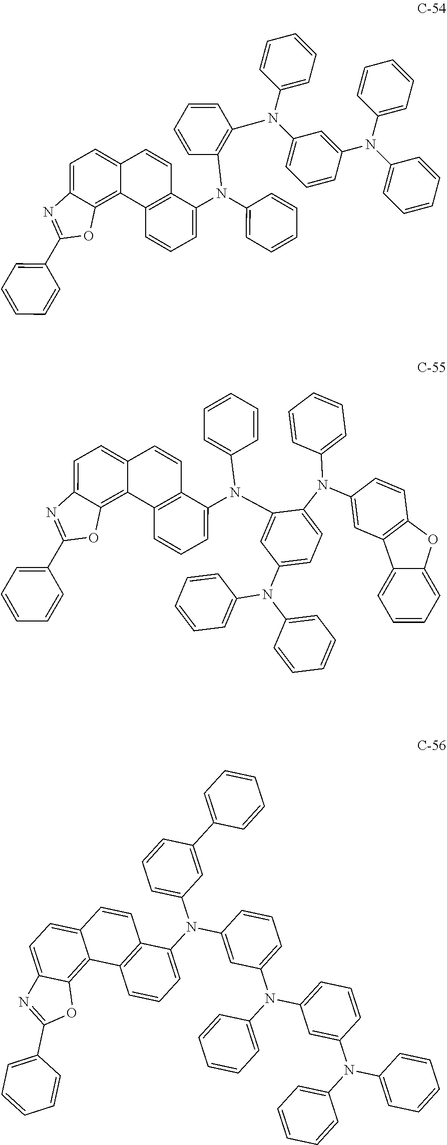 [SMILES: c1ccc(-c2nc3ccc4ccc5c(N(c6ccccc6)c6ccccc6N(c6ccccc6)c6cccc(N(c7ccccc7)c7ccccc7)c6)cccc5c4c3o2)cc1 ; c1ccc(-c2nc3ccc4ccc5c(N(c6ccccc6)c6cc(N(c7ccccc7)c7ccccc7)ccc6N(c6ccccc6)c6ccc7oc8ccccc8c7c6)cccc5c4c3o2)cc1 ; c1ccc(-c2cccc(N(c3cccc(N(c4ccccc4)c4cccc(N(c5ccccc5)c5ccccc5)c4)c3)c3cccc4c3ccc3ccc5nc(-c6ccccc6)oc5c34)c2)cc1]

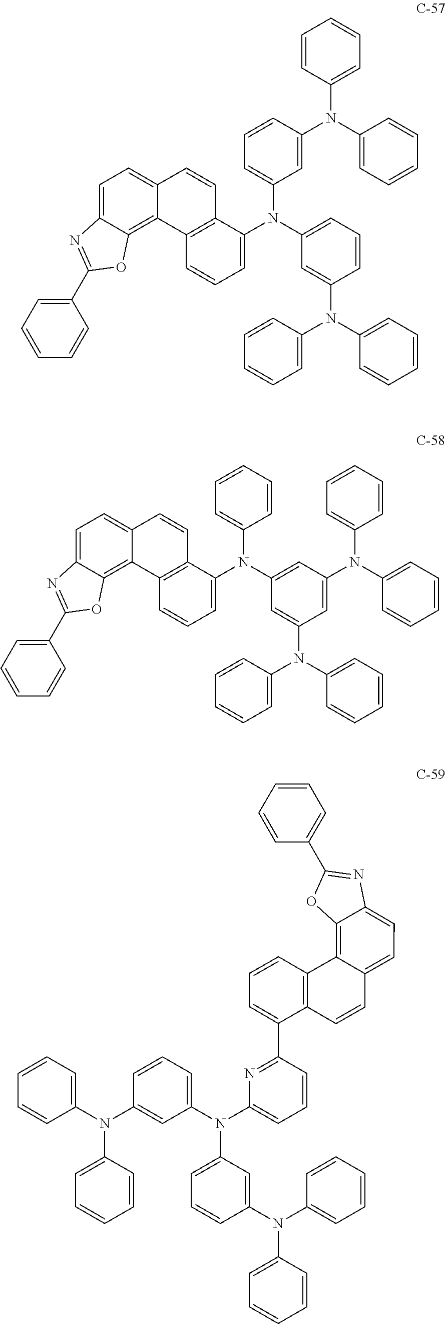 [SMILES: c1ccc(-c2nc3ccc4ccc5c(N(c6cccc(N(c7ccccc7)c7ccccc7)c6)c6cccc(N(c7ccccc7)c7ccccc7)c6)cccc5c4c3o2)cc1 ; c1ccc(-c2nc3ccc4ccc5c(N(c6ccccc6)c6cc(N(c7ccccc7)c7ccccc7)cc(N(c7ccccc7)c7ccccc7)c6)cccc5c4c3o2)cc1 ; c1ccc(-c2nc3ccc4ccc5c(-c6cccc(N(c7cccc(N(c8ccccc8)c8ccccc8)c7)c7cccc(N(c8ccccc8)c8ccccc8)c7)n6)cccc5c4c3o2)cc1]

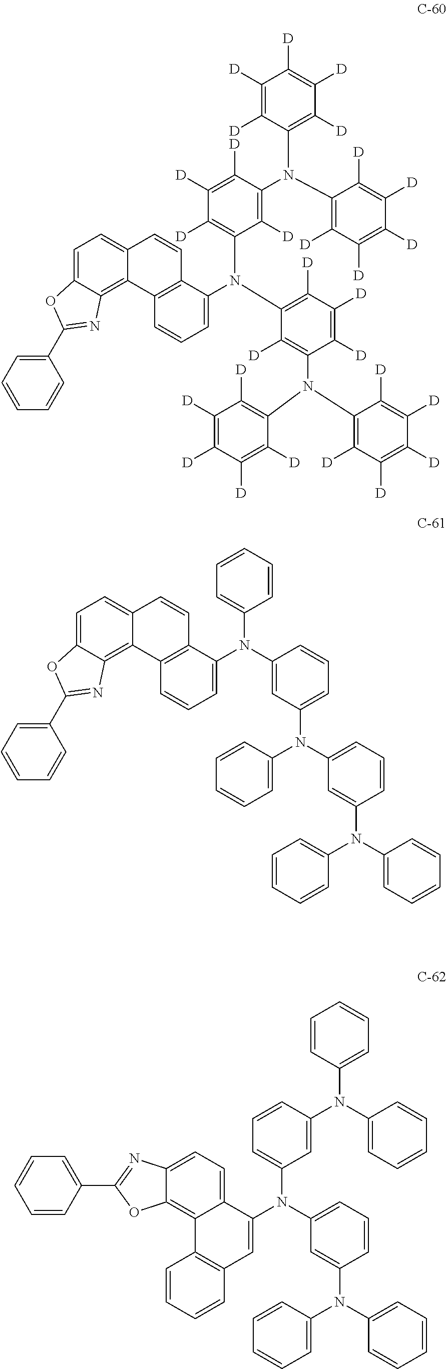 [SMILES: [2H]c1c([2H])c([2H])c(N(c2c([2H])c([2H])c([2H])c([2H])c2[2H])c2c([2H])c([2H])c([2H])c(N(c3c([2H])c([2H])c([2H])c(N(c4c([2H])c([2H])c([2H])c([2H])c4[2H])c4c([2H])c([2H])c([2H])c([2H])c4[2H])c3[2H])c3cccc4c3ccc3ccc5oc(-c6ccccc6)nc5c34)c2[2H])c([2H])c1[2H] ; c1ccc(-c2nc3c(ccc4ccc5c(N(c6ccccc6)c6cccc(N(c7ccccc7)c7cccc(N(c8ccccc8)c8ccccc8)c7)c6)cccc5c43)o2)cc1 ; c1ccc(-c2nc3ccc4c(N(c5cccc(N(c6ccccc6)c6ccccc6)c5)c5cccc(N(c6ccccc6)c6ccccc6)c5)cc5ccccc5c4c3o2)cc1]

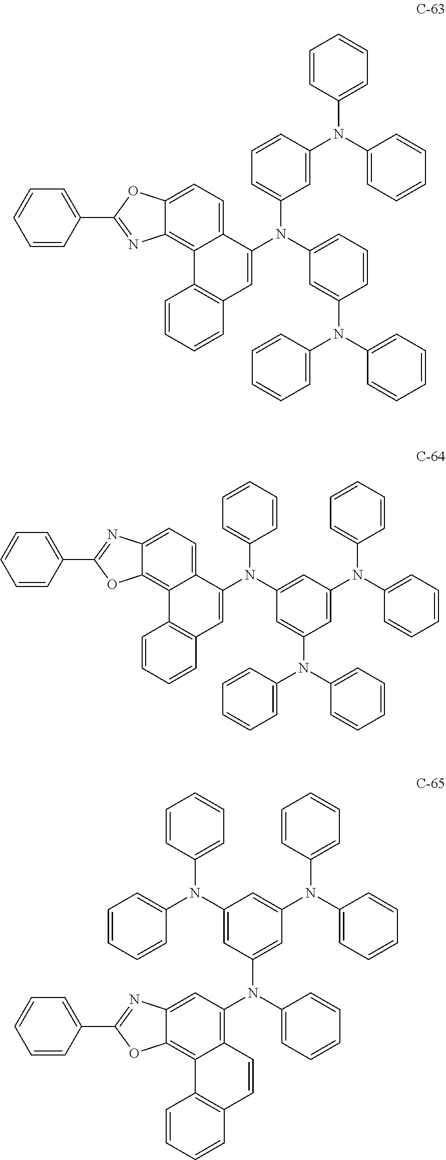 [SMILES: c1ccc(-c2nc3c(ccc4c(N(c5cccc(N(c6ccccc6)c6ccccc6)c5)c5cccc(N(c6ccccc6)c6ccccc6)c5)cc5ccccc5c43)o2)cc1 ; c1ccc(-c2nc3ccc4c(N(c5ccccc5)c5cc(N(c6ccccc6)c6ccccc6)cc(N(c6ccccc6)c6ccccc6)c5)cc5ccccc5c4c3o2)cc1 ; c1ccc(-c2nc3cc(N(c4ccccc4)c4cc(N(c5ccccc5)c5ccccc5)cc(N(c5ccccc5)c5ccccc5)c4)c4ccc5ccccc5c4c3o2)cc1]

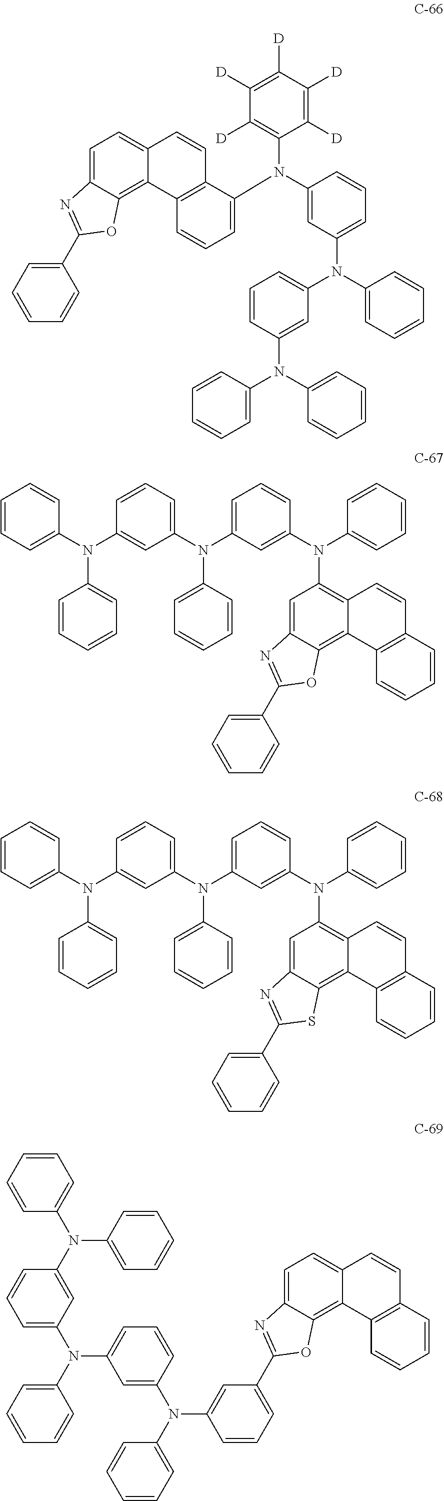 [SMILES: [2H]c1c([2H])c([2H])c(N(c2cccc(N(c3ccccc3)c3cccc(N(c4ccccc4)c4ccccc4)c3)c2)c2cccc3c2ccc2ccc4nc(-c5ccccc5)oc4c23)c([2H])c1[2H] ; c1ccc(-c2nc3cc(N(c4ccccc4)c4cccc(N(c5ccccc5)c5cccc(N(c6ccccc6)c6ccccc6)c5)c4)c4ccc5ccccc5c4c3o2)cc1 ; c1ccc(-c2nc3cc(N(c4ccccc4)c4cccc(N(c5ccccc5)c5cccc(N(c6ccccc6)c6ccccc6)c5)c4)c4ccc5ccccc5c4c3s2)cc1 ; c1ccc(N(c2ccccc2)c2cccc(N(c3ccccc3)c3cccc(N(c4ccccc4)c4cccc(-c5nc6ccc7ccc8ccccc8c7c6o5)c4)c3)c2)cc1]

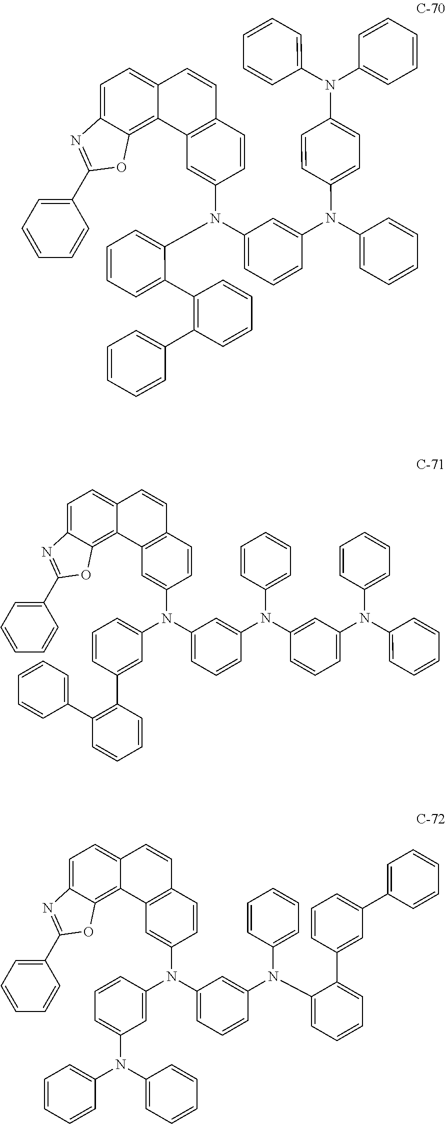 [SMILES: c1ccc(-c2nc3ccc4ccc5ccc(N(c6cccc(N(c7ccccc7)c7ccc(N(c8ccccc8)c8ccccc8)cc7)c6)c6ccccc6-c6ccccc6-c6ccccc6)cc5c4c3o2)cc1 ; c1ccc(-c2nc3ccc4ccc5ccc(N(c6cccc(-c7ccccc7-c7ccccc7)c6)c6cccc(N(c7ccccc7)c7cccc(N(c8ccccc8)c8ccccc8)c7)c6)cc5c4c3o2)cc1 ; c1ccc(-c2cccc(-c3ccccc3N(c3ccccc3)c3cccc(N(c4cccc(N(c5ccccc5)c5ccccc5)c4)c4ccc5ccc6ccc7nc(-c8ccccc8)oc7c6c5c4)c3)c2)cc1]

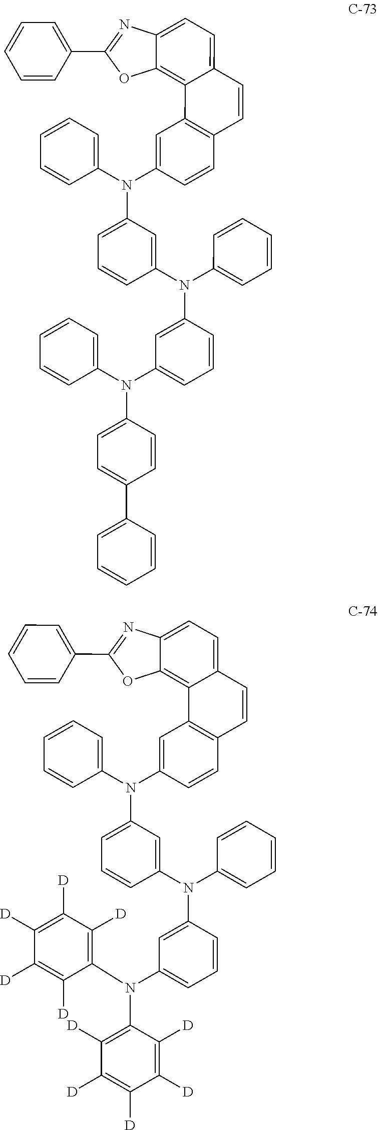

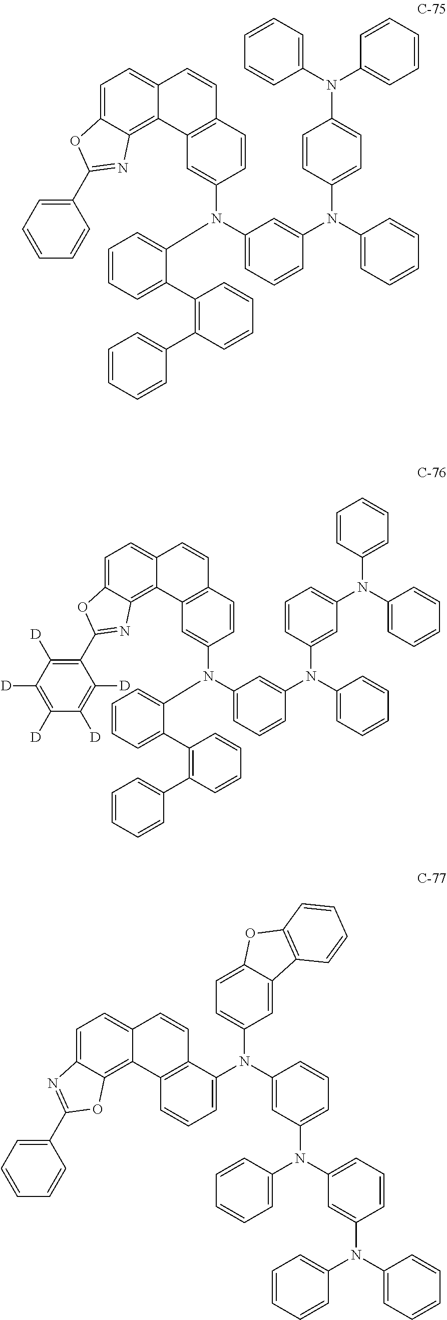 [SMILES: c1ccc(-c2nc3c(ccc4ccc5ccc(N(c6cccc(N(c7ccccc7)c7ccc(N(c8ccccc8)c8ccccc8)cc7)c6)c6ccccc6-c6ccccc6-c6ccccc6)cc5c43)o2)cc1 ; [2H]c1c([2H])c([2H])c(-c2nc3c(ccc4ccc5ccc(N(c6cccc(N(c7ccccc7)c7cccc(N(c8ccccc8)c8ccccc8)c7)c6)c6ccccc6-c6ccccc6-c6ccccc6)cc5c43)o2)c([2H])c1[2H] ; c1ccc(-c2nc3ccc4ccc5c(N(c6cccc(N(c7ccccc7)c7cccc(N(c8ccccc8)c8ccccc8)c7)c6)c6ccc7oc8ccccc8c7c6)cccc5c4c3o2)cc1]

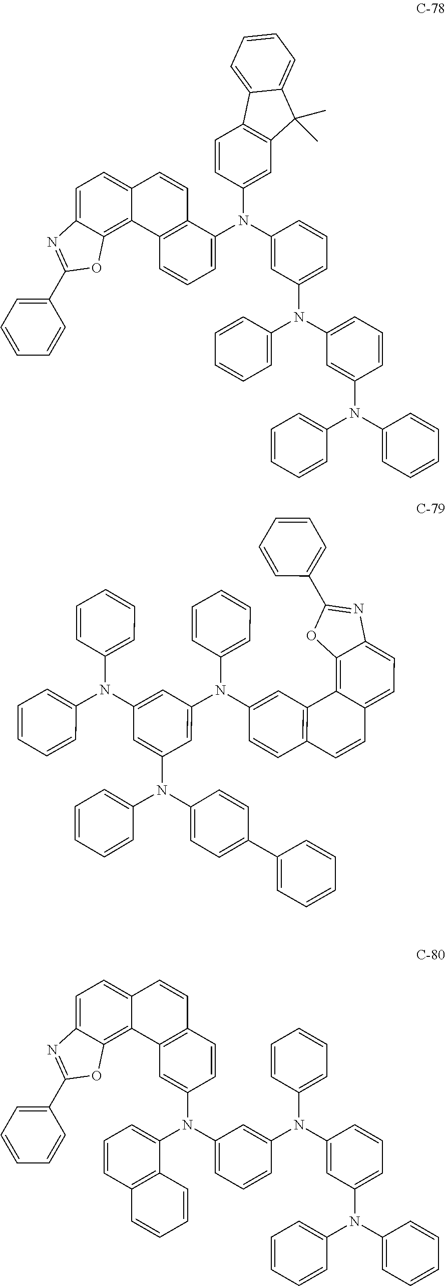

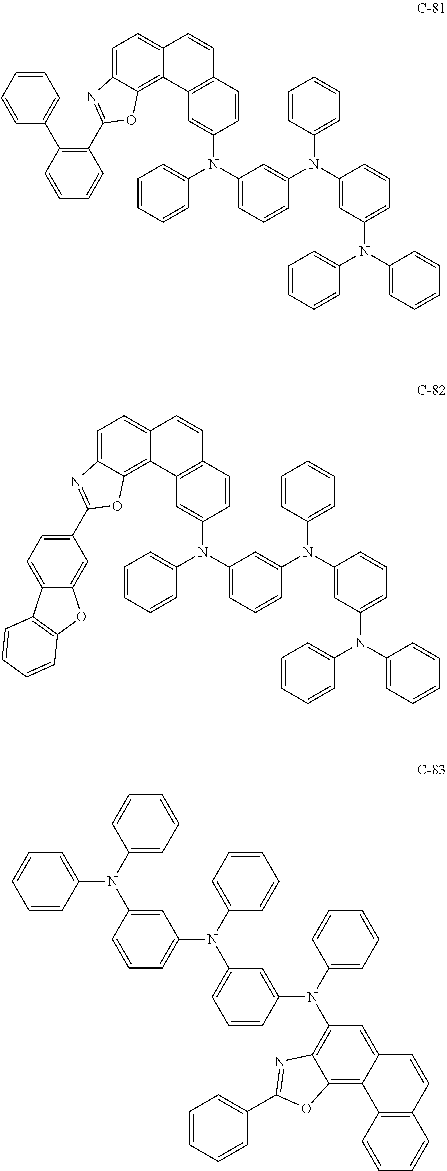 [SMILES: c1ccc(-c2ccccc2-c2nc3ccc4ccc5ccc(N(c6ccccc6)c6cccc(N(c7ccccc7)c7cccc(N(c8ccccc8)c8ccccc8)c7)c6)cc5c4c3o2)cc1 ; c1ccc(N(c2ccccc2)c2cccc(N(c3ccccc3)c3cccc(N(c4ccccc4)c4ccc5ccc6ccc7nc(-c8ccc9c(c8)oc8ccccc89)oc7c6c5c4)c3)c2)cc1 ; c1ccc(-c2nc3c(N(c4ccccc4)c4cccc(N(c5ccccc5)c5cccc(N(c6ccccc6)c6ccccc6)c5)c4)cc4ccc5ccccc5c4c3o2)cc1]

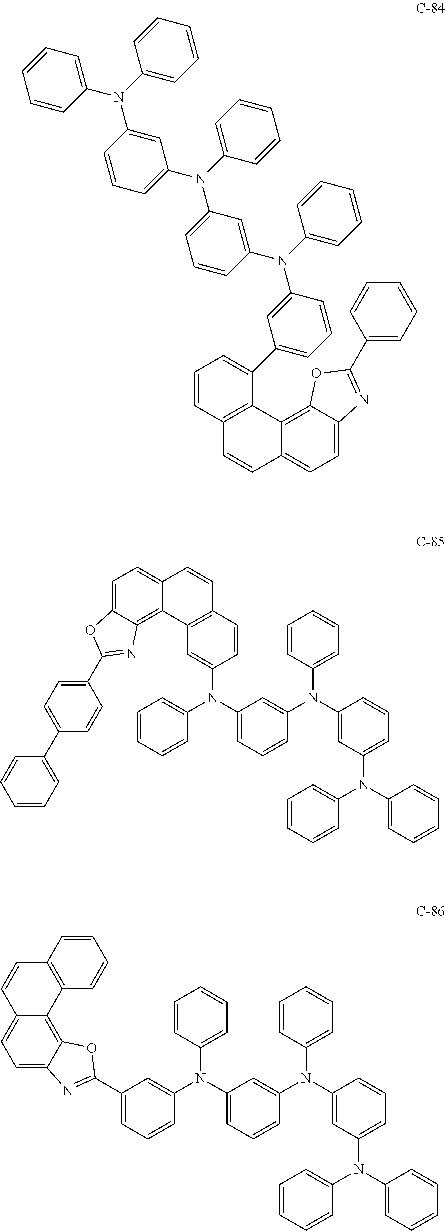 [SMILES: c1ccc(-c2nc3ccc4ccc5cccc(-c6cccc(N(c7ccccc7)c7cccc(N(c8ccccc8)c8cccc(N(c9ccccc9)c9ccccc9)c8)c7)c6)c5c4c3o2)cc1 ; c1ccc(-c2ccc(-c3nc4c(ccc5ccc6ccc(N(c7ccccc7)c7cccc(N(c8ccccc8)c8cccc(N(c9ccccc9)c9ccccc9)c8)c7)cc6c54)o3)cc2)cc1 ; c1ccc(N(c2ccccc2)c2cccc(N(c3ccccc3)c3cccc(N(c4ccccc4)c4cccc(-c5nc6ccc7ccc8ccccc8c7c6o5)c4)c3)c2)cc1]

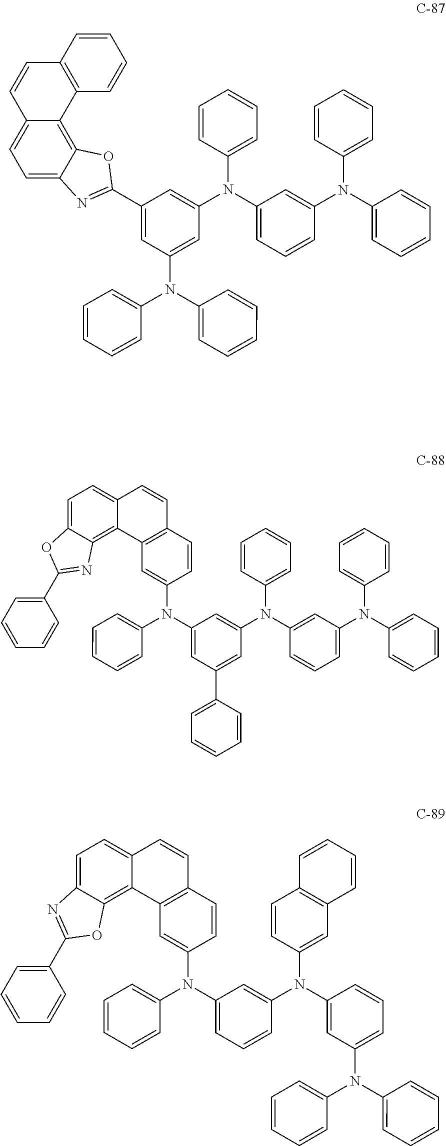 [SMILES: c1ccc(N(c2ccccc2)c2cccc(N(c3ccccc3)c3cc(-c4nc5ccc6ccc7ccccc7c6c5o4)cc(N(c4ccccc4)c4ccccc4)c3)c2)cc1 ; c1ccc(-c2cc(N(c3ccccc3)c3cccc(N(c4ccccc4)c4ccccc4)c3)cc(N(c3ccccc3)c3ccc4ccc5ccc6oc(-c7ccccc7)nc6c5c4c3)c2)cc1 ; c1ccc(-c2nc3ccc4ccc5ccc(N(c6ccccc6)c6cccc(N(c7cccc(N(c8ccccc8)c8ccccc8)c7)c7ccc8ccccc8c7)c6)cc5c4c3o2)cc1]

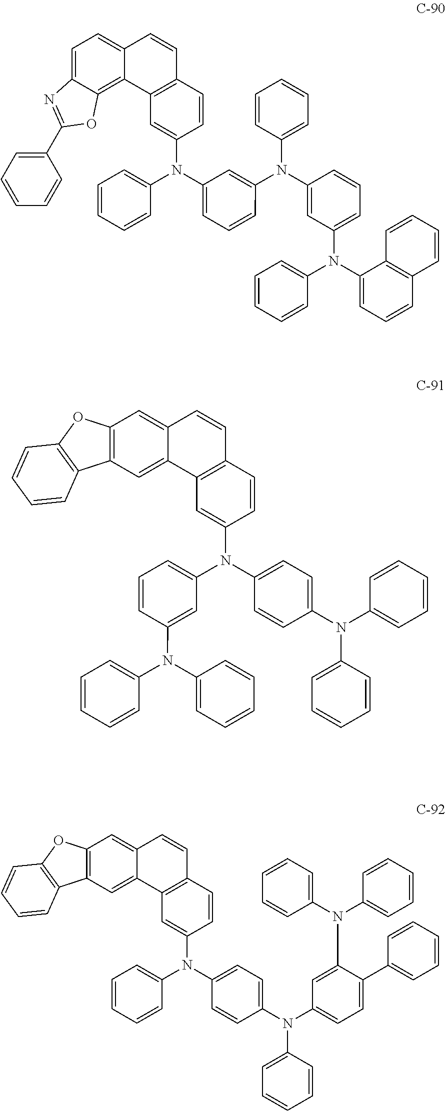 [SMILES: c1ccc(-c2nc3ccc4ccc5ccc(N(c6ccccc6)c6cccc(N(c7ccccc7)c7cccc(N(c8ccccc8)c8cccc9ccccc89)c7)c6)cc5c4c3o2)cc1 ; c1ccc(N(c2ccccc2)c2ccc(N(c3cccc(N(c4ccccc4)c4ccccc4)c3)c3ccc4ccc5cc6oc7ccccc7c6cc5c4c3)cc2)cc1 ; c1ccc(-c2ccc(N(c3ccccc3)c3ccc(N(c4ccccc4)c4ccc5ccc6cc7oc8ccccc8c7cc6c5c4)cc3)cc2N(c2ccccc2)c2ccccc2)cc1]

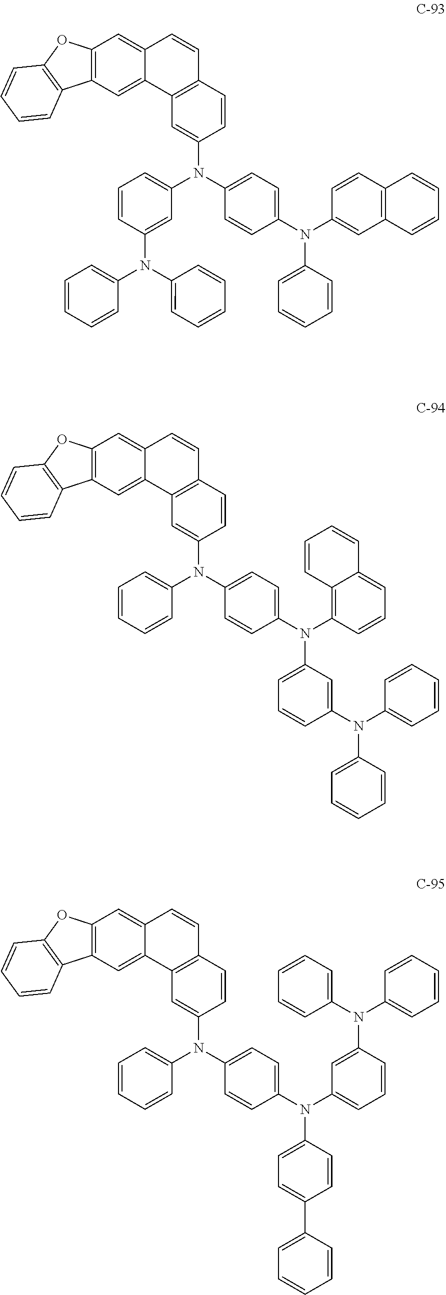 [SMILES: c1ccc(N(c2ccccc2)c2cccc(N(c3ccc(N(c4ccccc4)c4ccc5ccccc5c4)cc3)c3ccc4ccc5cc6oc7ccccc7c6cc5c4c3)c2)cc1 ; c1ccc(N(c2ccccc2)c2cccc(N(c3ccc(N(c4ccccc4)c4ccc5ccc6cc7oc8ccccc8c7cc6c5c4)cc3)c3cccc4ccccc34)c2)cc1 ; c1ccc(-c2ccc(N(c3ccc(N(c4ccccc4)c4ccc5ccc6cc7oc8ccccc8c7cc6c5c4)cc3)c3cccc(N(c4ccccc4)c4ccccc4)c3)cc2)cc1]

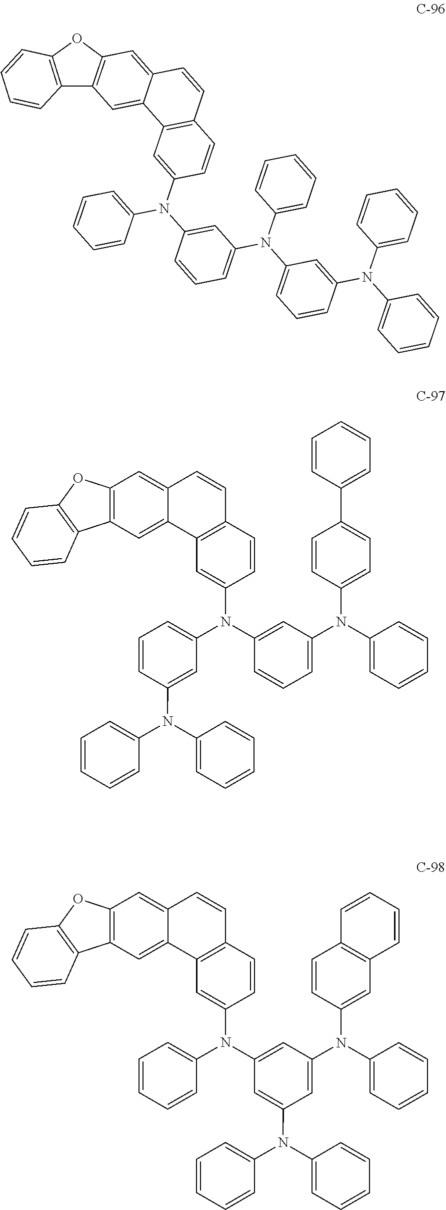 [SMILES: c1ccc(N(c2ccccc2)c2cccc(N(c3ccccc3)c3cccc(N(c4ccccc4)c4ccc5ccc6cc7oc8ccccc8c7cc6c5c4)c3)c2)cc1 ; c1ccc(-c2ccc(N(c3ccccc3)c3cccc(N(c4cccc(N(c5ccccc5)c5ccccc5)c4)c4ccc5ccc6cc7oc8ccccc8c7cc6c5c4)c3)cc2)cc1 ; c1ccc(N(c2ccccc2)c2cc(N(c3ccccc3)c3ccc4ccccc4c3)cc(N(c3ccccc3)c3ccc4ccc5cc6oc7ccccc7c6cc5c4c3)c2)cc1]

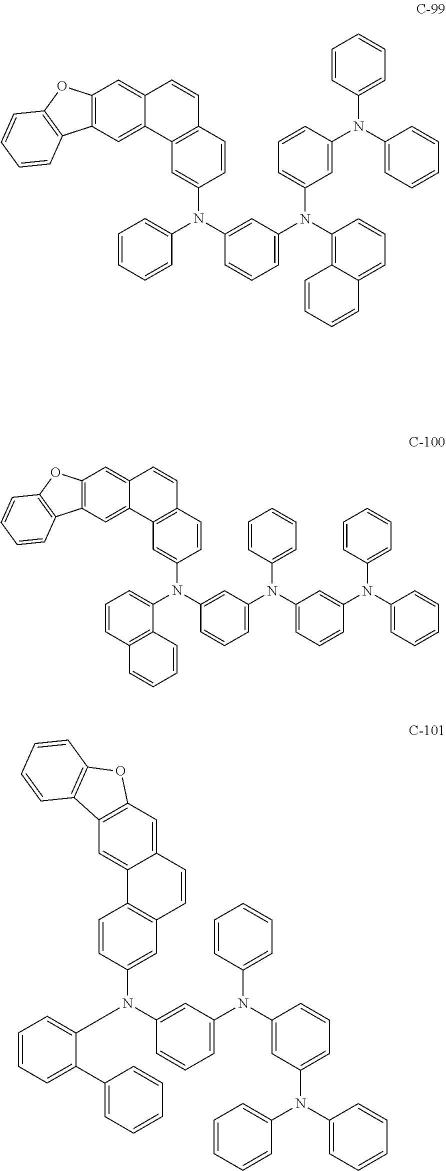 [SMILES: c1ccc(N(c2ccccc2)c2cccc(N(c3cccc(N(c4ccccc4)c4ccc5ccc6cc7oc8ccccc8c7cc6c5c4)c3)c3cccc4ccccc34)c2)cc1 ; c1ccc(N(c2ccccc2)c2cccc(N(c3ccccc3)c3cccc(N(c4ccc5ccc6cc7oc8ccccc8c7cc6c5c4)c4cccc5ccccc45)c3)c2)cc1 ; c1ccc(-c2ccccc2N(c2cccc(N(c3ccccc3)c3cccc(N(c4ccccc4)c4ccccc4)c3)c2)c2ccc3c(ccc4cc5oc6ccccc6c5cc43)c2)cc1]

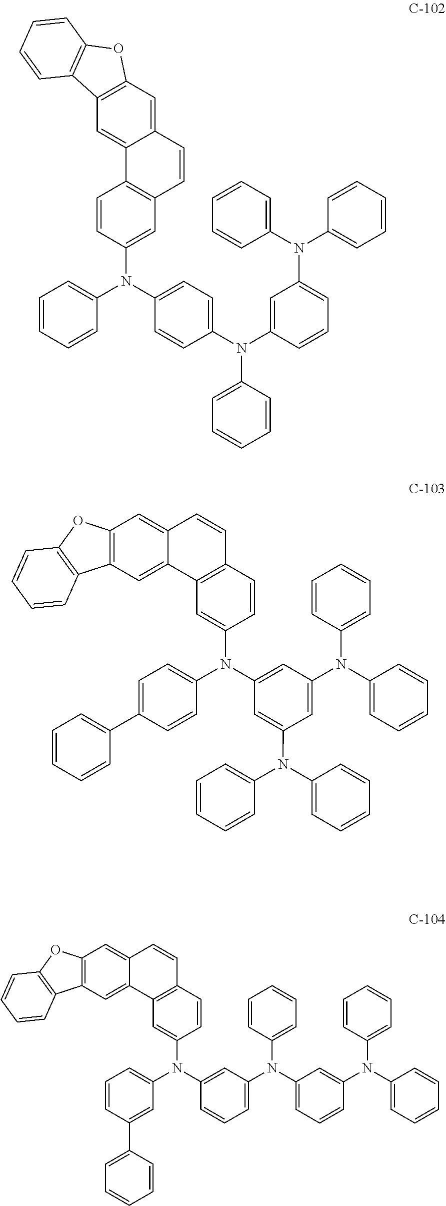 [SMILES: c1ccc(N(c2ccccc2)c2cccc(N(c3ccccc3)c3ccc(N(c4ccccc4)c4ccc5c(ccc6cc7oc8ccccc8c7cc65)c4)cc3)c2)cc1 ; c1ccc(-c2ccc(N(c3cc(N(c4ccccc4)c4ccccc4)cc(N(c4ccccc4)c4ccccc4)c3)c3ccc4ccc5cc6oc7ccccc7c6cc5c4c3)cc2)cc1 ; c1ccc(-c2cccc(N(c3cccc(N(c4ccccc4)c4cccc(N(c5ccccc5)c5ccccc5)c4)c3)c3ccc4ccc5cc6oc7ccccc7c6cc5c4c3)c2)cc1]

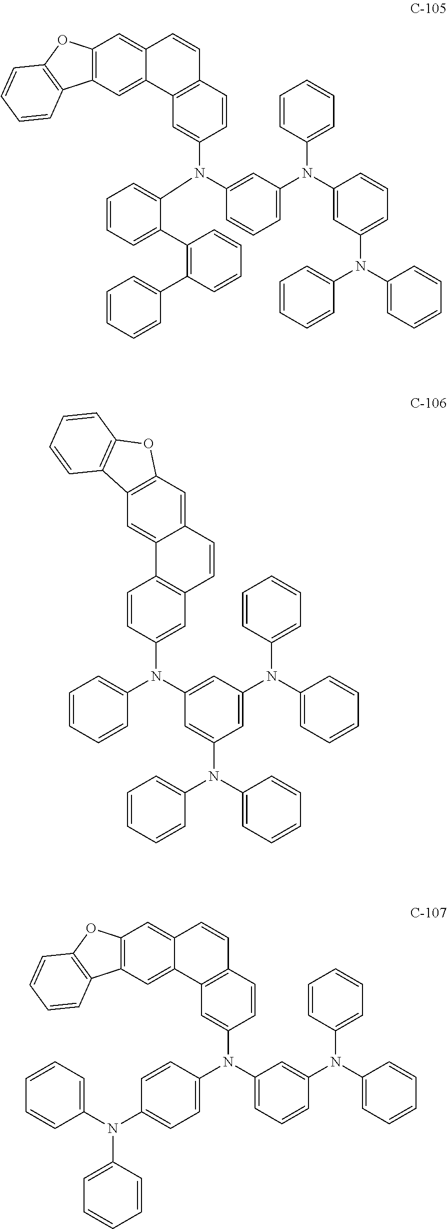 [SMILES: c1ccc(-c2ccccc2-c2ccccc2N(c2cccc(N(c3ccccc3)c3cccc(N(c4ccccc4)c4ccccc4)c3)c2)c2ccc3ccc4cc5oc6ccccc6c5cc4c3c2)cc1 ; c1ccc(N(c2ccccc2)c2cc(N(c3ccccc3)c3ccccc3)cc(N(c3ccccc3)c3ccc4c(ccc5cc6oc7ccccc7c6cc54)c3)c2)cc1 ; c1ccc(N(c2ccccc2)c2ccc(N(c3cccc(N(c4ccccc4)c4ccccc4)c3)c3ccc4ccc5cc6oc7ccccc7c6cc5c4c3)cc2)cc1]

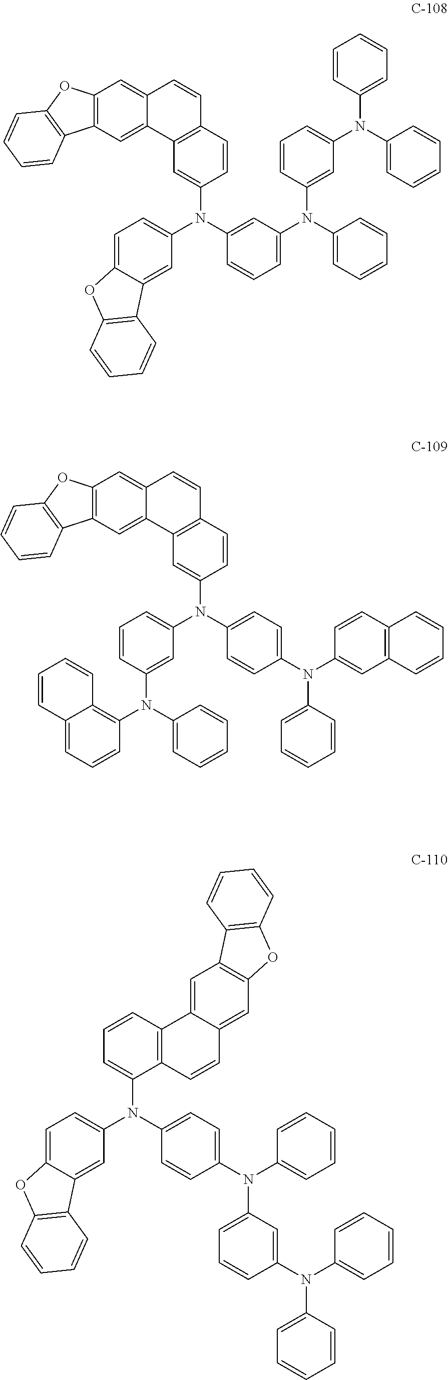 [SMILES: c1ccc(N(c2ccccc2)c2cccc(N(c3ccccc3)c3cccc(N(c4ccc5ccc6cc7oc8ccccc8c7cc6c5c4)c4ccc5oc6ccccc6c5c4)c3)c2)cc1 ; c1ccc(N(c2ccc(N(c3cccc(N(c4ccccc4)c4cccc5ccccc45)c3)c3ccc4ccc5cc6oc7ccccc7c6cc5c4c3)cc2)c2ccc3ccccc3c2)cc1 ; c1ccc(N(c2ccccc2)c2cccc(N(c3ccccc3)c3ccc(N(c4ccc5oc6ccccc6c5c4)c4cccc5c4ccc4cc6oc7ccccc7c6cc45)cc3)c2)cc1]

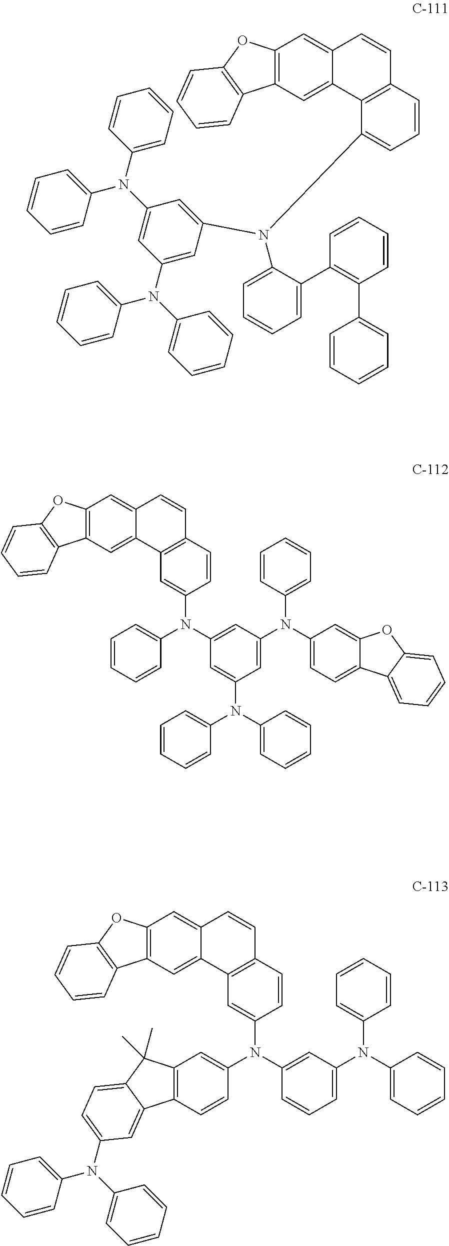 [SMILES: c1ccc(-c2ccccc2-c2ccccc2N(c2cc(N(c3ccccc3)c3ccccc3)cc(N(c3ccccc3)c3ccccc3)c2)c2cccc3ccc4cc5oc6ccccc6c5cc4c23)cc1 ; c1ccc(N(c2ccccc2)c2cc(N(c3ccccc3)c3ccc4c(c3)oc3ccccc34)cc(N(c3ccccc3)c3ccc4ccc5cc6oc7ccccc7c6cc5c4c3)c2)cc1 ; CC1(C)c2ccc(N(c3ccccc3)c3ccccc3)cc2-c2ccc(N(c3cccc(N(c4ccccc4)c4ccccc4)c3)c3ccc4ccc5cc6oc7ccccc7c6cc5c4c3)cc21]

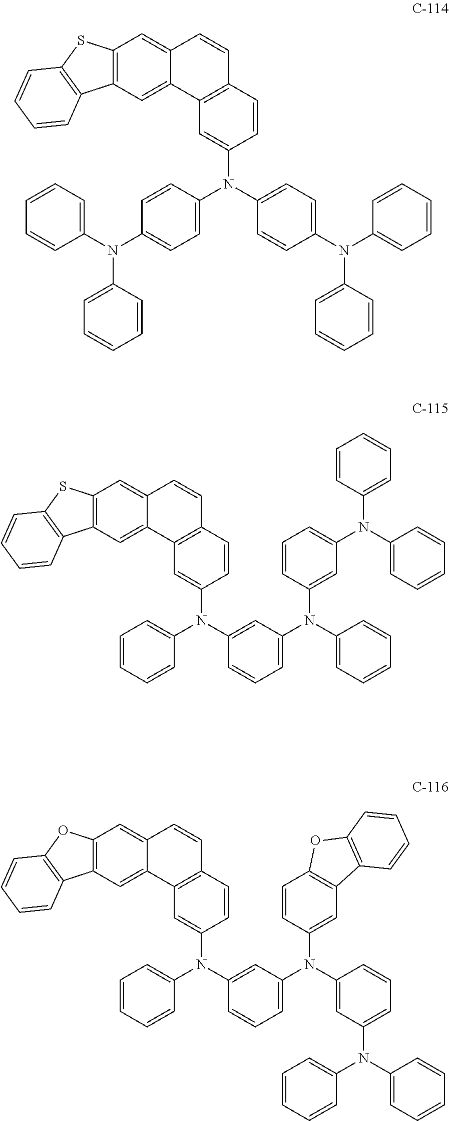 [SMILES: c1ccc(N(c2ccccc2)c2ccc(N(c3ccc(N(c4ccccc4)c4ccccc4)cc3)c3ccc4ccc5cc6sc7ccccc7c6cc5c4c3)cc2)cc1 ; c1ccc(N(c2ccccc2)c2cccc(N(c3ccccc3)c3cccc(N(c4ccccc4)c4ccc5ccc6cc7sc8ccccc8c7cc6c5c4)c3)c2)cc1 ; c1ccc(N(c2ccccc2)c2cccc(N(c3cccc(N(c4ccccc4)c4ccc5ccc6cc7oc8ccccc8c7cc6c5c4)c3)c3ccc4oc5ccccc5c4c3)c2)cc1]

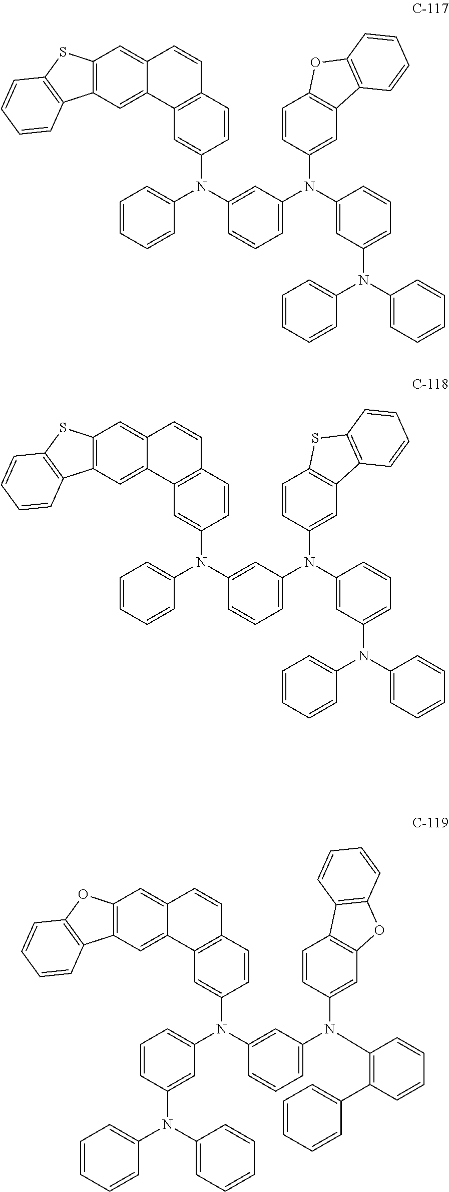 [SMILES: c1ccc(N(c2ccccc2)c2cccc(N(c3cccc(N(c4ccccc4)c4ccc5ccc6cc7sc8ccccc8c7cc6c5c4)c3)c3ccc4oc5ccccc5c4c3)c2)cc1 ; c1ccc(N(c2ccccc2)c2cccc(N(c3cccc(N(c4ccccc4)c4ccc5ccc6cc7sc8ccccc8c7cc6c5c4)c3)c3ccc4sc5ccccc5c4c3)c2)cc1 ; c1ccc(-c2ccccc2N(c2cccc(N(c3cccc(N(c4ccccc4)c4ccccc4)c3)c3ccc4ccc5cc6oc7ccccc7c6cc5c4c3)c2)c2ccc3c(c2)oc2ccccc23)cc1]

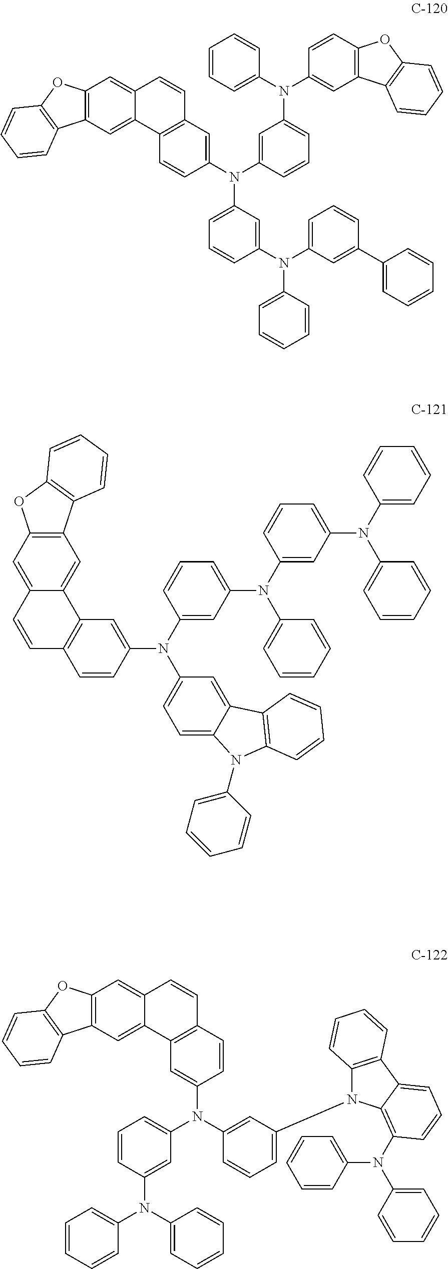 [SMILES: c1ccc(-c2cccc(N(c3ccccc3)c3cccc(N(c4cccc(N(c5ccccc5)c5ccc6oc7ccccc7c6c5)c4)c4ccc5c(ccc6cc7oc8ccccc8c7cc65)c4)c3)c2)cc1 ; c1ccc(N(c2ccccc2)c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3cccc(-n4c5ccccc5c5cccc(N(c6ccccc6)c6ccccc6)c54)c3)c3ccc4ccc5cc6oc7ccccc7c6cc5c4c3)c2)cc1]

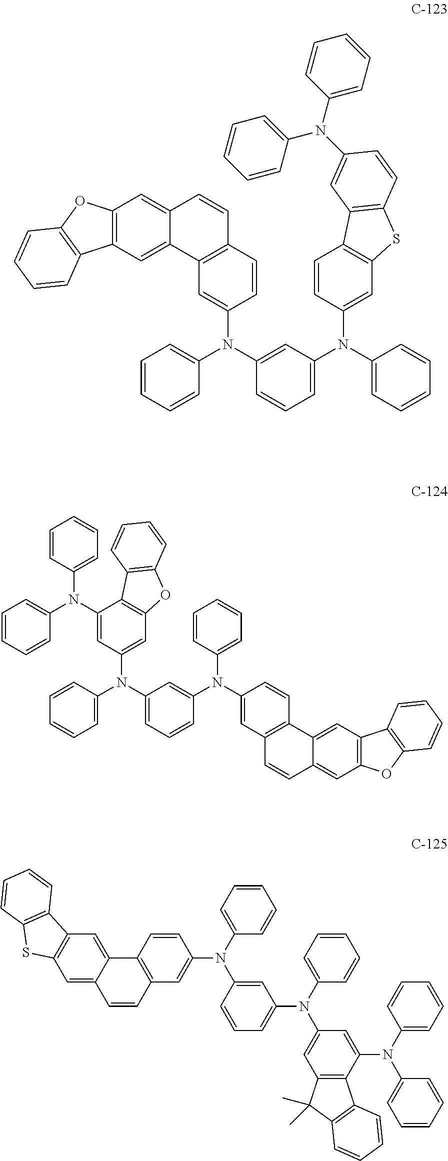 [SMILES: c1ccc(N(c2cccc(N(c3ccccc3)c3ccc4ccc5cc6oc7ccccc7c6cc5c4c3)c2)c2ccc3c(c2)sc2ccc(N(c4ccccc4)c4ccccc4)cc23)cc1 ; c1ccc(N(c2cccc(N(c3ccccc3)c3cc(N(c4ccccc4)c4ccccc4)c4c(c3)oc3ccccc34)c2)c2ccc3c(ccc4cc5oc6ccccc6c5cc43)c2)cc1 ; CC1(C)c2ccccc2-c2c(N(c3ccccc3)c3ccccc3)cc(N(c3ccccc3)c3cccc(N(c4ccccc4)c4ccc5c(ccc6cc7sc8ccccc8c7cc65)c4)c3)cc21]

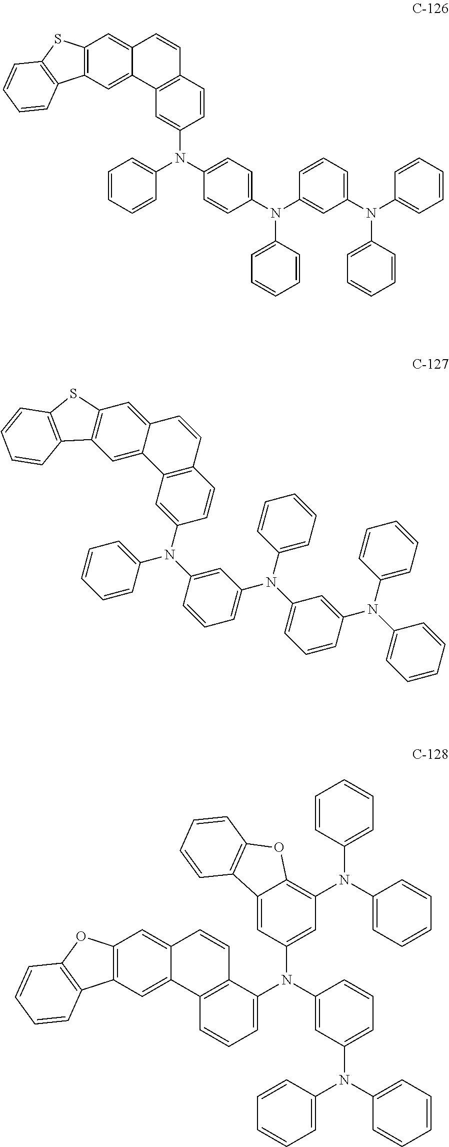 [SMILES: c1ccc(N(c2ccccc2)c2cccc(N(c3ccccc3)c3ccc(N(c4ccccc4)c4ccc5ccc6cc7sc8ccccc8c7cc6c5c4)cc3)c2)cc1 ; c1ccc(N(c2ccccc2)c2cccc(N(c3ccccc3)c3cccc(N(c4ccccc4)c4ccc5ccc6cc7sc8ccccc8c7cc6c5c4)c3)c2)cc1 ; c1ccc(N(c2ccccc2)c2cccc(N(c3cc(N(c4ccccc4)c4ccccc4)c4oc5ccccc5c4c3)c3cccc4c3ccc3cc5oc6ccccc6c5cc34)c2)cc1]

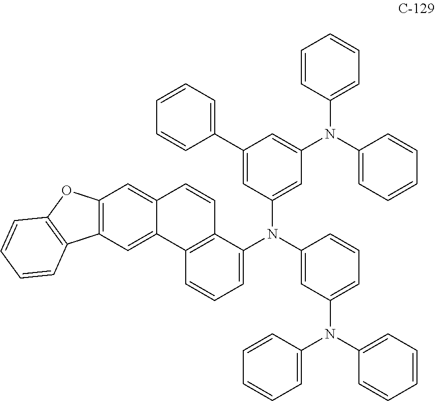 [SMILES: c1ccc(-c2cc(N(c3ccccc3)c3ccccc3)cc(N(c3cccc(N(c4ccccc4)c4ccccc4)c3)c3cccc4c3ccc3cc5oc6ccccc6c5cc34)c2)cc1]

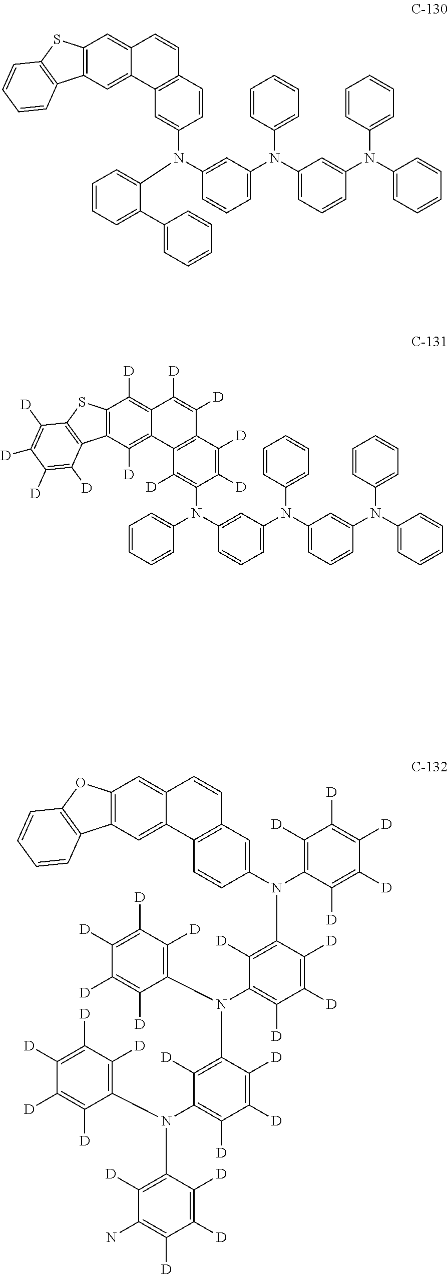

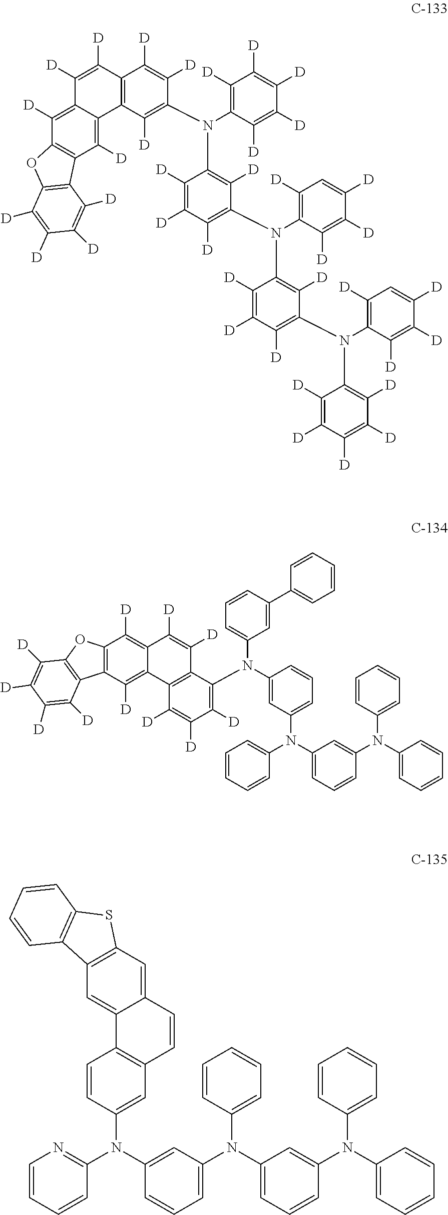

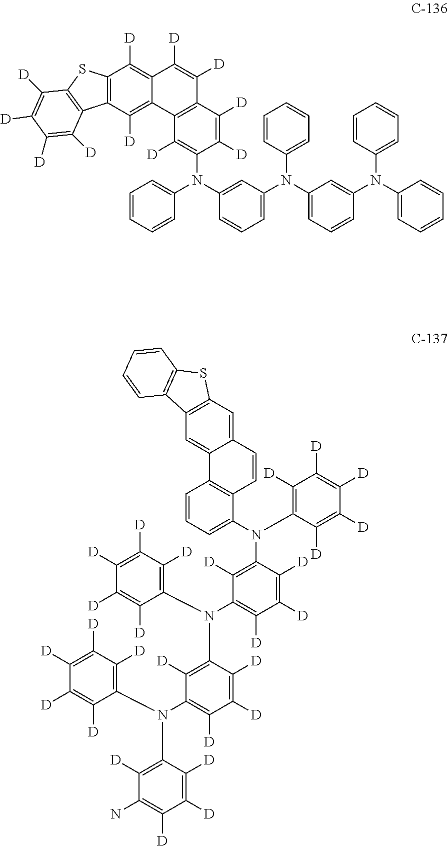 [SMILES: [2H]c1c([2H])c([2H])c2c(sc3c([2H])c4c([2H])c([2H])c5c([2H])c([2H])c(N(c6ccccc6)c6cccc(N(c7ccccc7)c7cccc(N(c8ccccc8)c8ccccc8)c7)c6)c([2H])c5c4c([2H])c32)c1[2H] ; [2H]c1c([2H])c([2H])c(N(c2c([2H])c([2H])c([2H])c(N)c2[2H])c2c([2H])c([2H])c([2H])c(N(c3c([2H])c([2H])c([2H])c([2H])c3[2H])c3c([2H])c([2H])c([2H])c(N(c4c([2H])c([2H])c([2H])c([2H])c4[2H])c4cccc5c4ccc4cc6sc7ccccc7c6cc45)c3[2H])c2[2H])c([2H])c1[2H]]

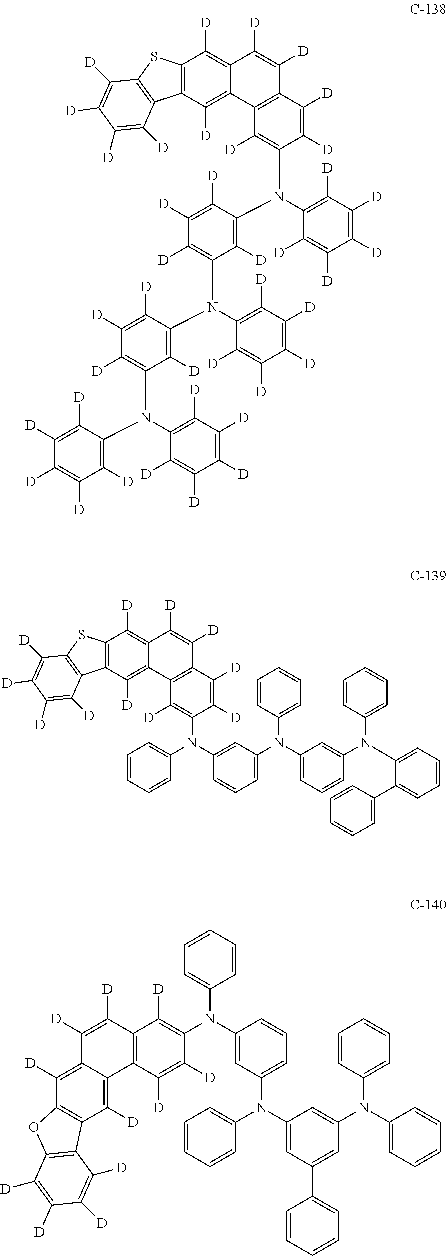

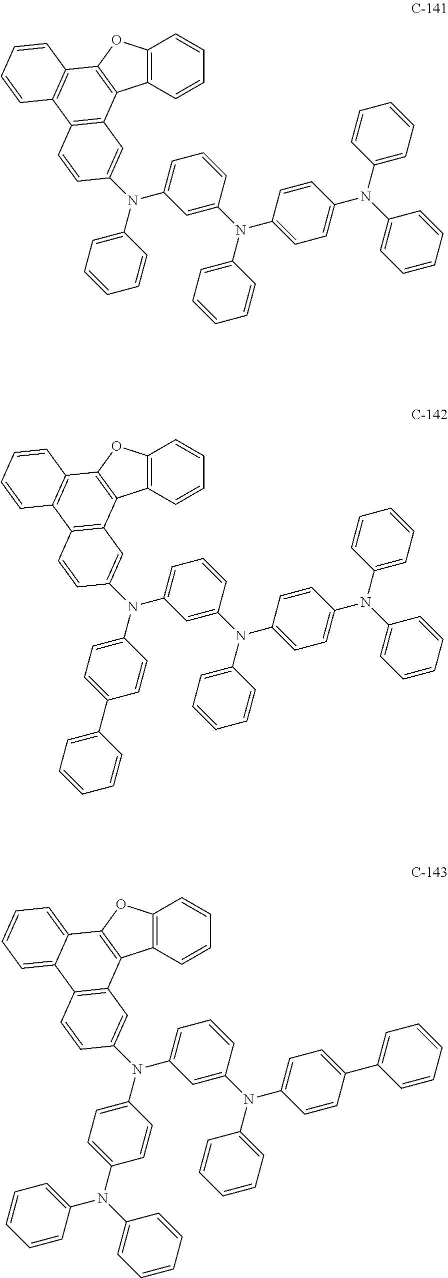 [SMILES: c1ccc(N(c2ccccc2)c2ccc(N(c3ccccc3)c3cccc(N(c4ccccc4)c4ccc5c6ccccc6c6oc7ccccc7c6c5c4)c3)cc2)cc1 ; c1ccc(-c2ccc(N(c3cccc(N(c4ccccc4)c4ccc(N(c5ccccc5)c5ccccc5)cc4)c3)c3ccc4c5ccccc5c5oc6ccccc6c5c4c3)cc2)cc1 ; c1ccc(-c2ccc(N(c3ccccc3)c3cccc(N(c4ccc(N(c5ccccc5)c5ccccc5)cc4)c4ccc5c6ccccc6c6oc7ccccc7c6c5c4)c3)cc2)cc1]

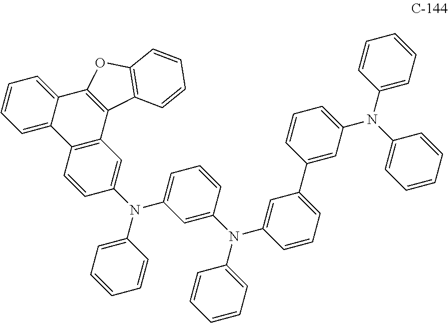 [SMILES: c1ccc(N(c2ccccc2)c2cccc(-c3cccc(N(c4ccccc4)c4cccc(N(c5ccccc5)c5ccc6c7ccccc7c7oc8ccccc8c7c6c5)c4)c3)c2)cc1]

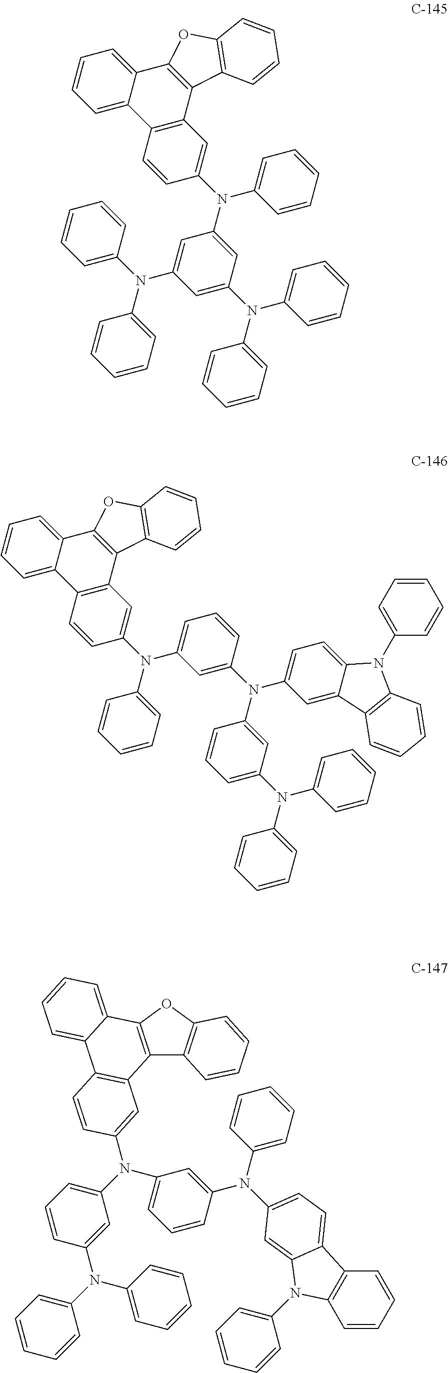 [SMILES: c1ccc(N(c2ccccc2)c2cc(N(c3ccccc3)c3ccccc3)cc(N(c3ccccc3)c3ccc4c5ccccc5c5oc6ccccc6c5c4c3)c2)cc1 ; c1ccc(N(c2ccccc2)c2cccc(N(c3cccc(N(c4ccccc4)c4ccc5c6ccccc6c6oc7ccccc7c6c5c4)c3)c3ccc4c(c3)c3ccccc3n4-c3ccccc3)c2)cc1 ; c1ccc(N(c2ccccc2)c2cccc(N(c3cccc(N(c4ccccc4)c4ccc5c6ccccc6n(-c6ccccc6)c5c4)c3)c3ccc4c5ccccc5c5oc6ccccc6c5c4c3)c2)cc1]

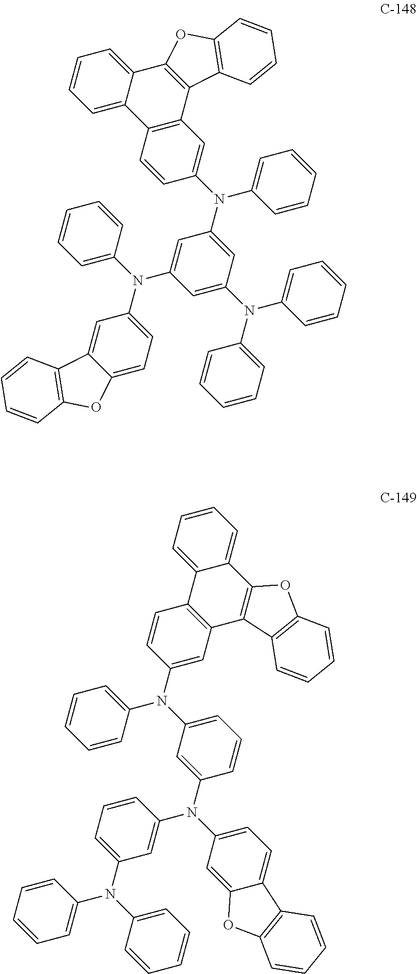 [SMILES: c1ccc(N(c2ccccc2)c2cc(N(c3ccccc3)c3ccc4oc5ccccc5c4c3)cc(N(c3ccccc3)c3ccc4c5ccccc5c5oc6ccccc6c5c4c3)c2)cc1 ; c1ccc(N(c2ccccc2)c2cccc(N(c3cccc(N(c4ccccc4)c4ccc5c6ccccc6c6oc7ccccc7c6c5c4)c3)c3ccc4c(c3)oc3ccccc34)c2)cc1]

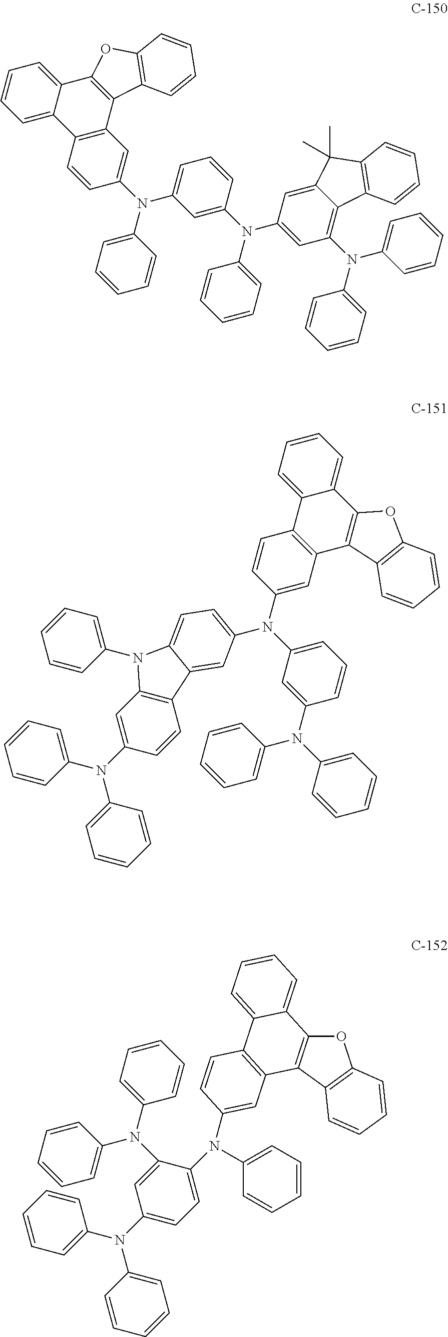 [SMILES: CC1(C)c2ccccc2-c2c(N(c3ccccc3)c3ccccc3)cc(N(c3ccccc3)c3cccc(N(c4ccccc4)c4ccc5c6ccccc6c6oc7ccccc7c6c5c4)c3)cc21 ; c1ccc(N(c2ccccc2)c2cccc(N(c3ccc4c5ccccc5c5oc6ccccc6c5c4c3)c3ccc4c(c3)c3ccc(N(c5ccccc5)c5ccccc5)cc3n4-c3ccccc3)c2)cc1 ; c1ccc(N(c2ccccc2)c2ccc(N(c3ccccc3)c3ccc4c5ccccc5c5oc6ccccc6c5c4c3)c(N(c3ccccc3)c3ccccc3)c2)cc1]

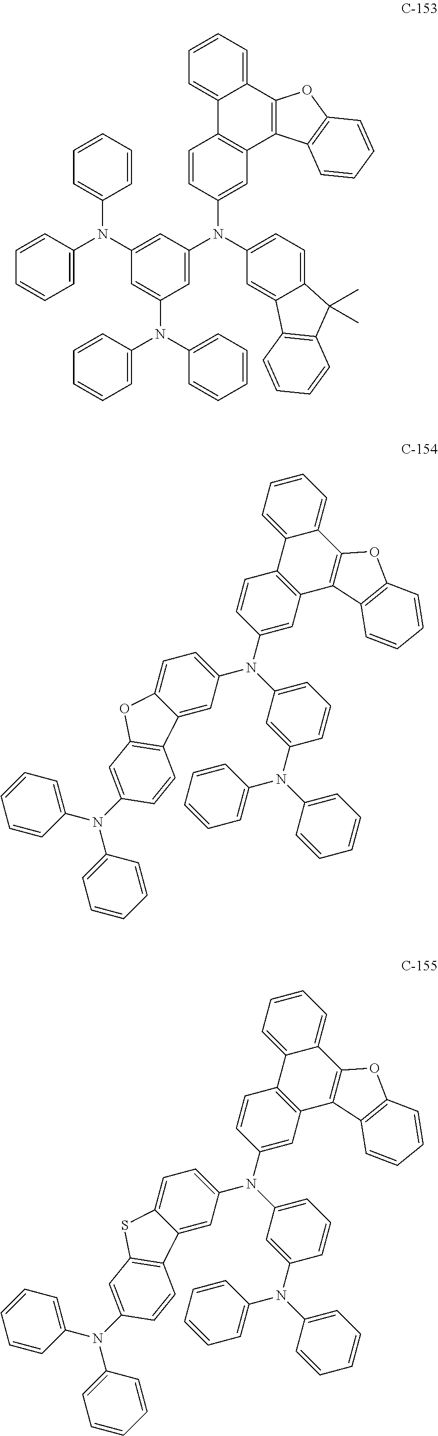 [SMILES: CC1(C)c2ccccc2-c2cc(N(c3cc(N(c4ccccc4)c4ccccc4)cc(N(c4ccccc4)c4ccccc4)c3)c3ccc4c5ccccc5c5oc6ccccc6c5c4c3)ccc21 ; c1ccc(N(c2ccccc2)c2cccc(N(c3ccc4oc5cc(N(c6ccccc6)c6ccccc6)ccc5c4c3)c3ccc4c5ccccc5c5oc6ccccc6c5c4c3)c2)cc1 ; c1ccc(N(c2ccccc2)c2cccc(N(c3ccc4sc5cc(N(c6ccccc6)c6ccccc6)ccc5c4c3)c3ccc4c5ccccc5c5oc6ccccc6c5c4c3)c2)cc1]

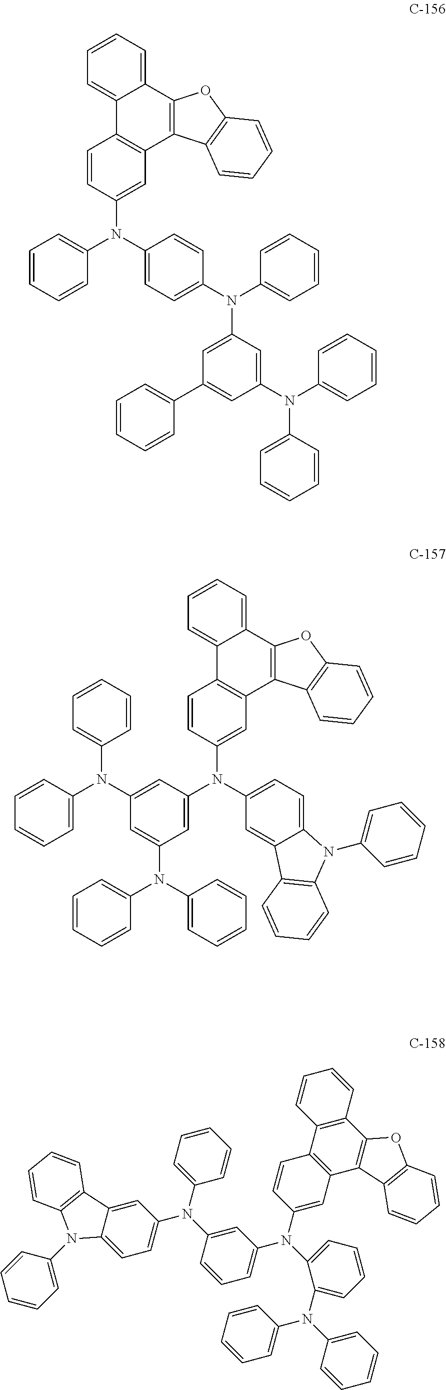 [SMILES: c1ccc(-c2cc(N(c3ccccc3)c3ccccc3)cc(N(c3ccccc3)c3ccc(N(c4ccccc4)c4ccc5c6ccccc6c6oc7ccccc7c6c5c4)cc3)c2)cc1 ; c1ccc(N(c2ccccc2)c2cc(N(c3ccccc3)c3ccccc3)cc(N(c3ccc4c5ccccc5c5oc6ccccc6c5c4c3)c3ccc4c(c3)c3ccccc3n4-c3ccccc3)c2)cc1 ; c1ccc(N(c2cccc(N(c3ccc4c5ccccc5c5oc6ccccc6c5c4c3)c3ccccc3N(c3ccccc3)c3ccccc3)c2)c2ccc3c(c2)c2ccccc2n3-c2ccccc2)cc1]

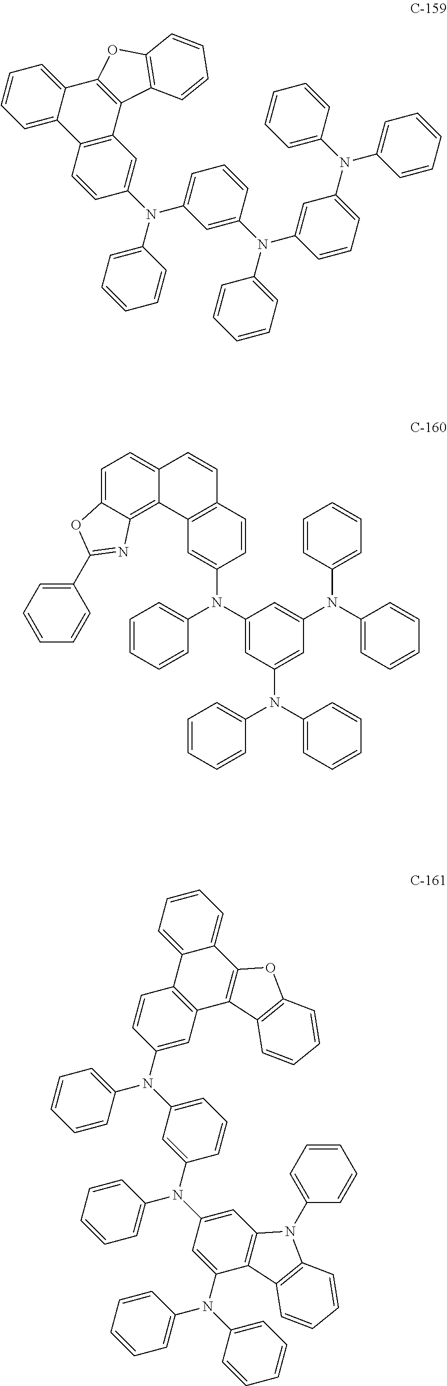 [SMILES: c1ccc(N(c2ccccc2)c2cccc(N(c3ccccc3)c3cccc(N(c4ccccc4)c4ccc5c6ccccc6c6oc7ccccc7c6c5c4)c3)c2)cc1 ; c1ccc(-c2nc3c(ccc4ccc5ccc(N(c6ccccc6)c6cc(N(c7ccccc7)c7ccccc7)cc(N(c7ccccc7)c7ccccc7)c6)cc5c43)o2)cc1 ; c1ccc(N(c2cccc(N(c3ccccc3)c3cc(N(c4ccccc4)c4ccccc4)c4c5ccccc5n(-c5ccccc5)c4c3)c2)c2ccc3c4ccccc4c4oc5ccccc5c4c3c2)cc1]

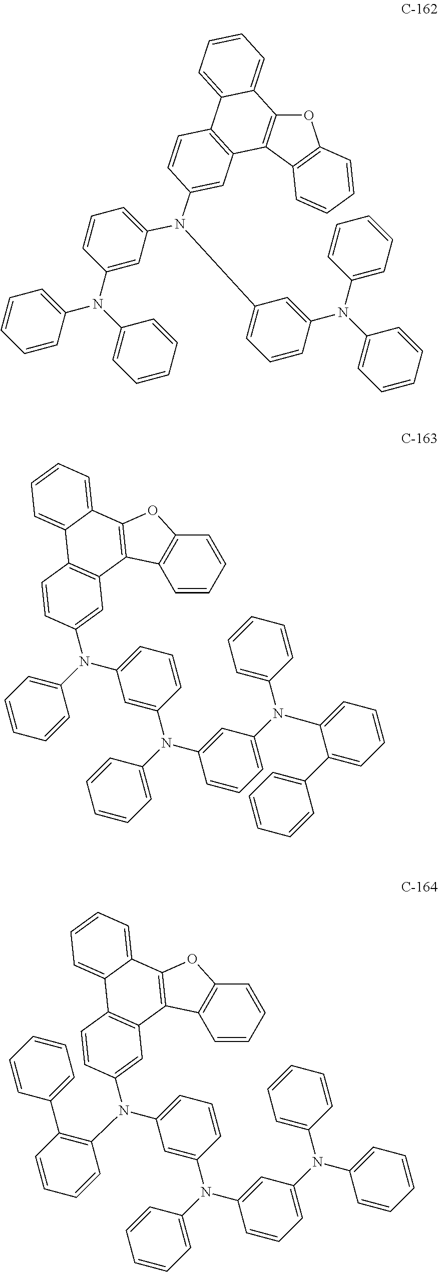 [SMILES: c1ccc(N(c2ccccc2)c2cccc(N(c3cccc(N(c4ccccc4)c4ccccc4)c3)c3ccc4c5ccccc5c5oc6ccccc6c5c4c3)c2)cc1 ; c1ccc(-c2ccccc2N(c2ccccc2)c2cccc(N(c3ccccc3)c3cccc(N(c4ccccc4)c4ccc5c6ccccc6c6oc7ccccc7c6c5c4)c3)c2)cc1 ; c1ccc(-c2ccccc2N(c2cccc(N(c3ccccc3)c3cccc(N(c4ccccc4)c4ccccc4)c3)c2)c2ccc3c4ccccc4c4oc5ccccc5c4c3c2)cc1]

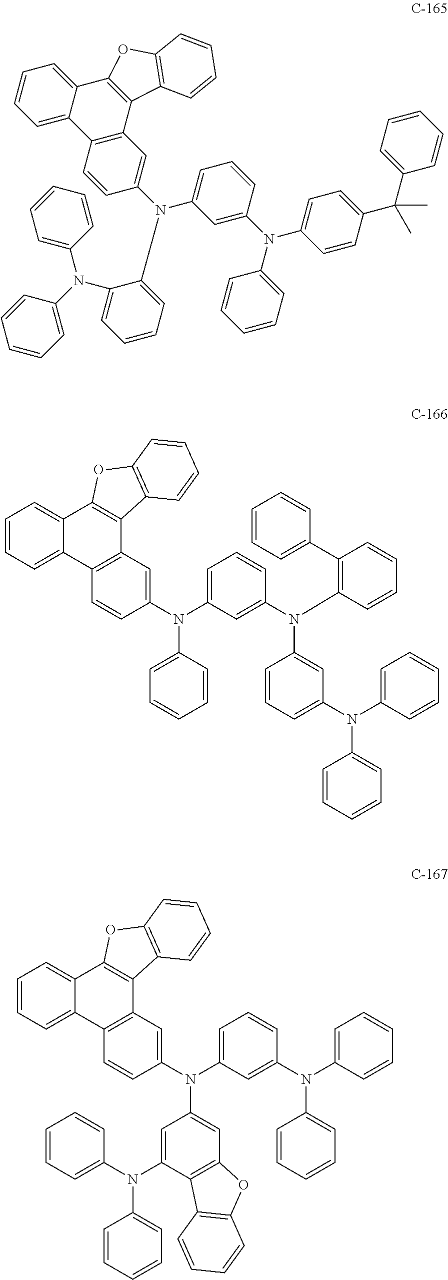 [SMILES: CC(C)(c1ccccc1)c1ccc(N(c2ccccc2)c2cccc(N(c3ccc4c5ccccc5c5oc6ccccc6c5c4c3)c3ccccc3N(c3ccccc3)c3ccccc3)c2)cc1 ; c1ccc(-c2ccccc2N(c2cccc(N(c3ccccc3)c3ccccc3)c2)c2cccc(N(c3ccccc3)c3ccc4c5ccccc5c5oc6ccccc6c5c4c3)c2)cc1 ; c1ccc(N(c2ccccc2)c2cccc(N(c3cc(N(c4ccccc4)c4ccccc4)c4c(c3)oc3ccccc34)c3ccc4c5ccccc5c5oc6ccccc6c5c4c3)c2)cc1]

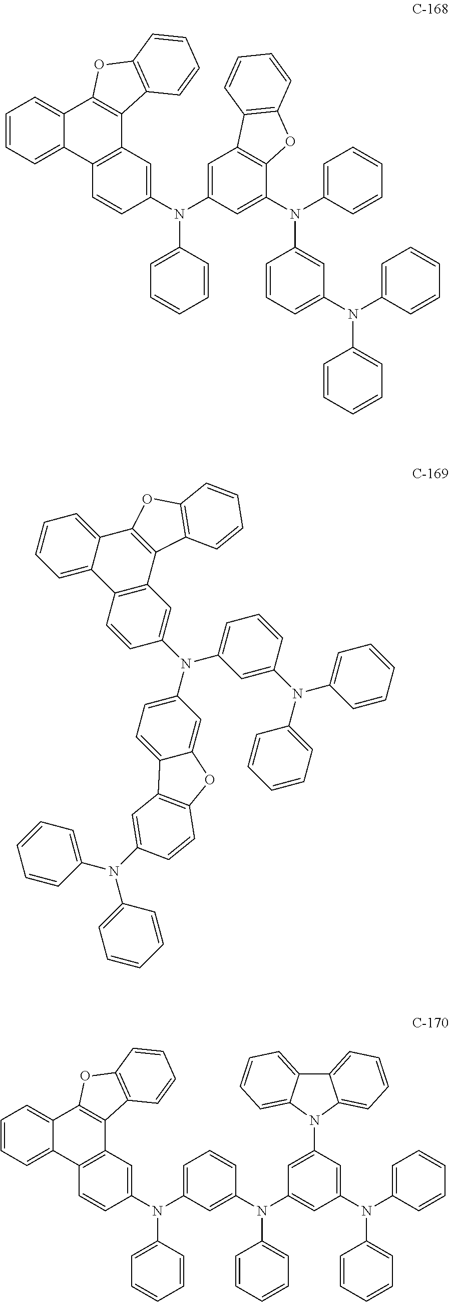 [SMILES: c1ccc(N(c2ccccc2)c2cccc(N(c3ccccc3)c3cc(N(c4ccccc4)c4ccc5c6ccccc6c6oc7ccccc7c6c5c4)cc4c3oc3ccccc34)c2)cc1 ; c1ccc(N(c2ccccc2)c2cccc(N(c3ccc4c(c3)oc3ccc(N(c5ccccc5)c5ccccc5)cc34)c3ccc4c5ccccc5c5oc6ccccc6c5c4c3)c2)cc1 ; c1ccc(N(c2ccccc2)c2cc(N(c3ccccc3)c3cccc(N(c4ccccc4)c4ccc5c6ccccc6c6oc7ccccc7c6c5c4)c3)cc(-n3c4ccccc4c4ccccc43)c2)cc1]

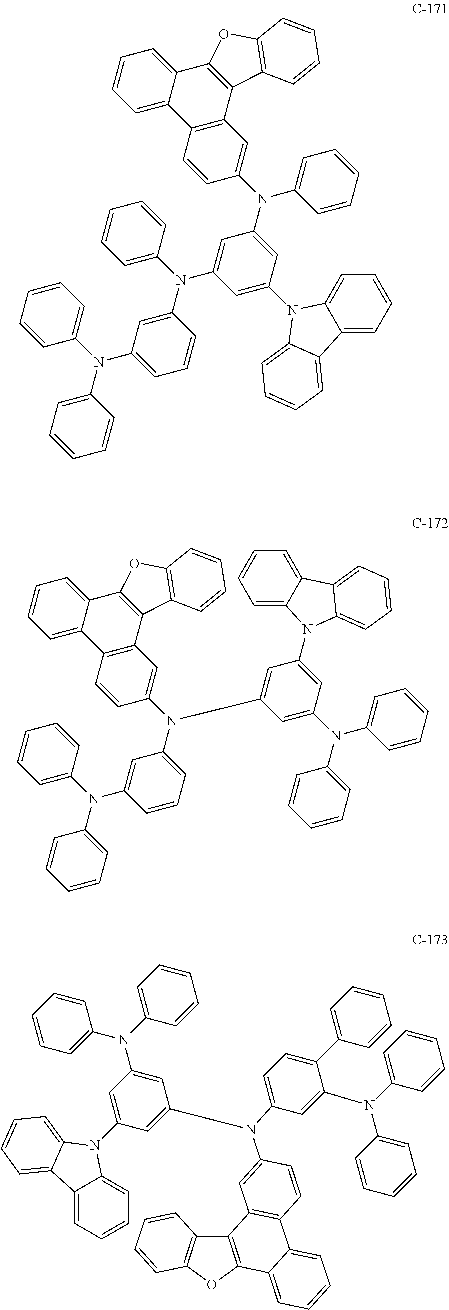 [SMILES: c1ccc(N(c2ccccc2)c2cccc(N(c3ccccc3)c3cc(N(c4ccccc4)c4ccc5c6ccccc6c6oc7ccccc7c6c5c4)cc(-n4c5ccccc5c5ccccc54)c3)c2)cc1 ; c1ccc(N(c2ccccc2)c2cccc(N(c3cc(N(c4ccccc4)c4ccccc4)cc(-n4c5ccccc5c5ccccc54)c3)c3ccc4c5ccccc5c5oc6ccccc6c5c4c3)c2)cc1 ; c1ccc(-c2ccc(N(c3cc(N(c4ccccc4)c4ccccc4)cc(-n4c5ccccc5c5ccccc54)c3)c3ccc4c5ccccc5c5oc6ccccc6c5c4c3)cc2N(c2ccccc2)c2ccccc2)cc1]

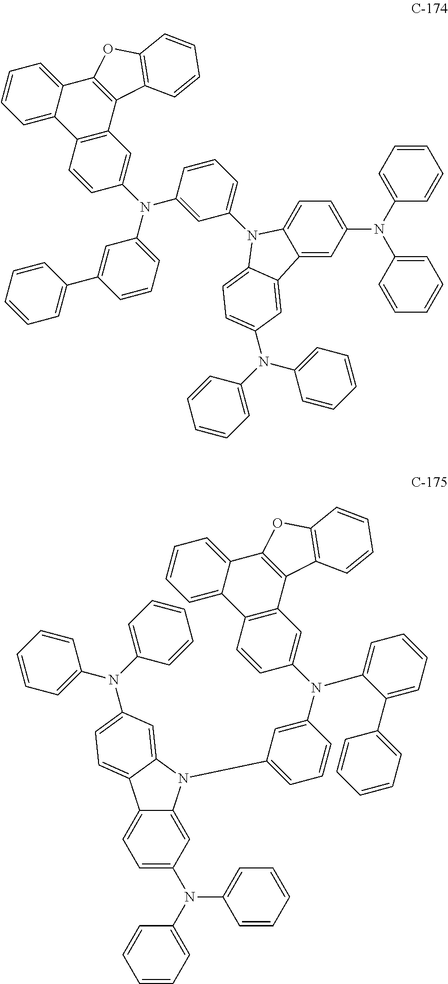 [SMILES: c1ccc(-c2cccc(N(c3cccc(-n4c5ccc(N(c6ccccc6)c6ccccc6)cc5c5cc(N(c6ccccc6)c6ccccc6)ccc54)c3)c3ccc4c5ccccc5c5oc6ccccc6c5c4c3)c2)cc1 ; c1ccc(-c2ccccc2N(c2cccc(-n3c4cc(N(c5ccccc5)c5ccccc5)ccc4c4ccc(N(c5ccccc5)c5ccccc5)cc43)c2)c2ccc3c4ccccc4c4oc5ccccc5c4c3c2)cc1]

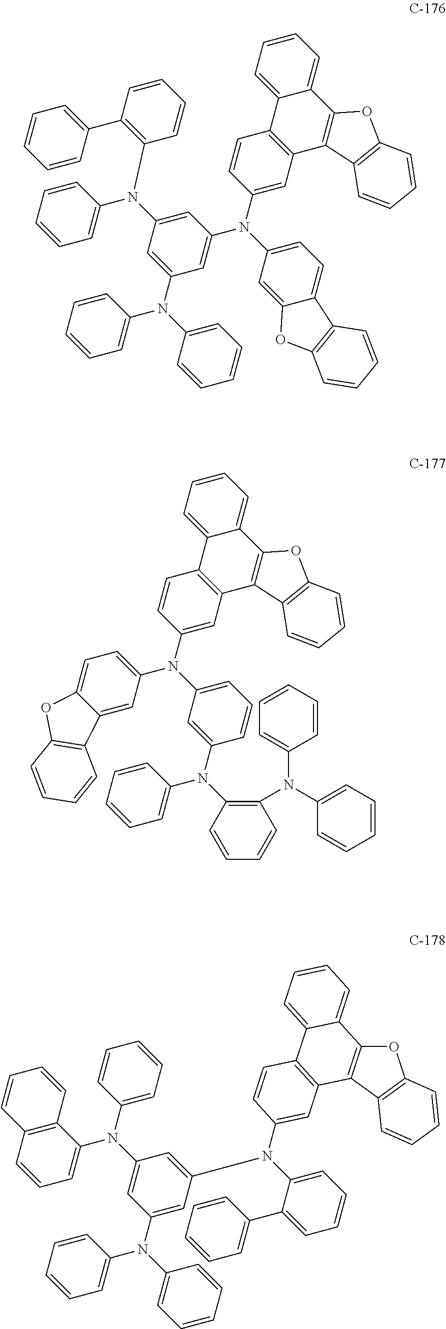 [SMILES: c1ccc(-c2ccccc2N(c2ccccc2)c2cc(N(c3ccccc3)c3ccccc3)cc(N(c3ccc4c(c3)oc3ccccc34)c3ccc4c5ccccc5c5oc6ccccc6c5c4c3)c2)cc1 ; c1ccc(N(c2ccccc2)c2ccccc2N(c2ccccc2)c2cccc(N(c3ccc4oc5ccccc5c4c3)c3ccc4c5ccccc5c5oc6ccccc6c5c4c3)c2)cc1 ; c1ccc(-c2ccccc2N(c2cc(N(c3ccccc3)c3ccccc3)cc(N(c3ccccc3)c3cccc4ccccc34)c2)c2ccc3c4ccccc4c4oc5ccccc5c4c3c2)cc1]

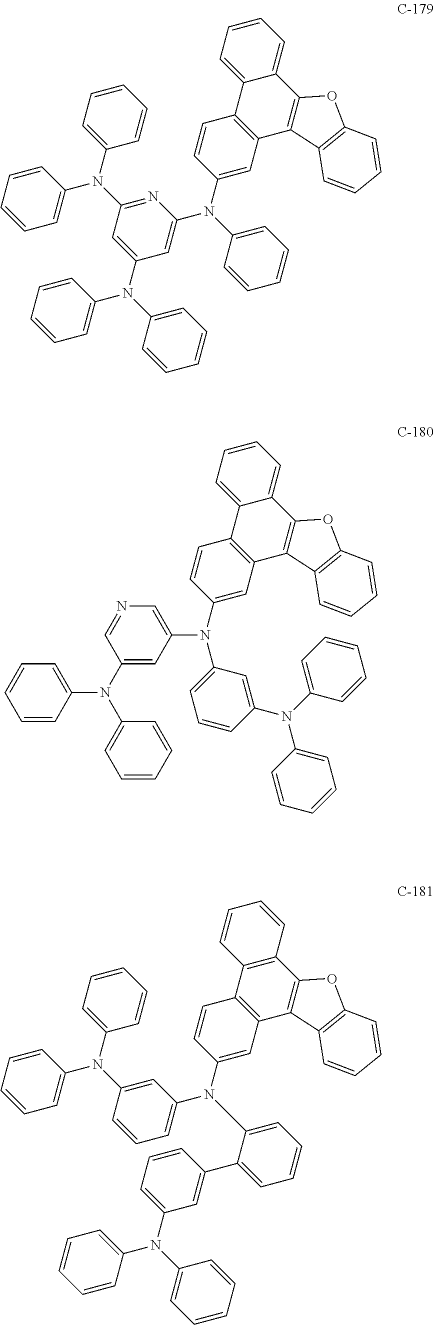 [SMILES: c1ccc(N(c2ccccc2)c2cc(N(c3ccccc3)c3ccccc3)nc(N(c3ccccc3)c3ccc4c5ccccc5c5oc6ccccc6c5c4c3)c2)cc1 ; c1ccc(N(c2ccccc2)c2cccc(N(c3cncc(N(c4ccccc4)c4ccccc4)c3)c3ccc4c5ccccc5c5oc6ccccc6c5c4c3)c2)cc1 ; c1ccc(N(c2ccccc2)c2cccc(-c3ccccc3N(c3cccc(N(c4ccccc4)c4ccccc4)c3)c3ccc4c5ccccc5c5oc6ccccc6c5c4c3)c2)cc1]

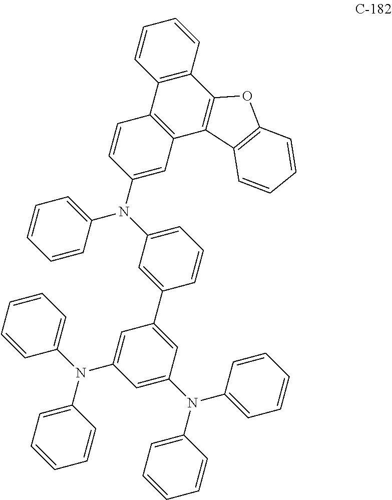 [SMILES: c1ccc(N(c2ccccc2)c2cc(-c3cccc(N(c4ccccc4)c4ccc5c6ccccc6c6oc7ccccc7c6c5c4)c3)cc(N(c3ccccc3)c3ccccc3)c2)cc1]

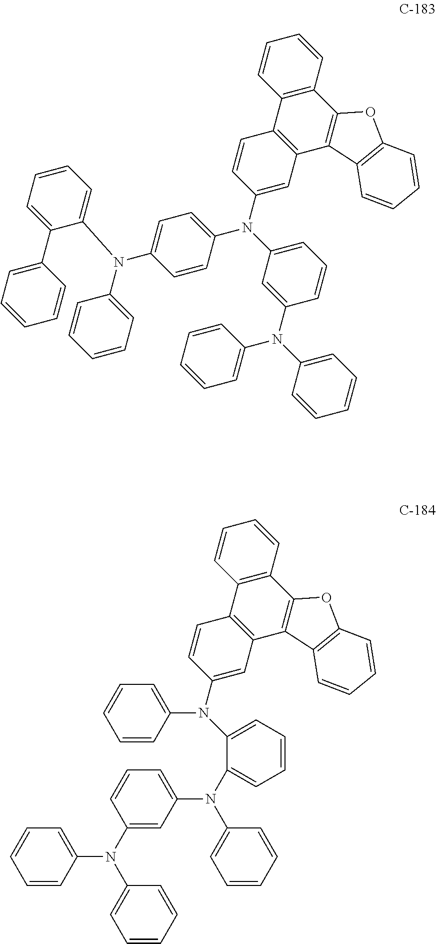 [SMILES: c1ccc(-c2ccccc2N(c2ccccc2)c2ccc(N(c3cccc(N(c4ccccc4)c4ccccc4)c3)c3ccc4c5ccccc5c5oc6ccccc6c5c4c3)cc2)cc1 ; c1ccc(N(c2ccccc2)c2cccc(N(c3ccccc3)c3ccccc3N(c3ccccc3)c3ccc4c5ccccc5c5oc6ccccc6c5c4c3)c2)cc1]

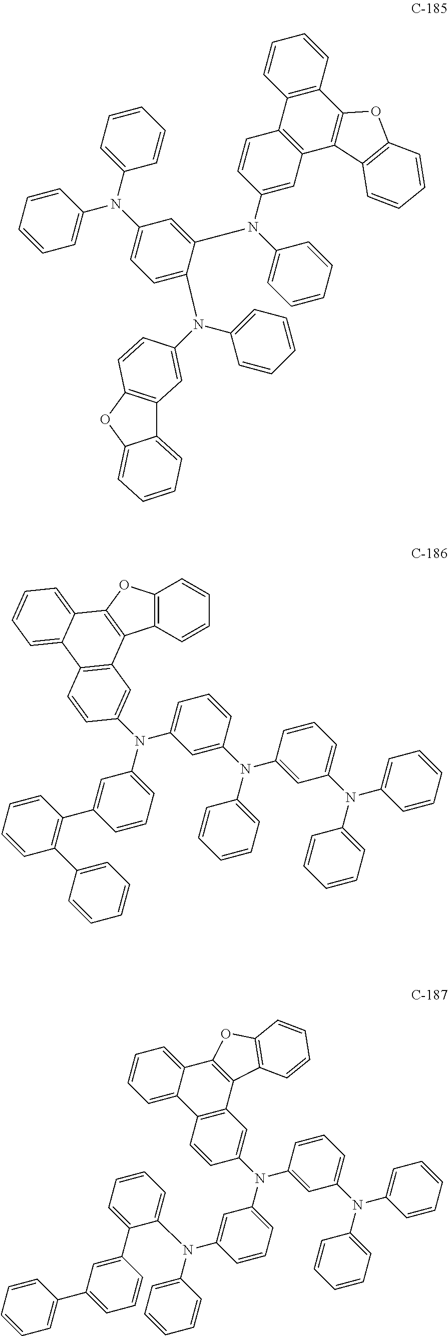 [SMILES: c1ccc(N(c2ccccc2)c2ccc(N(c3ccccc3)c3ccc4oc5ccccc5c4c3)c(N(c3ccccc3)c3ccc4c5ccccc5c5oc6ccccc6c5c4c3)c2)cc1 ; c1ccc(-c2ccccc2-c2cccc(N(c3cccc(N(c4ccccc4)c4cccc(N(c5ccccc5)c5ccccc5)c4)c3)c3ccc4c5ccccc5c5oc6ccccc6c5c4c3)c2)cc1 ; c1ccc(-c2cccc(-c3ccccc3N(c3ccccc3)c3cccc(N(c4cccc(N(c5ccccc5)c5ccccc5)c4)c4ccc5c6ccccc6c6oc7ccccc7c6c5c4)c3)c2)cc1]

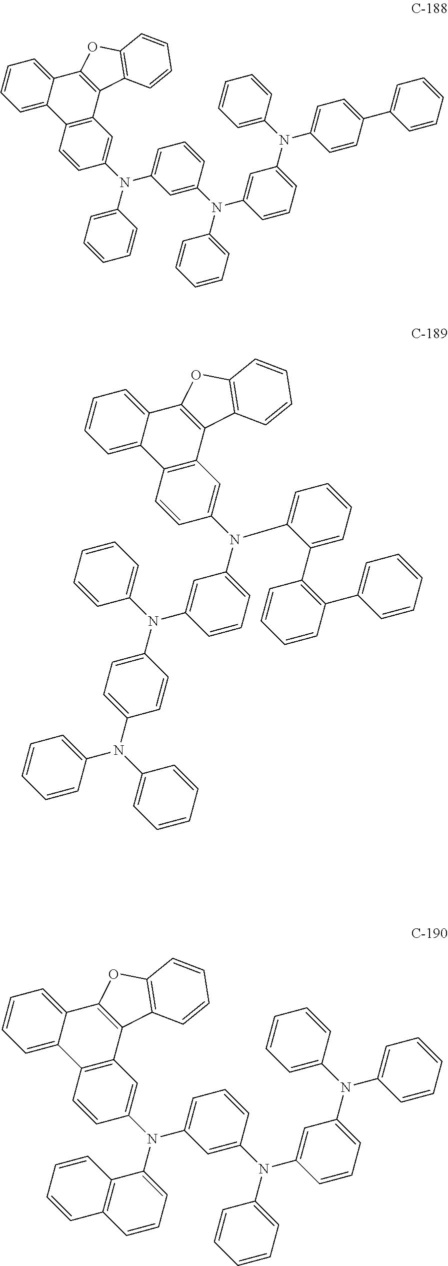 [SMILES: c1ccc(-c2ccc(N(c3ccccc3)c3cccc(N(c4ccccc4)c4cccc(N(c5ccccc5)c5ccc6c7ccccc7c7oc8ccccc8c7c6c5)c4)c3)cc2)cc1 ; c1ccc(-c2ccccc2-c2ccccc2N(c2cccc(N(c3ccccc3)c3ccc(N(c4ccccc4)c4ccccc4)cc3)c2)c2ccc3c4ccccc4c4oc5ccccc5c4c3c2)cc1 ; c1ccc(N(c2ccccc2)c2cccc(N(c3ccccc3)c3cccc(N(c4ccc5c6ccccc6c6oc7ccccc7c6c5c4)c4cccc5ccccc45)c3)c2)cc1]

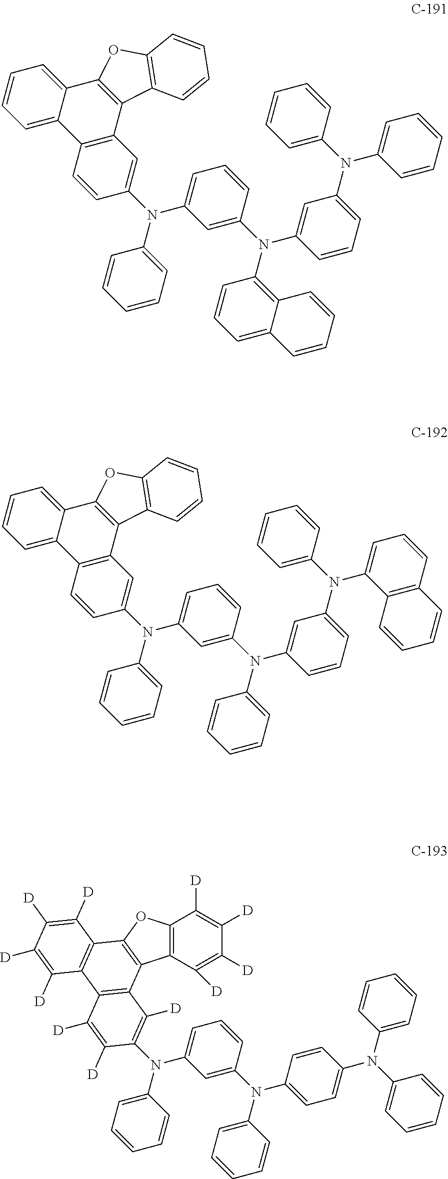 [SMILES: c1ccc(N(c2ccccc2)c2cccc(N(c3cccc(N(c4ccccc4)c4ccc5c6ccccc6c6oc7ccccc7c6c5c4)c3)c3cccc4ccccc34)c2)cc1 ; c1ccc(N(c2cccc(N(c3ccccc3)c3ccc4c5ccccc5c5oc6ccccc6c5c4c3)c2)c2cccc(N(c3ccccc3)c3cccc4ccccc34)c2)cc1 ; [2H]c1c([2H])c([2H])c2c(oc3c4c([2H])c([2H])c([2H])c([2H])c4c4c([2H])c([2H])c(N(c5ccccc5)c5cccc(N(c6ccccc6)c6ccc(N(c7ccccc7)c7ccccc7)cc6)c5)c([2H])c4c32)c1[2H]]

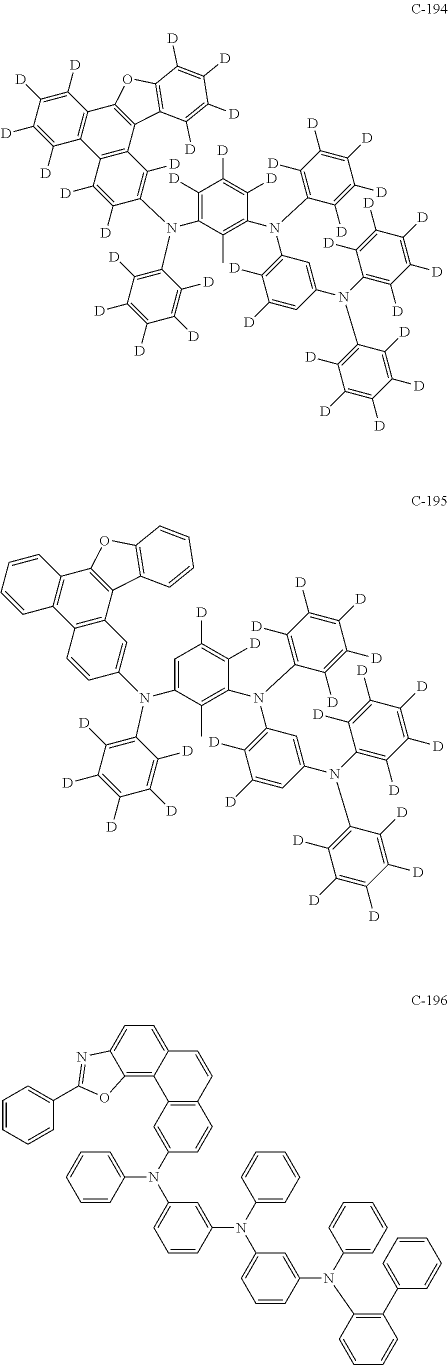

The present disclosure provides an organic electroluminescent material comprising the organic electroluminescent compound represented by formula 1, and an organic electroluminescent device comprising the same.

The organic electroluminescent material may consist of the organic electroluminescent compound of the present disclosure alone, and may further comprise conventional materials included in an organic electroluminescent material.

The organic electroluminescent compound represented by formula 1 may comprised in at least one layer selected from the group consisting of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer, and preferably in at least one layer of a light-emitting layer (host material), a hole transport layer, an electron blocking layer and a light-emitting auxiliary layer.

The plurality of host materials of the present disclosure comprises a first host material and a second host material, wherein the first host material comprises at least one compound represented by formula 1, and the second host material comprises at least one compound represented by formula 2.

Hereinafter, the compound represented by formula 2 will be described in more detail.

In formula 2, X₁ to X₃, each independently, represent CR′ or N; with the proviso that at least two of X₁ to X₃ represent N. For example, two of X₁ to X₃ may be N, or all of X 1 to X 3 may be N.

In formula 2, R′ represents hydrogen or deuterium. For example, R′ may be hydrogen.

In formula 2, L₁ to L₃, each independently, represent a single bond, a substituted or unsubstituted (C6-C30)arylene, or a substituted or unsubstituted (3- to 30-membered)heteroarylene. According to one embodiment of the present disclosure, L₁ to L₃, each independently, represent a single bond, or a (C6-C20)arylene unsubstituted or substituted with a (C6-C18)aryl(s). According to another embodiment of the present disclosure, L₁ to L₃, each independently, represent a single bond, or a (C6-C15)arylene unsubstituted or substituted with a (C6-C10)aryl(s). For example, L₁ to L₃, each independently, may be a single bond, a phenylene unsubstituted or substituted with a phenyl(s), a biphenylene, or a naphthylene.

In formula 2, Ar₁ to Ar₃, each independently, represent a substituted or unsubstituted (C6-C30)aryl, or a substituted or unsubstituted (3- to 30-membered)heteroaryl. According to one embodiment of the present disclosure, Ar₁ to Ar₃, each independently, represent a (C6-C25)aryl unsubstituted or substituted with at least one of a (C1-C10)alkyl(s) and (C6-C18)aryl(s), or a (6- to 25-membered)heteroaryl unsubstituted or substituted with a (C6-C20)aryl(s). According to another embodiment of the present disclosure, Ar₁ to Ar₃, each independently, represent a (C6-C20)aryl unsubstituted or substituted with at least one of a (C1-C6)alkyl(s) and (C6-C15)aryl(s), or a (6- to 20-membered)heteroaryl unsubstituted or substituted with a (C6-C15)aryl(s). For example, Ar₁ to Ar₃, each independently, may be a phenyl unsubstituted or substituted with a naphthyl(s), a biphenyl, a terphenyl, a naphthyl unsubstituted or substituted with a phenyl(s), a phenanthrenyl, a benzophenanthrenyl unsubstituted or substituted with a phenyl(s), a dimethylfluorenyl, a phenylfluorenyl, a di phenylfluorenyl, a dimethylbenzofluorenyl, a di phenyl benzofluorenyl, a chrysenyl unsubstituted or substituted with a phenyl(s), a triphenylenyl, a tetramethyldihydrophenanthrenyl, a dibenzofuranyl unsubstituted or substituted with at least one of a phenyl(s) and a biphenyl(s), a dibenzothiophenyl or a benzonaphthofuranyl.

According to one embodiment of the present disclosure, in formula 2, Ar₁ to Ar₃, each independently, represent a (C6-C30)aryl unsubstituted or substituted with at least one of deuterium, a (C1-C30)alkyl(s), and a (C6-C30)aryl(s). According to another embodiment of the present disclosure, Ar₁ to Ar₃, each independently, represent a (C6-C25)aryl unsubstituted or substituted with at least one of deuterium, a (C1-C10)alkyl(s), and a (C6-C18)aryl(s). According to still another embodiment of the present disclosure, Ar₁ to Ar₃, each independently, represent a (C6-C20)aryl unsubstituted or substituted with at least one of deuterium, a (C1-C6)alkyl(s), and a (C6-C15)aryl(s). For example, Ar₁ to Ar₃, each independently, may be a phenyl, a naphthylphenyl, a biphenyl, a terphenyl, a naphthyl, a phenylnaphthyl, a naphthylphenyl, a phenanthrenyl, a benzophenanthrenyl unsubstituted or substituted with a phenyl(s), a dimethylfluorenyl, a phenylfluorenyl, a diphenylfluorenyl, a dimethylbenzofluorenyl, a diphenylbenzofluorenyl, a chrysenyl unsubstituted or substituted with a phenyl(s), a triphenylenyl, or a tetramethyldihydrophenanthrenyl.

In formula 2, o to q, each independently, represent an integer of 1 or 2; in which if o to q represent an integer of 2 or more, each of L₁ to each of L₃ may be the same or different.

According to one embodiment of the present disclosure, in formula 2, at least one of Ar₁ to Ar₃ is represented by the following formula 2-1.

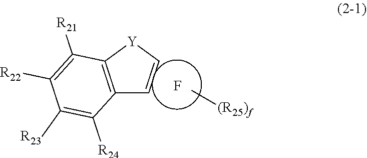

In formula 2-1, Y represents O, S, N(R₃₁), or C(R₃₂)(R₃₃). For example, Y may be O, S or C(R₃₂)(R₃₃)

In formula 2-1, R₃₁ is a position connected to any one of L₁ to L₃, or a substituted or unsubstituted (C6-C30)aryl.

In formula 2-1, R₃₂ and R₃₃, each independently, are a position connected to any one of L₁ to L₃, or represent a substituted or unsubstituted (C1-C30)alkyl, a substituted or unsubstituted (C6-C30)aryl, or a substituted or unsubstituted (3- to 30-membered)heteroaryl. According to one embodiment of the present disclosure, R₃₂ and R₃₃, each independently, are a position connected to any one of L₁ to L₃, or represent an unsubstituted (C1-C10)alkyl, or an unsubstituted (C6-C12)aryl. For example, R₃₂ and R₃₃, each independently, may be a position connected to any one of L₁ to L₃, or may be a methyl or a phenyl.

In formula 2-1, F represents a benzene ring or a naphthalene ring, and f represents an integer of 4 to 6.

In formula 2-1, R₂₁ to R₂₅, each independently, are a position connected to any one of L₁ to L₃; or represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to an adjacent substituent(s) to form a ring(s). According to one embodiment of the present disclosure, R₂₁ to R₂₅, each independently, are a position connected to any one of L₁ to L₃; or represent hydrogen, deuterium or an unsubstituted (C6-C18)aryl. According to another embodiment of the present disclosure, R₂₁ to R₂₅, each independently, are a position connected to any one of L₁ to L₃; or represent hydrogen, deuterium or an unsubstituted (C6-C15)aryl. For example, R₂₁ to R₂₅, each independently, may be hydrogen, deuterium, a phenyl or a biphenyl.

The compound represented by formula 2 may be selected from the group consisting of the following compounds, but is not limited thereto.

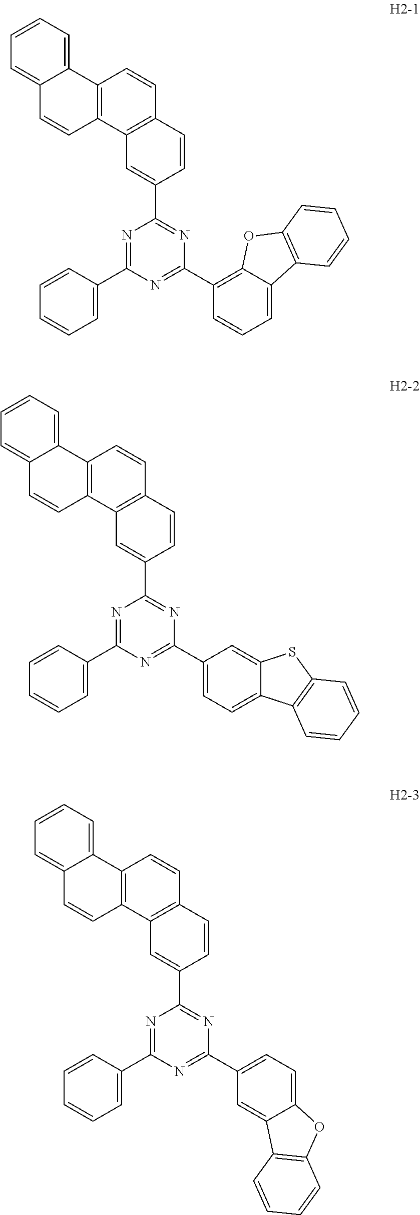 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

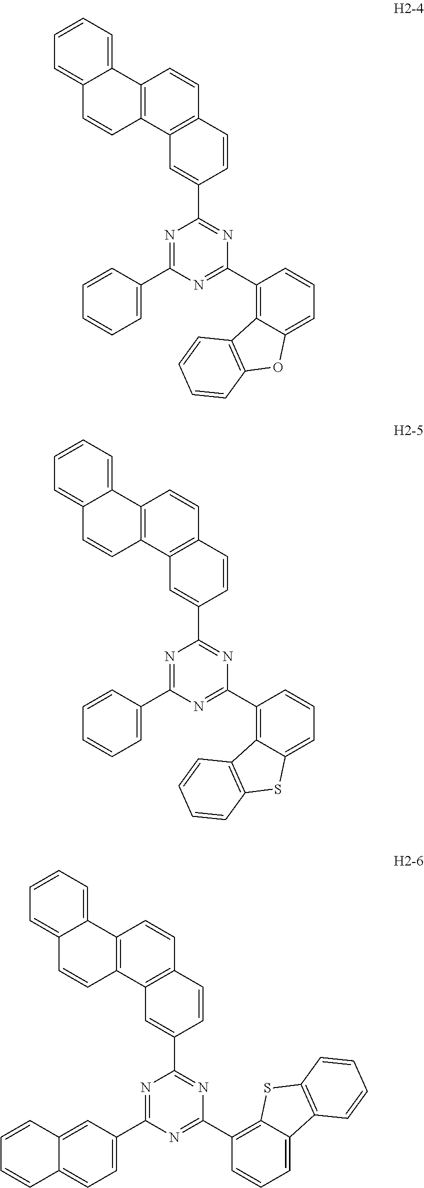 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

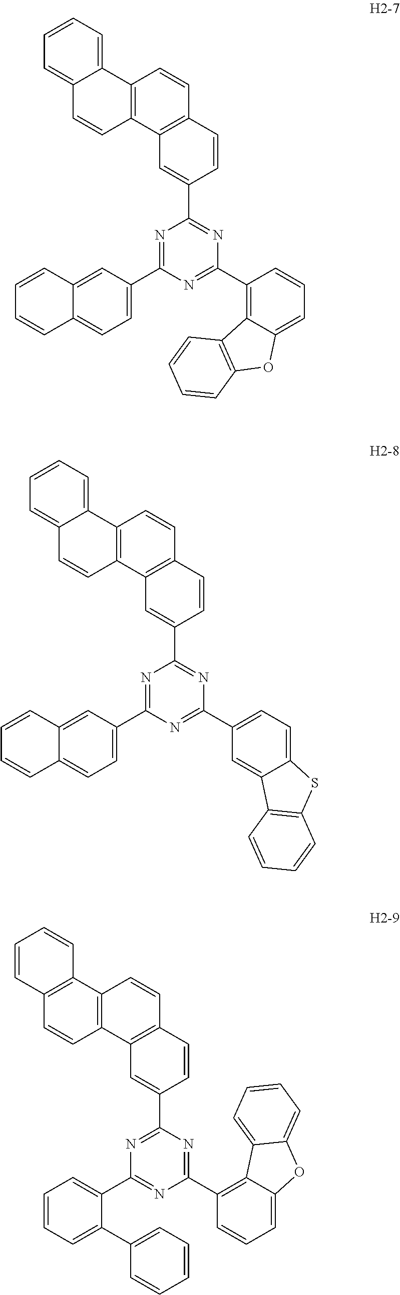 [SMILES: c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1 ; c1ccc(-c2ccccc2-c2nc(-c3ccc4ccc5c6ccccc6ccc5c4c3)nc(-c3cccc4oc5ccccc5c34)n2)cc1]

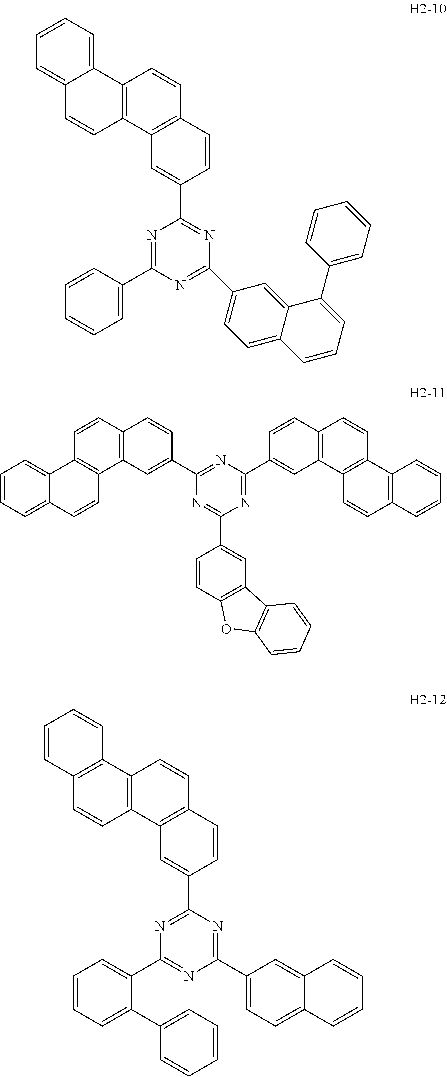 [SMILES: c1ccc(-c2nc(-c3ccc4cccc(-c5ccccc5)c4c3)nc(-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21 ; c1ccc(-c2ccccc2-c2nc(-c3ccc4ccccc4c3)nc(-c3ccc4ccc5c6ccccc6ccc5c4c3)n2)cc1]

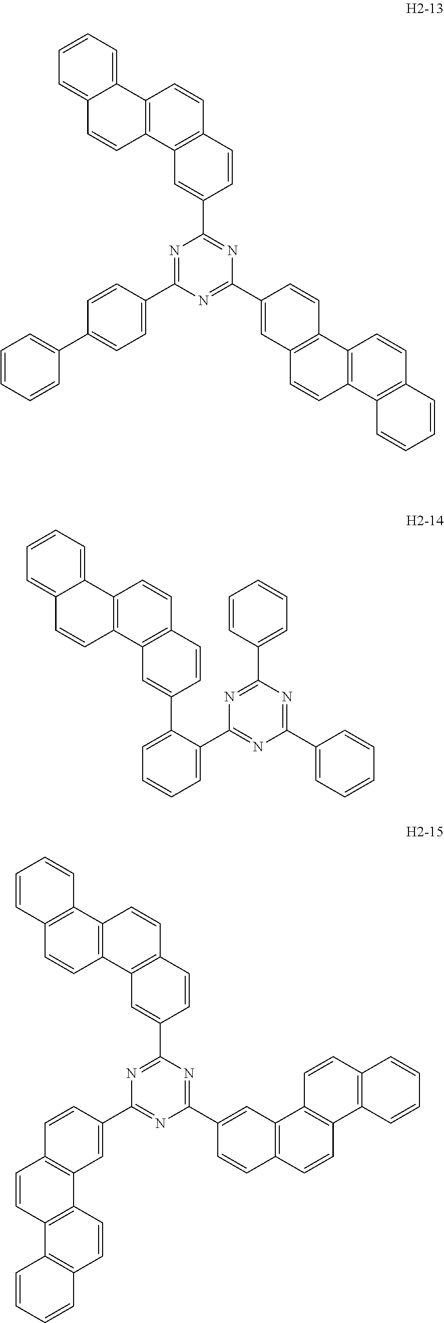 [SMILES: c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21]

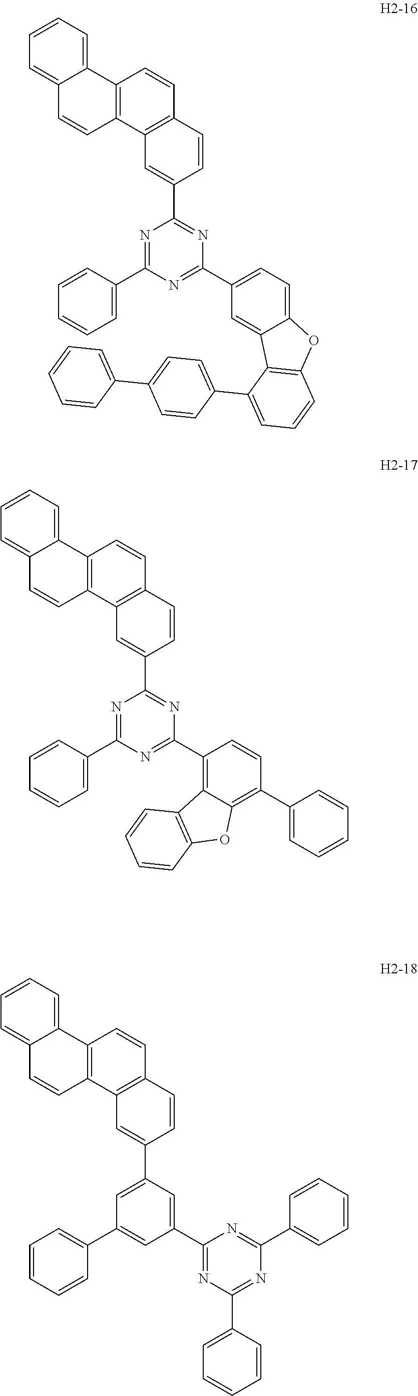 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1]

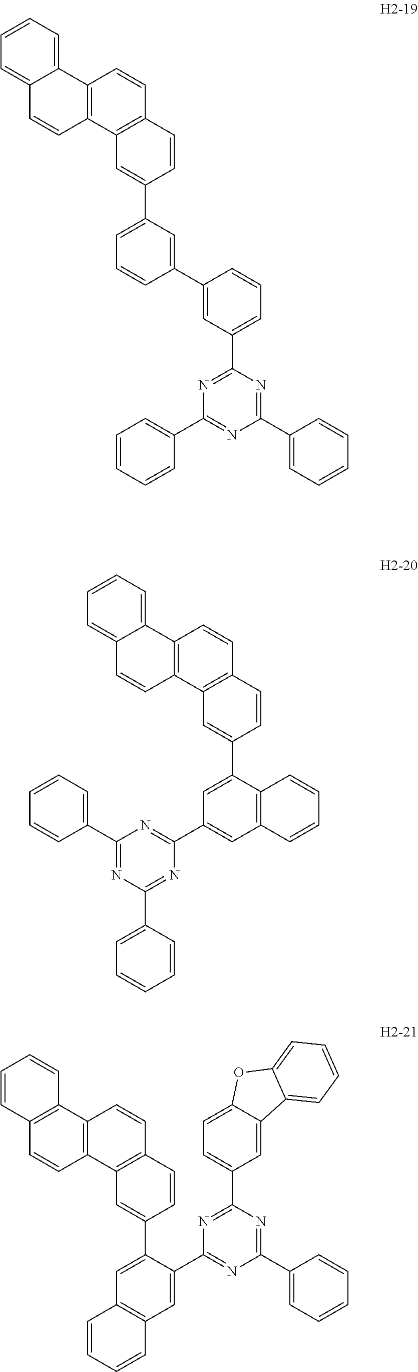 [SMILES: c1ccc(-c2nc(-c3ccccc3)nc(-c3cccc(-c4cccc(-c5ccc6ccc7c8ccccc8ccc7c6c5)c4)c3)n2)cc1 ; c1ccc(-c2nc(-c3ccccc3)nc(-c3cc(-c4ccc5ccc6c7ccccc7ccc6c5c4)c4ccccc4c3)n2)cc1 ; c1ccc(-c2nc(-c3ccc4oc5ccccc5c4c3)nc(-c3cc4ccccc4cc3-c3ccc4ccc5c6ccccc6ccc5c4c3)n2)cc1]

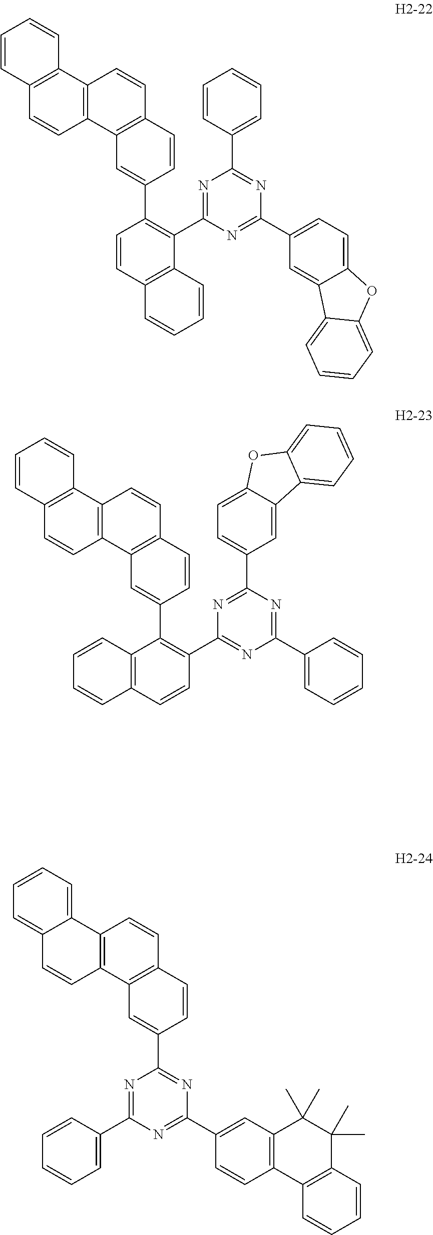 [SMILES: c1ccc(-c2nc(-c3ccc4oc5ccccc5c4c3)nc(-c3c(-c4ccc5ccc6c7ccccc7ccc6c5c4)ccc4ccccc34)n2)cc1 ; c1ccc(-c2nc(-c3ccc4oc5ccccc5c4c3)nc(-c3ccc4ccccc4c3-c3ccc4ccc5c6ccccc6ccc5c4c3)n2)cc1 ; CC1(C)c2ccccc2-c2ccc(-c3nc(-c4ccccc4)nc(-c4ccc5ccc6c7ccccc7ccc6c5c4)n3)cc2C1(C)C]

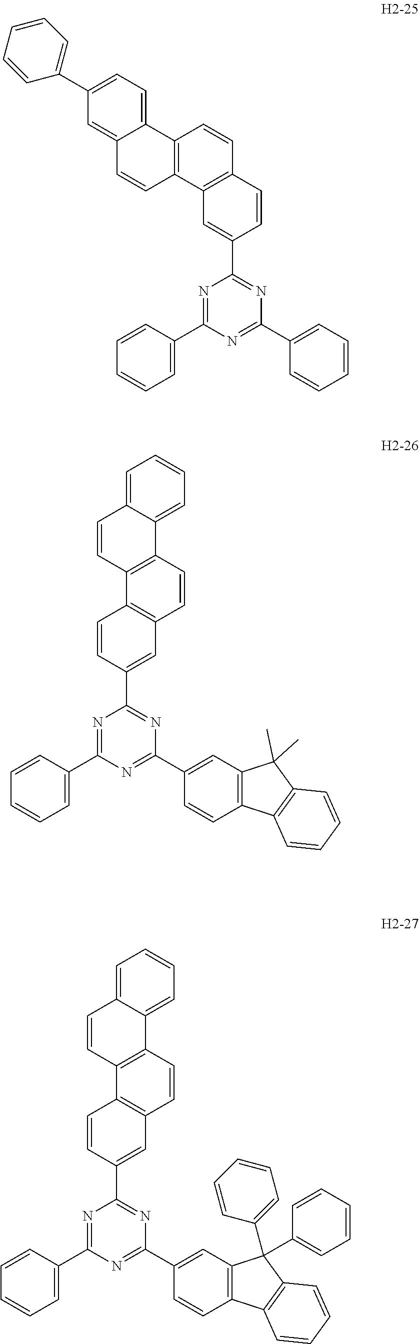 [SMILES: c1ccc(-c2ccc3c(ccc4c5cc(-c6nc(-c7ccccc7)nc(-c7ccccc7)n6)ccc5ccc34)c2)cc1 ; CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1]

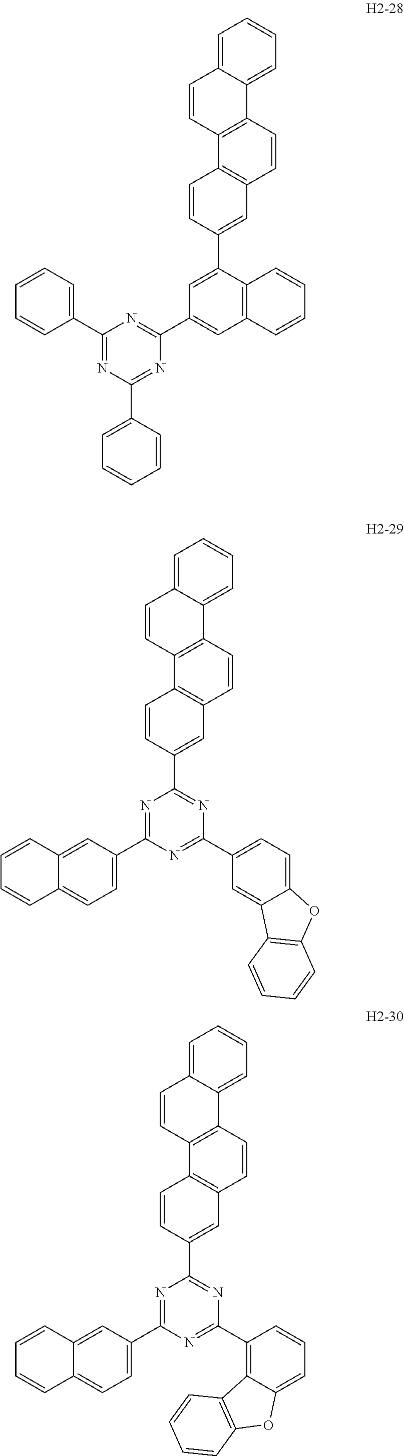 [SMILES: c1ccc(-c2nc(-c3ccccc3)nc(-c3cc(-c4ccc5c(ccc6c7ccccc7ccc56)c4)c4ccccc4c3)n2)cc1 ; c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1]

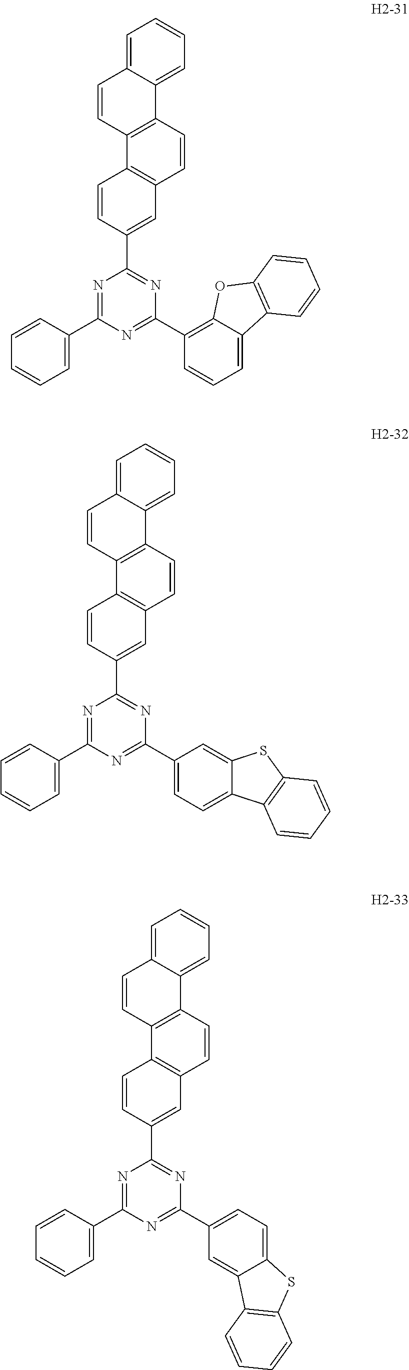 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

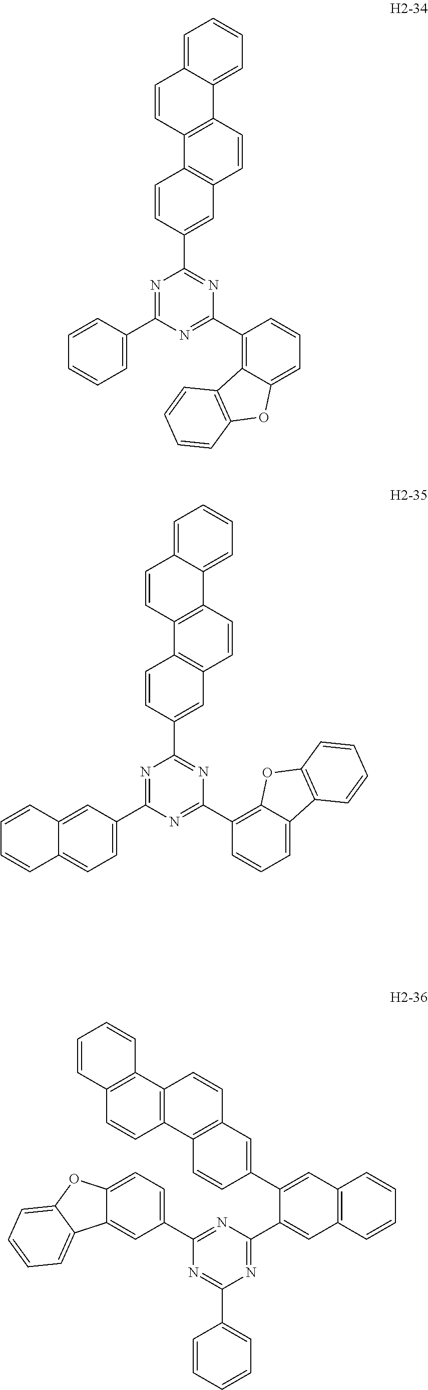 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1 ; c1ccc(-c2nc(-c3ccc4oc5ccccc5c4c3)nc(-c3cc4ccccc4cc3-c3ccc4c(ccc5c6ccccc6ccc45)c3)n2)cc1]

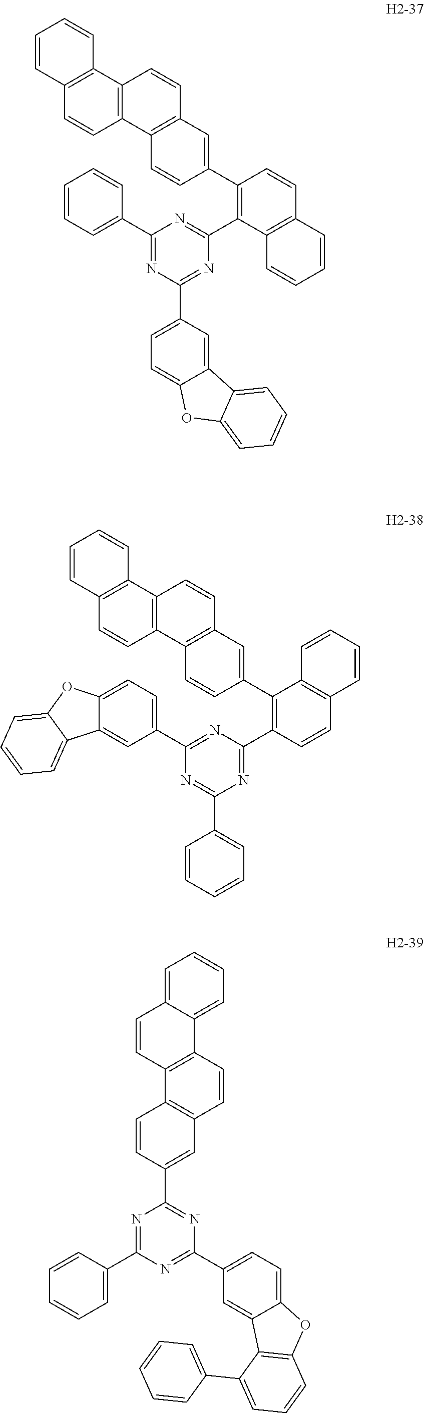 [SMILES: c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1]

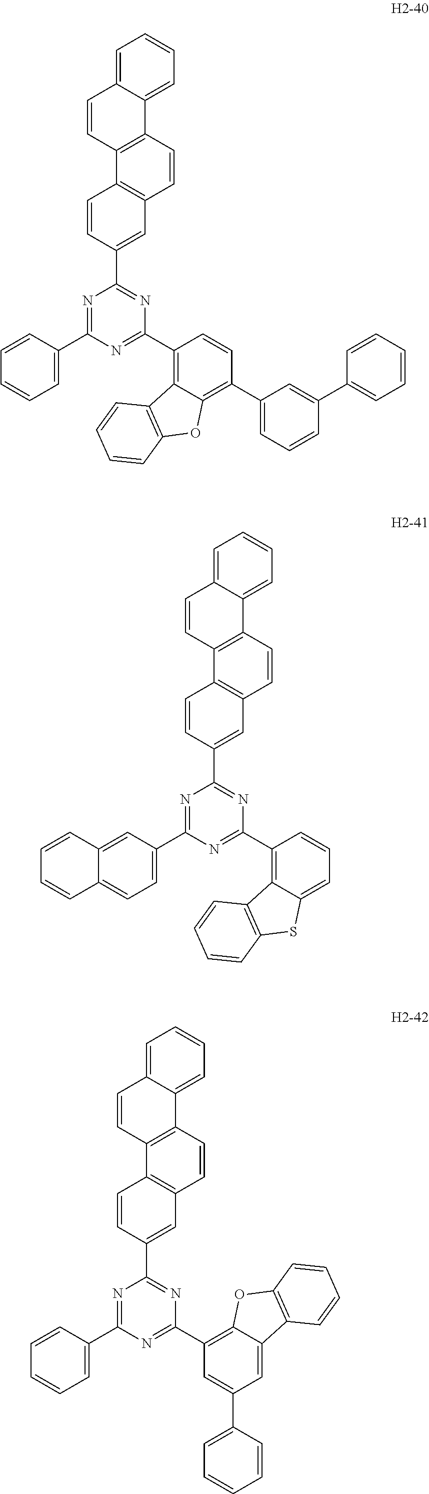 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1]

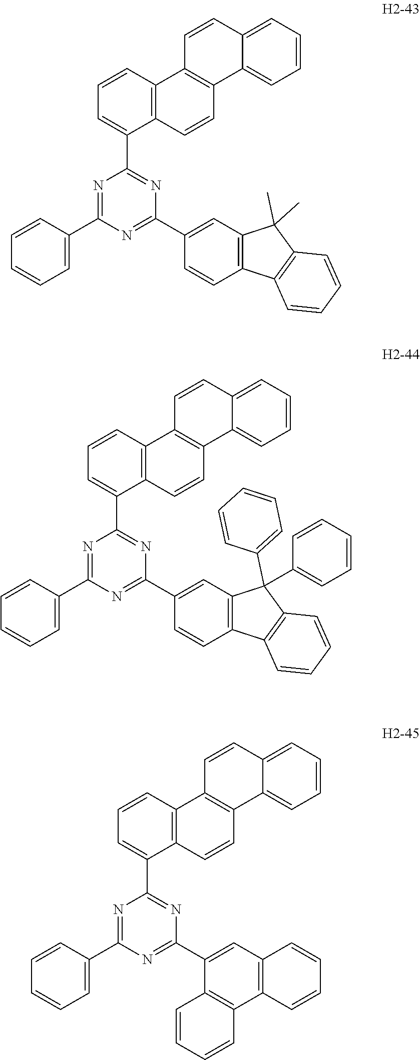 [SMILES: CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1 ; c1ccc(-c2nc(-c3cc4ccccc4c4ccccc34)nc(-c3cccc4c3ccc3c5ccccc5ccc43)n2)cc1]

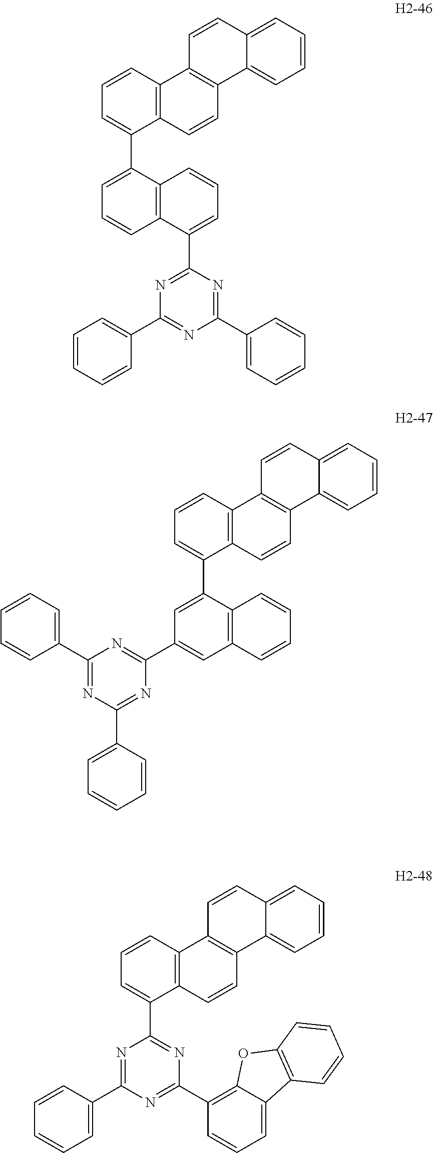 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1]

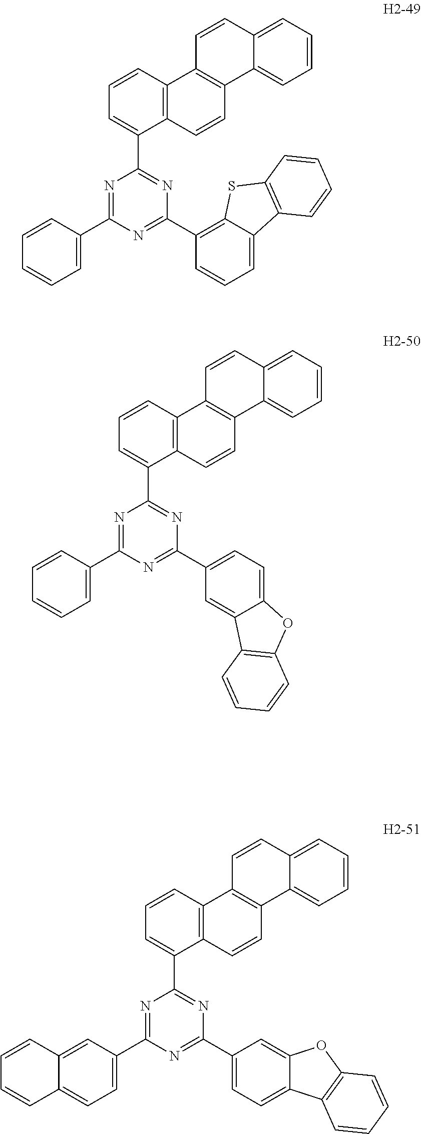 [SMILES: c1ccc(-c2nc(-c3cccc4c3ccc3c5ccccc5ccc43)nc(-c3cccc4c3sc3ccccc34)n2)cc1 ; c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1]

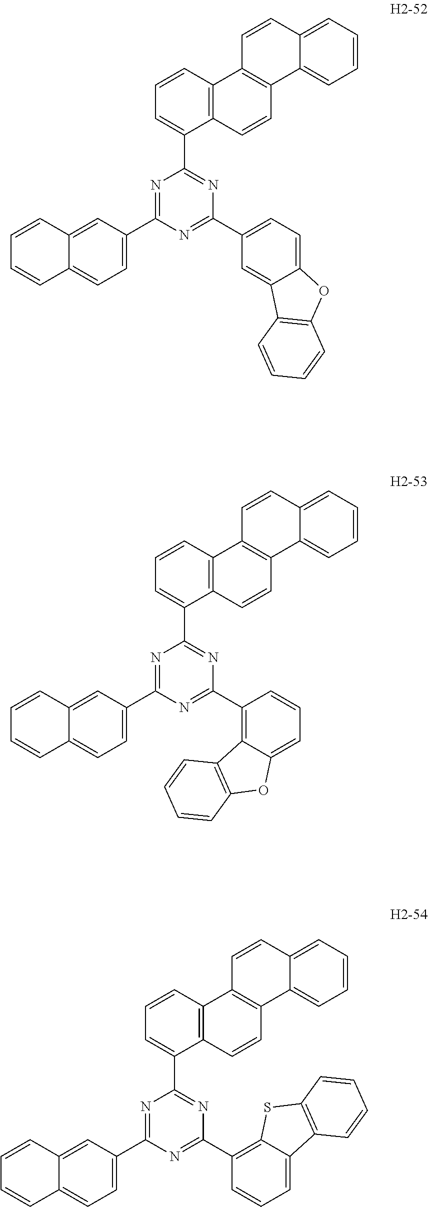 [SMILES: c1ccc2cc(-c3nc(-c4ccc5oc6ccccc6c5c4)nc(-c4cccc5c4ccc4c6ccccc6ccc54)n3)ccc2c1 ; c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1]

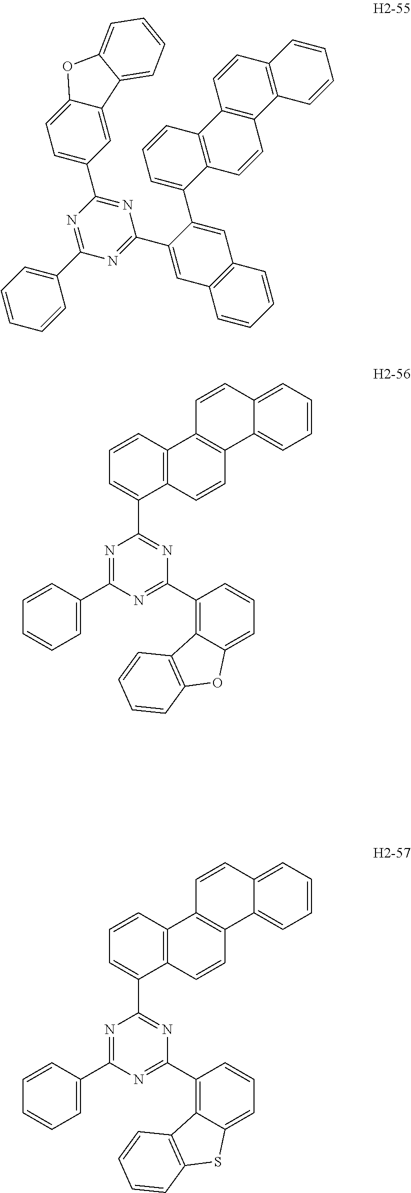 [SMILES: c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1]

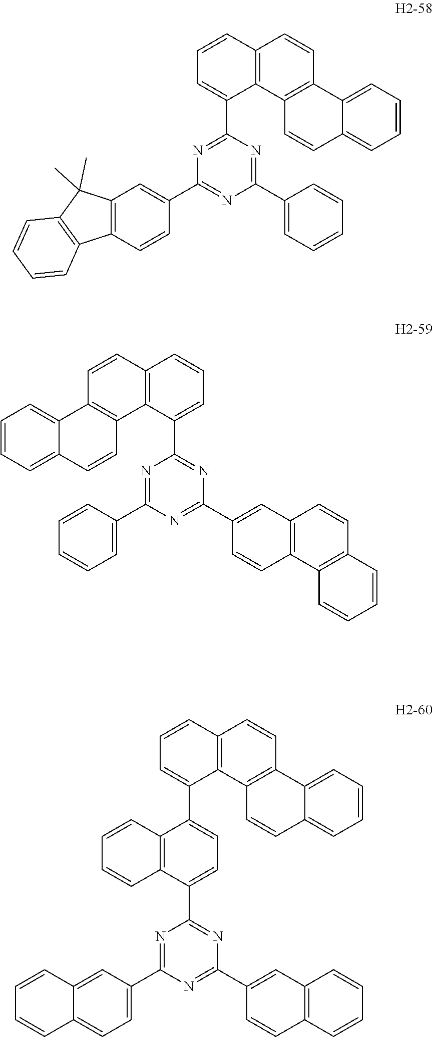 [SMILES: CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1 ; c1ccc2cc(-c3nc(-c4ccc5ccccc5c4)nc(-c4ccc(-c5cccc6ccc7c8ccccc8ccc7c56)c5ccccc45)n3)ccc2c1]

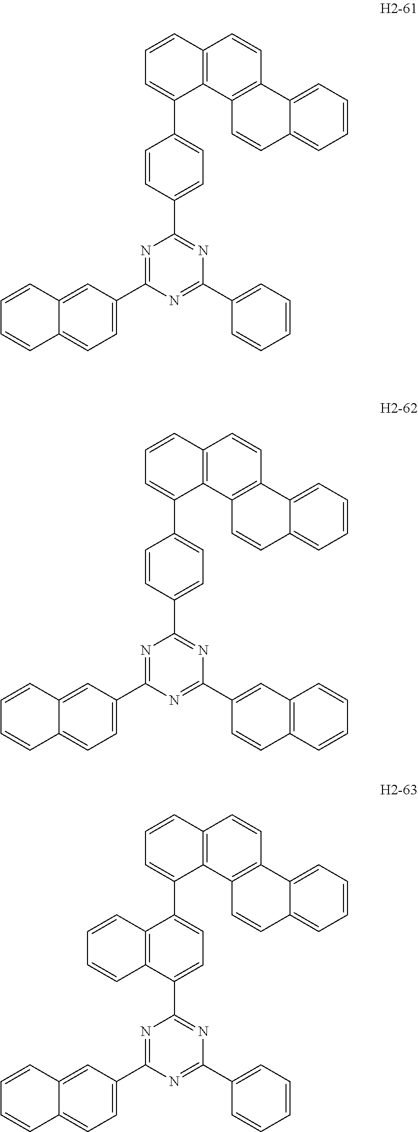 [SMILES: c1ccc(-c2nc(-c3ccc(-c4cccc5ccc6c7ccccc7ccc6c45)cc3)nc(-c3ccc4ccccc4c3)n2)cc1 ; c1ccc2cc(-c3nc(-c4ccc(-c5cccc6ccc7c8ccccc8ccc7c56)cc4)nc(-c4ccc5ccccc5c4)n3)ccc2c1 ; c1ccc(-c2nc(-c3ccc4ccccc4c3)nc(-c3ccc(-c4cccc5ccc6c7ccccc7ccc6c45)c4ccccc34)n2)cc1]

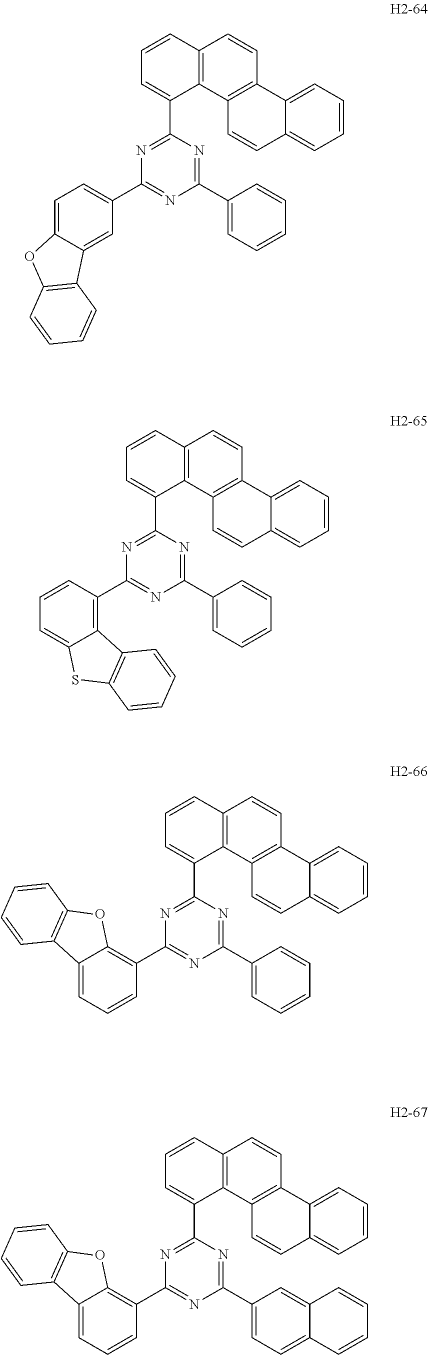 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1]

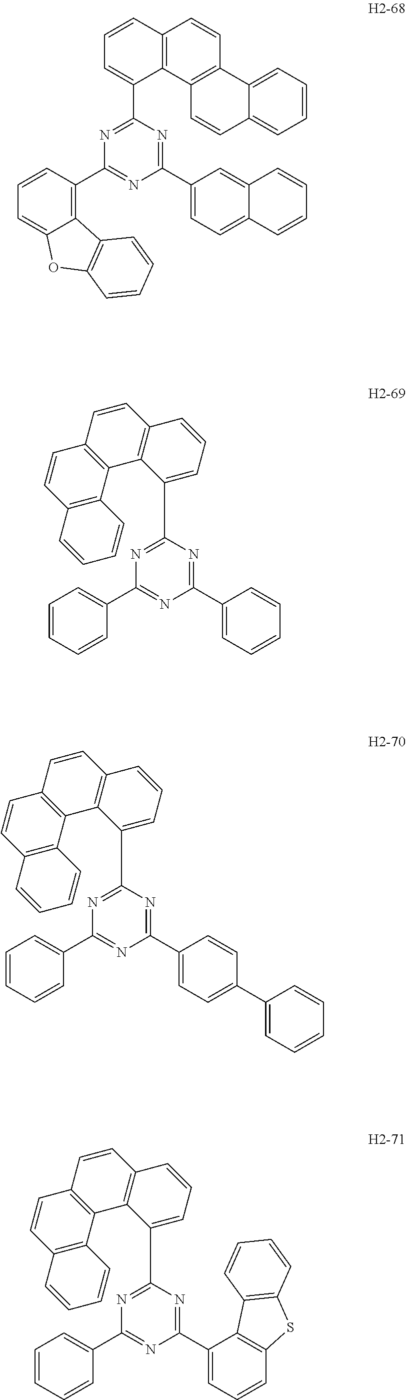 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3cccc4sc5ccccc5c34)nc(-c3cccc4ccc5ccc6ccccc6c5c34)n2)cc1]

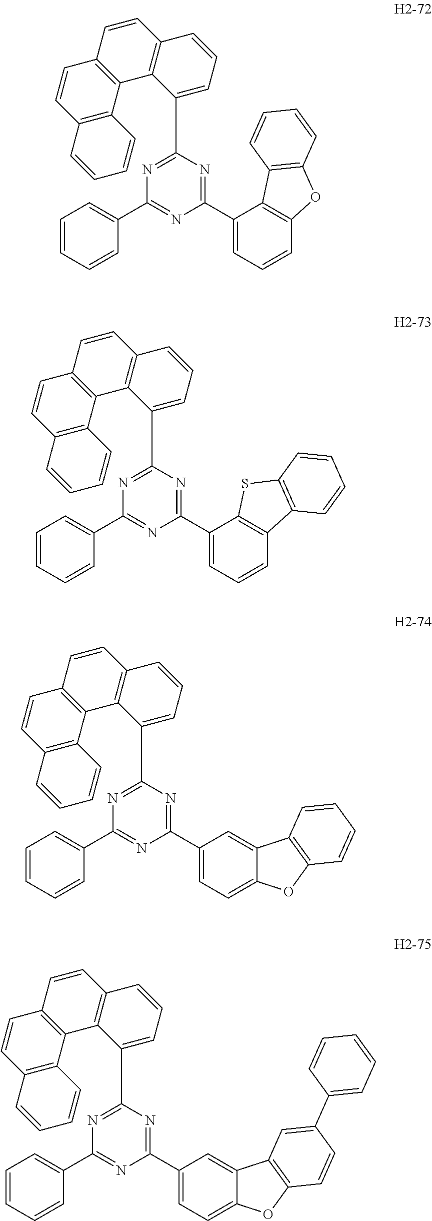 [SMILES: c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1 ; c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1]

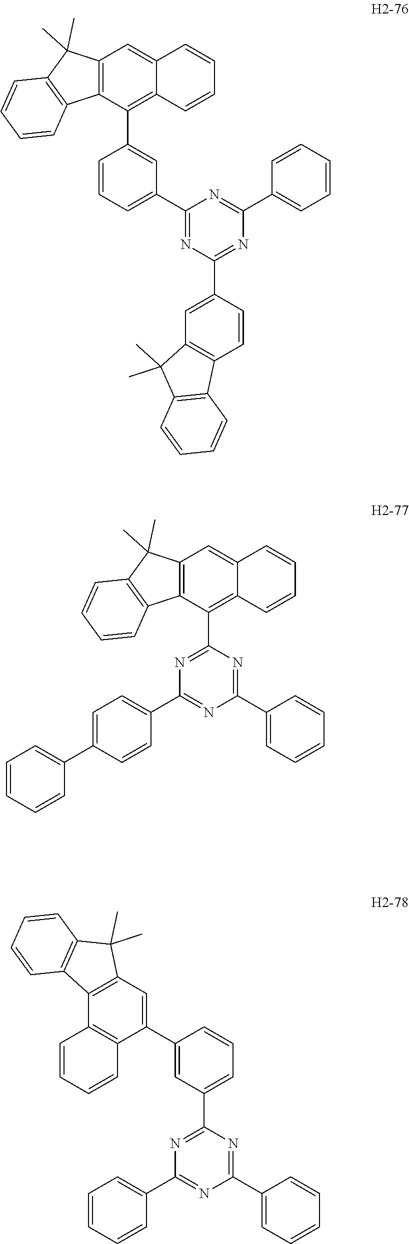 [SMILES: CC1(C)c2ccccc2-c2ccc(-c3nc(-c4ccccc4)nc(-c4cccc(-c5c6c(cc7ccccc57)C(C)(C)c5ccccc5-6)c4)n3)cc21 ; CC1(C)c2ccccc2-c2c1cc1ccccc1c2-c1nc(-c2ccccc2)nc(-c2ccc(-c3ccccc3)cc2)n1 ; CC1(C)c2ccccc2-c2c1cc(-c1cccc(-c3nc(-c4ccccc4)nc(-c4ccccc4)n3)c1)c1ccccc21]

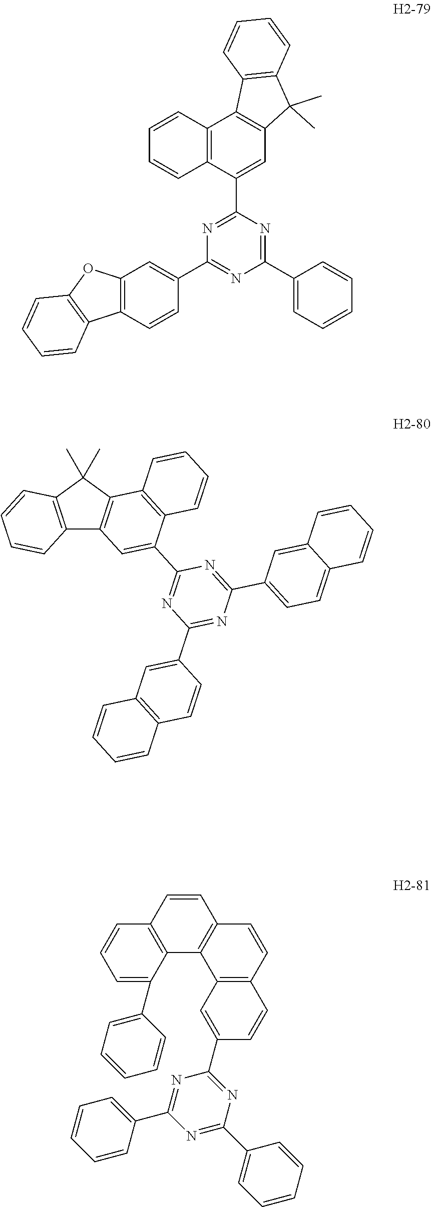 [SMILES: CC1(C)c2ccccc2-c2c1cc(-c1nc(-c3ccccc3)nc(-c3ccc4c(c3)oc3ccccc34)n1)c1ccccc21 ; CC1(C)c2ccccc2-c2cc(-c3nc(-c4ccc5ccccc5c4)nc(-c4ccc5ccccc5c4)n3)c3ccccc3c21 ; c1ccc(-c2nc(-c3ccccc3)nc(-c3ccc4ccc5ccc6cccc(-c7ccccc7)c6c5c4c3)n2)cc1]

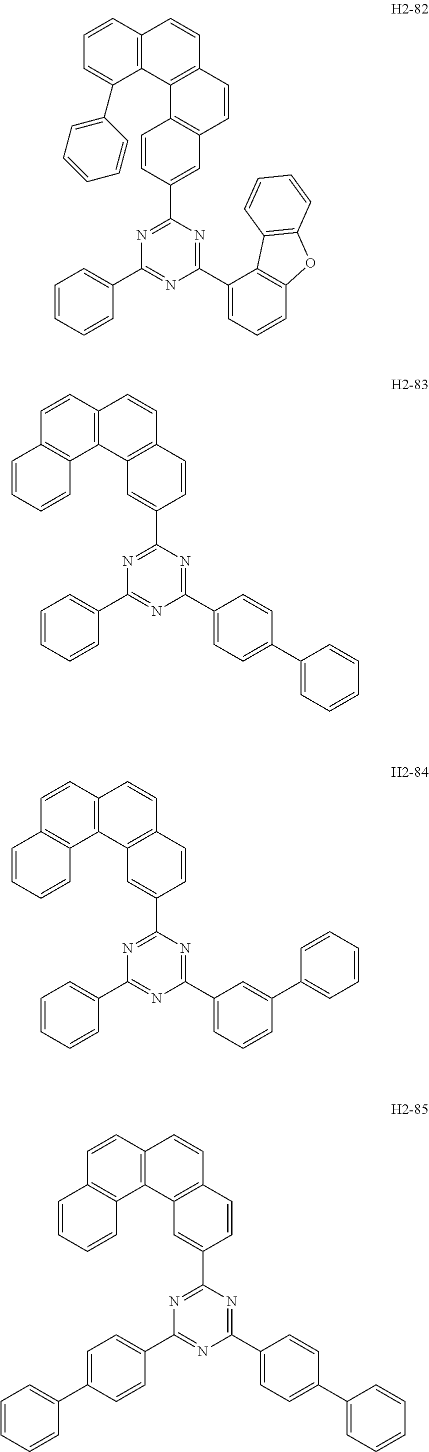 [SMILES: c1ccc(-c2nc(-c3ccc4c(ccc5ccc6cccc(-c7ccccc7)c6c54)c3)nc(-c3cccc4oc5ccccc5c34)n2)cc1 ; c1ccc(-c2ccc(-c3nc(-c4ccccc4)nc(-c4ccc5ccc6ccc7ccccc7c6c5c4)n3)cc2)cc1 ; c1ccc(-c2cccc(-c3nc(-c4ccccc4)nc(-c4ccc5ccc6ccc7ccccc7c6c5c4)n3)c2)cc1 ; c1ccc(-c2ccc(-c3nc(-c4ccc(-c5ccccc5)cc4)nc(-c4ccc5ccc6ccc7ccccc7c6c5c4)n3)cc2)cc1]

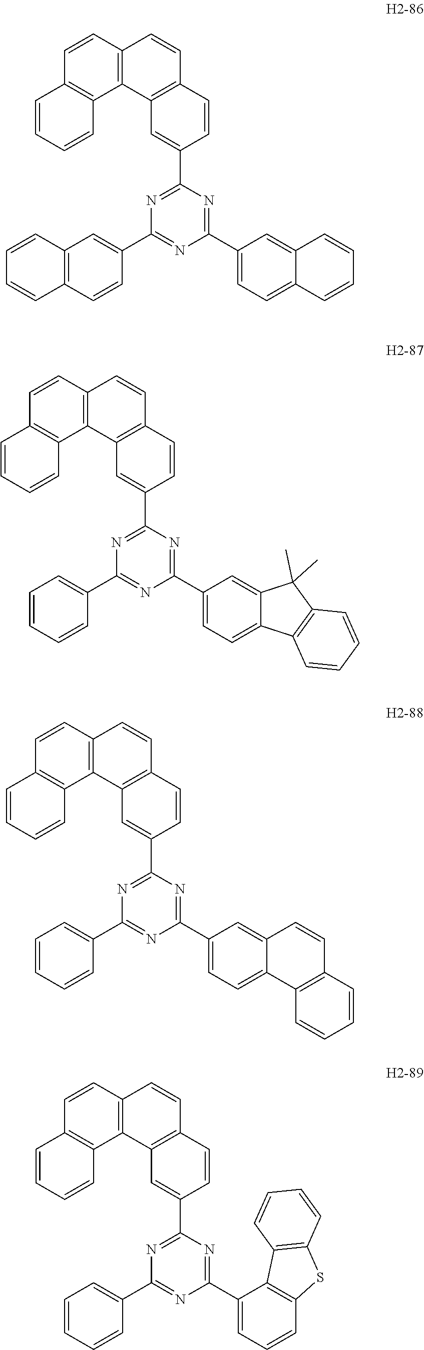 [SMILES: c1ccc2cc(-c3nc(-c4ccc5ccccc5c4)nc(-c4ccc5ccc6ccc7ccccc7c6c5c4)n3)ccc2c1 ; CC1(C)c2ccccc2-c2ccc(-c3nc(-c4ccccc4)nc(-c4ccc5ccc6ccc7ccccc7c6c5c4)n3)cc21 ; c1ccc(-c2nc(-c3ccc4c(ccc5ccccc54)c3)nc(-c3ccc4ccc5ccc6ccccc6c5c4c3)n2)cc1 ; c1ccc(-c2nc(-c3ccc4ccc5ccc6ccccc6c5c4c3)nc(-c3cccc4sc5ccccc5c34)n2)cc1]

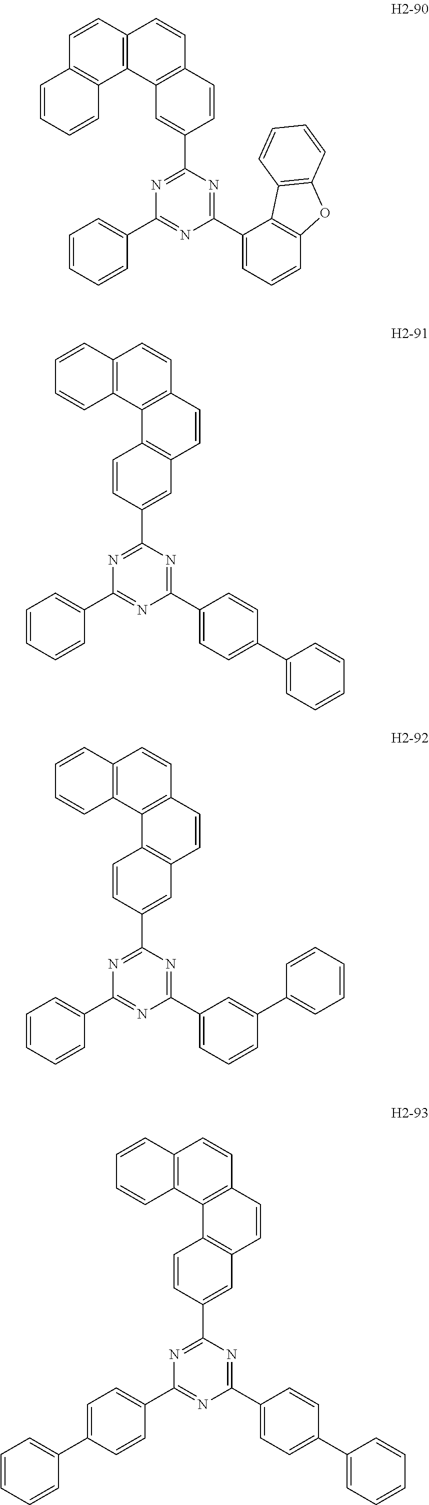 [SMILES: c1ccc(-c2nc(-c3ccc4ccc5ccc6ccccc6c5c4c3)nc(-c3cccc4oc5ccccc5c34)n2)cc1 ; c1ccc(-c2ccc(-c3nc(-c4ccccc4)nc(-c4ccc5c(ccc6ccc7ccccc7c65)c4)n3)cc2)cc1 ; c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1]

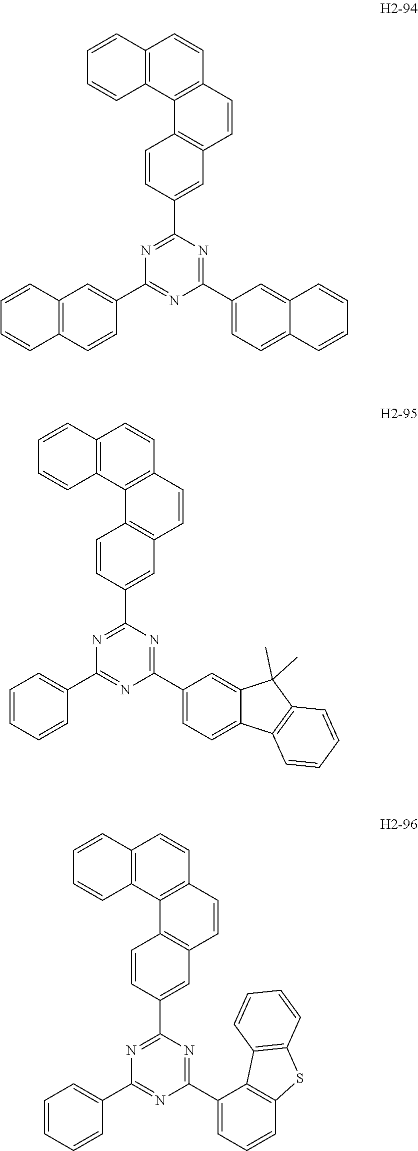 [SMILES: c1ccc2cc(-c3nc(-c4ccc5ccccc5c4)nc(-c4ccc5c(ccc6ccc7ccccc7c65)c4)n3)ccc2c1 ; CC1(C)c2ccccc2-c2ccc(-c3nc(-c4ccccc4)nc(-c4ccc5c(ccc6ccc7ccccc7c65)c4)n3)cc21 ; c1ccc(-c2nc(-c3ccc4c(ccc5ccc6ccccc6c54)c3)nc(-c3cccc4sc5ccccc5c34)n2)cc1]

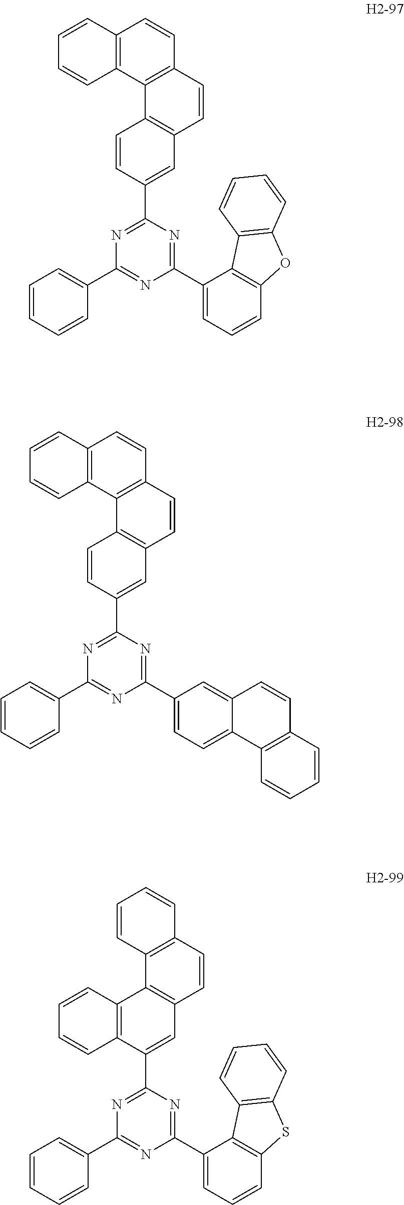 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3ccc4c(ccc5ccccc54)c3)nc(-c3ccc4c(ccc5ccc6ccccc6c54)c3)n2)cc1 ; c1ccc(-c2nc(-c3cc4ccc5ccccc5c4c4ccccc34)nc(-c3cccc4sc5ccccc5c34)n2)cc1]

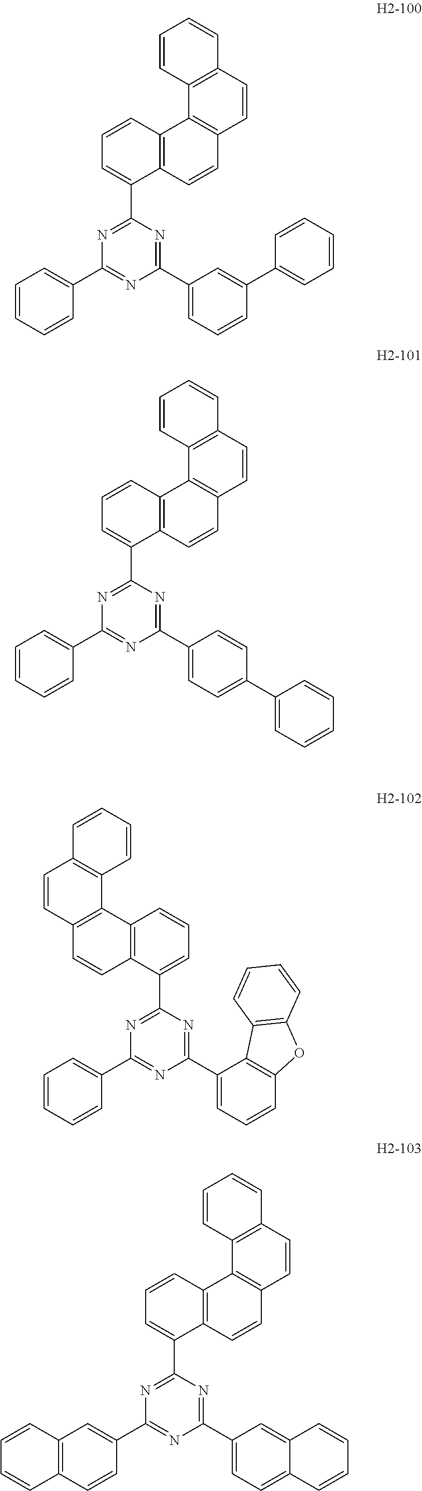 [SMILES: c1ccc(-c2cccc(-c3nc(-c4ccccc4)nc(-c4cccc5c4ccc4ccc6ccccc6c45)n3)c2)cc1 ; c1ccc(-c2ccc(-c3nc(-c4ccccc4)nc(-c4cccc5c4ccc4ccc6ccccc6c45)n3)cc2)cc1 ; c1ccc(-c2nc(-c3cccc4c3ccc3ccc5ccccc5c34)nc(-c3cccc4oc5ccccc5c34)n2)cc1 ; c1ccc2cc(-c3nc(-c4ccc5ccccc5c4)nc(-c4cccc5c4ccc4ccc6ccccc6c45)n3)ccc2c1]

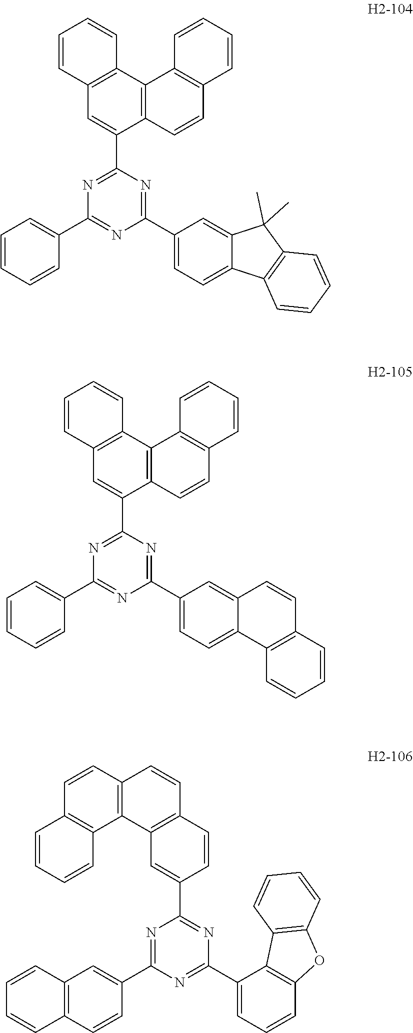 [SMILES: CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1 ; c1ccc2cc(-c3nc(-c4ccc5ccc6ccc7ccccc7c6c5c4)nc(-c4cccc5oc6ccccc6c45)n3)ccc2c1]

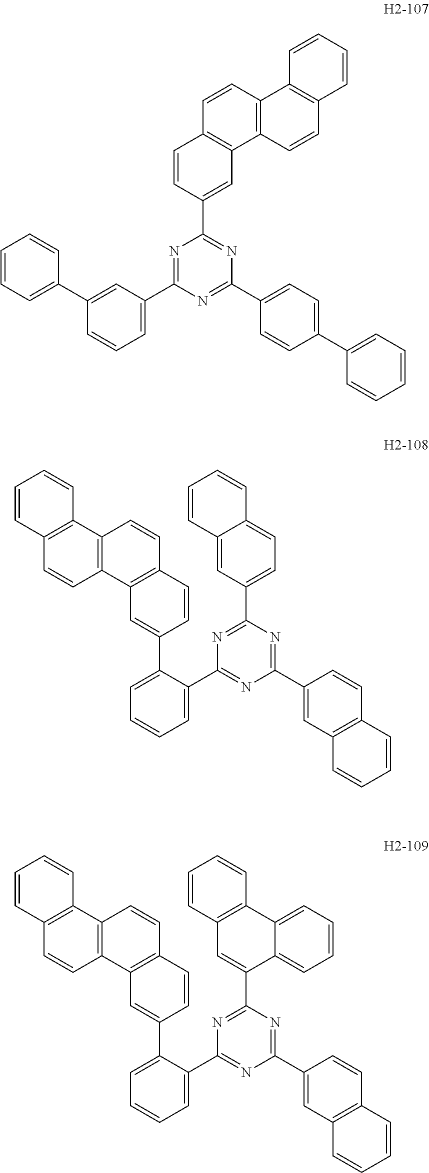 [SMILES: c1ccc(-c2ccc(-c3nc(-c4cccc(-c5ccccc5)c4)nc(-c4ccc5ccc6c7ccccc7ccc6c5c4)n3)cc2)cc1 ; c1ccc(-c2nc(-c3ccc4ccccc4c3)nc(-c3ccc4ccccc4c3)n2)c(-c2ccc3ccc4c5ccccc5ccc4c3c2)c1 ; c1ccc(-c2nc(-c3ccc4ccccc4c3)nc(-c3cc4ccccc4c4ccccc34)n2)c(-c2ccc3ccc4c5ccccc5ccc4c3c2)c1]

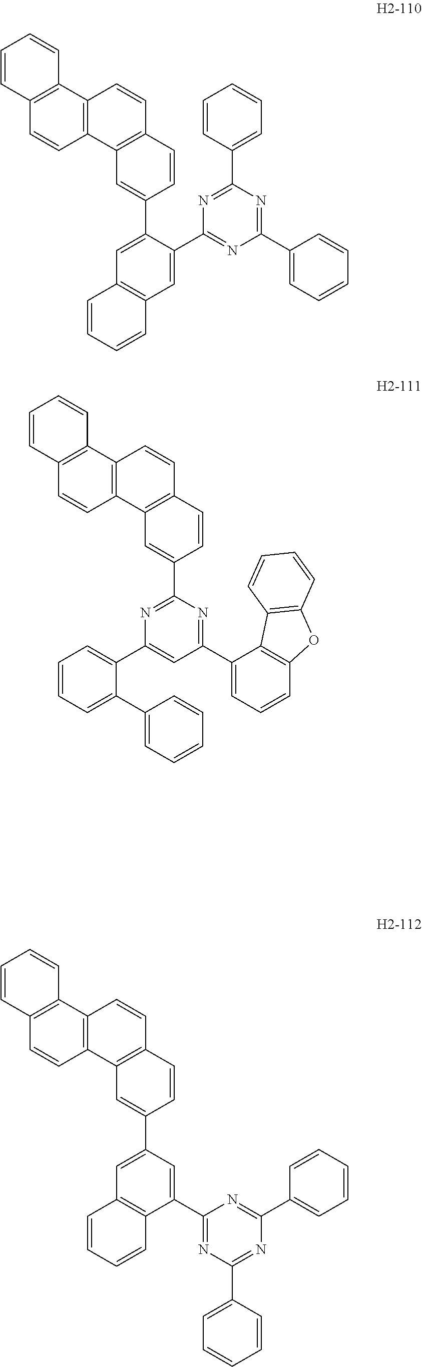 [SMILES: c1ccc(-c2nc(-c3ccccc3)nc(-c3cc4ccccc4cc3-c3ccc4ccc5c6ccccc6ccc5c4c3)n2)cc1 ; c1ccc(-c2ccccc2-c2cc(-c3cccc4oc5ccccc5c34)nc(-c3ccc4ccc5c6ccccc6ccc5c4c3)n2)cc1 ; c1ccc(-c2nc(-c3ccccc3)nc(-c3cc(-c4ccc5ccc6c7ccccc7ccc6c5c4)cc4ccccc34)n2)cc1]

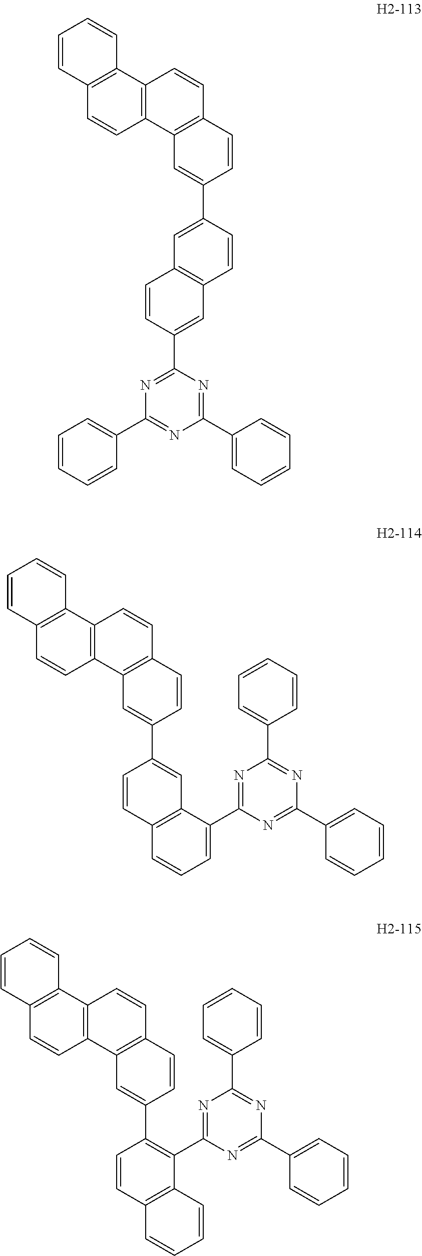 [SMILES: c1ccc(-c2nc(-c3ccccc3)nc(-c3ccc4cc(-c5ccc6ccc7c8ccccc8ccc7c6c5)ccc4c3)n2)cc1 ; c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1]

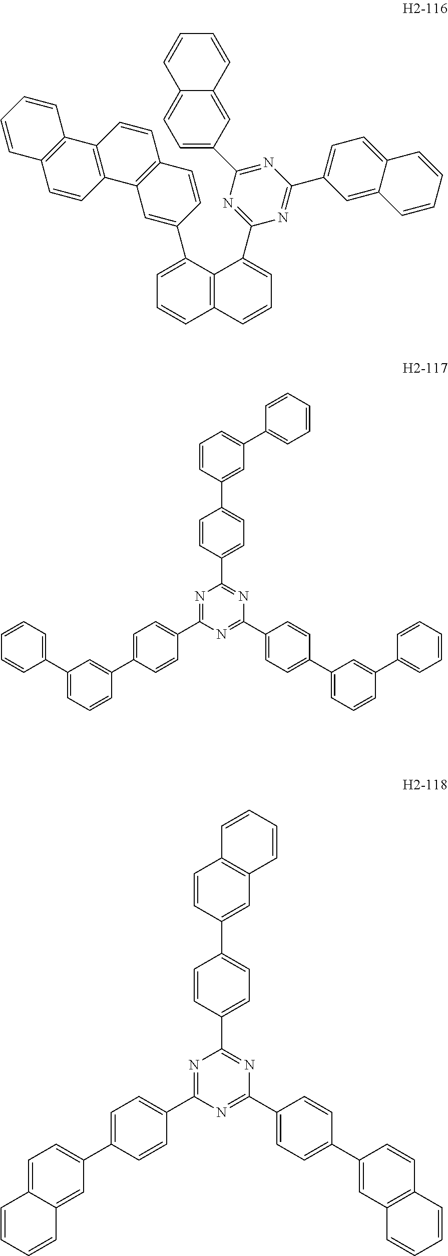 [SMILES: c1ccc2cc(-c3nc(-c4ccc5ccccc5c4)nc(-c4cccc5cccc(-c6ccc7ccc8c9ccccc9ccc8c7c6)c45)n3)ccc2c1 ; c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4nc(-c5ccc(-c6ccc7ccccc7c6)cc5)nc(-c5ccc(-c6ccc7ccccc7c6)cc5)n4)cc3)ccc2c1]

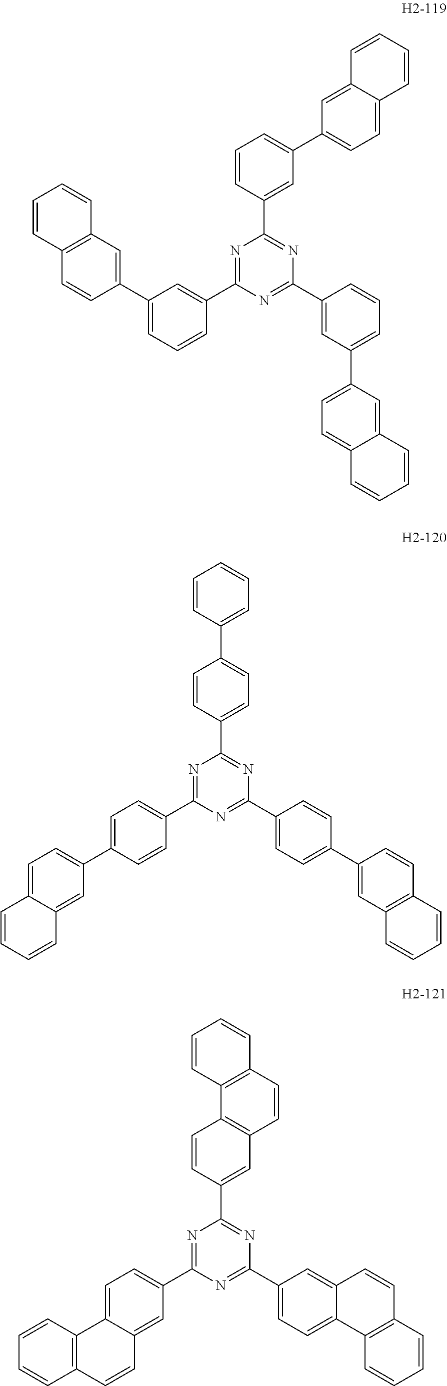 [SMILES: c1cc(-c2ccc3ccccc3c2)cc(-c2nc(-c3cccc(-c4ccc5ccccc5c4)c3)nc(-c3cccc(-c4ccc5ccccc5c4)c3)n2)c1 ; c1ccc(-c2ccc(-c3nc(-c4ccc(-c5ccc6ccccc6c5)cc4)nc(-c4ccc(-c5ccc6ccccc6c5)cc4)n3)cc2)cc1 ; c1ccc2c(c1)ccc1cc(-c3nc(-c4ccc5c(ccc6ccccc65)c4)nc(-c4ccc5c(ccc6ccccc65)c4)n3)ccc12]

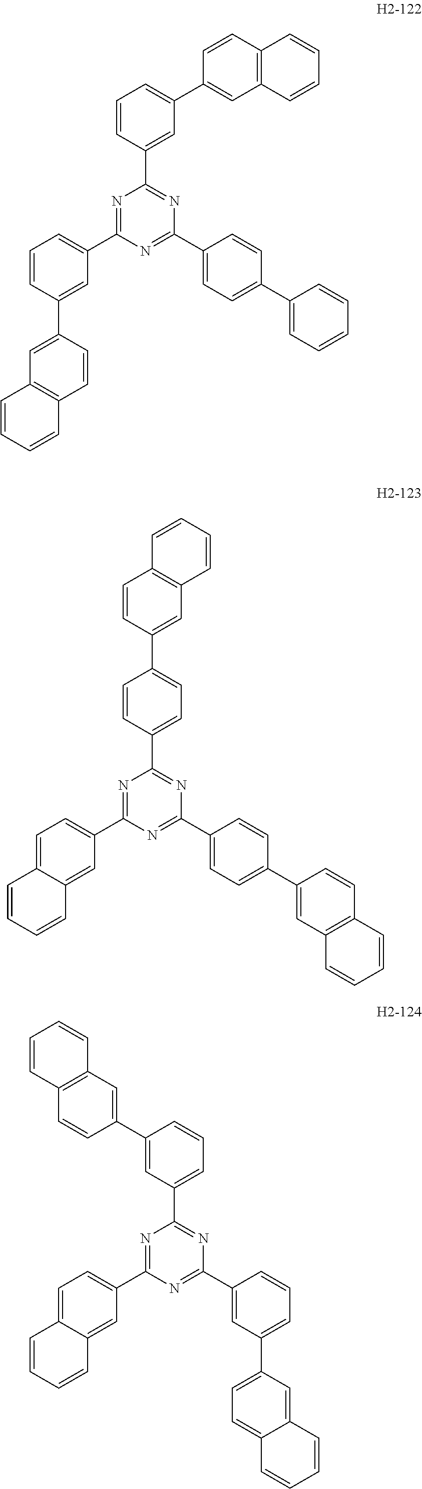 [SMILES: c1ccc(-c2ccc(-c3nc(-c4cccc(-c5ccc6ccccc6c5)c4)nc(-c4cccc(-c5ccc6ccccc6c5)c4)n3)cc2)cc1 ; c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1]

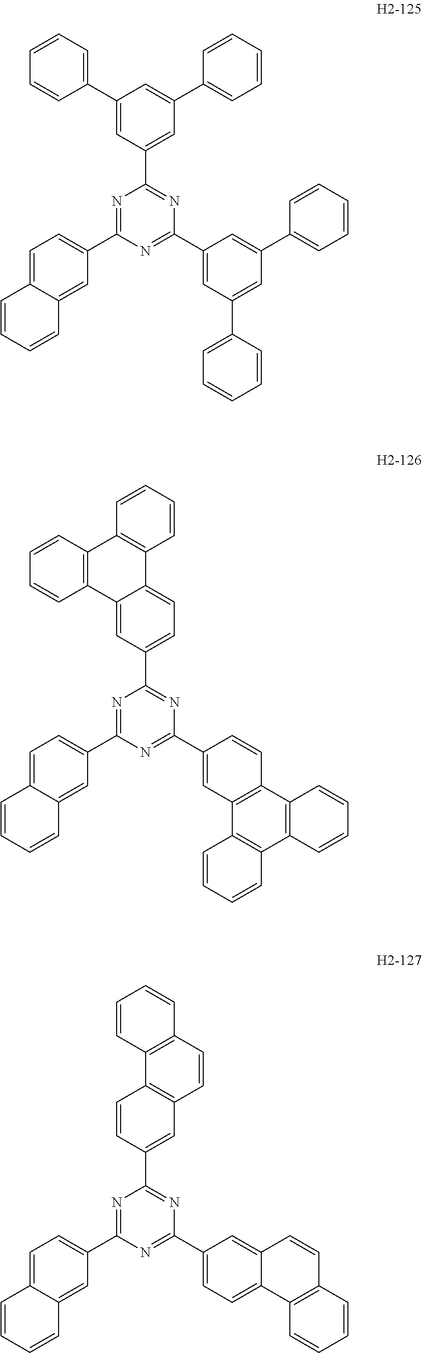 [SMILES: c1ccc(-c2cc(-c3ccccc3)cc(-c3nc(-c4cc(-c5ccccc5)cc(-c5ccccc5)c4)nc(-c4ccc5ccccc5c4)n3)c2)cc1 ; c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1]

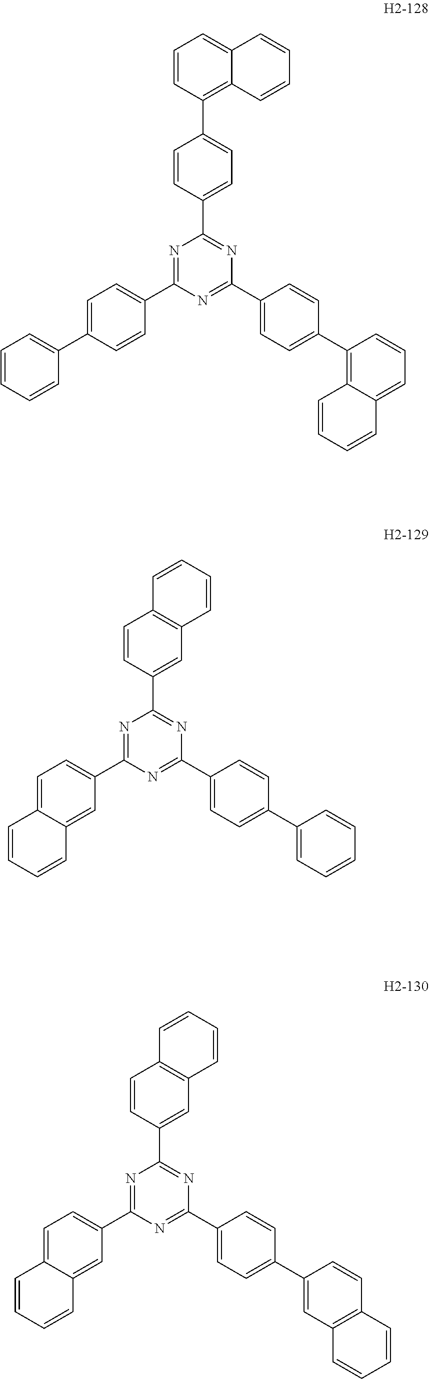 [SMILES: c1ccc(-c2ccc(-c3nc(-c4ccc(-c5cccc6ccccc56)cc4)nc(-c4ccc(-c5cccc6ccccc56)cc4)n3)cc2)cc1 ; c1ccc(-c2ccc(-c3nc(-c4ccc5ccccc5c4)nc(-c4ccc5ccccc5c4)n3)cc2)cc1 ; c1ccc2cc(-c3ccc(-c4nc(-c5ccc6ccccc6c5)nc(-c5ccc6ccccc6c5)n4)cc3)ccc2c1]

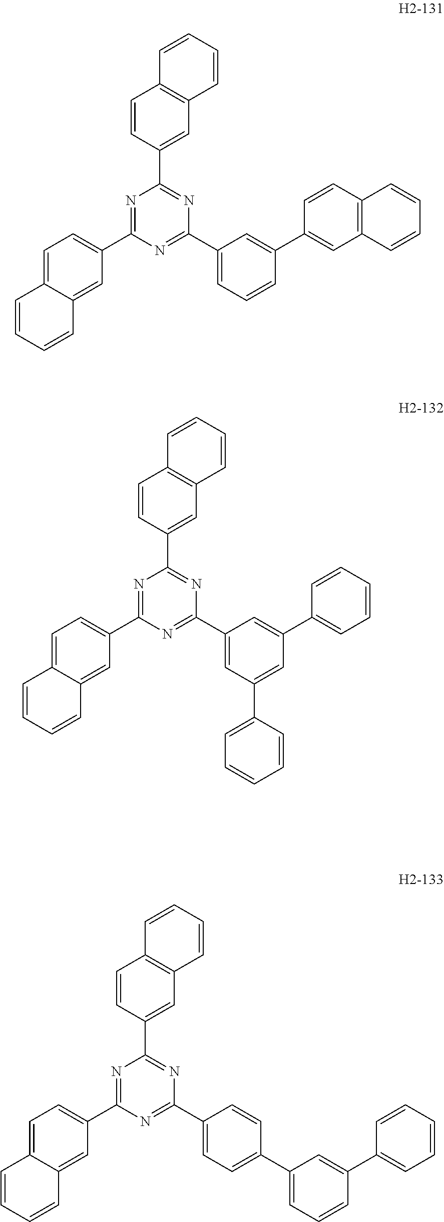 [SMILES: c1cc(-c2ccc3ccccc3c2)cc(-c2nc(-c3ccc4ccccc4c3)nc(-c3ccc4ccccc4c3)n2)c1 ; c1ccc(-c2cc(-c3ccccc3)cc(-c3nc(-c4ccc5ccccc5c4)nc(-c4ccc5ccccc5c4)n3)c2)cc1 ; c1ccc(-c2cccc(-c3ccc(-c4nc(-c5ccc6ccccc6c5)nc(-c5ccc6ccccc6c5)n4)cc3)c2)cc1]

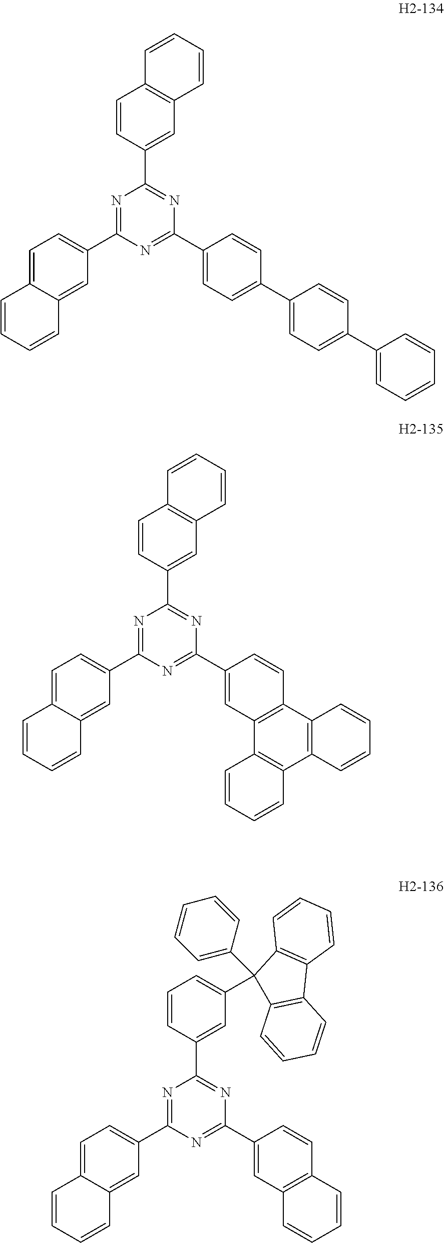 [SMILES: c1ccc(-c2ccc(-c3ccc(-c4nc(-c5ccc6ccccc6c5)nc(-c5ccc6ccccc6c5)n4)cc3)cc2)cc1 ; c1ccc2cc(-c3nc(-c4ccc5ccccc5c4)nc(-c4ccc5c6ccccc6c6ccccc6c5c4)n3)ccc2c1 ; c1ccc(C2(c3cccc(-c4nc(-c5ccc6ccccc6c5)nc(-c5ccc6ccccc6c5)n4)c3)c3ccccc3-c3ccccc32)cc1]

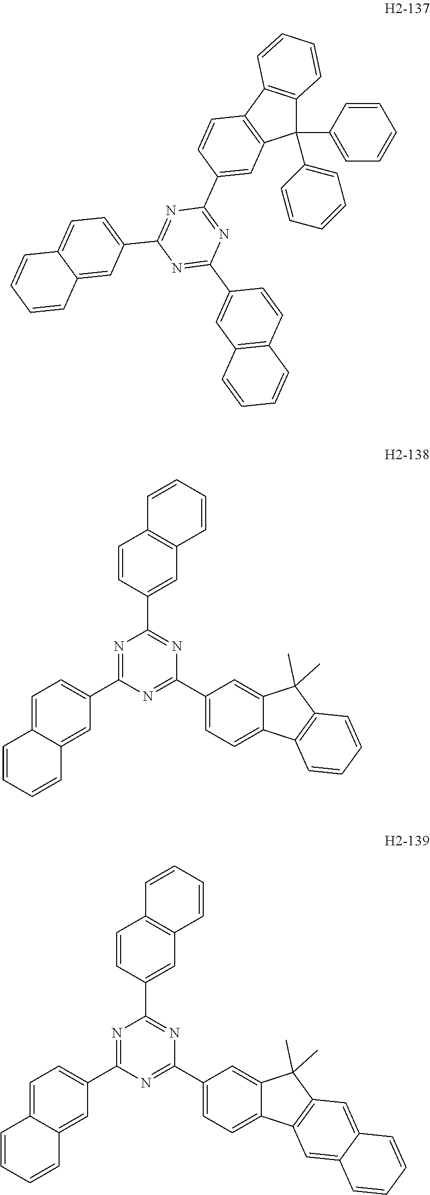 [SMILES: c1ccc(C2(c3ccccc3)c3ccccc3-c3ccc(-c4nc(-c5ccc6ccccc6c5)nc(-c5ccc6ccccc6c5)n4)cc32)cc1 ; CC1(C)c2ccccc2-c2ccc(-c3nc(-c4ccc5ccccc5c4)nc(-c4ccc5ccccc5c4)n3)cc21 ; CC1(C)c2cc(-c3nc(-c4ccc5ccccc5c4)nc(-c4ccc5ccccc5c4)n3)ccc2-c2cc3ccccc3cc21]

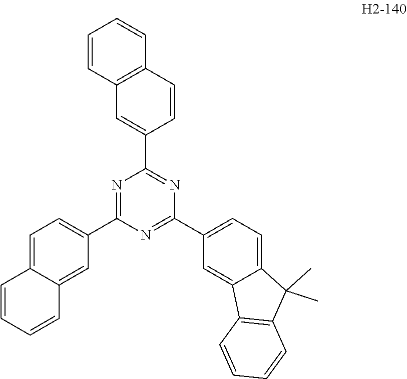 [SMILES: CC1(C)c2ccccc2-c2cc(-c3nc(-c4ccc5ccccc5c4)nc(-c4ccc5ccccc5c4)n3)ccc21]

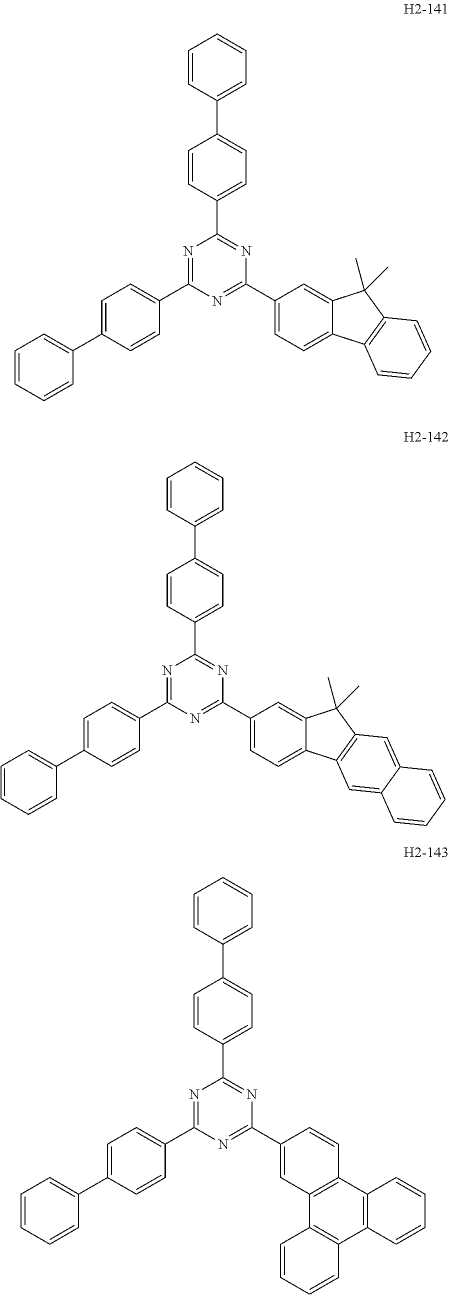 [SMILES: CC1(C)c2ccccc2-c2ccc(-c3nc(-c4ccc(-c5ccccc5)cc4)nc(-c4ccc(-c5ccccc5)cc4)n3)cc21 ; CC1(C)c2cc(-c3nc(-c4ccc(-c5ccccc5)cc4)nc(-c4ccc(-c5ccccc5)cc4)n3)ccc2-c2cc3ccccc3cc21 ; c1ccc(-c2ccc(-c3nc(-c4ccc(-c5ccccc5)cc4)nc(-c4ccc5c6ccccc6c6ccccc6c5c4)n3)cc2)cc1]

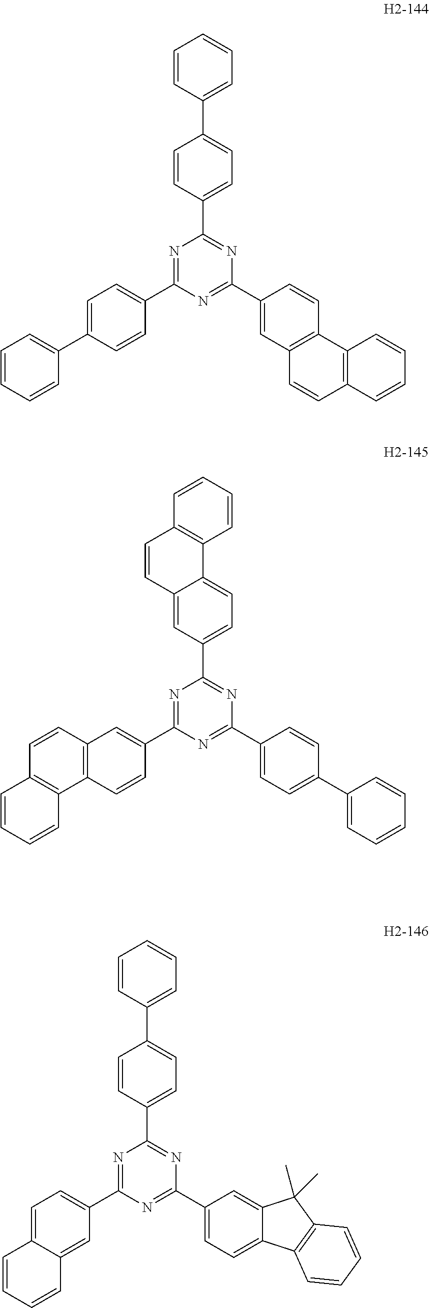 [SMILES: c1ccc(-c2ccc(-c3nc(-c4ccc(-c5ccccc5)cc4)nc(-c4ccc5c(ccc6ccccc65)c4)n3)cc2)cc1 ; c1ccc(-c2ccc(-c3nc(-c4ccc5c(ccc6ccccc65)c4)nc(-c4ccc5c(ccc6ccccc65)c4)n3)cc2)cc1 ; CC1(C)c2ccccc2-c2ccc(-c3nc(-c4ccc(-c5ccccc5)cc4)nc(-c4ccc5ccccc5c4)n3)cc21]

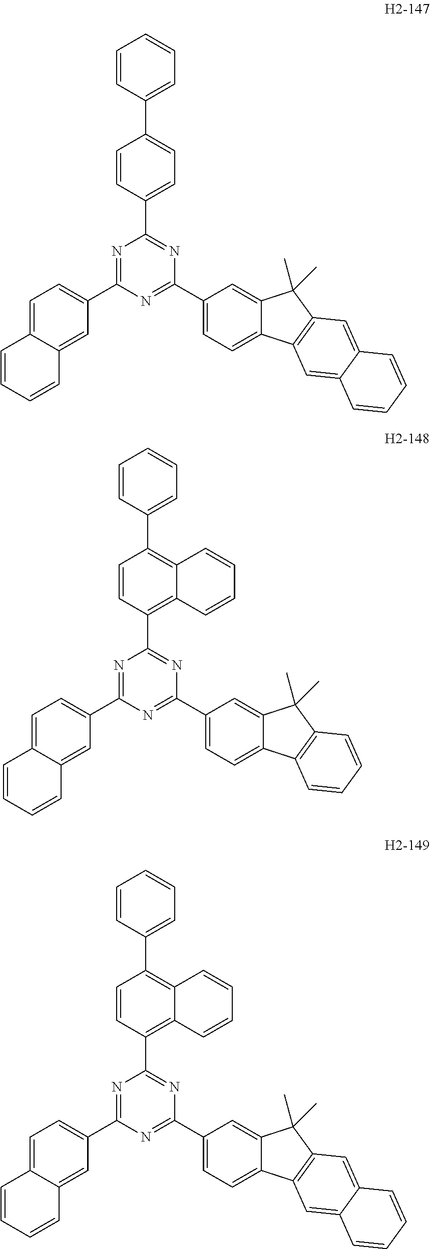 [SMILES: CC1(C)c2cc(-c3nc(-c4ccc(-c5ccccc5)cc4)nc(-c4ccc5ccccc5c4)n3)ccc2-c2cc3ccccc3cc21 ; CC1(C)c2ccccc2-c2ccc(-c3nc(-c4ccc5ccccc5c4)nc(-c4ccc(-c5ccccc5)c5ccccc45)n3)cc21 ; CC1(C)c2cc(-c3nc(-c4ccc5ccccc5c4)nc(-c4ccc(-c5ccccc5)c5ccccc45)n3)ccc2-c2cc3ccccc3cc21]

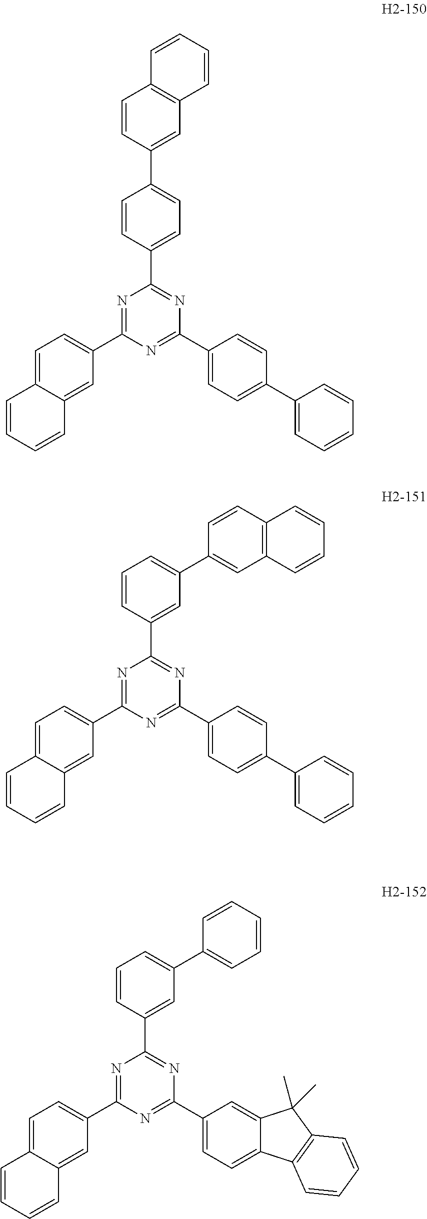 [SMILES: c1ccc(-c2ccc(-c3nc(-c4ccc(-c5ccc6ccccc6c5)cc4)nc(-c4ccc5ccccc5c4)n3)cc2)cc1 ; c1ccc(-c2ccc(-c3nc(-c4cccc(-c5ccc6ccccc6c5)c4)nc(-c4ccc5ccccc5c4)n3)cc2)cc1 ; CC1(C)c2ccccc2-c2ccc(-c3nc(-c4cccc(-c5ccccc5)c4)nc(-c4ccc5ccccc5c4)n3)cc21]

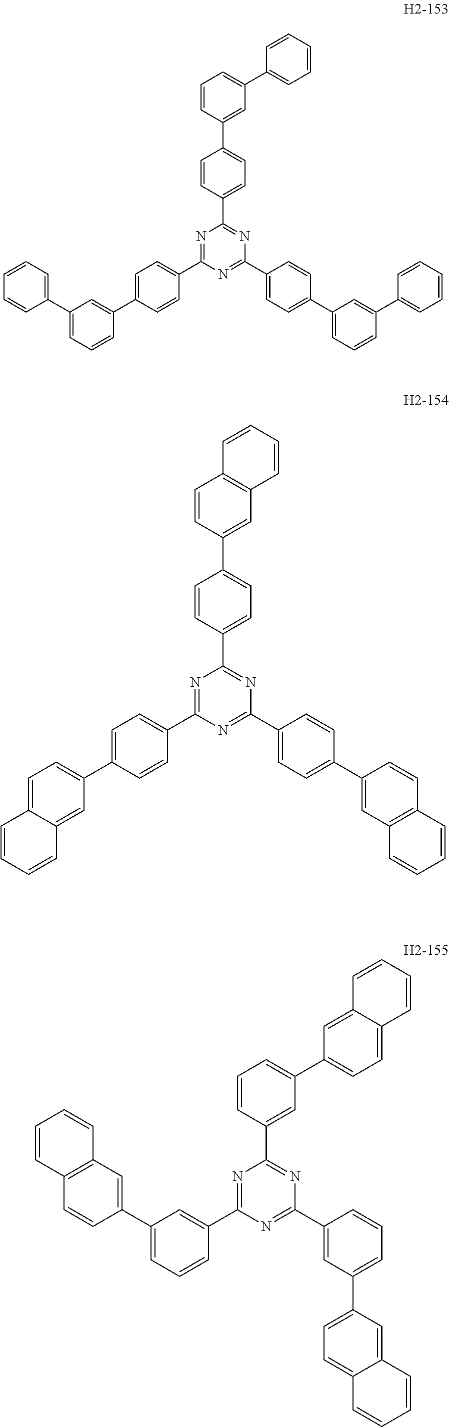 [SMILES: c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4nc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2nc(-c3cccc(-c4ccc5ccccc5c4)c3)nc(-c3cccc(-c4ccc5ccccc5c4)c3)n2)c1]

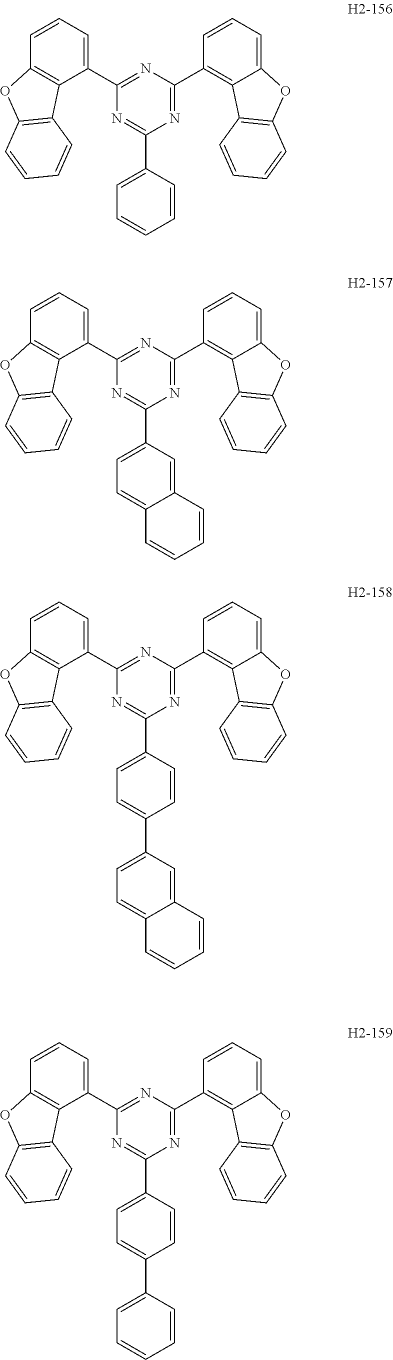 [SMILES: c1ccc(-c2nc(-c3cccc4oc5ccccc5c34)nc(-c3cccc4oc5ccccc5c34)n2)cc1 ; c1ccc2cc(-c3nc(-c4cccc5oc6ccccc6c45)nc(-c4cccc5oc6ccccc6c45)n3)ccc2c1 ; c1ccc2cc(-c3ccc(-c4nc(-c5cccc6oc7ccccc7c56)nc(-c5cccc6oc7ccccc7c56)n4)cc3)ccc2c1 ; c1ccc(-c2ccc(-c3nc(-c4cccc5oc6ccccc6c45)nc(-c4cccc5oc6ccccc6c45)n3)cc2)cc1]

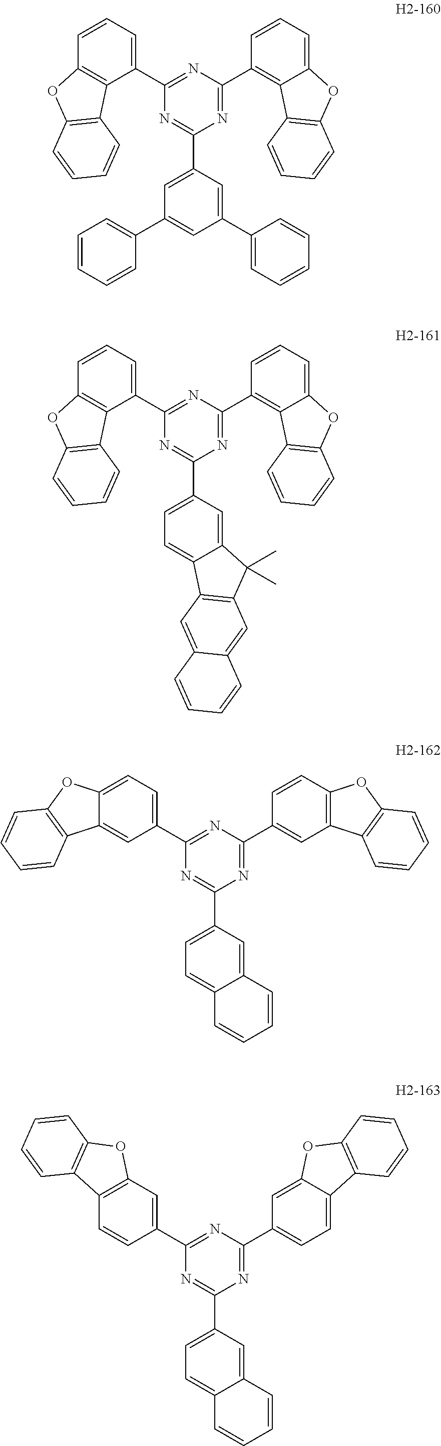 [SMILES: c1ccc(-c2cc(-c3ccccc3)cc(-c3nc(-c4cccc5oc6ccccc6c45)nc(-c4cccc5oc6ccccc6c45)n3)c2)cc1 ; CC1(C)c2cc(-c3nc(-c4cccc5oc6ccccc6c45)nc(-c4cccc5oc6ccccc6c45)n3)ccc2-c2cc3ccccc3cc21 ; c1ccc2cc(-c3nc(-c4ccc5oc6ccccc6c5c4)nc(-c4ccc5oc6ccccc6c5c4)n3)ccc2c1 ; c1ccc2cc(-c3nc(-c4ccc5c(c4)oc4ccccc45)nc(-c4ccc5c(c4)oc4ccccc45)n3)ccc2c1]

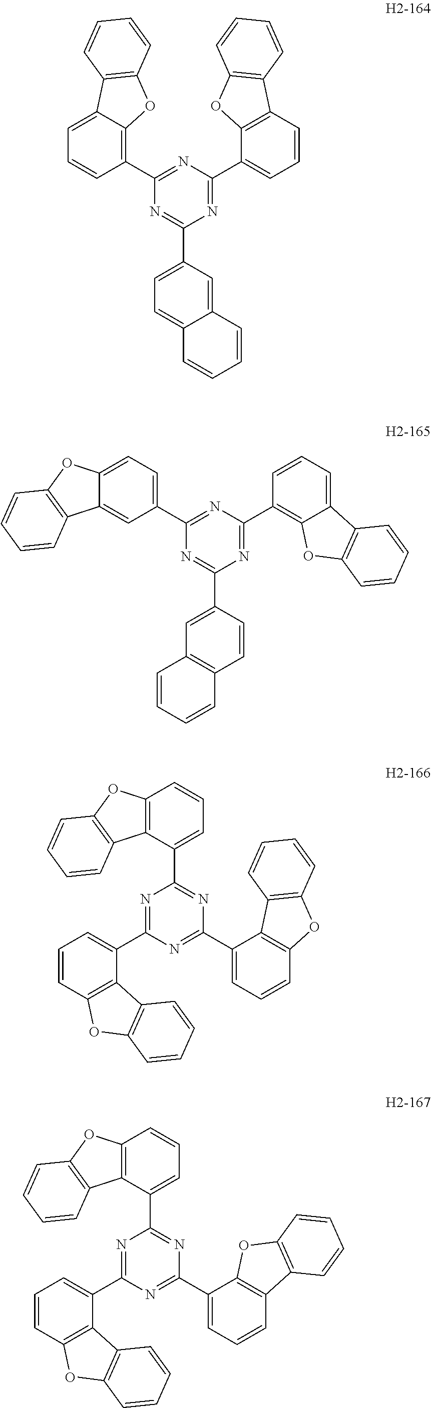 [SMILES: c1ccc2cc(-c3nc(-c4cccc5c4oc4ccccc45)nc(-c4cccc5c4oc4ccccc45)n3)ccc2c1 ; c1ccc2cc(-c3nc(-c4ccc5oc6ccccc6c5c4)nc(-c4cccc5c4oc4ccccc45)n3)ccc2c1 ; c1ccc2c(c1)oc1cccc(-c3nc(-c4cccc5oc6ccccc6c45)nc(-c4cccc5oc6ccccc6c45)n3)c12 ; c1ccc2c(c1)oc1c(-c3nc(-c4cccc5oc6ccccc6c45)nc(-c4cccc5oc6ccccc6c45)n3)cccc12]

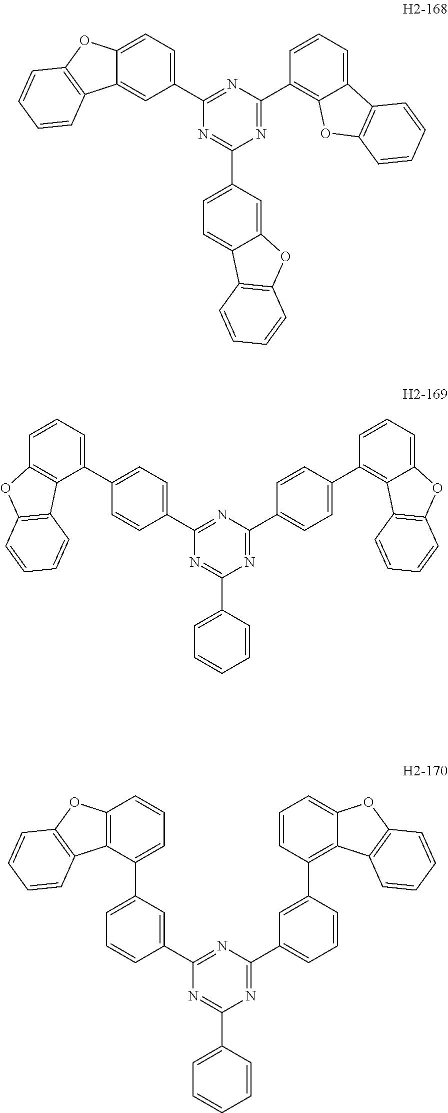 [SMILES: c1ccc2c(c1)oc1cc(-c3nc(-c4ccc5oc6ccccc6c5c4)nc(-c4cccc5c4oc4ccccc45)n3)ccc12 ; c1ccc(-c2nc(-c3ccc(-c4cccc5oc6ccccc6c45)cc3)nc(-c3ccc(-c4cccc5oc6ccccc6c45)cc3)n2)cc1 ; c1ccc(-c2nc(-c3cccc(-c4cccc5oc6ccccc6c45)c3)nc(-c3cccc(-c4cccc5oc6ccccc6c45)c3)n2)cc1]

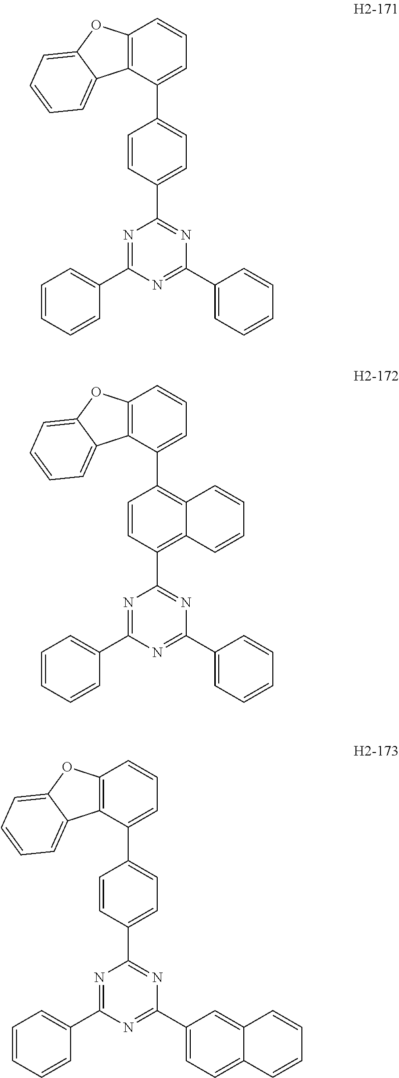 [SMILES: c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1]

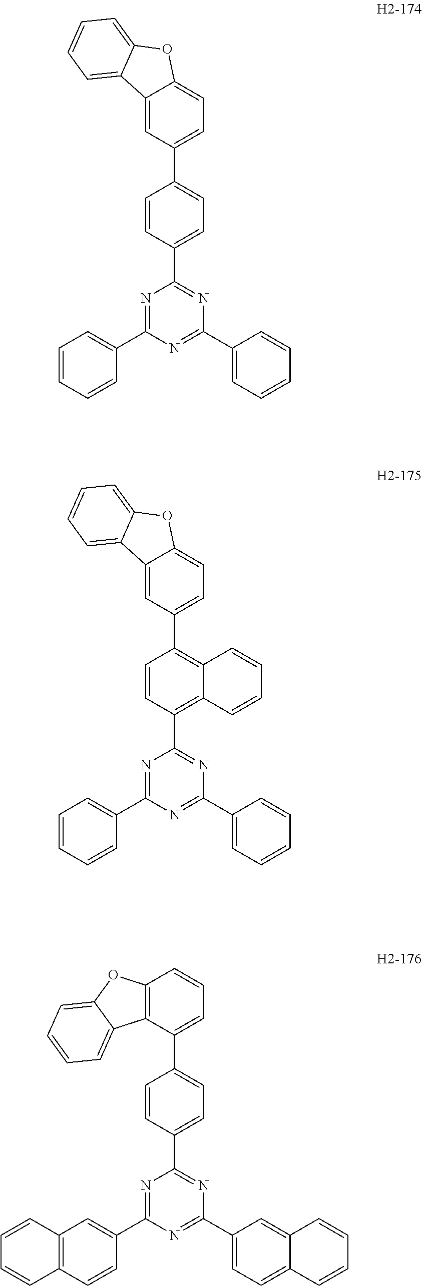 [SMILES: c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1 ; c1ccc2cc(-c3nc(-c4ccc(-c5cccc6oc7ccccc7c56)cc4)nc(-c4ccc5ccccc5c4)n3)ccc2c1]

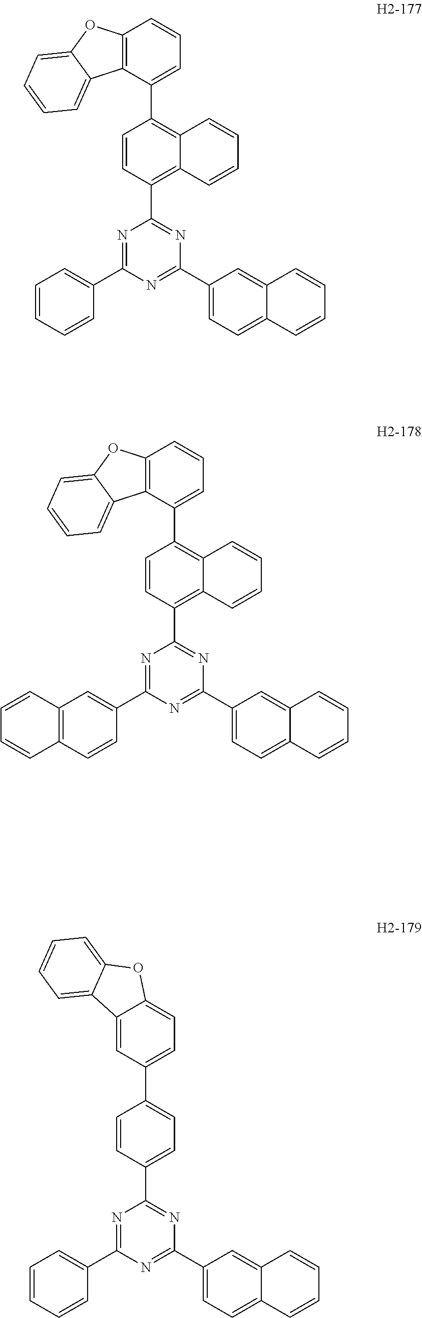 [SMILES: c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1]

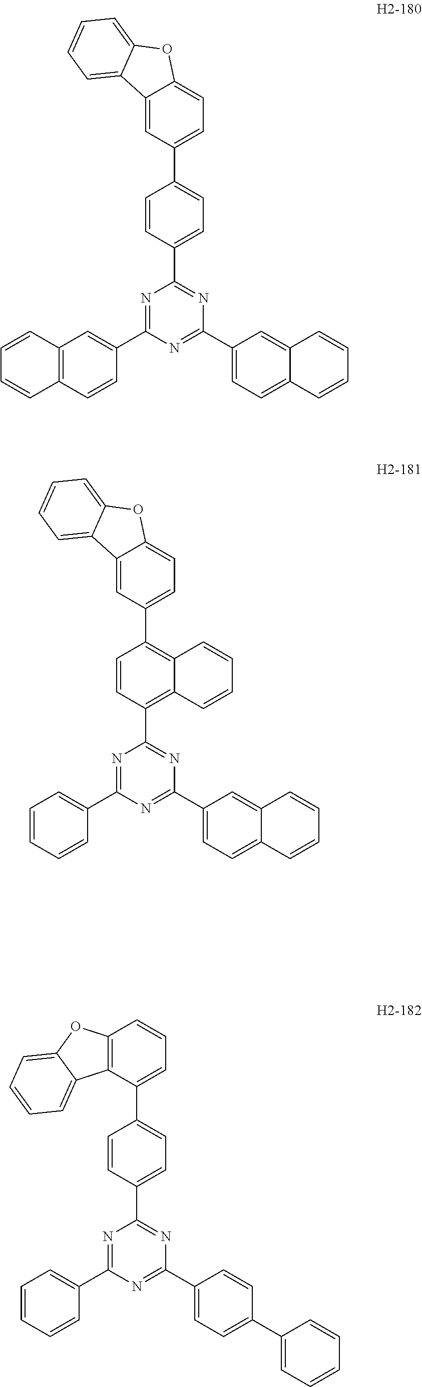 [SMILES: c1ccc2cc(-c3nc(-c4ccc(-c5ccc6oc7ccccc7c6c5)cc4)nc(-c4ccc5ccccc5c4)n3)ccc2c1 ; c1ccc(-c2nc(-c3ccc4ccccc4c3)nc(-c3ccc(-c4ccc5oc6ccccc6c5c4)c4ccccc34)n2)cc1 ; c1ccc(-c2ccc(-c3nc(-c4ccccc4)nc(-c4ccc(-c5cccc6oc7ccccc7c56)cc4)n3)cc2)cc1]

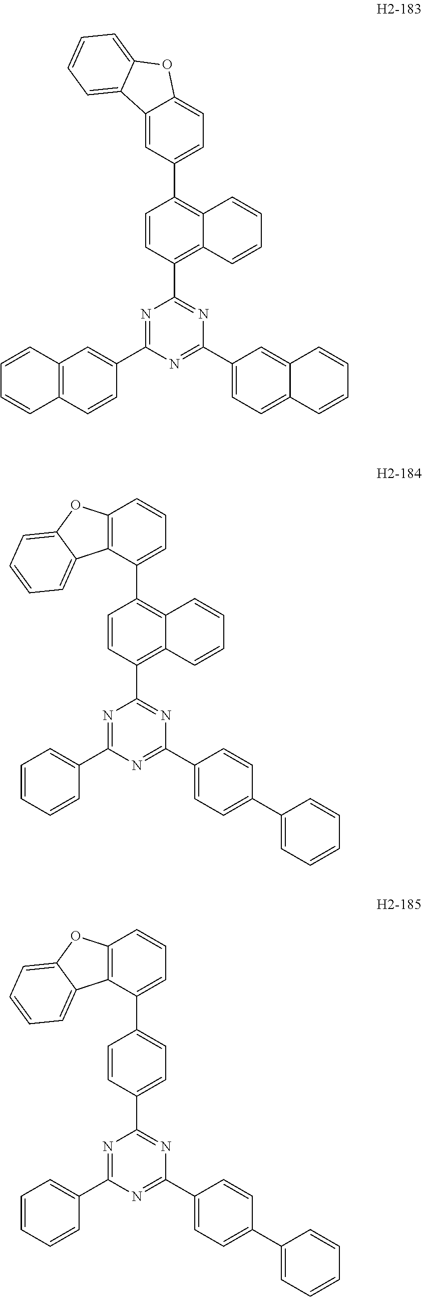 [SMILES: c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc(-c5cccc6oc7ccccc7c56)cc4)n3)cc2)cc1]

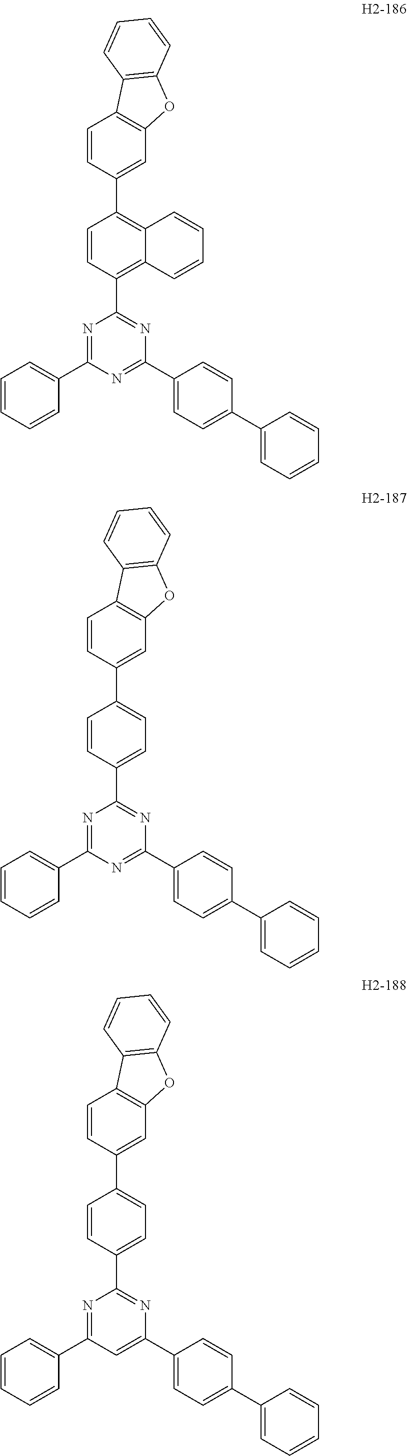 [SMILES: c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3cc(-c4ccccc4)nc(-c4ccc(-c5ccc6c(c5)oc5ccccc56)cc4)n3)cc2)cc1]

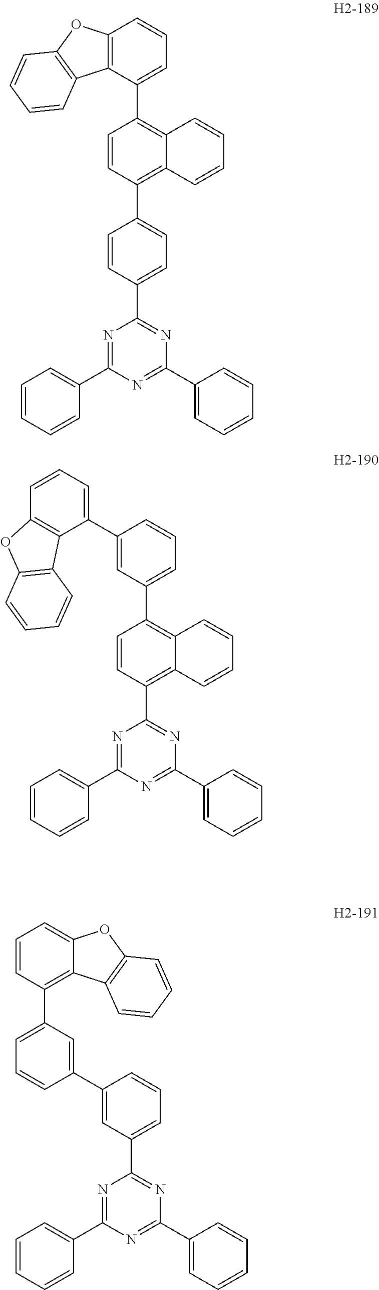

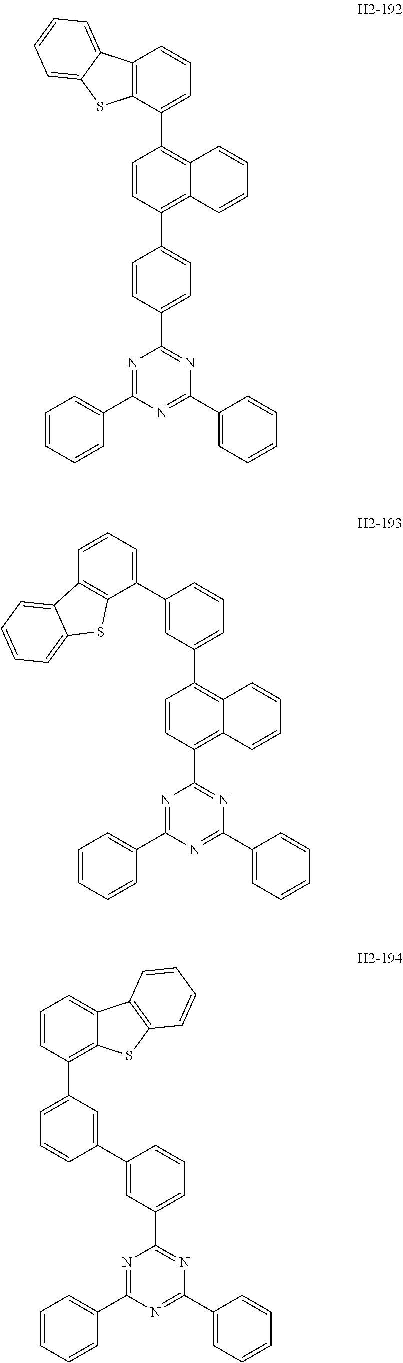 [SMILES: c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1 ; c1ccc(-c2nc(-c3ccccc3)nc(-c3cccc(-c4cccc(-c5cccc6c5sc5ccccc56)c4)c3)n2)cc1]

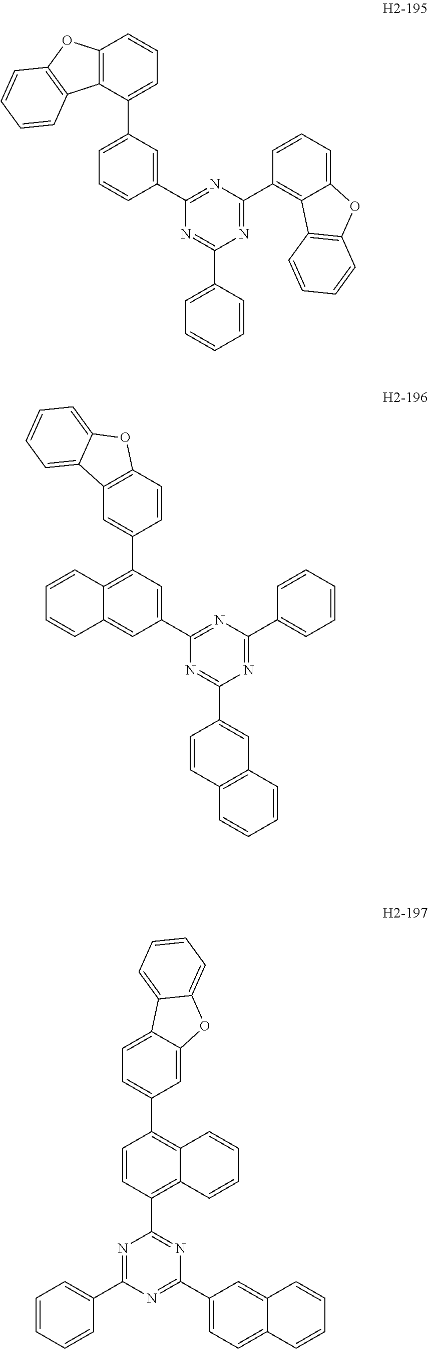 [SMILES: c1ccc(-c2nc(-c3cccc(-c4cccc5oc6ccccc6c45)c3)nc(-c3cccc4oc5ccccc5c34)n2)cc1 ; c1ccc(-c2nc(-c3ccc4ccccc4c3)nc(-c3cc(-c4ccc5oc6ccccc6c5c4)c4ccccc4c3)n2)cc1 ; c1ccc(-c2nc(-c3ccc4ccccc4c3)nc(-c3ccc(-c4ccc5c(c4)oc4ccccc45)c4ccccc34)n2)cc1]

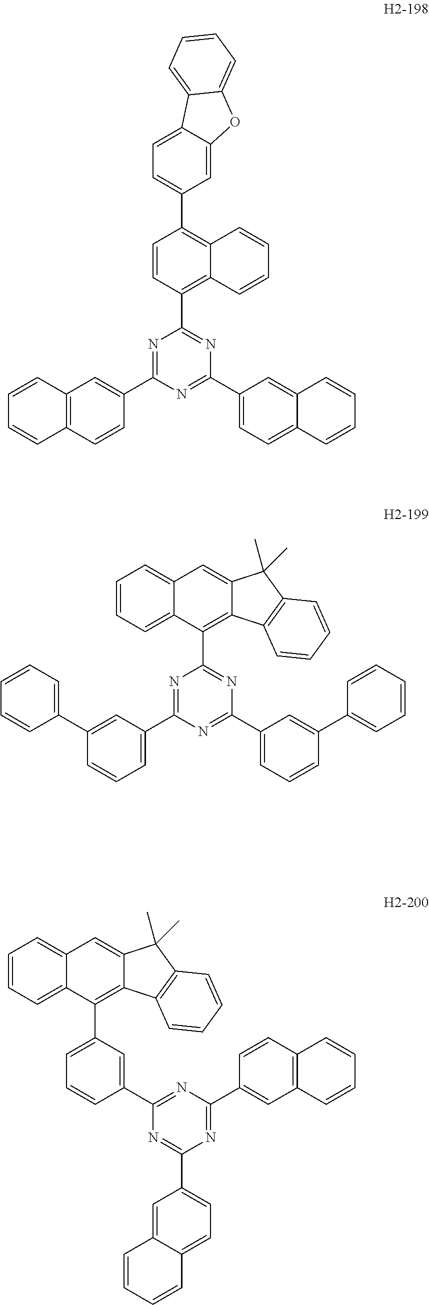 [SMILES: c1ccc2cc(-c3nc(-c4ccc5ccccc5c4)nc(-c4ccc(-c5ccc6c(c5)oc5ccccc56)c5ccccc45)n3)ccc2c1 ; CC1(C)c2ccccc2-c2c1cc1ccccc1c2-c1nc(-c2cccc(-c3ccccc3)c2)nc(-c2cccc(-c3ccccc3)c2)n1 ; CC1(C)c2ccccc2-c2c1cc1ccccc1c2-c1cccc(-c2nc(-c3ccc4ccccc4c3)nc(-c3ccc4ccccc4c3)n2)c1]

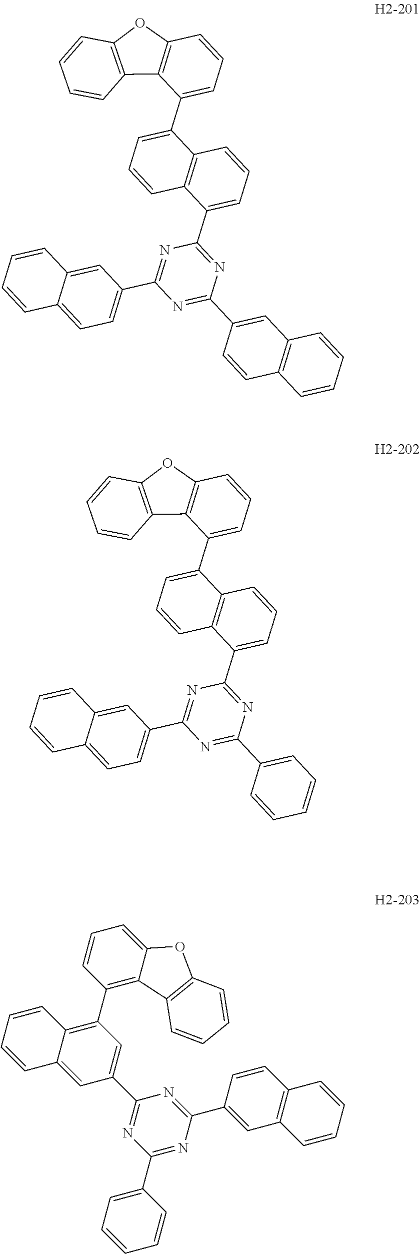 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1]

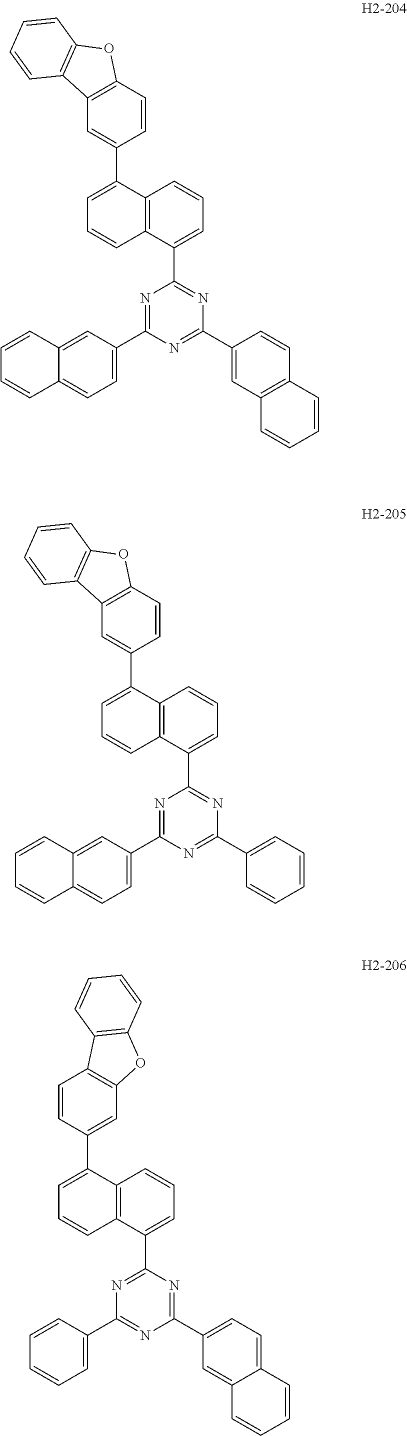 [SMILES: c1ccc2cc(-c3nc(-c4ccc5ccccc5c4)nc(-c4cccc5c(-c6ccc7oc8ccccc8c7c6)cccc45)n3)ccc2c1 ; c1ccc(-c2nc(-c3ccc4ccccc4c3)nc(-c3cccc4c(-c5ccc6oc7ccccc7c6c5)cccc34)n2)cc1 ; c1ccc(-c2nc(-c3ccc4ccccc4c3)nc(-c3cccc4c(-c5ccc6c(c5)oc5ccccc56)cccc34)n2)cc1]

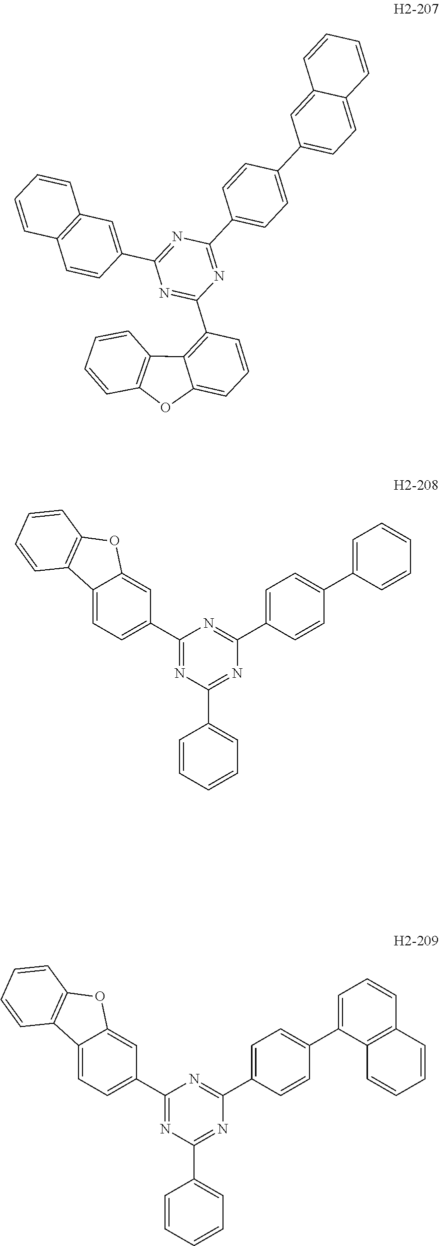 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1]

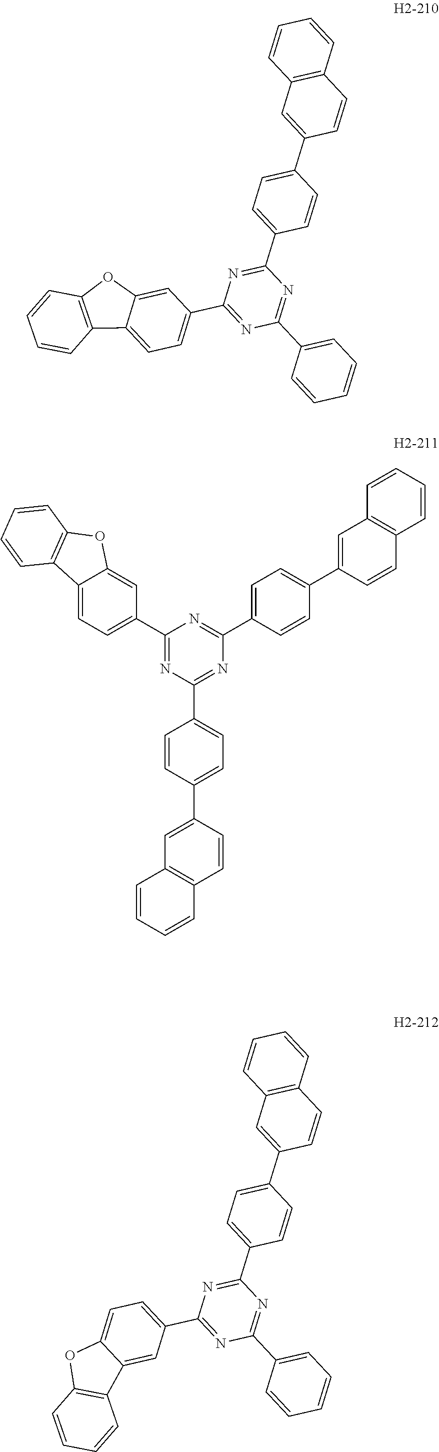 [SMILES: c1ccc(-c2nc(-c3ccc(-c4ccc5ccccc5c4)cc3)nc(-c3ccc4c(c3)oc3ccccc34)n2)cc1 ; c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1]

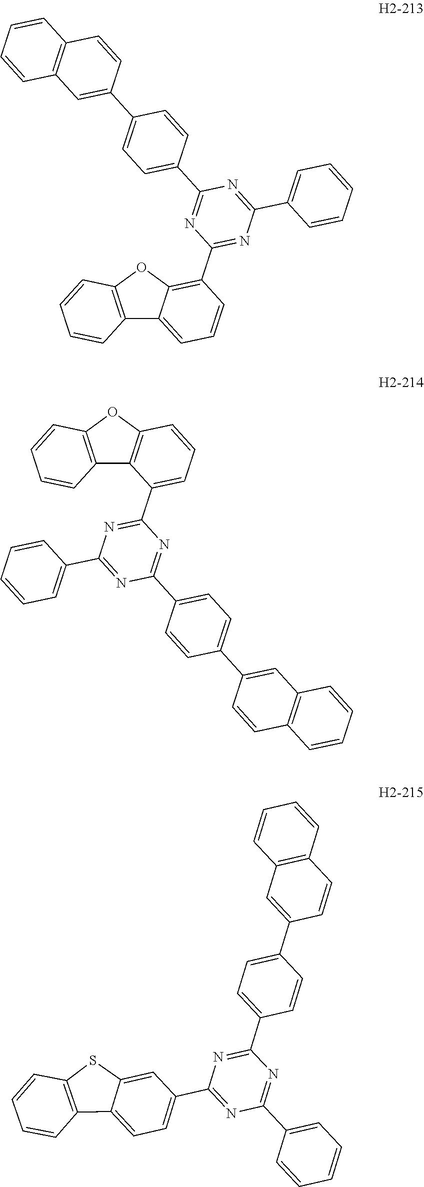 [SMILES: c1ccc(-c2nc(-c3ccc(-c4ccc5ccccc5c4)cc3)nc(-c3cccc4c3oc3ccccc34)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc4c(c3)sc3ccccc34)n2)cc1]

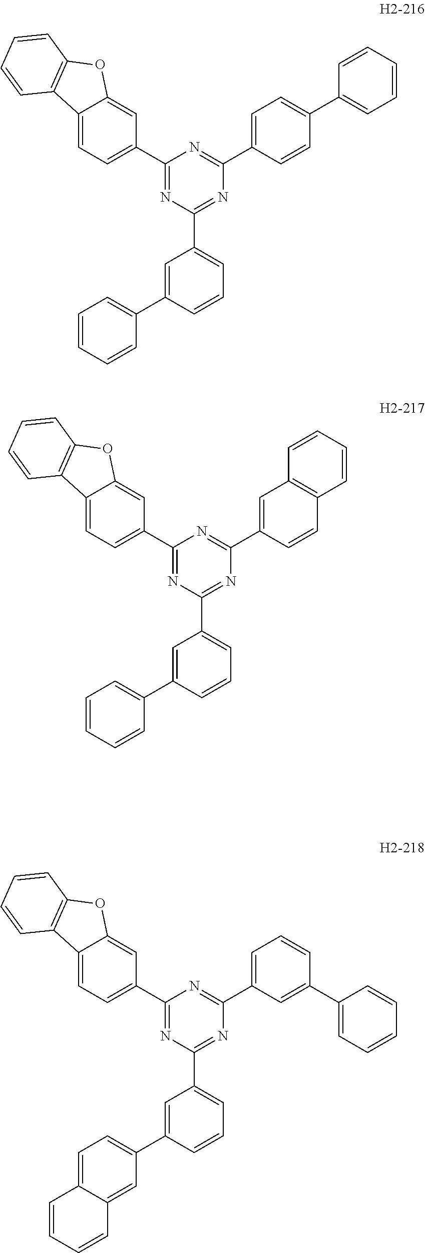 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1]

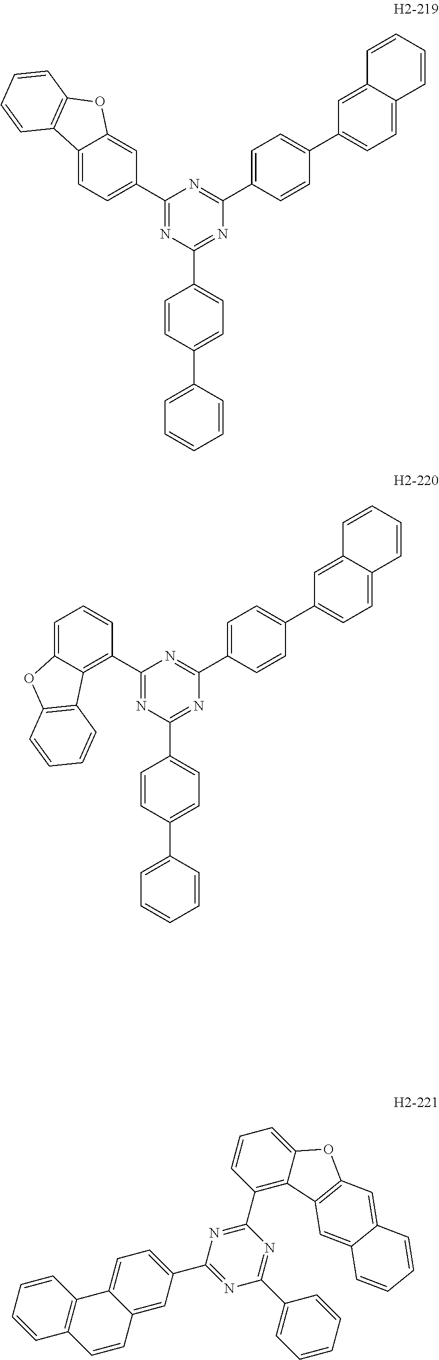 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2nc(-c3ccc4c(ccc5ccccc54)c3)nc(-c3cccc4oc5cc6ccccc6cc5c34)n2)cc1]

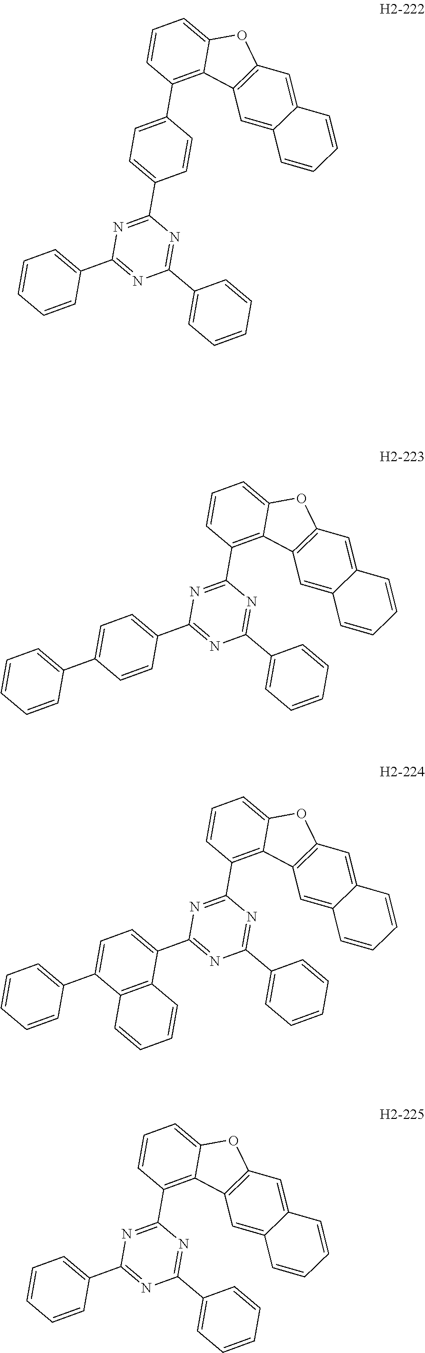 [SMILES: c1ccc(-c2nc(-c3ccccc3)nc(-c3ccc(-c4cccc5oc6cc7ccccc7cc6c45)cc3)n2)cc1 ; c1ccc(-c2ccc(-c3nc(-c4ccccc4)nc(-c4cccc5oc6cc7ccccc7cc6c45)n3)cc2)cc1 ; c1ccc(-c2nc(-c3ccc(-c4ccccc4)c4ccccc34)nc(-c3cccc4oc5cc6ccccc6cc5c34)n2)cc1 ; c1ccc(-c2nc(-c3ccccc3)nc(-c3cccc4oc5cc6ccccc6cc5c34)n2)cc1]

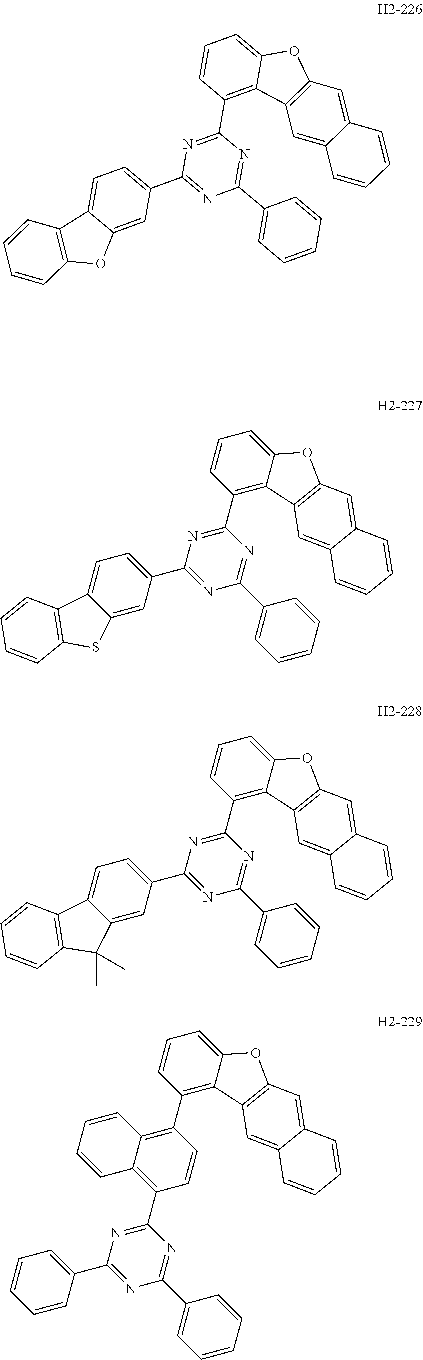 [SMILES: c1ccc(-c2nc(-c3ccc4c(c3)oc3ccccc34)nc(-c3cccc4oc5cc6ccccc6cc5c34)n2)cc1 ; c1ccc(-c2nc(-c3ccc4c(c3)sc3ccccc34)nc(-c3cccc4oc5cc6ccccc6cc5c34)n2)cc1 ; CC1(C)c2ccccc2-c2ccc(-c3nc(-c4ccccc4)nc(-c4cccc5oc6cc7ccccc7cc6c45)n3)cc21 ; c1ccc(-c2nc(-c3ccccc3)nc(-c3ccc(-c4cccc5oc6cc7ccccc7cc6c45)c4ccccc34)n2)cc1]

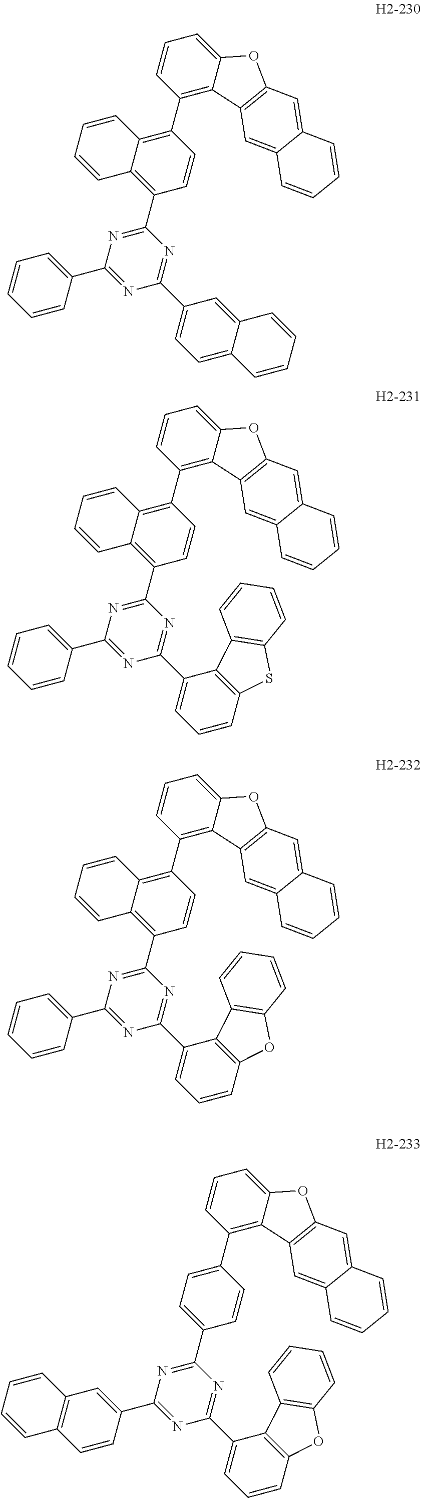

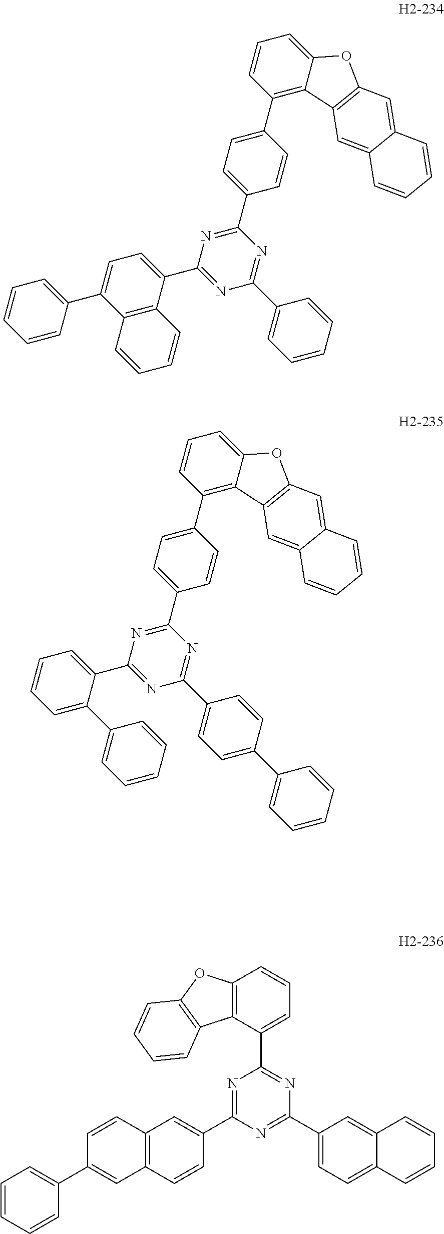 [SMILES: c1ccc(-c2nc(-c3ccc(-c4cccc5oc6cc7ccccc7cc6c45)cc3)nc(-c3ccc(-c4ccccc4)c4ccccc34)n2)cc1 ; c1ccc(-c2ccc(-c3nc(-c4ccc(-c5cccc6oc7cc8ccccc8cc7c56)cc4)nc(-c4ccccc4-c4ccccc4)n3)cc2)cc1 ; c1ccc(-c2ccc3cc(-c4nc(-c5ccc6ccccc6c5)nc(-c5cccc6oc7ccccc7c56)n4)ccc3c2)cc1]

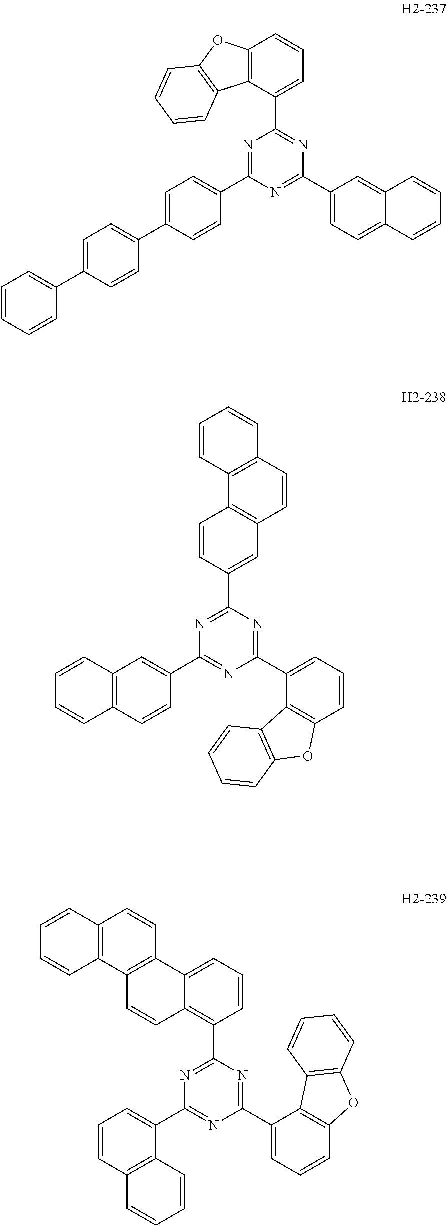 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc2cc(-c3nc(-c4ccc5c(ccc6ccccc65)c4)nc(-c4cccc5oc6ccccc6c45)n3)ccc2c1 ; c1ccc2c(-c3nc(-c4cccc5c4ccc4c6ccccc6ccc54)nc(-c4cccc5oc6ccccc6c45)n3)cccc2c1]

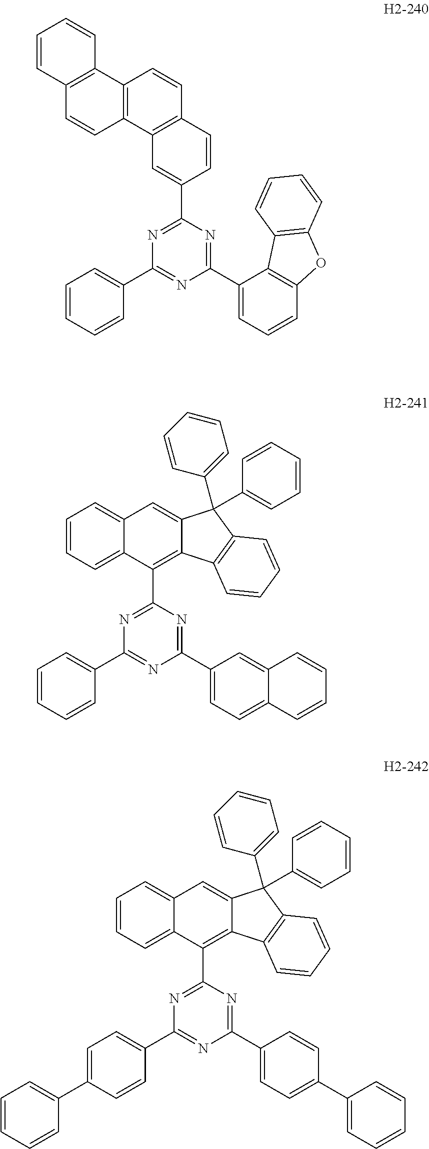 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccccc4c3)nc(-c3c4c(cc5ccccc35)C(c3ccccc3)(c3ccccc3)c3ccccc3-4)n2)cc1 ; c1ccc(-c2ccc(-c3nc(-c4ccc(-c5ccccc5)cc4)nc(-c4c5c(cc6ccccc46)C(c4ccccc4)(c4ccccc4)c4ccccc4-5)n3)cc2)cc1]

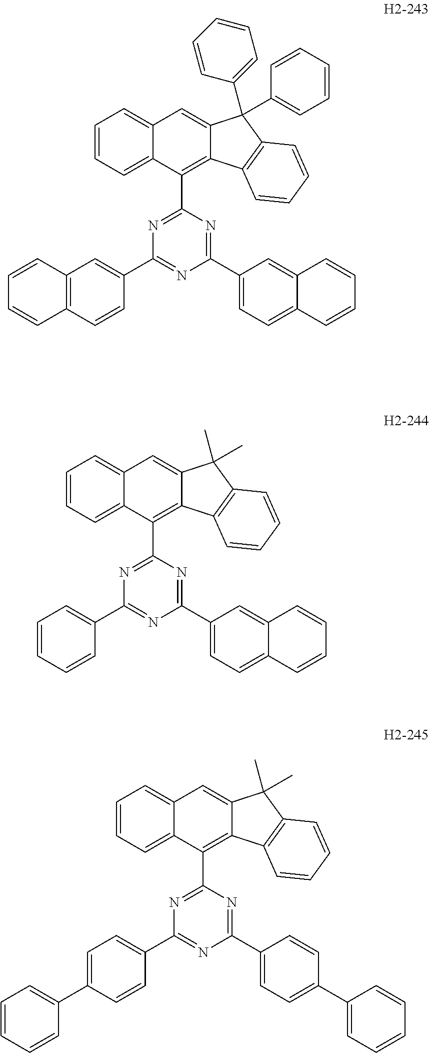 [SMILES: c1ccc(C2(c3ccccc3)c3ccccc3-c3c2cc2ccccc2c3-c2nc(-c3ccc4ccccc4c3)nc(-c3ccc4ccccc4c3)n2)cc1 ; CC1(C)c2ccccc2-c2c1cc1ccccc1c2-c1nc(-c2ccccc2)nc(-c2ccc3ccccc3c2)n1 ; CC1(C)c2ccccc2-c2c1cc1ccccc1c2-c1nc(-c2ccc(-c3ccccc3)cc2)nc(-c2ccc(-c3ccccc3)cc2)n1]

The combination of at least one of Compounds C-1 to C-196 and at least one of Compounds H2-1 to H2-245 may be used in an organic electroluminescent device.

The compound represented by formula 1 according to the present disclosure may be produced by referring to the following reaction schemes 1 to 3, but is not limited thereto.

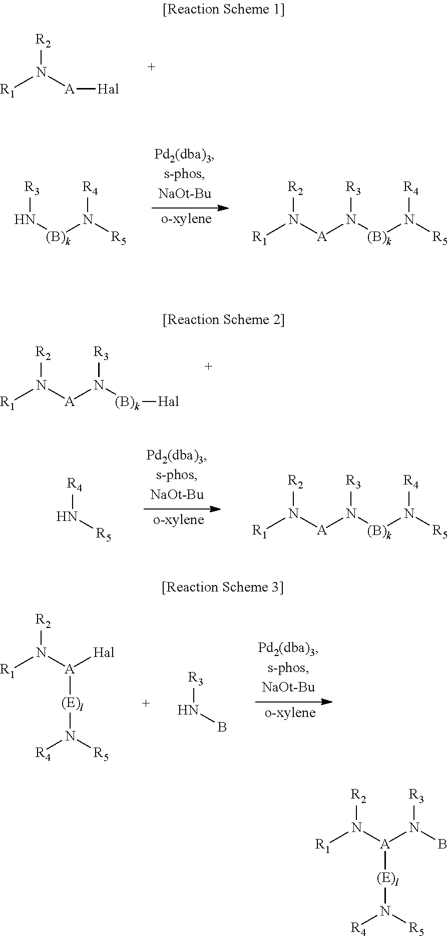

In reaction schemes 1 to 3, R₁ to R₅, A, B, E, k and l are as defined in formula 1, and Hal is Br, CI or I.

The compound represented by formula 2 according to the present disclosure may be produced by a synthetic method known to one skilled in the art, and in particular by using the synthetic methods disclosed in a number of patent literatures, for example, by referring to the methods disclosed in Korean Patent Application Laying-Open No. 2021-0124018, and Korean Patent Application Laying-Open No. 2021-0098316, etc., but is not limited thereto.

Although illustrative synthesis examples of the compounds represented by formulas 1 and 2 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN1 substitution reaction, an SN2 substitution reaction, and a Phosphine-mediated reductive cyclization reaction, etc., and the reactions above proceed even when substituents which are defined in formulas 1 and 2 above, but are not specified in the specific synthesis examples, are bonded.

In addition, the deuterated compounds of formula 1 may be prepared in a similar manner by using deuterated precursor materials, or more generally may be prepared by treating the non-deuterated compound with a deuterated solvent or D6-benzene in the presence of an H/D exchange catalyst such as a Lewis acid, e.g., aluminum trichloride or ethyl aluminum chloride. In addition, the degree of deuteration can be controlled by changing the reaction conditions such as the reaction temperature. For example, the number of deuterium in formula 1 can be controlled by adjusting the reaction temperature and time, the equivalent of the acid, etc.

The present disclosure provides an organic electroluminescent device comprising an anode, a cathode, and at least one light-emitting layer between the anode and cathode in which the at least one light-emitting layer comprises a plurality of host materials according to the present disclosure. The first host material and the second host material according to the present disclosure may be comprised in one light-emitting layer, or may be respectively comprised in different light-emitting layers. In the plurality of host materials of the present disclosure, the ratio of the compound represented by formula 1 and the compound represented by formula 2 is about 1:99 to about 99:1, preferably about 10:90 to about 90:10, more preferably about 30:70 to about 70:30. In addition, the compound represented by formula 1 and the compound represented by formula 2 in a desired ratio may be combined by mixing them in a shaker, by dissolving them in a glass tube by heat, or by dissolving them in a solvent, etc.

According to one embodiment of the present disclosure, the doping concentration of the dopant compound with respect to the host compound in the light-emitting layer may be less than 20 wt %. The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the group consisting of the metallated complex compounds of iridium (Ir), osmium (Os), copper (Cu), and platinum (Pt), more preferably selected from the group consisting of ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may be a compound represented by the following formula 101, but is not limited thereto.

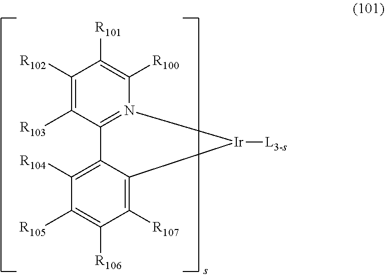

In formula 101,
L is selected from the following structures 1 to 3:

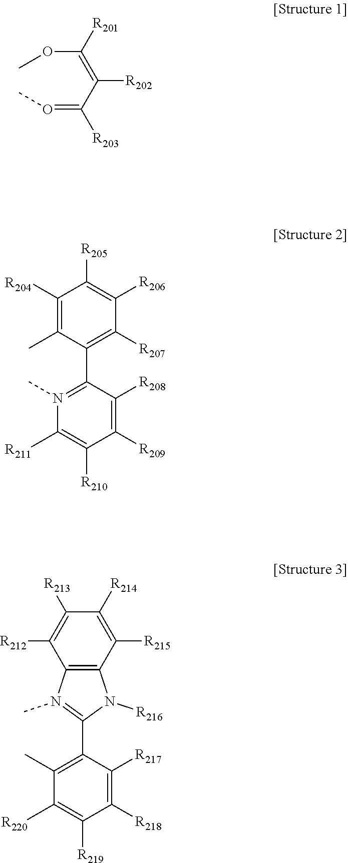

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

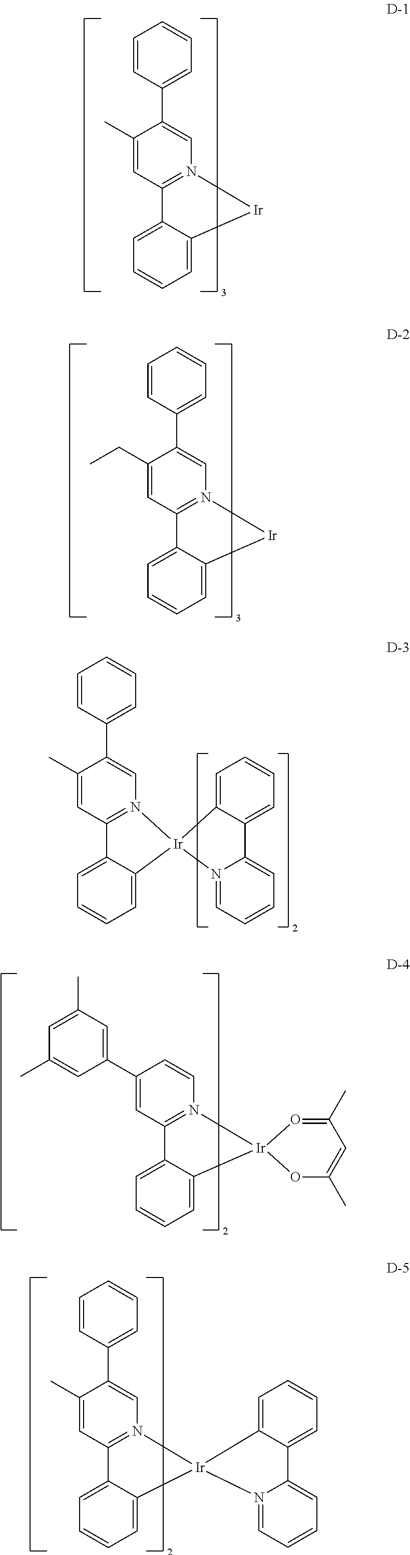

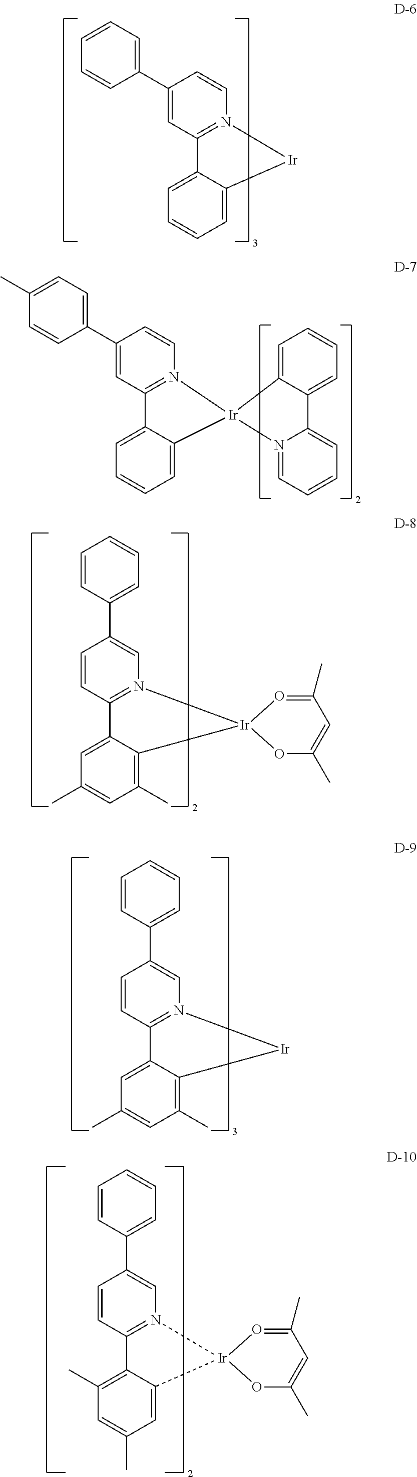

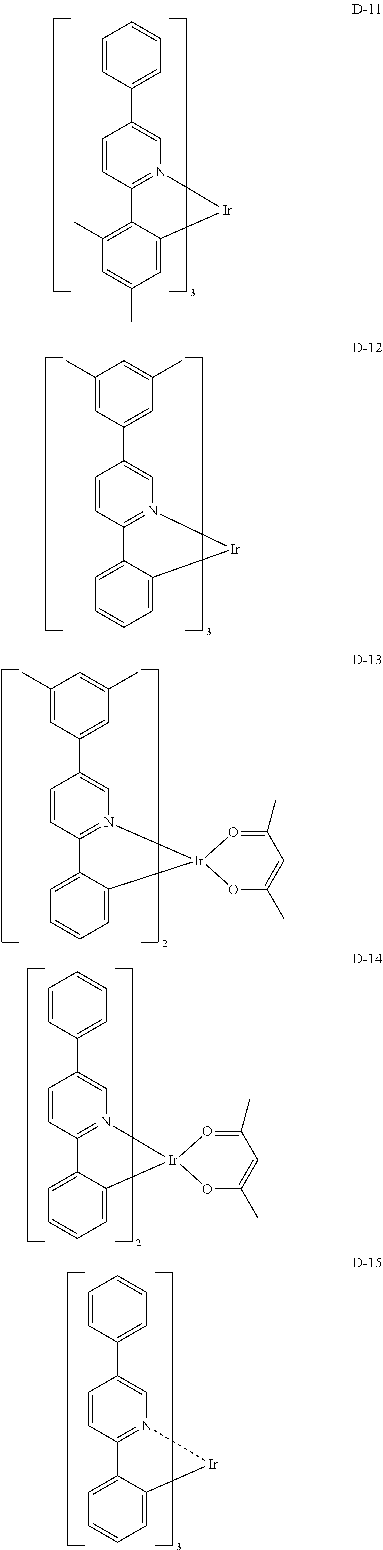

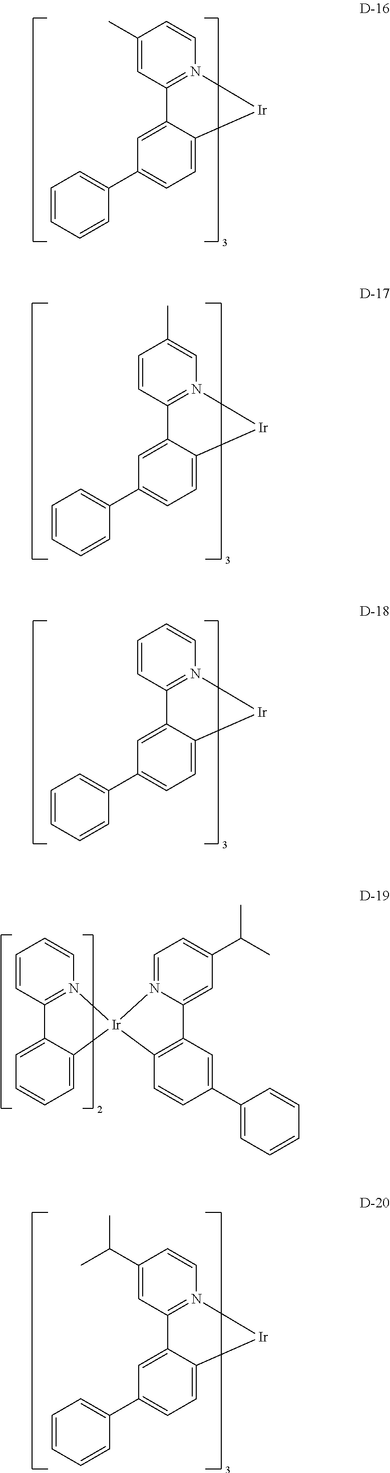

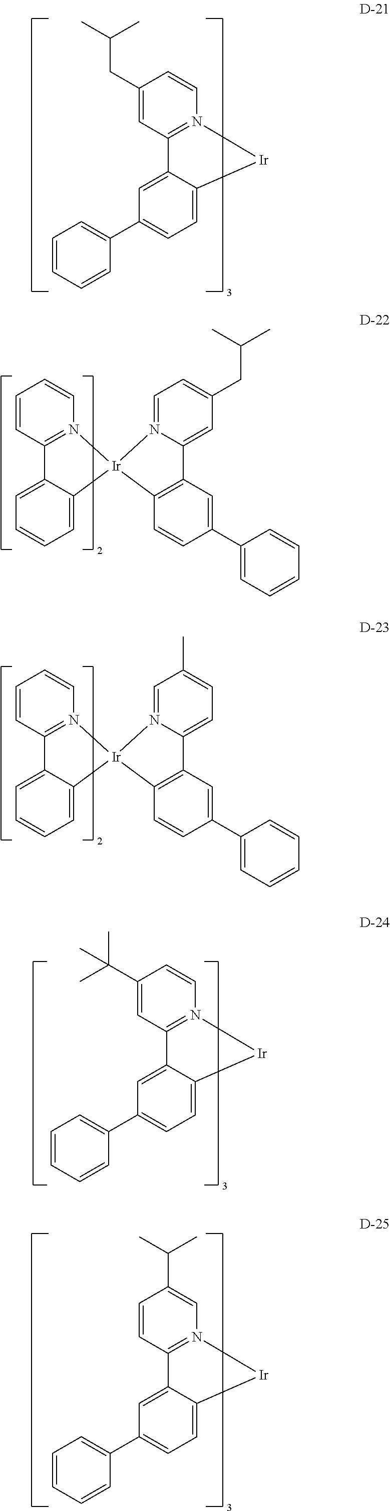

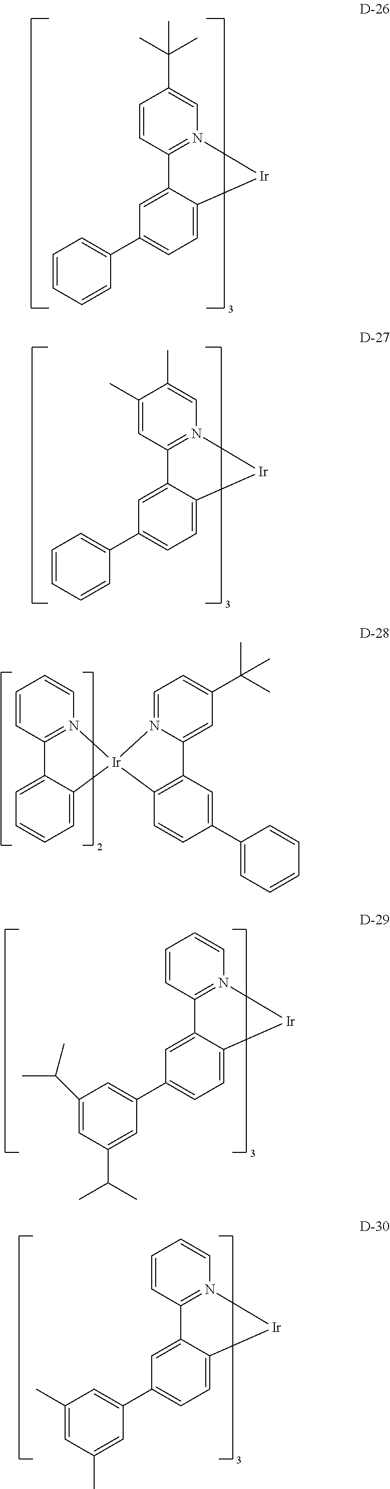

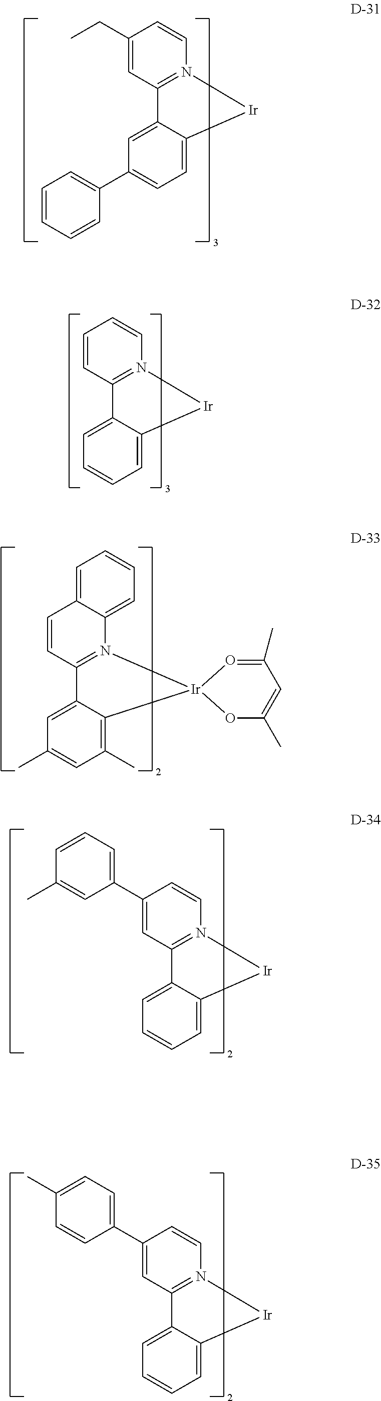

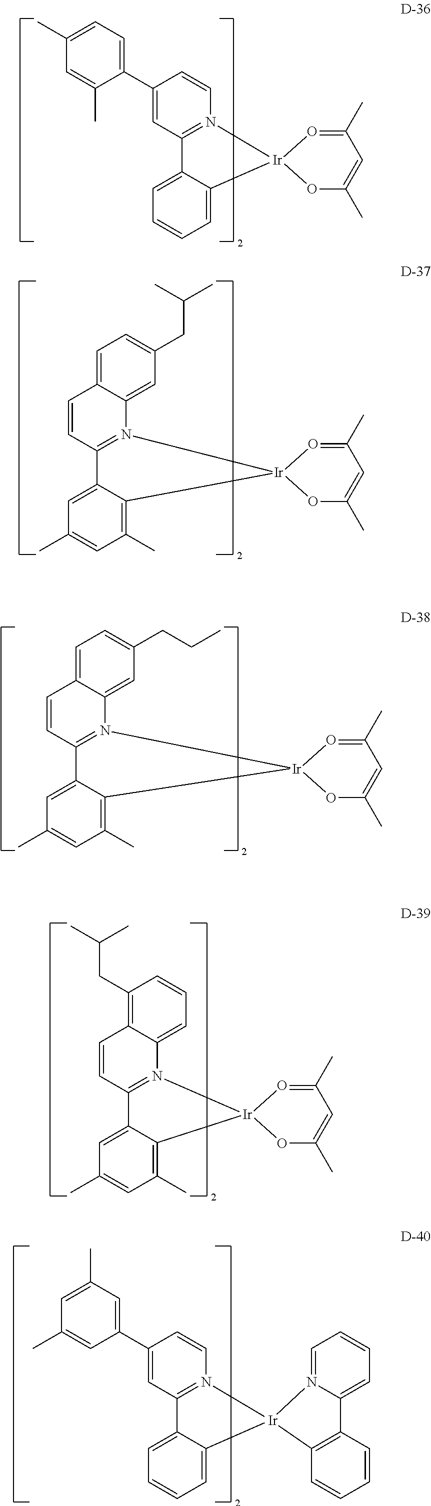

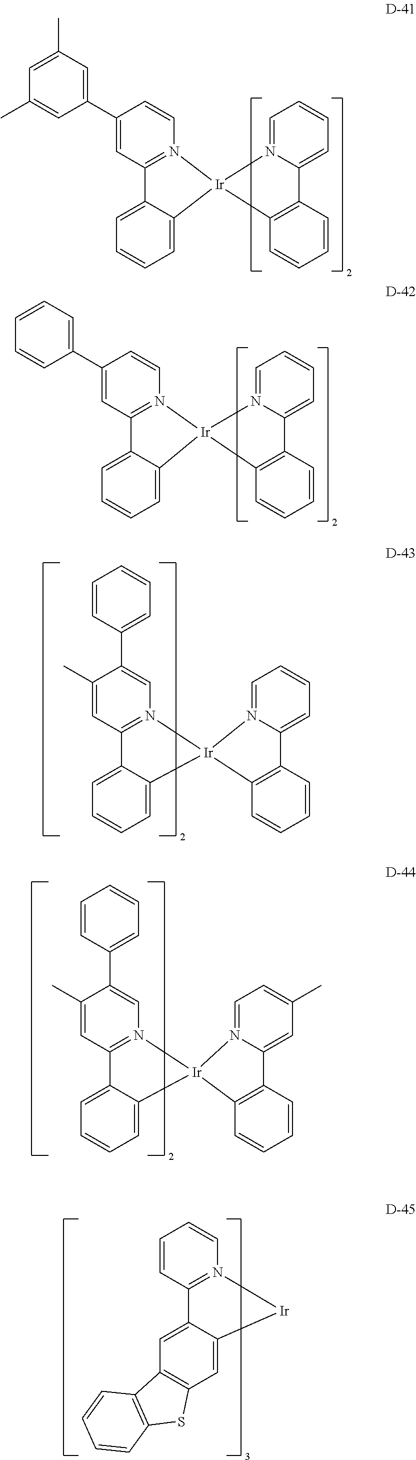

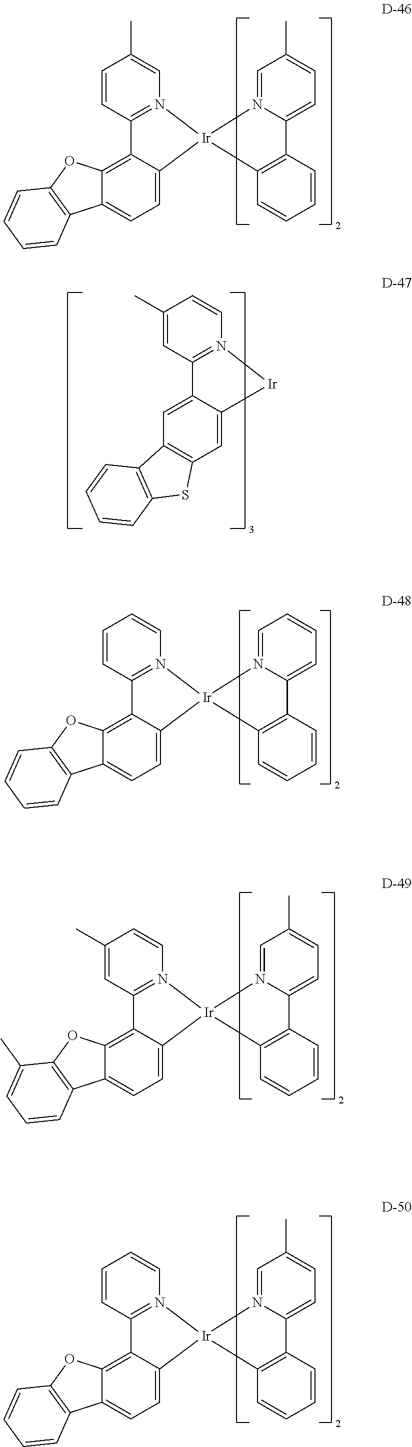

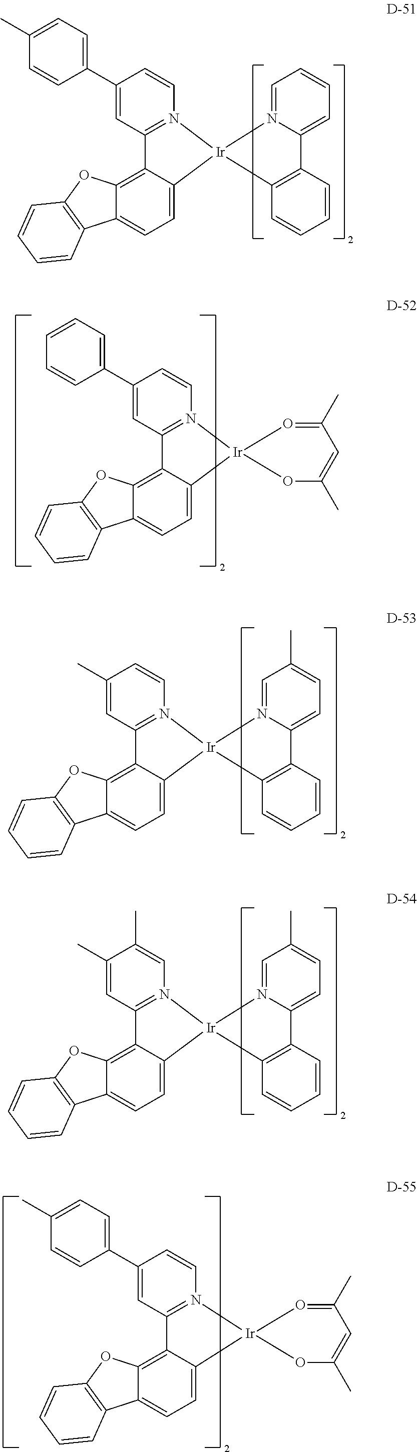

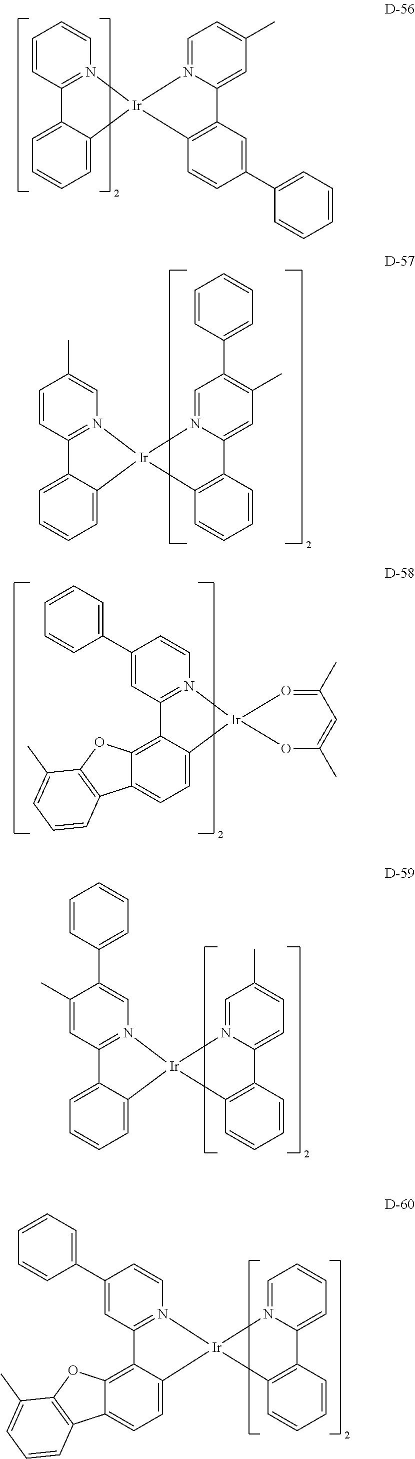

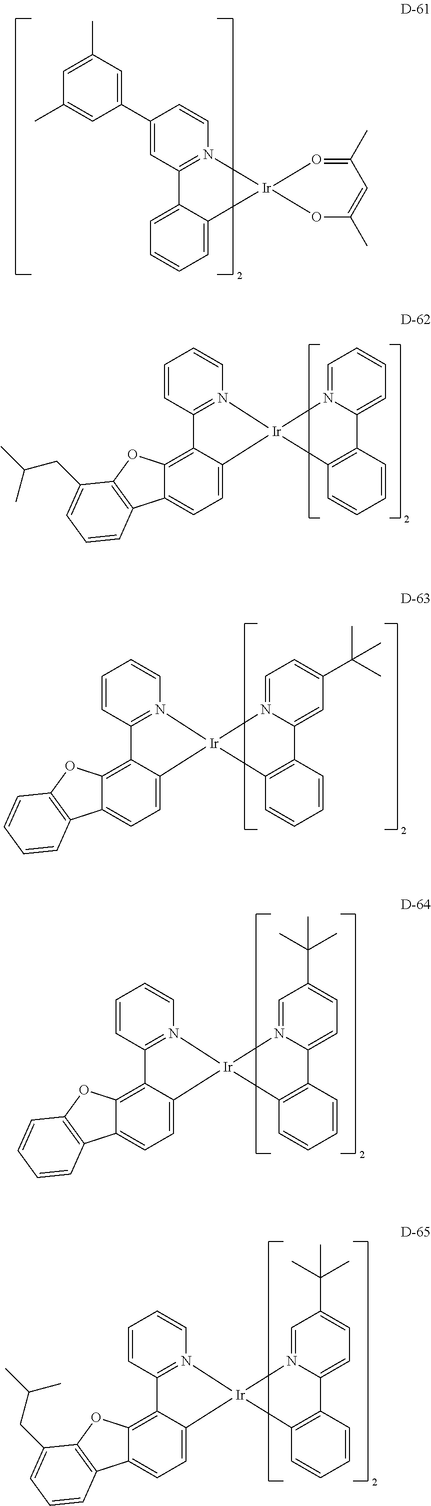

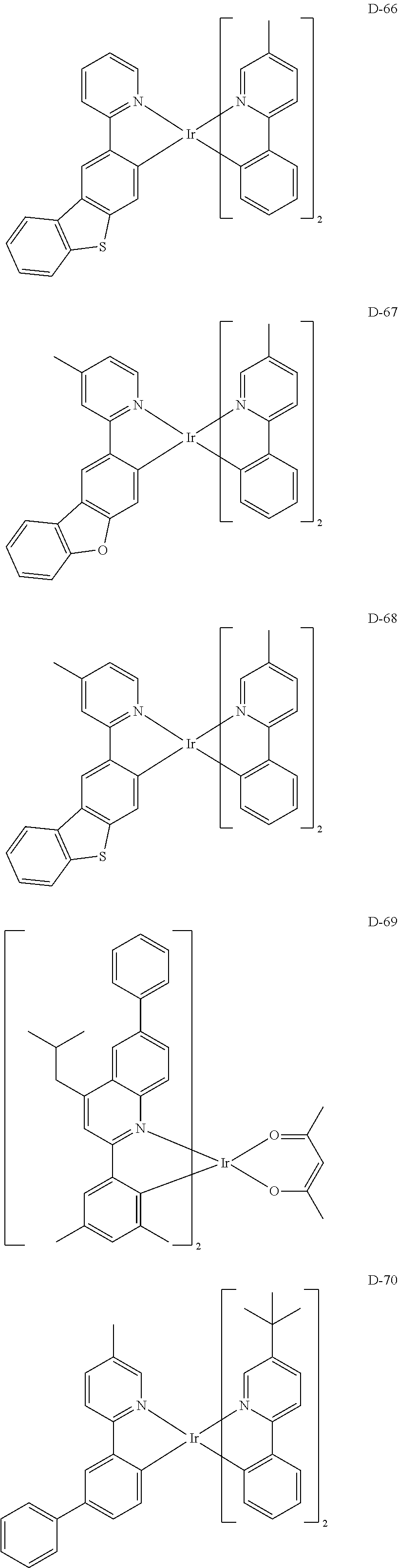

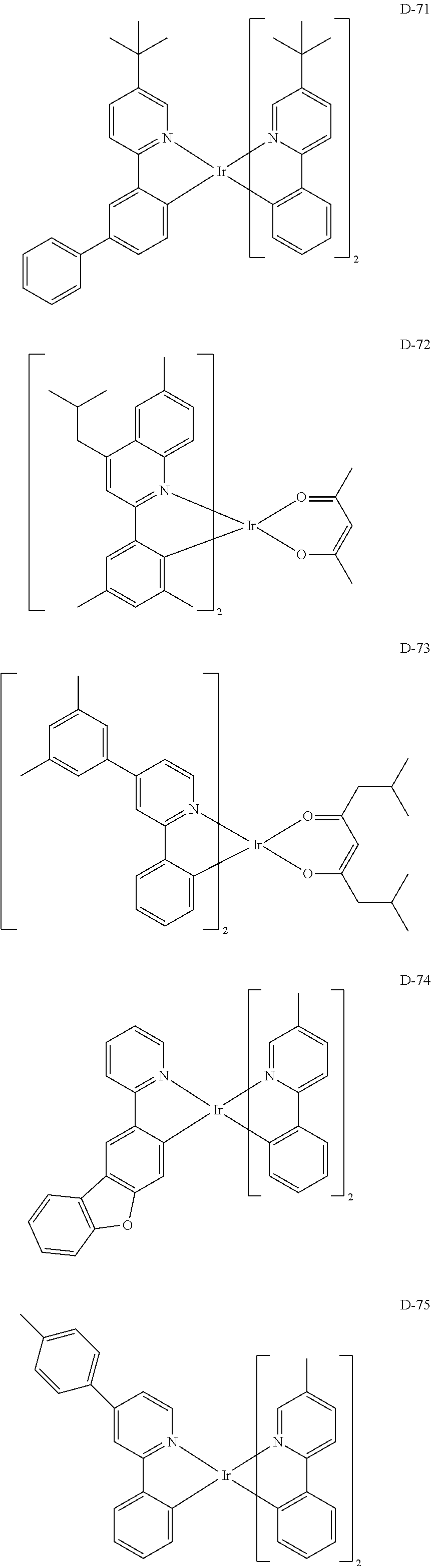

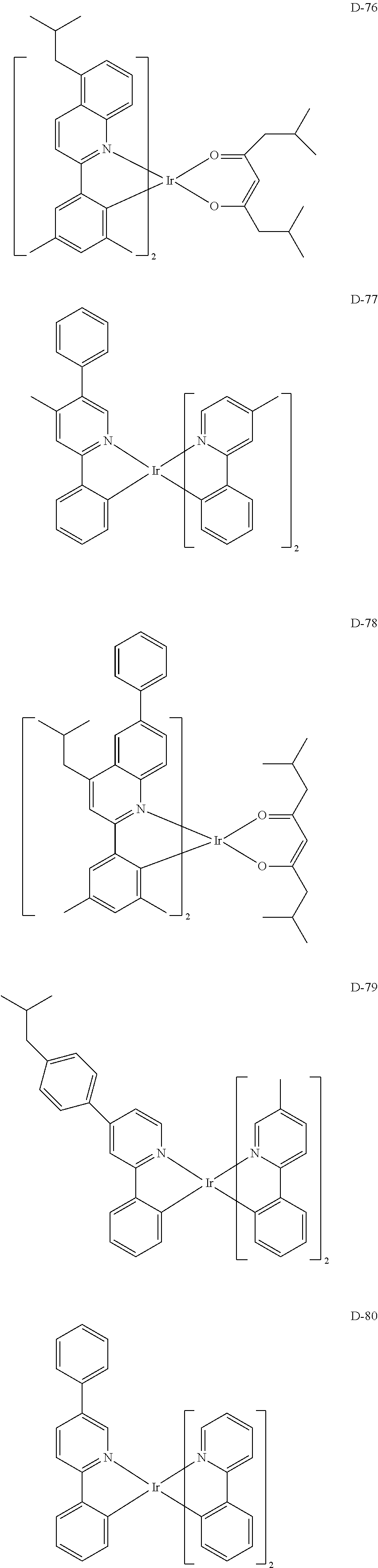

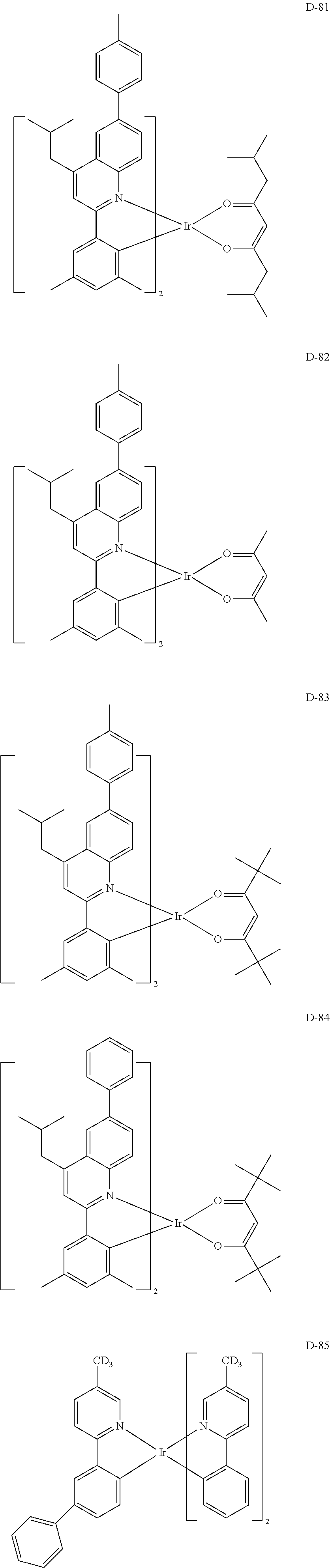

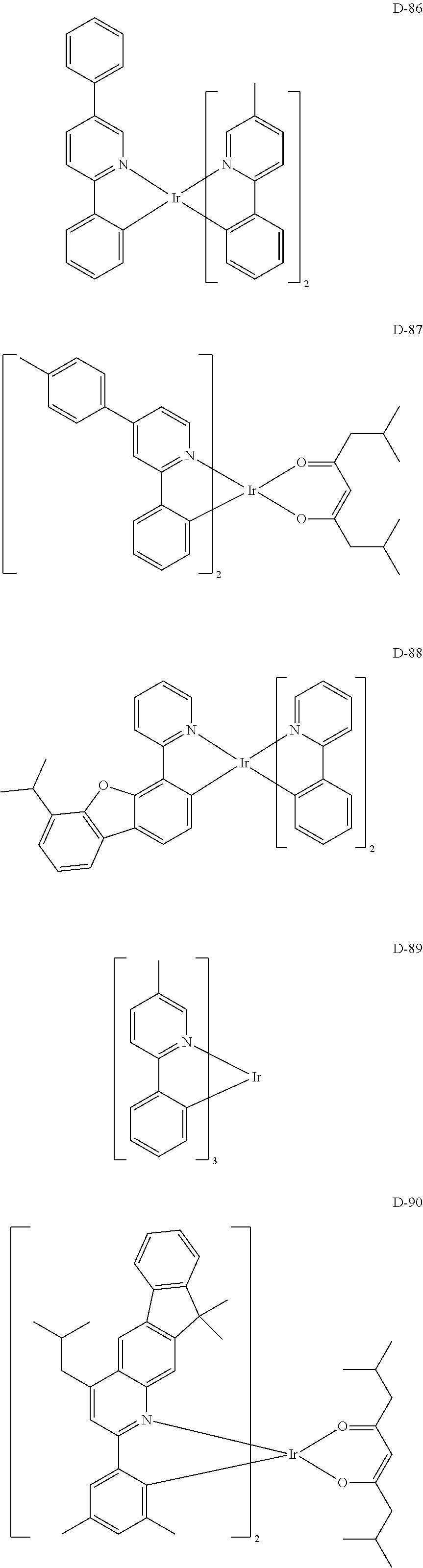

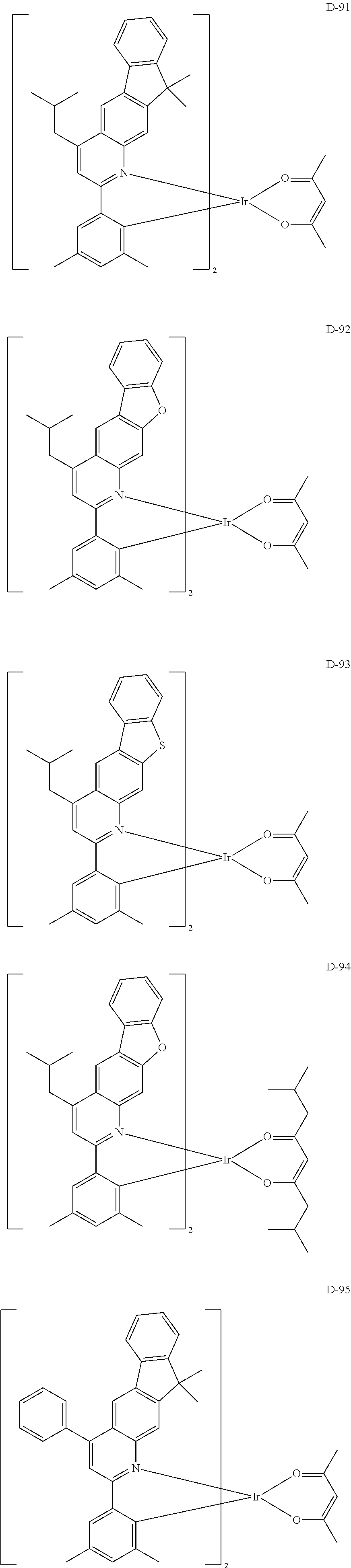

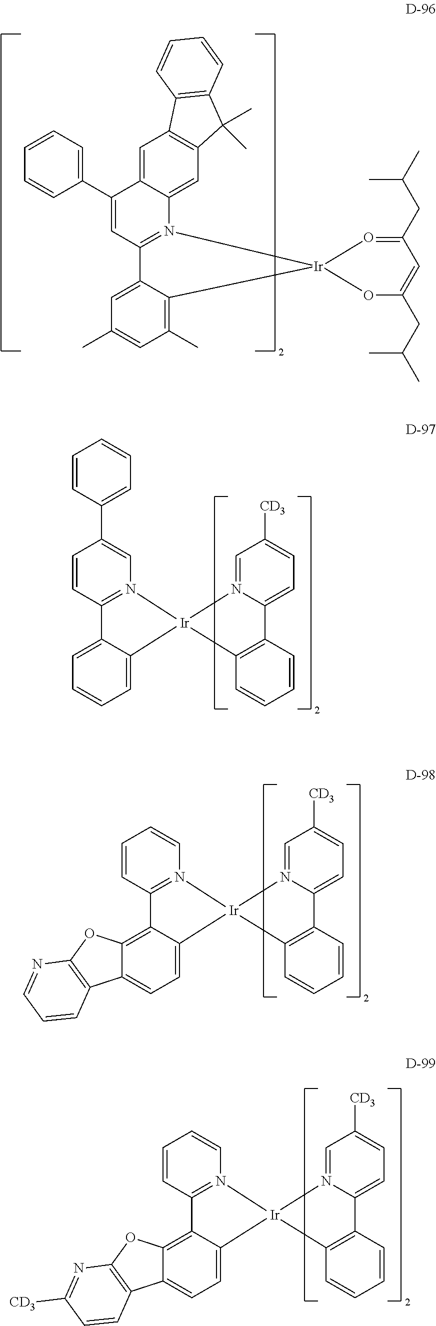

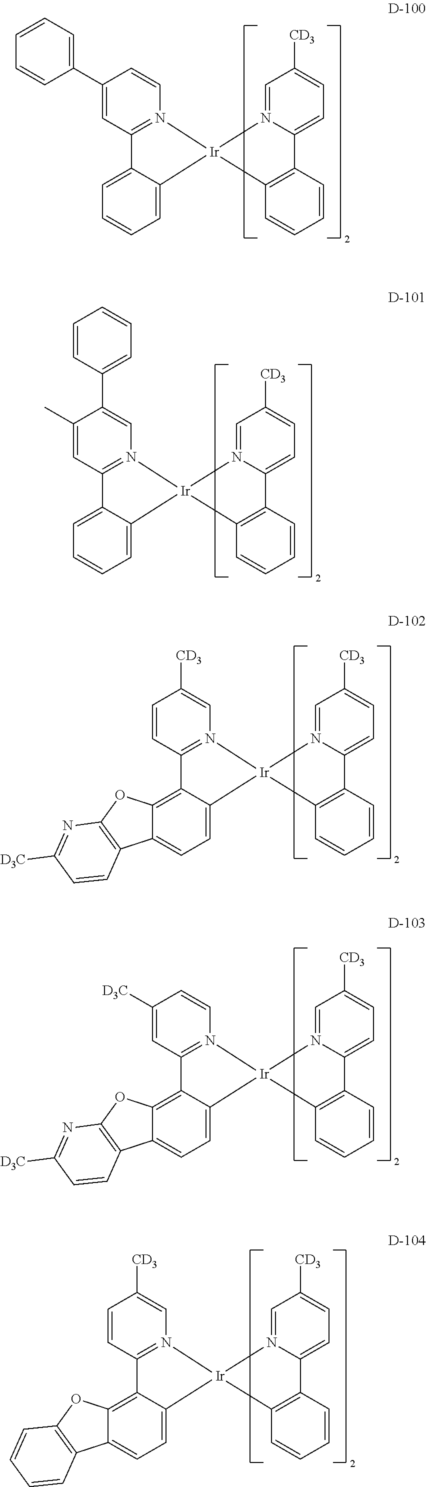

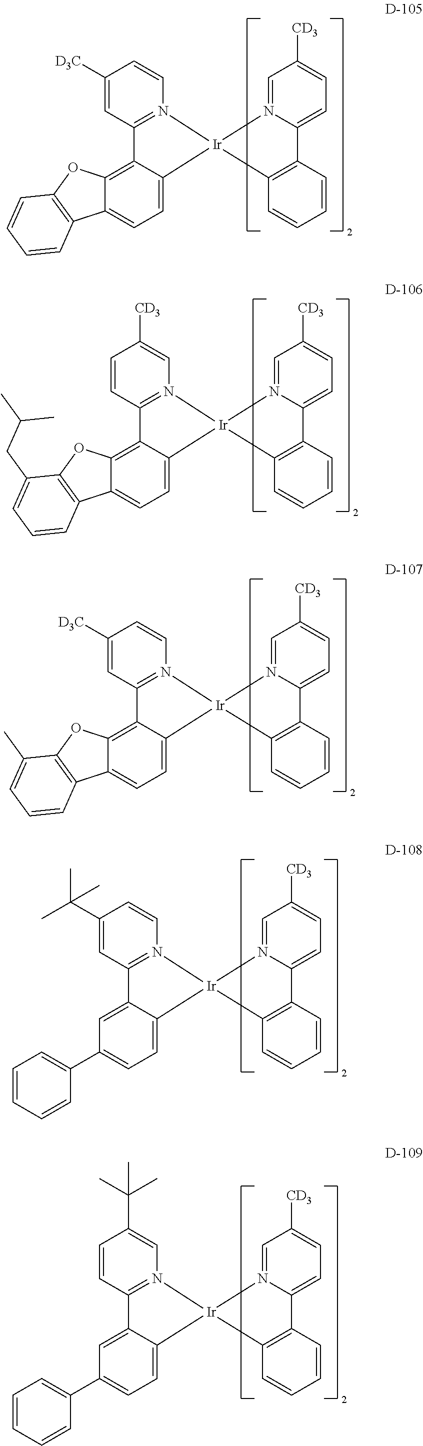

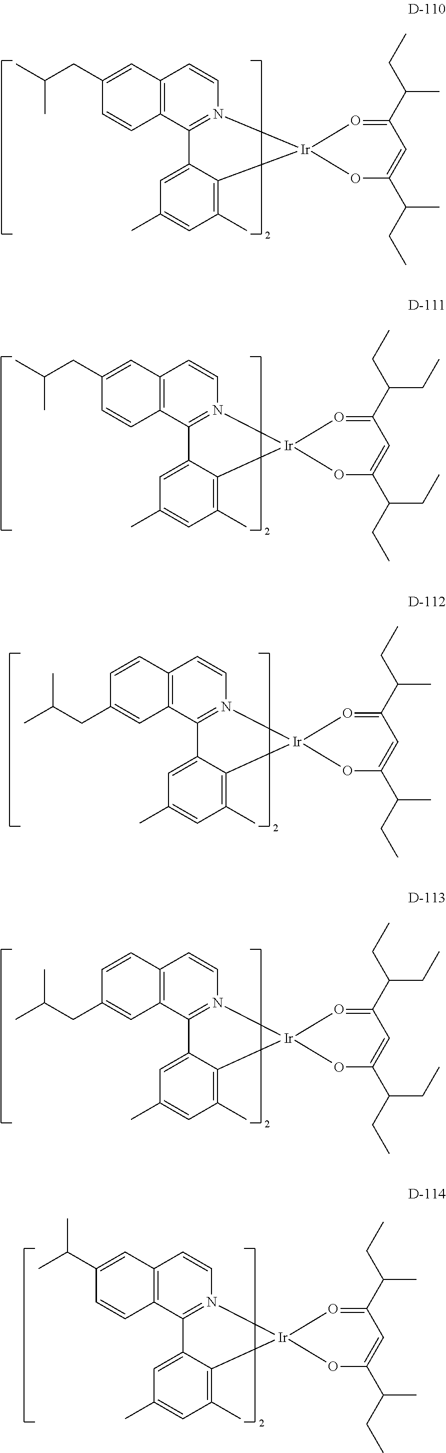

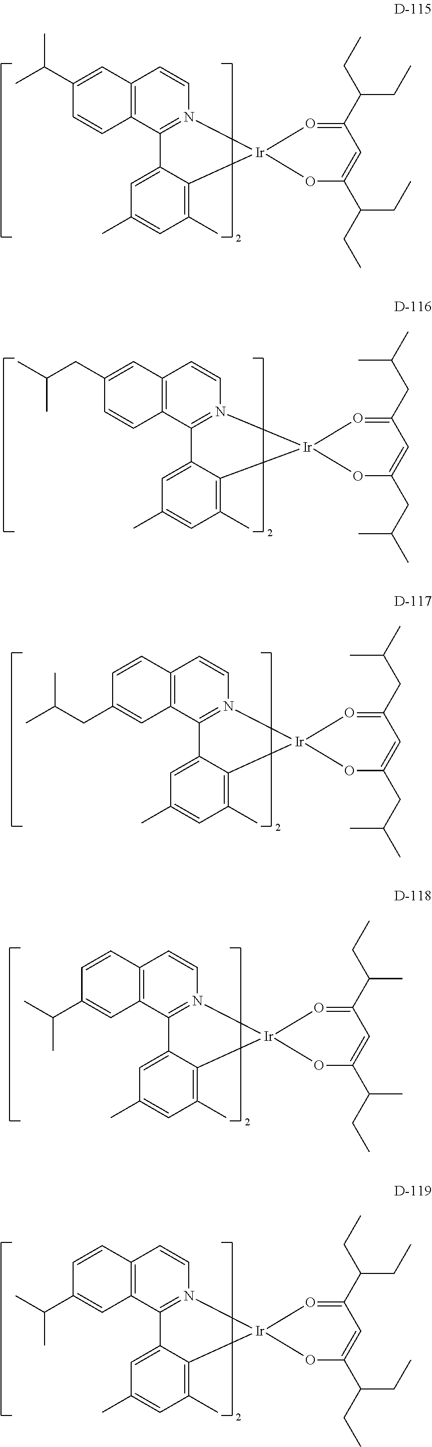

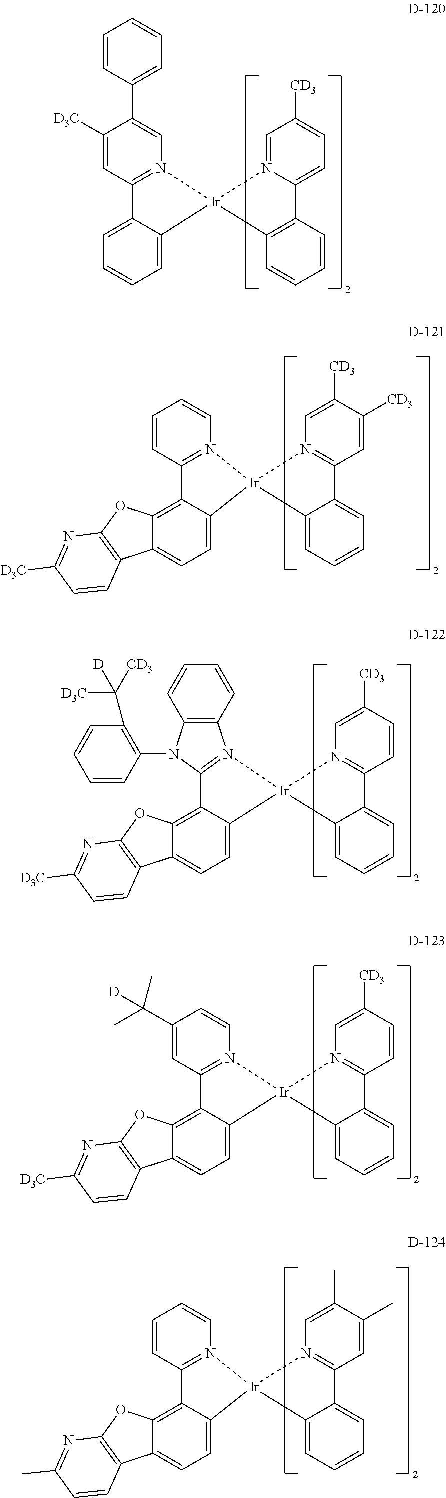

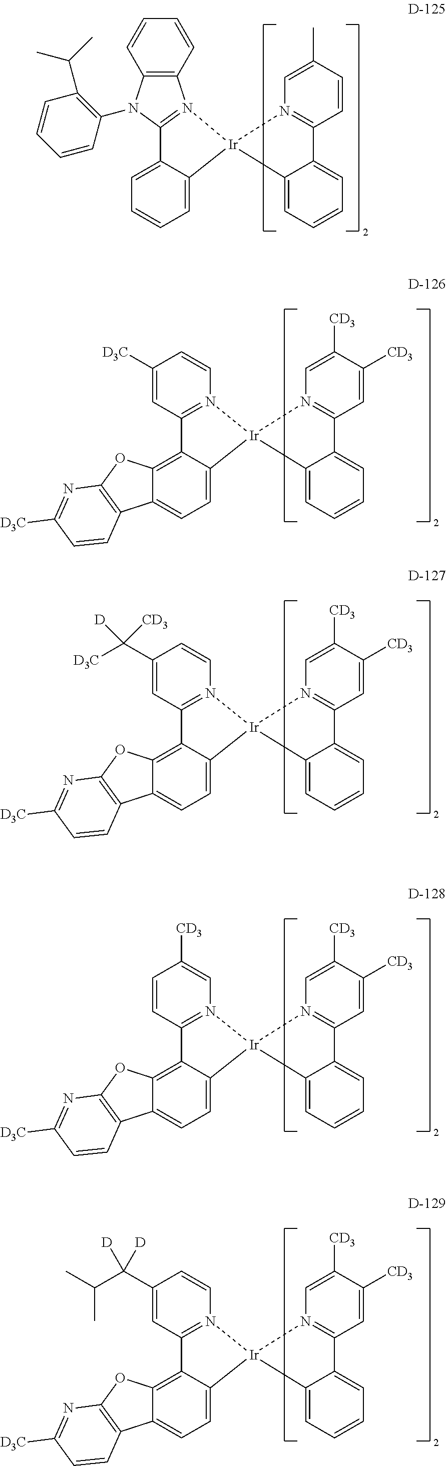

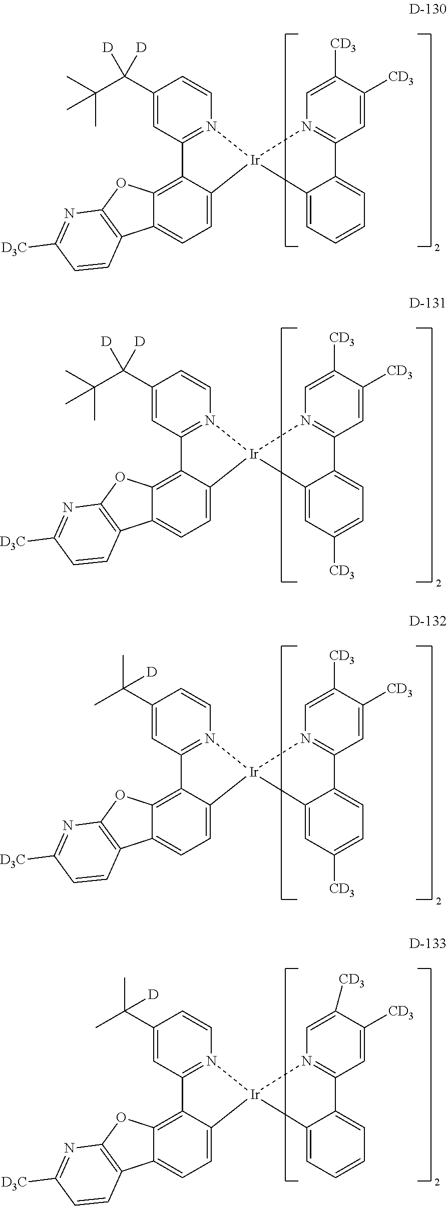

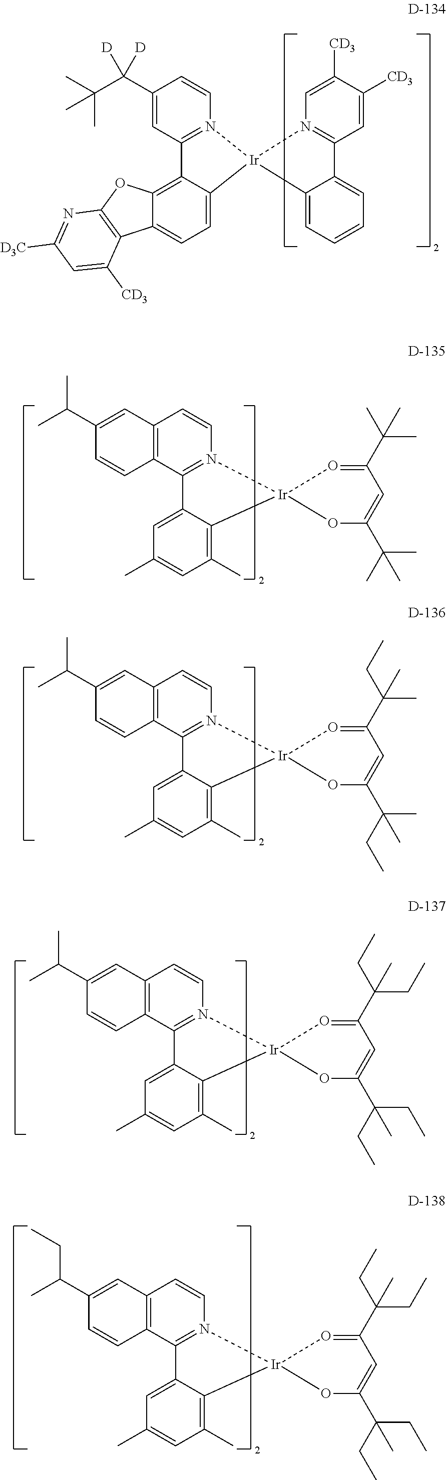

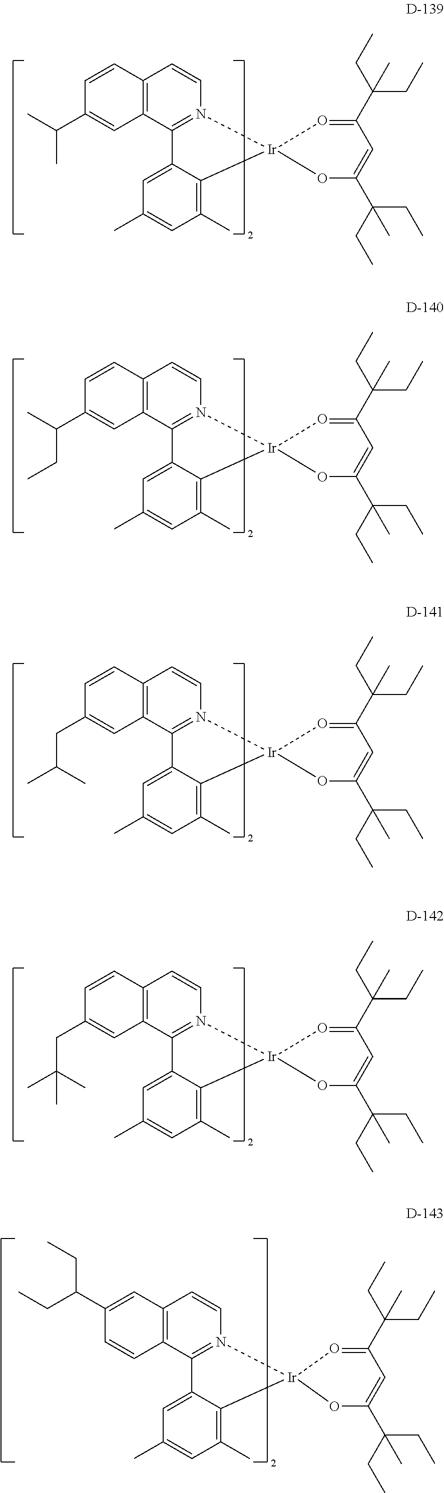

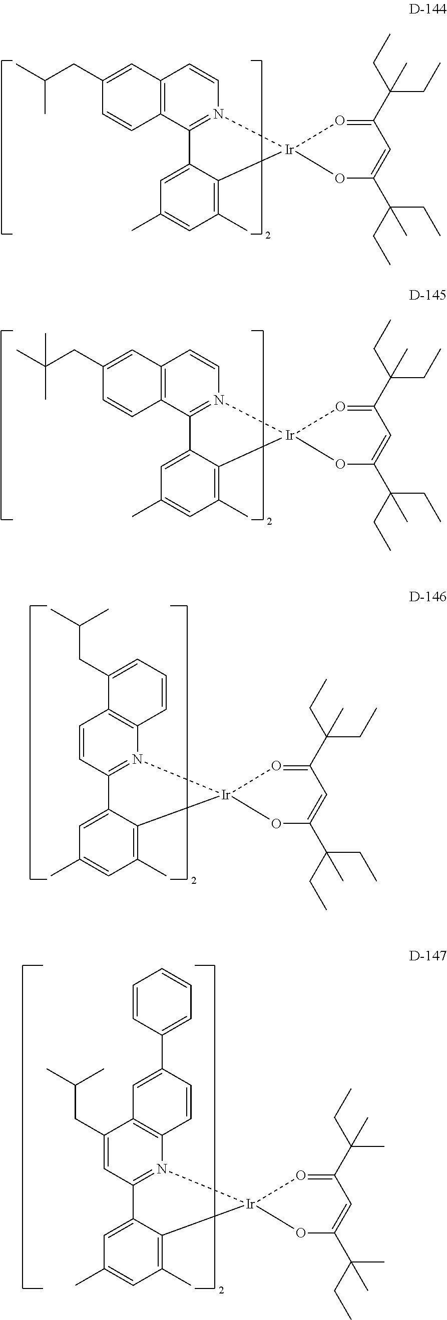

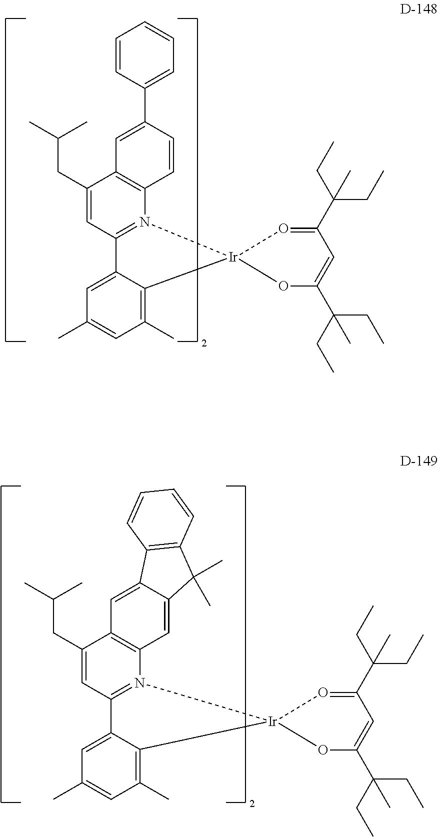

An organic electroluminescent device according to the present disclosure has an anode, a cathode, and at least one organic layer between the anode and the cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds. Further, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiO_{X}(1≤X≤2), AlO_{X}(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

The organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The organic electroluminescent material according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When the first and second host compounds of the present disclosure are used to form a film, a co-evaporation process or a mixture-evaporation process is carried out.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof, and the driving voltage and the luminous efficiency of an organic electroluminescent device (OLED) comprising a plurality of host materials according to the present disclosure will be explained in detail with reference to the representative compounds of the present disclosure. However, the following examples only describe the properties of the compound according to the present disclosure and the OLED comprising the same, and the present disclosure is not limited to the following examples.

### Example 1: Preparation of Compound C-1

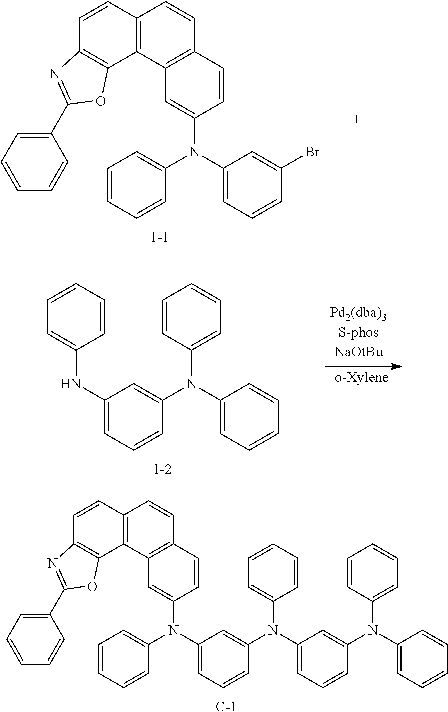

Compound 1-1 (6 g, 11.08 mmol), Compound 1-2 (4.1 g, 12.189 mmol), tris(dibenzylideneacetone)dipalladium(0) (0.12 g, 0.554 mmol), sodium tert-butoxide (1.6 g, 16.62 mmol), S-phos (0.22 g, 1.108 mmol) and 55 mL of o-xylene were added to a flask, and stirred under reflux for 1 hour. After completion of the reaction, the reactant was cooled to room temperature and filtered through celite. The filtrate was distilled under reduced pressure, and separated by column chromatography to obtain Compound C-1 (2.3 g, yield: 26%).

| | MW | M.P. |
|---|---|---|
| C-1 | 796.97 | 145.8° C. |

### Example 2: Preparation of Compound C-96

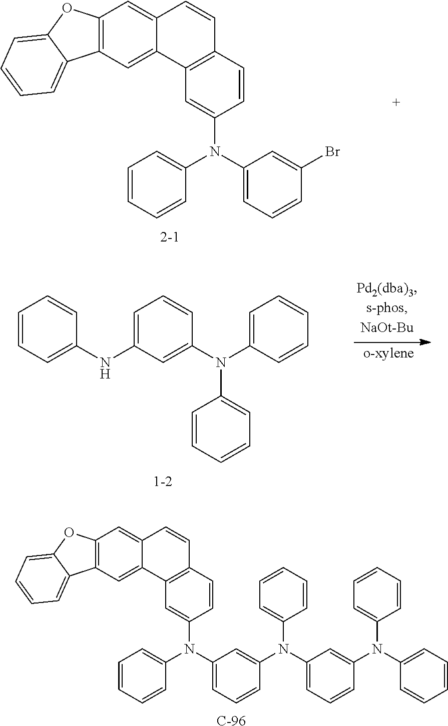

Compound 2-1 (9.17 g, 17.83 mmol), Compound 1-2 (5 g, 14.86 mmol), Pd 2 (dba) 3 (0.68 g, 0.743 mmol), S-phos (0.61 g, 1.486 mmol), and sodium tert-butoxide (2.1 g, 22.29 mmol) were added to a flask, dissolved in 75 mL of o-xylene, and stirred under reflux for 2 hours. After completion of the reaction, the reactant was cooled to room temperature and filtered through celite. The filtrate was distilled under reduced pressure, extracted with MC/Hex, and separated by column chromatography to obtain Compound C-96 (6.2 g, yield: 54%).

| | MW | M.P. |
|---|---|---|
| C-96 | 769.9 | 195.3° C. |

### Example 3: Preparation of Compound C-159

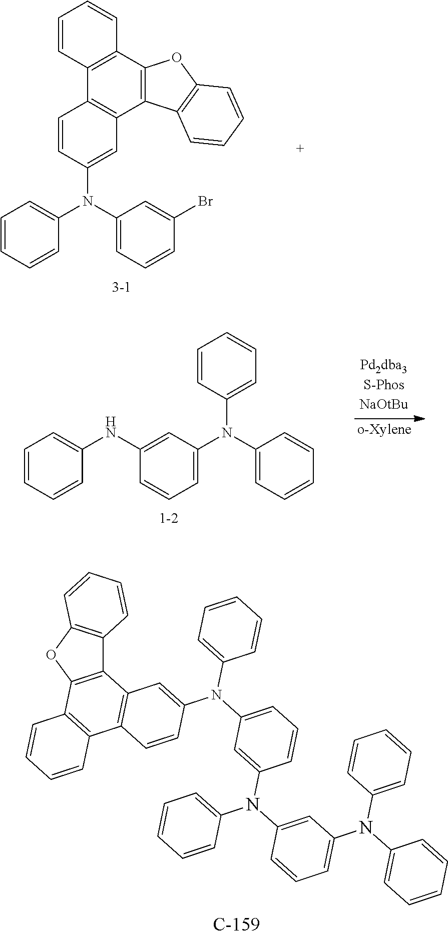 [SMILES: Brc1cccc(N(c2ccccc2)c2ccc3c4ccccc4c4oc5ccccc5c4c3c2)c1 ; c1ccc(Nc2cccc(N(c3ccccc3)c3ccccc3)c2)cc1 ; c1ccc(N(c2ccccc2)c2cccc(N(c3ccccc3)c3cccc(N(c4ccccc4)c4ccc5c6ccccc6c6oc7ccccc7c6c5c4)c3)c2)cc1 ; "C-159"]

Compound 3-1 (5.6 g, 10.886 mmol), Compound 1-2 (4.0 g, 11.975 mmol), Pd₂(dba)₃ (0.49 g, 0.544 mmol), sodium tert-butoxide (2.1 g, 21.77 mmol), and S-phos (0.44 g, 1.088 mmol) were added to a flask, dissolved in 55 mL of o-xylene, and stirred under reflux for 1 hour. After completion of the reaction, the reactant was cooled to room temperature and filtered through celite. The filtrate was distilled under reduced pressure, and separated by column chromatography to obtain Compound C-159 (3.2 g, yield: 38%).

| | MW | M.P. |
|---|---|---|
| C-159 | 769.93 | 188.8° C. |

### Example 4: Preparation of Compound C-35

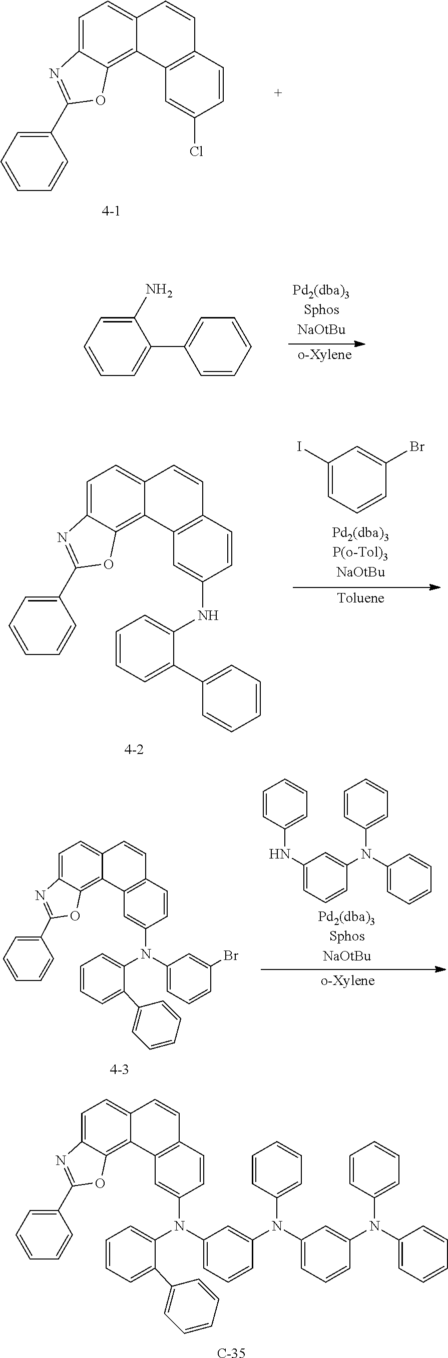

### 1) Synthesis of Compound 4-2

Compound 4-1 (24.4 g, 73.9 mmol), 2-aminobiphenyl (12.5 g, 73.9 mmol), Pd₂(dba)₃ (3.38 g, 3.69 mmol), S-phos (3.04 g, 7.39 mmol) and sodium tent-butoxide (10.6 g, 111 mmol) were added to a flask, dissolved in 375 mL of o-xylene, and stirred under reflux for 3 hours. After completion of the reaction, the organic layer was extracted with ethyl acetate and washed with distilled water. The organic solution was filtered with silica gel to obtain Compound 4-2 (28.0 g, yield: 81.9%).

### 2) Synthesis of Compound 4-3

Compound 4-2 (12.0 g, 26.0 mmol), 1-bromo-3-iodobenzene (10.9 g, 28.5 mmol), Pd₂(dba)₃ (1.19 g, 1.30 mmol), tri-o-tolyl phosphine (1.58 g, 5.19 mmol) and sodium tert-butoxide (3.74 g, 38.9 mmol) were added to a flask, dissolved in 130 mL of toluene, and stirred under reflux for 2 hours. After completion of the reaction, the reactant was cooled to room temperature, the organic layer was extracted with dichloromethane, washed with distilled water, distilled under reduced pressure, and separated by column chromatography to obtain Compound 4-3 (11.2 g, yield: 70.0%).

### 3) Synthesis of Compound C-35

Compound 4-3 (5.0 g, 8.1 mmol), N1,N1,N3-triphenylbenzene-1,3-diamine (2.7 g, 8.1 mmol), Pd₂(dba)₃ (371 mg, 0.405 mmol), S-phos (333 mg, 0.810 mmol) and sodium tert-butoxide (1.17 g, 12.2 mmol) were added to a flask, dissolved in 40 mL of o-xylene, and stirred under reflux for 18 hours. After completion of the reaction, the organic layer was extracted with dichloromethane, washed with distilled water, distilled under reduced pressure, and separated by column chromatography to obtain Compound C-35 (2.20 g, yield: 31.1%).

| | MW | M.P. |
|---|---|---|
| C-35 | 873.0 | 174° C. |

### Example 5: Preparation of Compound C-196

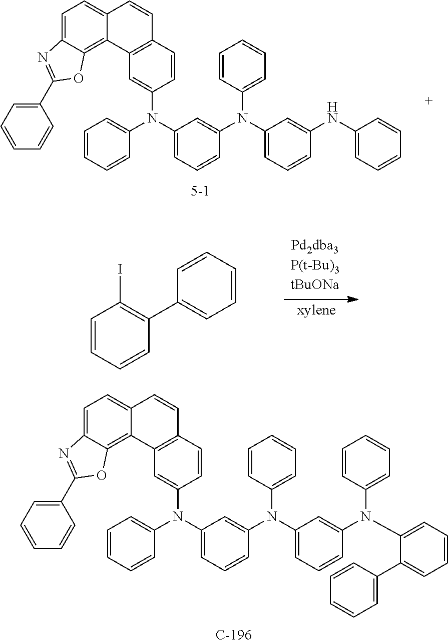

Compound 5-1 (8 g, 11.09 mmol), 2-iodo-1,1′-biphenyl (2.3 mL, 13.3 mmol), Pd₂ (dba)₃ (508 mg, 0.55 mmol), P(t-Bu)₃ (0.5 ml, 1.1 mmol) and sodium tert-butoxide (2.7 g, 27.74 mmol) were added to a flask, dissolved in 55 mL of o-xylene, and stirred at 160° C. for 12 hours. After the reaction was completed, the reactant was cooled to room temperature, and the organic layer was extracted with ethyl acetate. After the residual moisture was removed using magnesium sulfate, the residue was dried and separated by column chromatography to obtain Compound C-196 (1.9 g, yield: 19%).

| | MW | M.P. |
|---|---|---|
| C-196 | 872.35 | 125.7° C. |

### Device Example 1: Producing an OLED Comprising a Compound According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 shown in Table 3 below was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 3 wt % based on the total amount of Compound HI-1 and Compound HT-1 to form a hole injection layer having a thickness of 10 nm on the ITO substrate. Next, Compound HT-1 was deposited on the hole injection layer to form a first hole transport layer having a thickness of 80 nm. The compound shown in Table 1 below was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: the first host compound Host 1 and the second host compound H2-56 were introduced into two cells of the vacuum vapor deposition apparatus as hosts, and Compound D-39 was introduced into another cell as a dopant. The two host materials were evaporated at a different rate of 1:1 and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Compound ET-1 and Compound EI-1 were evaporated in a weight ratio of 50:50 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 1: Producing an OLED Comprising a Comparative Compound

An OLED was produced in the same manner as in Device Example 1, except that the compound shown in Table 1 was used as the second hole transport layer material.

The driving voltage, luminous efficiency, and light-emitting color at a luminance of 1,000 nit, and the time taken for luminance to decrease from 100% to 95% at a luminance of 10,000 nit (lifetime; T95) of the OLEDs produced in Device Example 1 and Comparative Example 1 are provided in Table 1 below.

TABLE 1

| | Second | | | | Life- |
|---|---|---|---|---|---|
| | Hole | Driving | Luminous | Light- | time |
| | Transport | Voltage | Efficiency | Emitting | T95 |
| | Layer | (V) | (cd/A) | Color | (hr) |
| Device Example 1 | C-1 | 2.9 | 35.2 | Red | 90 |
| Comparative | HTL2 | 3.0 | 31.1 | Red | 3 |
| Example 1 | | | | | |

As shown in Table 1 above, it can be confirmed that the OLED comprising the hole transport compound according to the present disclosure (Device Example 1) exhibits low driving voltage, high luminous efficiency, and improved lifetime properties, compared to the OLED comprising the conventional compound (Comparative Example 1).

### Device Examples 2 to 7: Producing an OLED Comprising a Plurality of Host Materials According to the Present Disclosure

OLEDs were produced in the same manner as in Device Example 1, except that Compound HT-2 was used as the second hole transport layer material, and the host compounds shown in Table 2 below were used as host materials of the light-emitting layer.

### Comparative Examples 2 and 3: Producing an OLED Comprising a Comparative Compound as a Host

OLEDs were produced in the same manner as in each Device Examples 2 to 7, except that the second host compound shown in Table 2 below was used alone as a host of the light-emitting layer.

The driving voltage, luminous efficiency, and light-emitting color at a luminance of 1,000 nit, and the time taken for luminance to decrease from 100% to 95% at a luminance of 10,000 nit (lifetime; T95) of the OLEDs produced in Device Examples 2 to 7 and Comparative Examples 2 and 3 are provided in Table 2 below.

TABLE 2

| | First | Second | Driving | Luminous | Light- | Lifetime |
|---|---|---|---|---|---|---|
| | Host | Host | Voltage | Efficiency | Emitting | T95 |
| | Compound | Compound | (V) | (cd/A) | Color | (hr) |
| Device | C-1 | H2-56 | 3.0 | 35.9 | Red | 195 |
| Example 2 | | | | | | |
| Device | C-1 | H2-236 | 2.9 | 33.6 | Red | 135 |
| Example 3 | | | | | | |
| Device | C-96 | H2-56 | 3.0 | 36.3 | Red | 144 |
| Example 4 | | | | | | |
| Device | C-96 | H2-236 | 2.9 | 34.6 | Red | 108 |
| Example 5 | | | | | | |
| Device | C-35 | H2-56 | 3.0 | 35.6 | Red | 179 |
| Example 6 | | | | | | |
| Device | C-196 | H2-56 | 3.0 | 35.7 | Red | 217 |
| Example 7 | | | | | | |
| Comparative | — | H2-56 | 3.5 | 31.8 | Red | 18 |
| Example 2 | | | | | | |
| Comparative | — | H2-236 | 3.0 | 25.3 | Red | 17 |
| Example 3 | | | | | | |

As shown in Table 2 above, it can be confirmed that the OLEDs comprising the plurality of host materials according to the present disclosure (Device Examples 2 to 7) exhibit low driving voltage, high luminous efficiency, and improved lifetime properties, compared to the OLEDs comprising the conventional compound (Comparative Examples 2 and 3).

The compounds used in the Device Examples and the Comparative Examples are shown in Table 3 below.

TABLE 3

| | |
|---|---|
| Hole Injection Layer/ Hole Transport Layer | 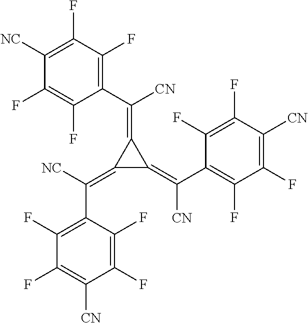 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 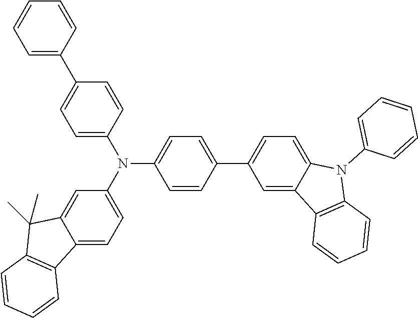 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 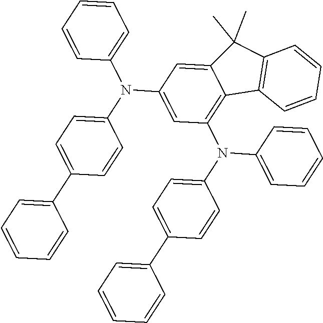 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2 |
| | 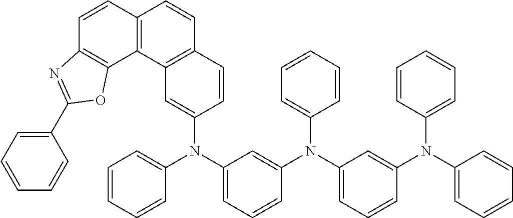 [SMILES: c1ccc(-c2nc3ccc4ccc5ccc(N(c6ccccc6)c6cccc(N(c7ccccc7)c7cccc(N(c8ccccc8)c8ccccc8)c7)c6)cc5c4c3o2)cc1] |
| | C-1 |
| | 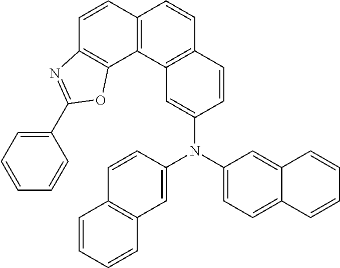 [SMILES: c1ccc(-c2nc3ccc4ccc5ccc(N(c6ccc7ccccc7c6)c6ccc7ccccc7c6)cc5c4c3o2)cc1] |
| | HTL2 |
| Light-Emitting Layer | 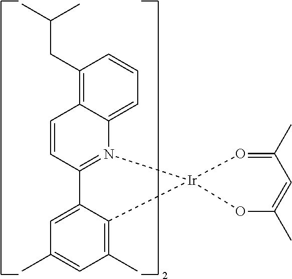 |
| | D-39 |
| | 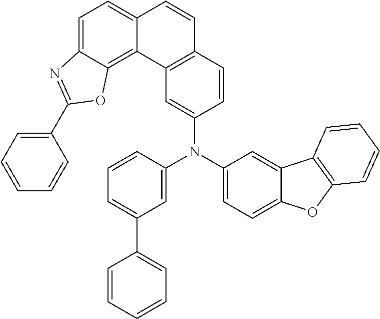 [SMILES: c1ccc(-c2cccc(N(c3ccc4oc5ccccc5c4c3)c3ccc4ccc5ccc6nc(-c7ccccc7)oc6c5c4c3)c2)cc1] |
| | Host1 |
| | 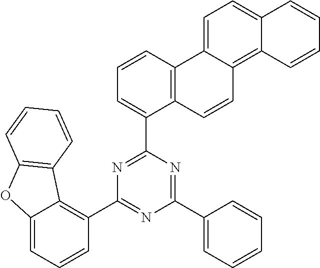 [SMILES: c1ccc(-c2nc(-c3cccc4c3ccc3c5ccccc5ccc43)nc(-c3cccc4oc5ccccc5c34)n2)cc1] |
| | H2-56 |
| | 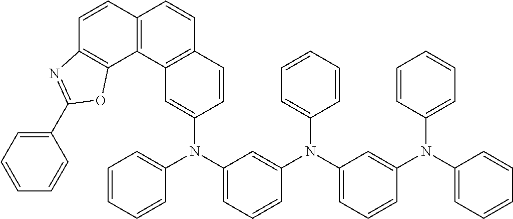 [SMILES: c1ccc(-c2nc3ccc4ccc5ccc(N(c6ccccc6)c6cccc(N(c7ccccc7)c7cccc(N(c8ccccc8)c8ccccc8)c7)c6)cc5c4c3o2)cc1] |
| | C-1 |
| | 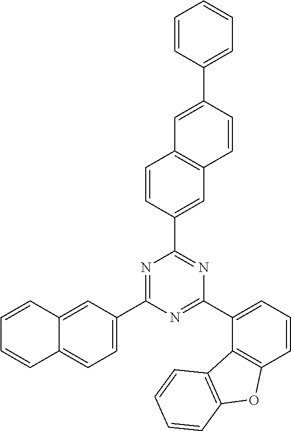 [SMILES: c1ccc(-c2ccc3cc(-c4nc(-c5ccc6ccccc6c5)nc(-c5cccc6oc7ccccc7c56)n4)ccc3c2)cc1] |
| | H2-236 |
| | 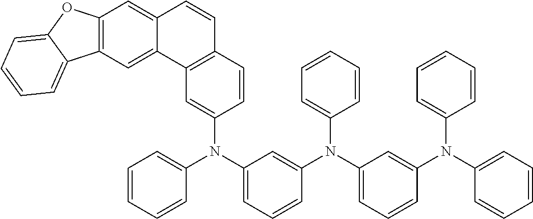 [SMILES: c1ccc(N(c2ccccc2)c2cccc(N(c3ccccc3)c3cccc(N(c4ccccc4)c4ccc5ccc6cc7oc8ccccc8c7cc6c5c4)c3)c2)cc1] |
| | C-96 |
| | 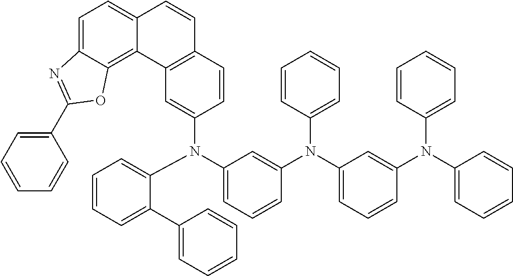 [SMILES: c1ccc(-c2nc3ccc4ccc5ccc(N(c6cccc(N(c7ccccc7)c7cccc(N(c8ccccc8)c8ccccc8)c7)c6)c6ccccc6-c6ccccc6)cc5c4c3o2)cc1] |
| | C-35 |
| | 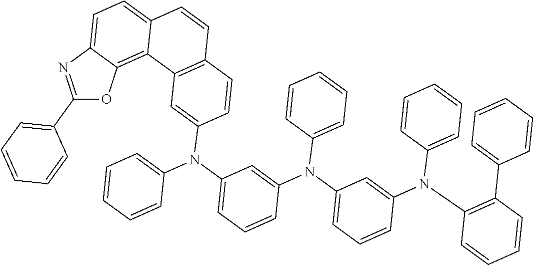 [SMILES: c1ccc(-c2nc3ccc4ccc5ccc(N(c6ccccc6)c6cccc(N(c7ccccc7)c7cccc(N(c8ccccc8)c8ccccc8-c8ccccc8)c7)c6)cc5c4c3o2)cc1] |
| | C-196 |
| Electron Transport Layer/ Electron Injection Layer | 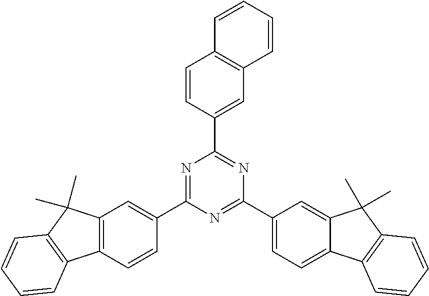 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ET-1 |
| | 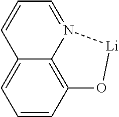 |
| | EI-1 |

1. An organic electroluminescent compound represented by the following formula 1: 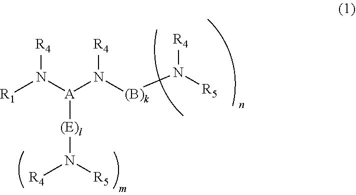
in formula 1,
R₁ to R₅, each independently, represent a substituted or unsubstituted (C6-C30)aryl, or a substituted or unsubstituted (3- to 30-membered)heteroaryl; with the proviso that at least one of R₁ to R₅ represents -L-Har;
L, each independently, represents a single bond, a substituted or unsubstituted (C6-C30)arylene, or a substituted or unsubstituted (3- to 30-membered)heteroarylene;
Har, each independently, represents a substituted or unsubstituted phenanthrooxazolyl, a substituted or unsubstituted phenanthrothiazolyl, a substituted or unsubstituted phenanthrobenzofuranyl, or a substituted or unsubstituted benzophenanthrothiophenyl;
A represents a substituted or unsubstituted (C6-C30)arentriyl, or a substituted or unsubstituted (3- to 30-membered) heteroarentriyl;
B and E, each independently, represent a substituted or unsubstituted (C6-C30)arylene, or a substituted or unsubstituted (3- to 30-membered)heteroarylene; and
k represents an integer of 1 or 2, and l to n, each independently, represent an integer of 0 or 1; in which if k represents an integer of 2, each of B may be the same or different; and
provided that m+n=1; if n represents an integer of 0, B has the same definition as R 1 , and if m represents an integer of 0, l represents an integer of 0, and A has the same definition as E.

2. The organic electroluminescent compound according to claim 1, wherein Har in formula 1, each independently, is represented by the following formula a: 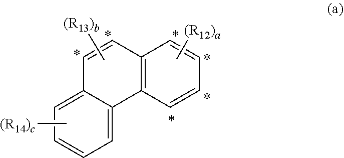
in formula a,
adjacent * is connected to the following formula a-1 or formula a-2; 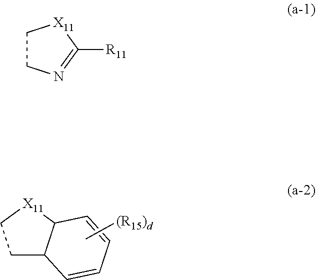
X₁₁, each independently, represents O or S;
any one of R₁₁ to R₁₅ is connected with L;
if R₁₁ is not connected with L, R₁₁ represents a substituted or unsubstituted (C6-C30)aryl, or a substituted or unsubstituted (3- to 30-membered)heteroaryl;
if R₁₂ to R₁₅ are not connected with L, R₁₂ to R₁₅, each independently, represent hydrogen, deuterium, a substituted or unsubstituted (C6-C30)aryl, or a substituted or unsubstituted (3- to 30-membered)heteroaryl; and
a, c and d, each independently, represent an integer of 1 to 4, and b, each independently, represents an integer of 0 to 2; in which if a to d represent an integer of 2 or more, each of R₁₂ to each of R₁₅, may be the same or different.

3. The organic electroluminescent compound according to claim 2, wherein Har in formula 1, each independently, is represented by the following formula 1-1 or 1-2: 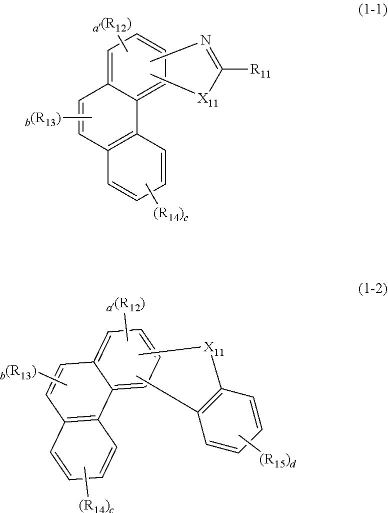
in formulas 1-1 and 1-2,
a′, each independently, represents an integer of 1 or 2; in which if a′ represents an integer of 2, each of R₁₂ may be the same or different; and
X₁₁, R₁₁ to R₁₅, and b to d are as defined in claim 2.

4. The organic electroluminescent compound according to claim 2, wherein Har in formula 1, each independently, is represented by any one of the following formulas 1-11 to 1-15: 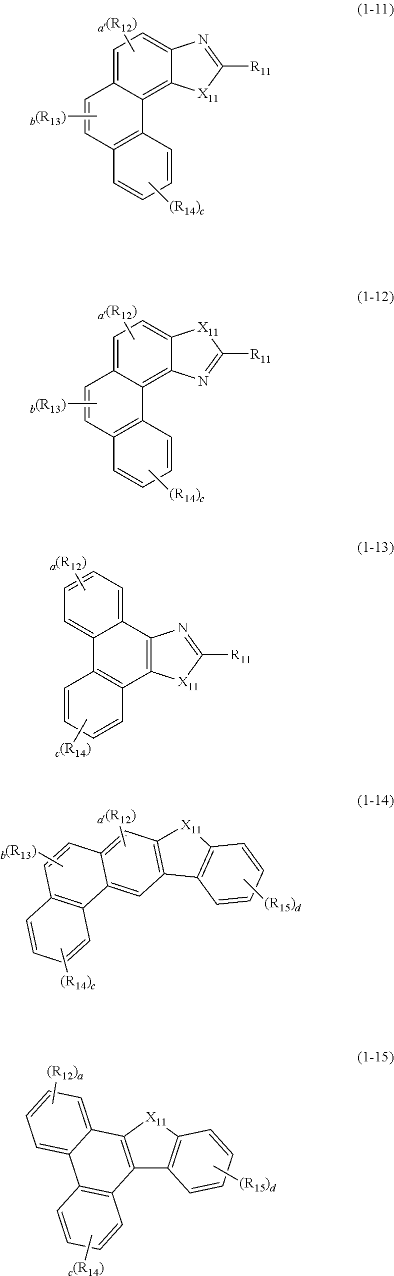
in formulas 1-11 to 1-15,
a′ represents an integer of 1 or 2, in which if a′ represents an integer of 2 or more, each of R12 may be the same or different; and
X₁₁, R₁₁ to R₁₅, and a to d are as defined in claim 2.

5. The organic electroluminescent compound according to claim 1, wherein when n represents an integer of 1 and m represents an integer of 0 in the formula 1, at least one of A and B is represented by any one of the following formulas: 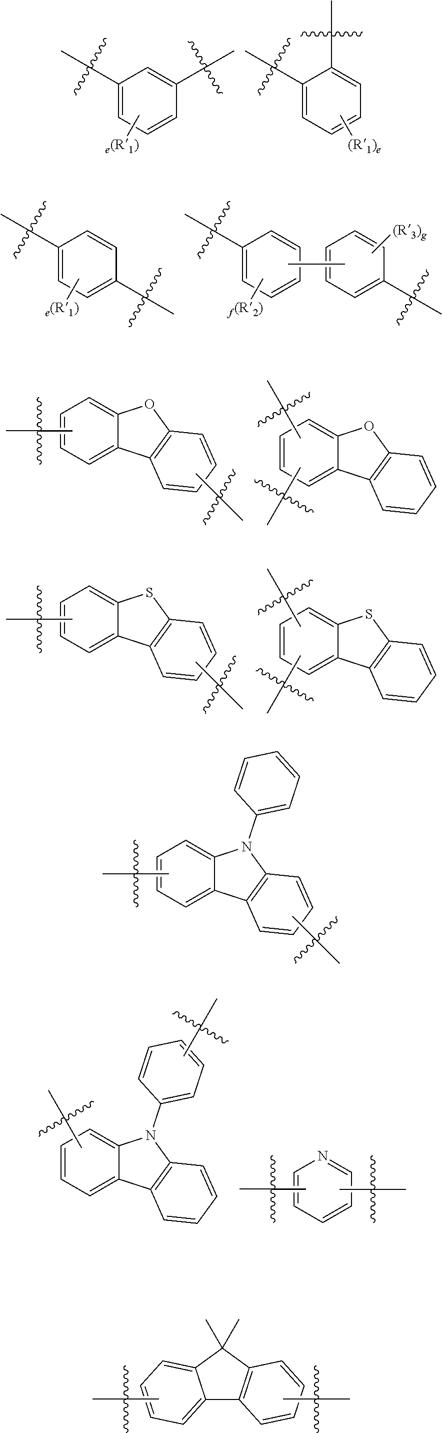
in the formulas,
hydrogen may be replaced by deuterium;
R′₁ to R′₃, each independently, represent hydrogen, deuterium, a substituted or unsubstituted (C6-C30)aryl, or a substituted or unsubstituted (3- to 30-membered)heteroaryl;
e to g, each independently, represent an integer of 1 to 4, in which if e to g represent an integer of 2 or more, each of R′₁ to each of R′₃ may be the same or different.

6. The organic electroluminescent compound according to claim 1, wherein when n represents an integer of 0 and m represents an integer of 1 in the formula 1, A is represented by any one of the following formulas: 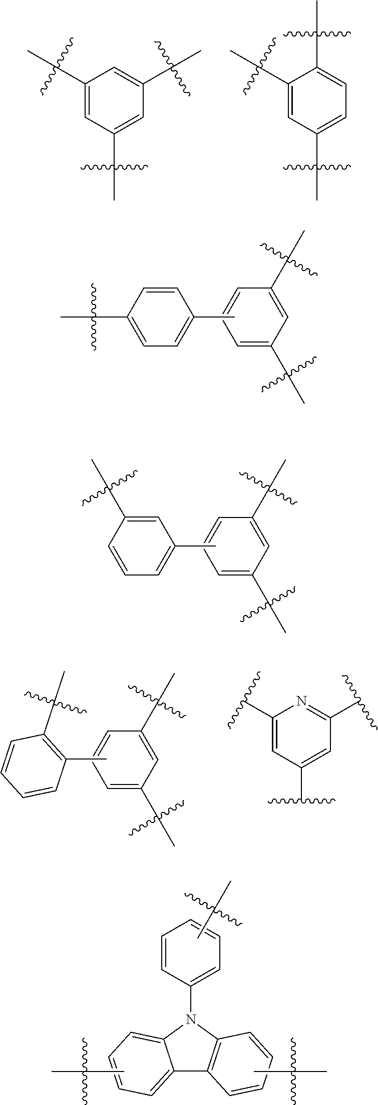
in the formulas, hydrogen may be replaced by deuterium.

7. The organic electroluminescent compound according to claim 1, wherein the substituent(s) of the substituted aryl, the substituted arylene, the substituted heteroaryl, the substituted heteroarylene, the substituted arentriyl, the substituted heteroarentriyl, each independently, are at least one selected from the group consisting of deuterium; a halogen, a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with deuterium; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C2-C30)alkenylamino; a mono- or di- (C6-C30)arylamino; a mono- or di- (3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (02-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and (C1-C30)alkyl(C6-C30)aryl.

8. The organic electroluminescent compound according to claim 1, wherein the compound represented by formula 1 is selected from the group consisting of the following compounds:

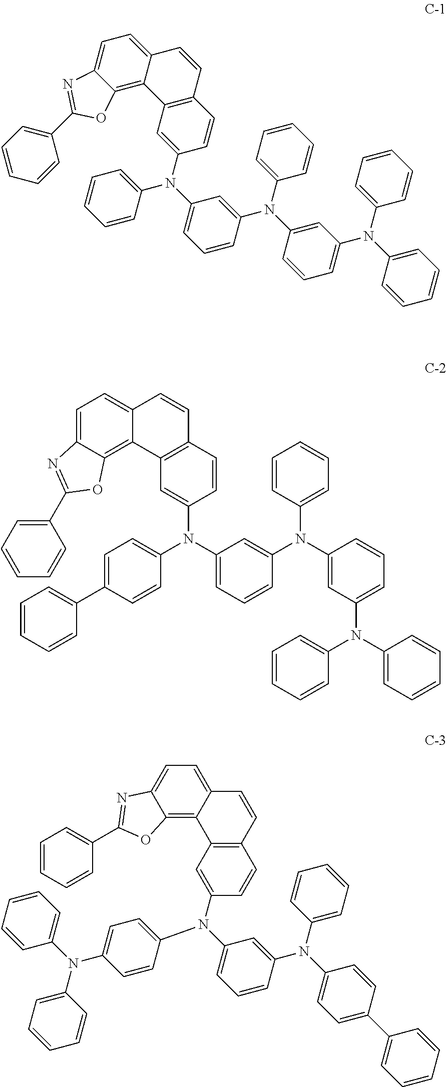 [SMILES: c1ccc(-c2nc3ccc4ccc5ccc(N(c6ccccc6)c6cccc(N(c7ccccc7)c7cccc(N(c8ccccc8)c8ccccc8)c7)c6)cc5c4c3o2)cc1 ; c1ccc(-c2ccc(N(c3cccc(N(c4ccccc4)c4cccc(N(c5ccccc5)c5ccccc5)c4)c3)c3ccc4ccc5ccc6nc(-c7ccccc7)oc6c5c4c3)cc2)cc1 ; c1ccc(-c2ccc(N(c3ccccc3)c3cccc(N(c4ccc(N(c5ccccc5)c5ccccc5)cc4)c4ccc5ccc6ccc7nc(-c8ccccc8)oc7c6c5c4)c3)cc2)cc1]

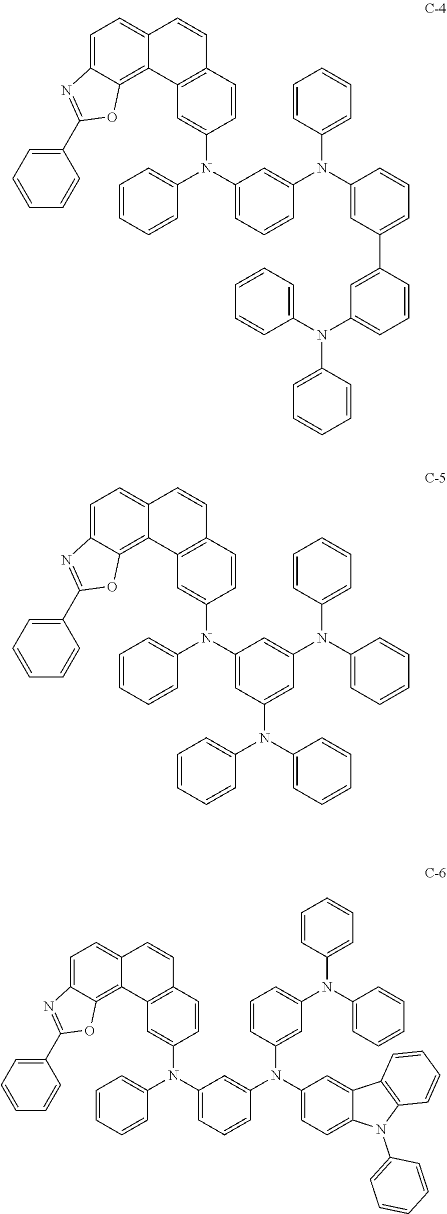 [SMILES: c1ccc(-c2nc3ccc4ccc5ccc(N(c6ccccc6)c6cccc(N(c7ccccc7)c7cccc(-c8cccc(N(c9ccccc9)c9ccccc9)c8)c7)c6)cc5c4c3o2)cc1 ; c1ccc(-c2nc3ccc4ccc5ccc(N(c6ccccc6)c6cc(N(c7ccccc7)c7ccccc7)cc(N(c7ccccc7)c7ccccc7)c6)cc5c4c3o2)cc1 ; c1ccc(-c2nc3ccc4ccc5ccc(N(c6ccccc6)c6cccc(N(c7cccc(N(c8ccccc8)c8ccccc8)c7)c7ccc8c(c7)c7ccccc7n8-c7ccccc7)c6)cc5c4c3o2)cc1]

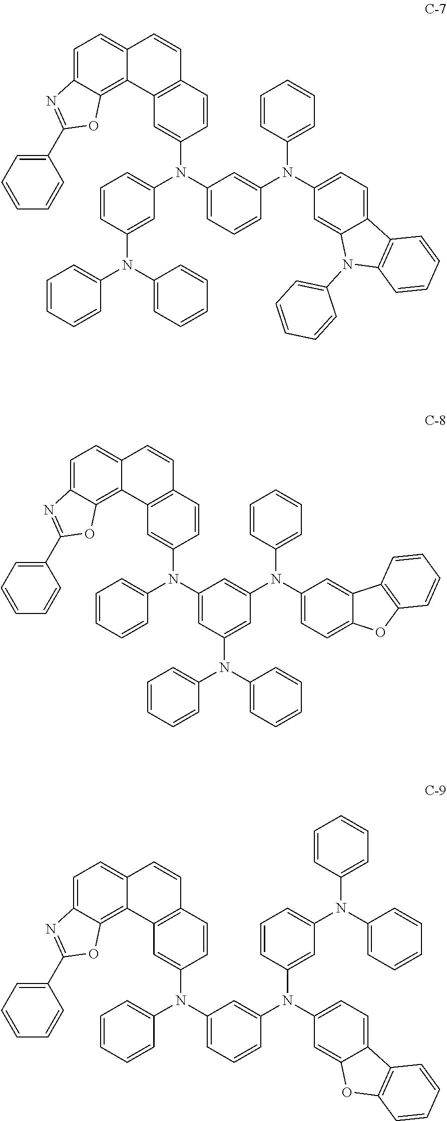 [SMILES: c1ccc(-c2nc3ccc4ccc5ccc(N(c6cccc(N(c7ccccc7)c7ccccc7)c6)c6cccc(N(c7ccccc7)c7ccc8c9ccccc9n(-c9ccccc9)c8c7)c6)cc5c4c3o2)cc1 ; c1ccc(-c2nc3ccc4ccc5ccc(N(c6ccccc6)c6cc(N(c7ccccc7)c7ccccc7)cc(N(c7ccccc7)c7ccc8oc9ccccc9c8c7)c6)cc5c4c3o2)cc1 ; c1ccc(-c2nc3ccc4ccc5ccc(N(c6ccccc6)c6cccc(N(c7cccc(N(c8ccccc8)c8ccccc8)c7)c7ccc8c(c7)oc7ccccc78)c6)cc5c4c3o2)cc1]

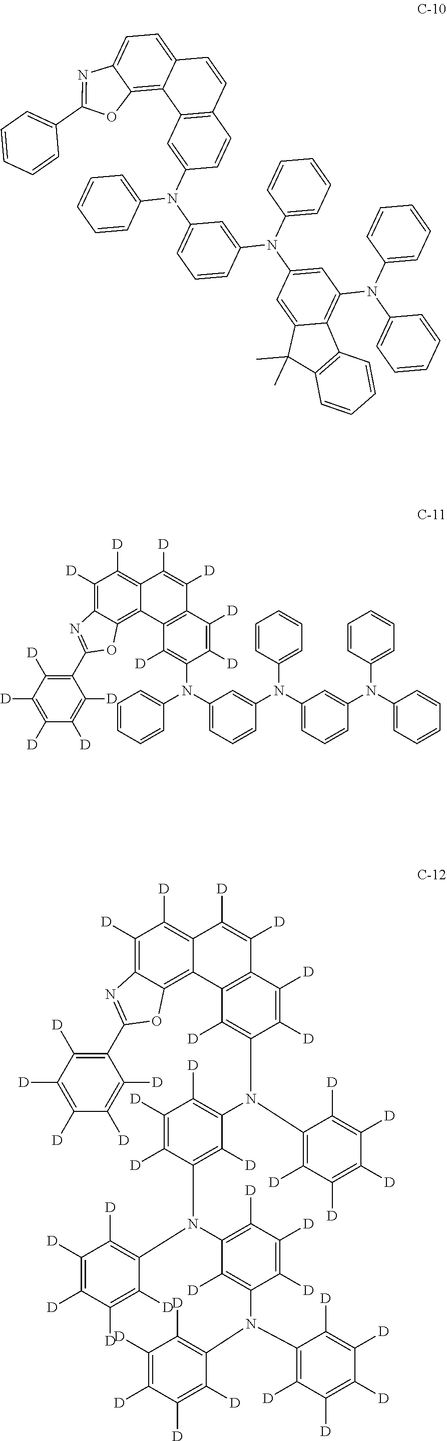

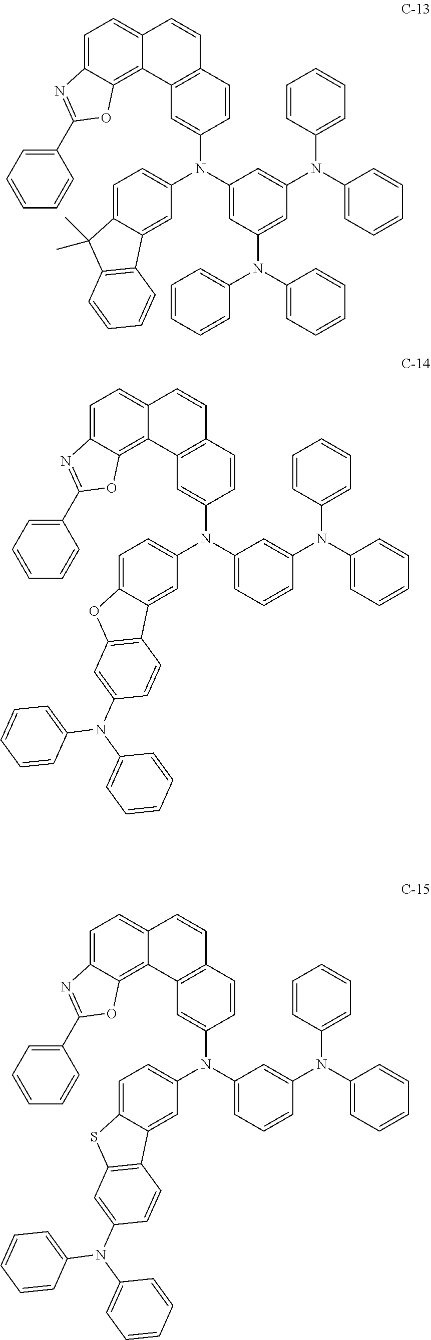 [SMILES: CC1(C)c2ccccc2-c2cc(N(c3cc(N(c4ccccc4)c4ccccc4)cc(N(c4ccccc4)c4ccccc4)c3)c3ccc4ccc5ccc6nc(-c7ccccc7)oc6c5c4c3)ccc21 ; c1ccc(-c2nc3ccc4ccc5ccc(N(c6cccc(N(c7ccccc7)c7ccccc7)c6)c6ccc7oc8cc(N(c9ccccc9)c9ccccc9)ccc8c7c6)cc5c4c3o2)cc1 ; c1ccc(-c2nc3ccc4ccc5ccc(N(c6cccc(N(c7ccccc7)c7ccccc7)c6)c6ccc7sc8cc(N(c9ccccc9)c9ccccc9)ccc8c7c6)cc5c4c3o2)cc1]

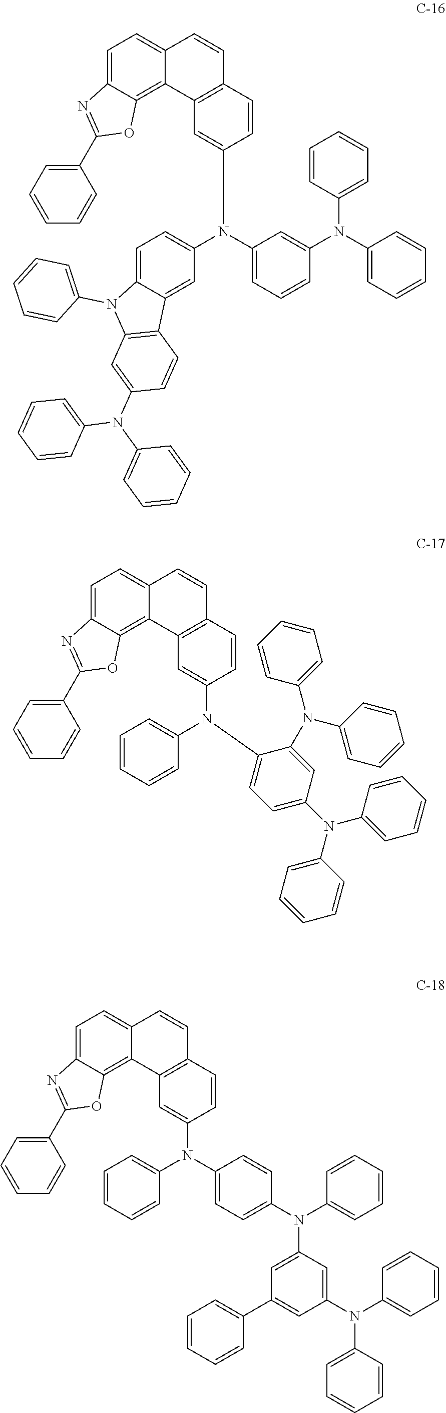 [SMILES: c1ccc(-c2nc3ccc4ccc5ccc(N(c6cccc(N(c7ccccc7)c7ccccc7)c6)c6ccc7c(c6)c6ccc(N(c8ccccc8)c8ccccc8)cc6n7-c6ccccc6)cc5c4c3o2)cc1 ; c1ccc(-c2nc3ccc4ccc5ccc(N(c6ccccc6)c6ccc(N(c7ccccc7)c7ccccc7)cc6N(c6ccccc6)c6ccccc6)cc5c4c3o2)cc1 ; c1ccc(-c2cc(N(c3ccccc3)c3ccccc3)cc(N(c3ccccc3)c3ccc(N(c4ccccc4)c4ccc5ccc6ccc7nc(-c8ccccc8)oc7c6c5c4)cc3)c2)cc1]

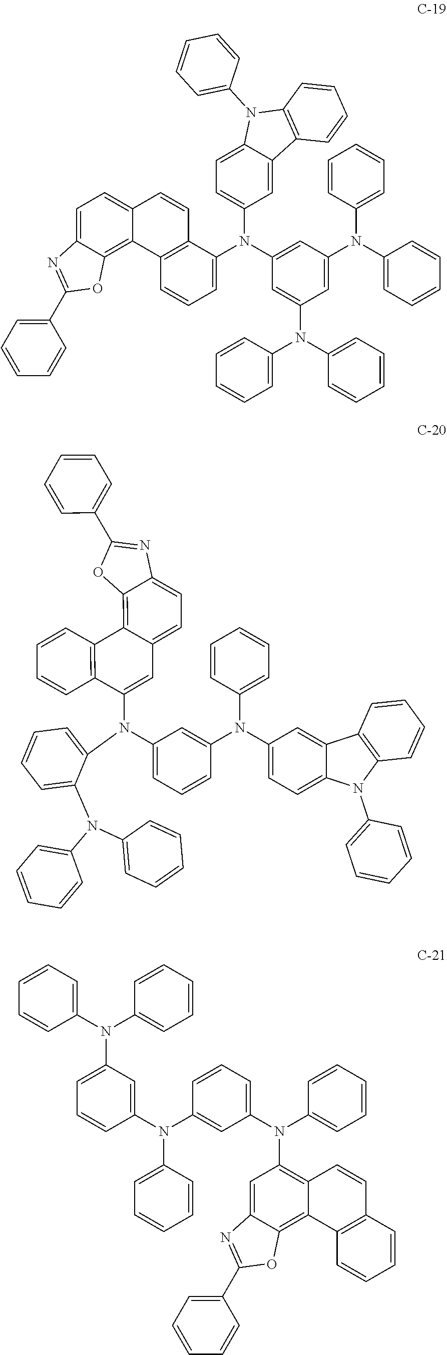 [SMILES: c1ccc(-c2nc3ccc4ccc5c(N(c6cc(N(c7ccccc7)c7ccccc7)cc(N(c7ccccc7)c7ccccc7)c6)c6ccc7c(c6)c6ccccc6n7-c6ccccc6)cccc5c4c3o2)cc1 ; c1ccc(-c2nc3ccc4cc(N(c5cccc(N(c6ccccc6)c6ccc7c(c6)c6ccccc6n7-c6ccccc6)c5)c5ccccc5N(c5ccccc5)c5ccccc5)c5ccccc5c4c3o2)cc1 ; c1ccc(-c2nc3cc(N(c4ccccc4)c4cccc(N(c5ccccc5)c5cccc(N(c6ccccc6)c6ccccc6)c5)c4)c4ccc5ccccc5c4c3o2)cc1]

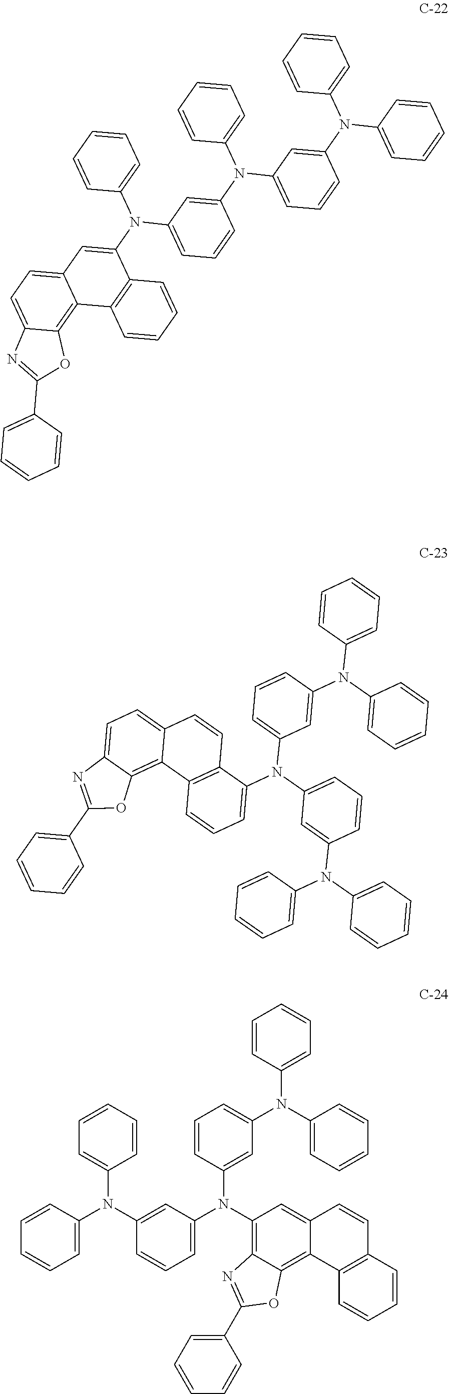 [SMILES: c1ccc(-c2nc3ccc4cc(N(c5ccccc5)c5cccc(N(c6ccccc6)c6cccc(N(c7ccccc7)c7ccccc7)c6)c5)c5ccccc5c4c3o2)cc1 ; c1ccc(-c2nc3ccc4ccc5c(N(c6cccc(N(c7ccccc7)c7ccccc7)c6)c6cccc(N(c7ccccc7)c7ccccc7)c6)cccc5c4c3o2)cc1 ; c1ccc(-c2nc3c(N(c4cccc(N(c5ccccc5)c5ccccc5)c4)c4cccc(N(c5ccccc5)c5ccccc5)c4)cc4ccc5ccccc5c4c3o2)cc1]

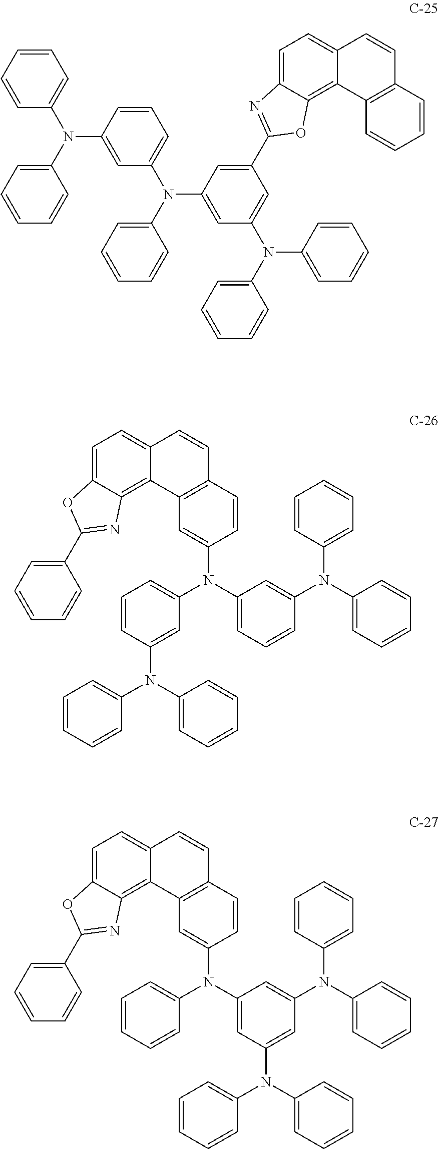 [SMILES: c1ccc(N(c2ccccc2)c2cccc(N(c3ccccc3)c3cc(-c4nc5ccc6ccc7ccccc7c6c5o4)cc(N(c4ccccc4)c4ccccc4)c3)c2)cc1 ; c1ccc(-c2nc3c(ccc4ccc5ccc(N(c6cccc(N(c7ccccc7)c7ccccc7)c6)c6cccc(N(c7ccccc7)c7ccccc7)c6)cc5c43)o2)cc1 ; c1ccc(-c2nc3c(ccc4ccc5ccc(N(c6ccccc6)c6cc(N(c7ccccc7)c7ccccc7)cc(N(c7ccccc7)c7ccccc7)c6)cc5c43)o2)cc1]

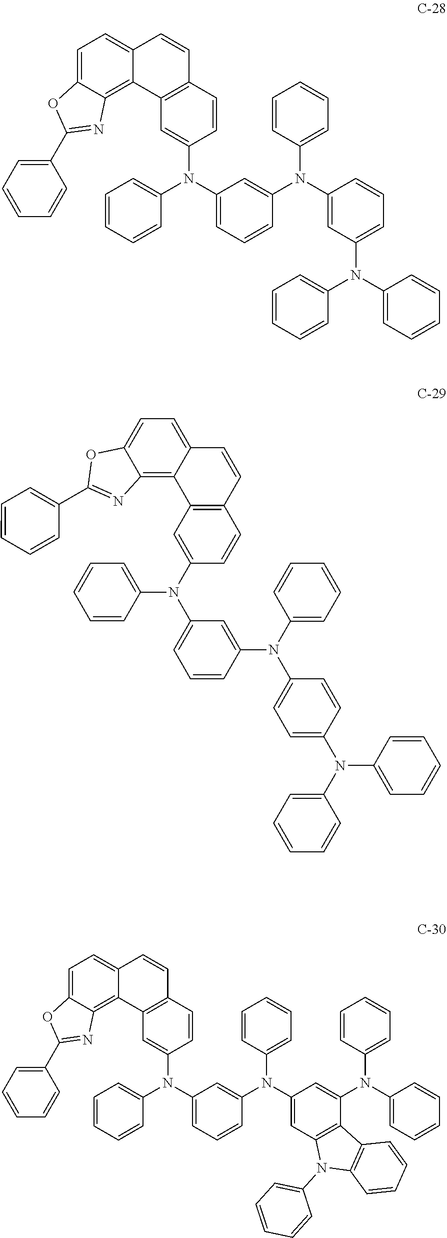 [SMILES: c1ccc(-c2nc3c(ccc4ccc5ccc(N(c6ccccc6)c6cccc(N(c7ccccc7)c7cccc(N(c8ccccc8)c8ccccc8)c7)c6)cc5c43)o2)cc1 ; c1ccc(-c2nc3c(ccc4ccc5ccc(N(c6ccccc6)c6cccc(N(c7ccccc7)c7ccc(N(c8ccccc8)c8ccccc8)cc7)c6)cc5c43)o2)cc1 ; c1ccc(-c2nc3c(ccc4ccc5ccc(N(c6ccccc6)c6cccc(N(c7ccccc7)c7cc(N(c8ccccc8)c8ccccc8)c8c9ccccc9n(-c9ccccc9)c8c7)c6)cc5c43)o2)cc1]

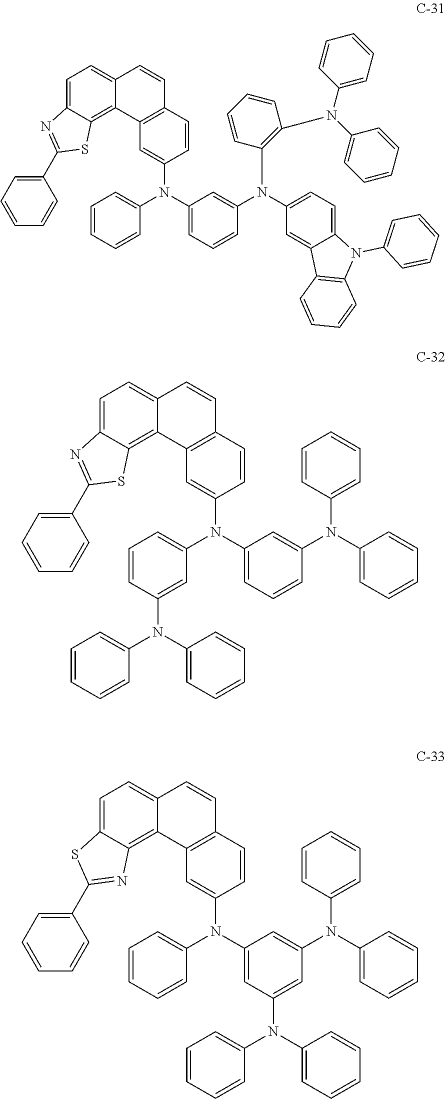 [SMILES: c1ccc(-c2nc3ccc4ccc5ccc(N(c6ccccc6)c6cccc(N(c7ccc8c(c7)c7ccccc7n8-c7ccccc7)c7ccccc7N(c7ccccc7)c7ccccc7)c6)cc5c4c3s2)cc1 ; c1ccc(-c2nc3ccc4ccc5ccc(N(c6cccc(N(c7ccccc7)c7ccccc7)c6)c6cccc(N(c7ccccc7)c7ccccc7)c6)cc5c4c3s2)cc1 ; c1ccc(-c2nc3c(ccc4ccc5ccc(N(c6ccccc6)c6cc(N(c7ccccc7)c7ccccc7)cc(N(c7ccccc7)c7ccccc7)c6)cc5c43)s2)cc1]

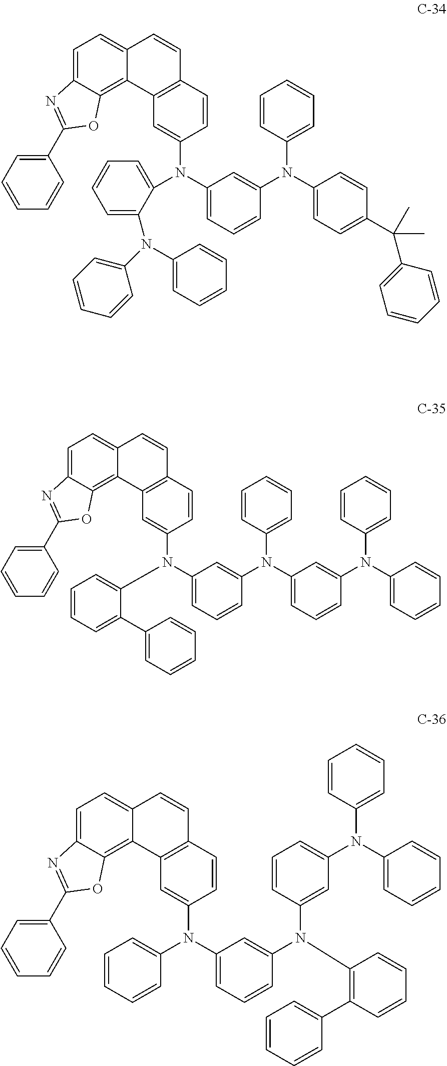 [SMILES: CC(C)(c1ccccc1)c1ccc(N(c2ccccc2)c2cccc(N(c3ccc4ccc5ccc6nc(-c7ccccc7)oc6c5c4c3)c3ccccc3N(c3ccccc3)c3ccccc3)c2)cc1 ; c1ccc(-c2nc3ccc4ccc5ccc(N(c6cccc(N(c7ccccc7)c7cccc(N(c8ccccc8)c8ccccc8)c7)c6)c6ccccc6-c6ccccc6)cc5c4c3o2)cc1 ; c1ccc(-c2nc3ccc4ccc5ccc(N(c6ccccc6)c6cccc(N(c7cccc(N(c8ccccc8)c8ccccc8)c7)c7ccccc7-c7ccccc7)c6)cc5c4c3o2)cc1]

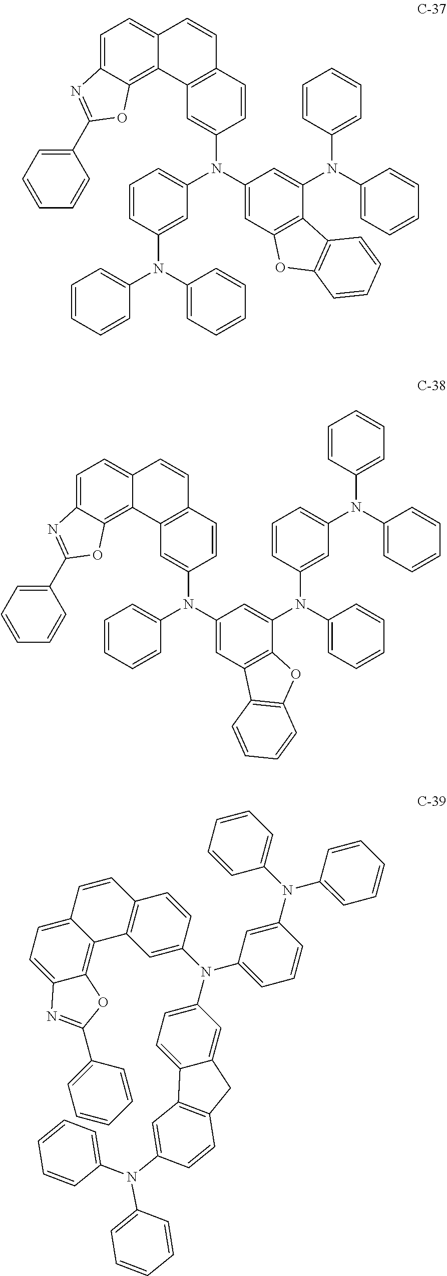 [SMILES: c1ccc(-c2nc3ccc4ccc5ccc(N(c6cccc(N(c7ccccc7)c7ccccc7)c6)c6cc(N(c7ccccc7)c7ccccc7)c7c(c6)oc6ccccc67)cc5c4c3o2)cc1 ; c1ccc(-c2nc3ccc4ccc5ccc(N(c6ccccc6)c6cc(N(c7ccccc7)c7cccc(N(c8ccccc8)c8ccccc8)c7)c7oc8ccccc8c7c6)cc5c4c3o2)cc1 ; c1ccc(-c2nc3ccc4ccc5ccc(N(c6cccc(N(c7ccccc7)c7ccccc7)c6)c6ccc7c(c6)Cc6ccc(N(c8ccccc8)c8ccccc8)cc6-7)cc5c4c3o2)cc1]

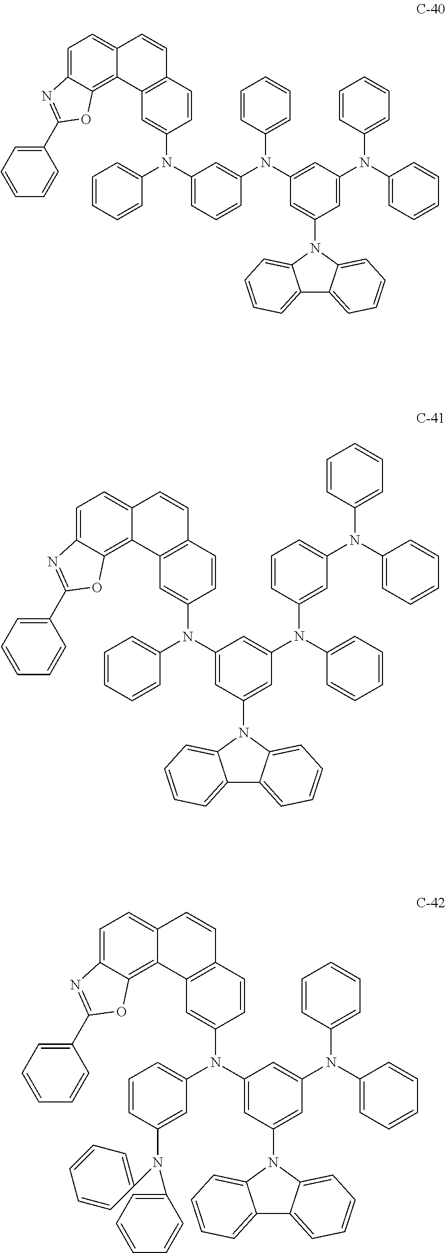 [SMILES: c1ccc(-c2nc3ccc4ccc5ccc(N(c6ccccc6)c6cccc(N(c7ccccc7)c7cc(N(c8ccccc8)c8ccccc8)cc(-n8c9ccccc9c9ccccc98)c7)c6)cc5c4c3o2)cc1 ; c1ccc(-c2nc3ccc4ccc5ccc(N(c6ccccc6)c6cc(N(c7ccccc7)c7cccc(N(c8ccccc8)c8ccccc8)c7)cc(-n7c8ccccc8c8ccccc87)c6)cc5c4c3o2)cc1 ; c1ccc(-c2nc3ccc4ccc5ccc(N(c6cccc(N(c7ccccc7)c7ccccc7)c6)c6cc(N(c7ccccc7)c7ccccc7)cc(-n7c8ccccc8c8ccccc87)c6)cc5c4c3o2)cc1]

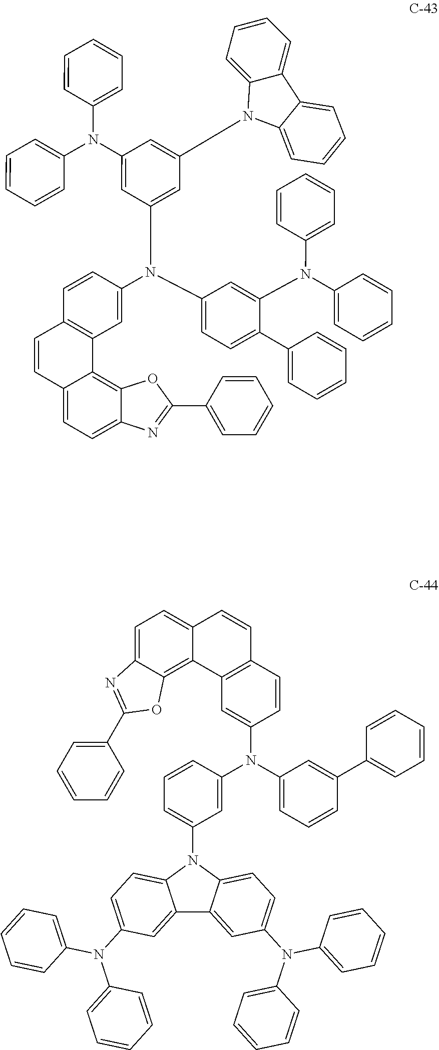 [SMILES: c1ccc(-c2nc3ccc4ccc5ccc(N(c6cc(N(c7ccccc7)c7ccccc7)cc(-n7c8ccccc8c8ccccc87)c6)c6ccc(-c7ccccc7)c(N(c7ccccc7)c7ccccc7)c6)cc5c4c3o2)cc1 ; c1ccc(-c2cccc(N(c3cccc(-n4c5ccc(N(c6ccccc6)c6ccccc6)cc5c5cc(N(c6ccccc6)c6ccccc6)ccc54)c3)c3ccc4ccc5ccc6nc(-c7ccccc7)oc6c5c4c3)c2)cc1]

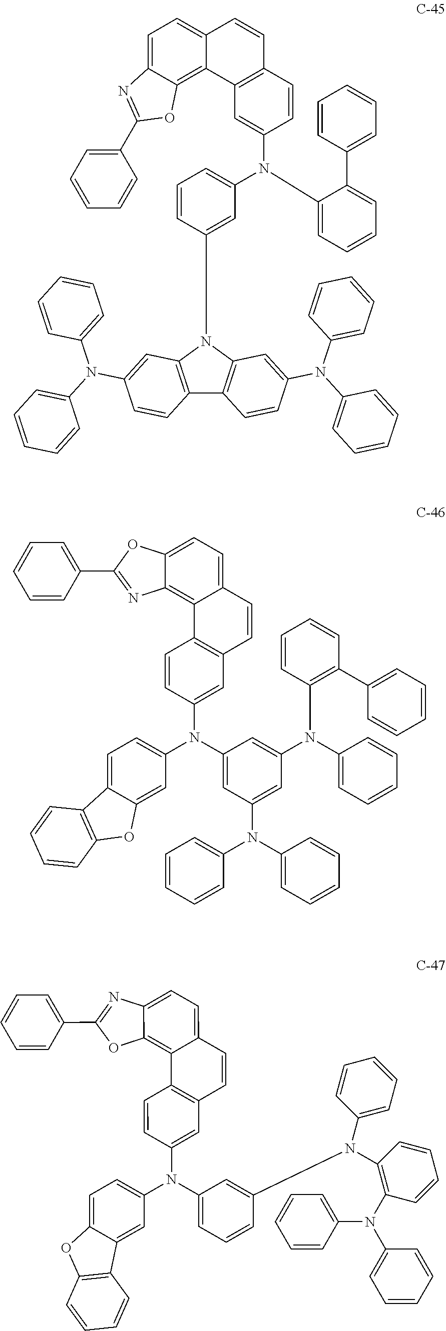 [SMILES: c1ccc(-c2nc3ccc4ccc5ccc(N(c6cccc(-n7c8cc(N(c9ccccc9)c9ccccc9)ccc8c8ccc(N(c9ccccc9)c9ccccc9)cc87)c6)c6ccccc6-c6ccccc6)cc5c4c3o2)cc1 ; c1ccc(-c2nc3c(ccc4ccc5cc(N(c6cc(N(c7ccccc7)c7ccccc7)cc(N(c7ccccc7)c7ccccc7-c7ccccc7)c6)c6ccc7c(c6)oc6ccccc67)ccc5c43)o2)cc1 ; c1ccc(-c2nc3ccc4ccc5cc(N(c6cccc(N(c7ccccc7)c7ccccc7N(c7ccccc7)c7ccccc7)c6)c6ccc7oc8ccccc8c7c6)ccc5c4c3o2)cc1]

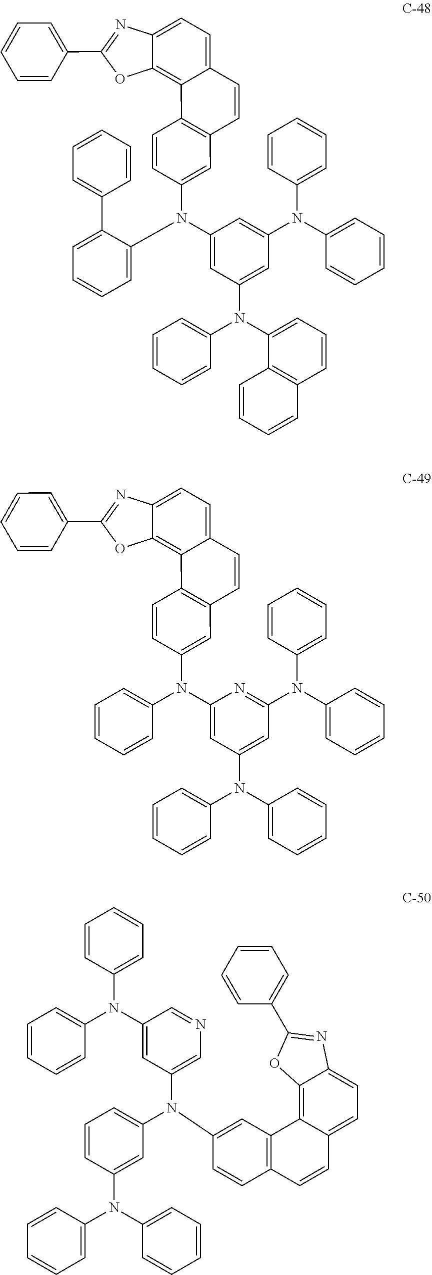 [SMILES: c1ccc(-c2nc3ccc4ccc5cc(N(c6cc(N(c7ccccc7)c7ccccc7)cc(N(c7ccccc7)c7cccc8ccccc78)c6)c6ccccc6-c6ccccc6)ccc5c4c3o2)cc1 ; c1ccc(-c2nc3ccc4ccc5cc(N(c6ccccc6)c6cc(N(c7ccccc7)c7ccccc7)cc(N(c7ccccc7)c7ccccc7)n6)ccc5c4c3o2)cc1 ; c1ccc(-c2nc3ccc4ccc5ccc(N(c6cccc(N(c7ccccc7)c7ccccc7)c6)c6cncc(N(c7ccccc7)c7ccccc7)c6)cc5c4c3o2)cc1]

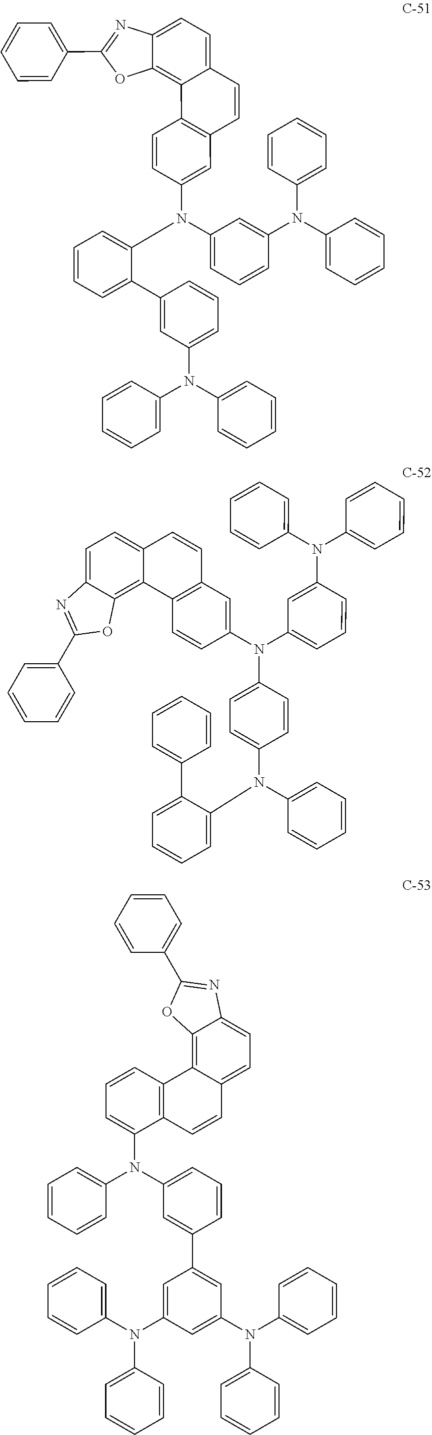

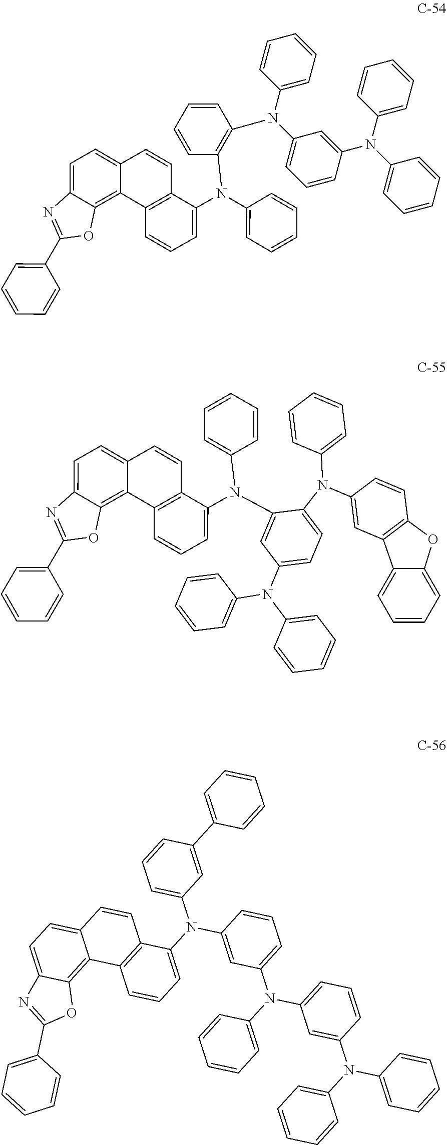 [SMILES: c1ccc(-c2nc3ccc4ccc5c(N(c6ccccc6)c6ccccc6N(c6ccccc6)c6cccc(N(c7ccccc7)c7ccccc7)c6)cccc5c4c3o2)cc1 ; c1ccc(-c2nc3ccc4ccc5c(N(c6ccccc6)c6cc(N(c7ccccc7)c7ccccc7)ccc6N(c6ccccc6)c6ccc7oc8ccccc8c7c6)cccc5c4c3o2)cc1 ; c1ccc(-c2cccc(N(c3cccc(N(c4ccccc4)c4cccc(N(c5ccccc5)c5ccccc5)c4)c3)c3cccc4c3ccc3ccc5nc(-c6ccccc6)oc5c34)c2)cc1]

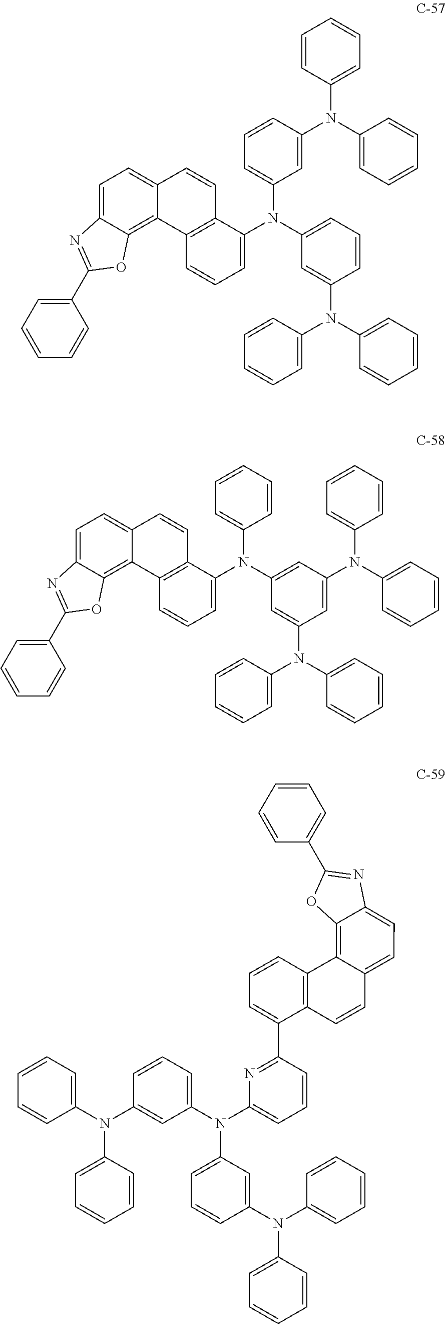 [SMILES: c1ccc(-c2nc3ccc4ccc5c(N(c6cccc(N(c7ccccc7)c7ccccc7)c6)c6cccc(N(c7ccccc7)c7ccccc7)c6)cccc5c4c3o2)cc1 ; c1ccc(-c2nc3ccc4ccc5c(N(c6ccccc6)c6cc(N(c7ccccc7)c7ccccc7)cc(N(c7ccccc7)c7ccccc7)c6)cccc5c4c3o2)cc1 ; c1ccc(-c2nc3ccc4ccc5c(-c6cccc(N(c7cccc(N(c8ccccc8)c8ccccc8)c7)c7cccc(N(c8ccccc8)c8ccccc8)c7)n6)cccc5c4c3o2)cc1]

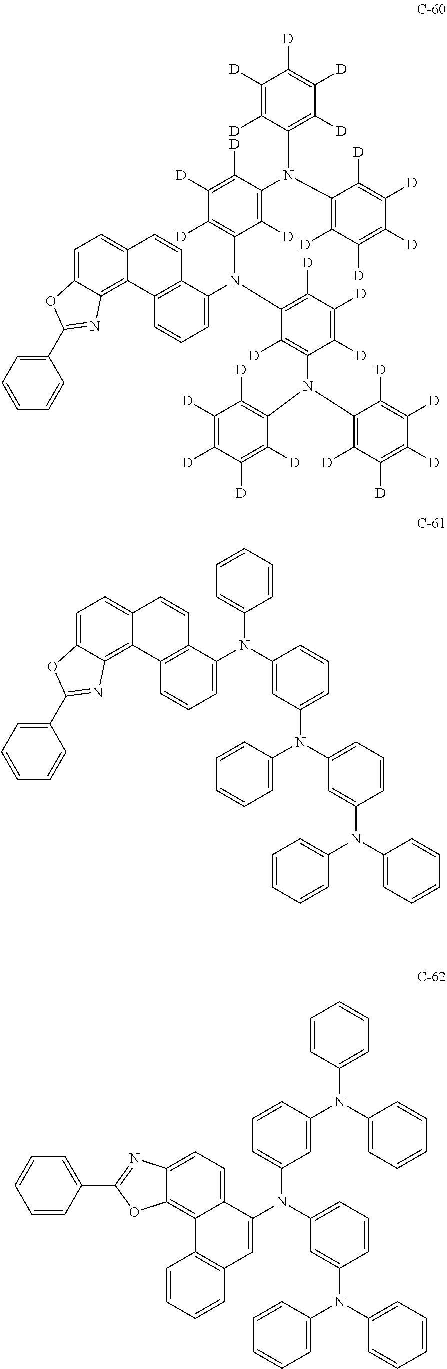 [SMILES: [2H]c1c([2H])c([2H])c(N(c2c([2H])c([2H])c([2H])c([2H])c2[2H])c2c([2H])c([2H])c([2H])c(N(c3c([2H])c([2H])c([2H])c(N(c4c([2H])c([2H])c([2H])c([2H])c4[2H])c4c([2H])c([2H])c([2H])c([2H])c4[2H])c3[2H])c3cccc4c3ccc3ccc5oc(-c6ccccc6)nc5c34)c2[2H])c([2H])c1[2H] ; c1ccc(-c2nc3c(ccc4ccc5c(N(c6ccccc6)c6cccc(N(c7ccccc7)c7cccc(N(c8ccccc8)c8ccccc8)c7)c6)cccc5c43)o2)cc1 ; c1ccc(-c2nc3ccc4c(N(c5cccc(N(c6ccccc6)c6ccccc6)c5)c5cccc(N(c6ccccc6)c6ccccc6)c5)cc5ccccc5c4c3o2)cc1]

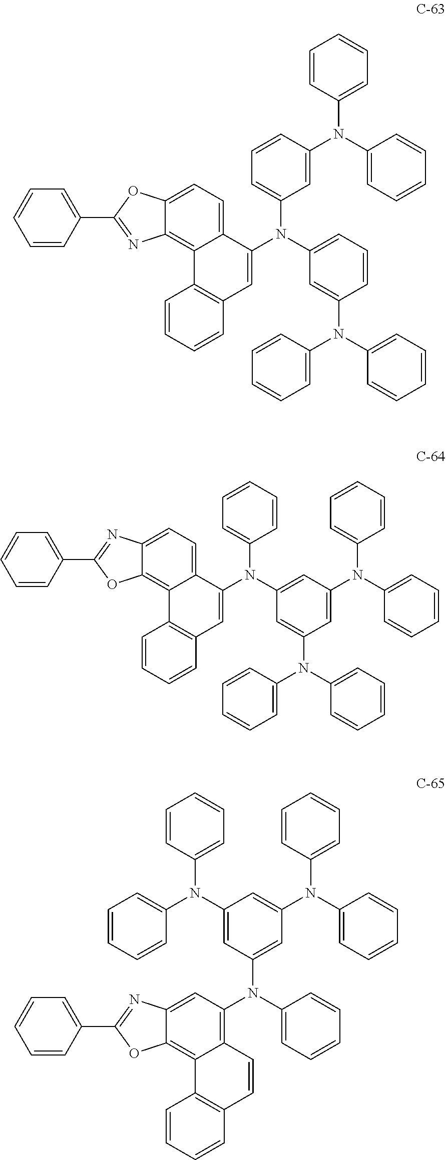 [SMILES: c1ccc(-c2nc3c(ccc4c(N(c5cccc(N(c6ccccc6)c6ccccc6)c5)c5cccc(N(c6ccccc6)c6ccccc6)c5)cc5ccccc5c43)o2)cc1 ; c1ccc(-c2nc3ccc4c(N(c5ccccc5)c5cc(N(c6ccccc6)c6ccccc6)cc(N(c6ccccc6)c6ccccc6)c5)cc5ccccc5c4c3o2)cc1 ; c1ccc(-c2nc3cc(N(c4ccccc4)c4cc(N(c5ccccc5)c5ccccc5)cc(N(c5ccccc5)c5ccccc5)c4)c4ccc5ccccc5c4c3o2)cc1]

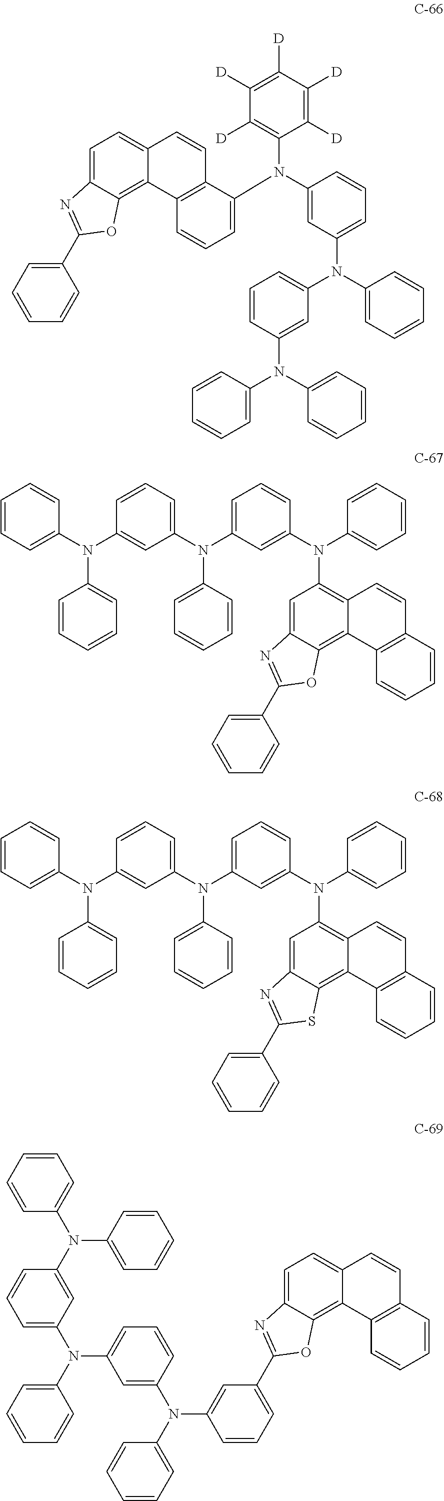 [SMILES: [2H]c1c([2H])c([2H])c(N(c2cccc(N(c3ccccc3)c3cccc(N(c4ccccc4)c4ccccc4)c3)c2)c2cccc3c2ccc2ccc4nc(-c5ccccc5)oc4c23)c([2H])c1[2H] ; c1ccc(-c2nc3cc(N(c4ccccc4)c4cccc(N(c5ccccc5)c5cccc(N(c6ccccc6)c6ccccc6)c5)c4)c4ccc5ccccc5c4c3o2)cc1 ; c1ccc(-c2nc3cc(N(c4ccccc4)c4cccc(N(c5ccccc5)c5cccc(N(c6ccccc6)c6ccccc6)c5)c4)c4ccc5ccccc5c4c3s2)cc1 ; c1ccc(N(c2ccccc2)c2cccc(N(c3ccccc3)c3cccc(N(c4ccccc4)c4cccc(-c5nc6ccc7ccc8ccccc8c7c6o5)c4)c3)c2)cc1]

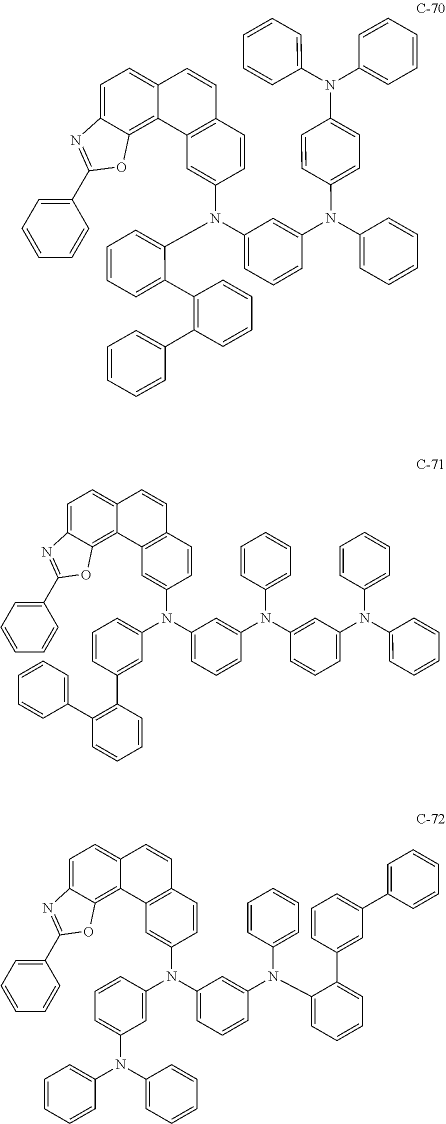 [SMILES: c1ccc(-c2nc3ccc4ccc5ccc(N(c6cccc(N(c7ccccc7)c7ccc(N(c8ccccc8)c8ccccc8)cc7)c6)c6ccccc6-c6ccccc6-c6ccccc6)cc5c4c3o2)cc1 ; c1ccc(-c2nc3ccc4ccc5ccc(N(c6cccc(-c7ccccc7-c7ccccc7)c6)c6cccc(N(c7ccccc7)c7cccc(N(c8ccccc8)c8ccccc8)c7)c6)cc5c4c3o2)cc1 ; c1ccc(-c2cccc(-c3ccccc3N(c3ccccc3)c3cccc(N(c4cccc(N(c5ccccc5)c5ccccc5)c4)c4ccc5ccc6ccc7nc(-c8ccccc8)oc7c6c5c4)c3)c2)cc1]

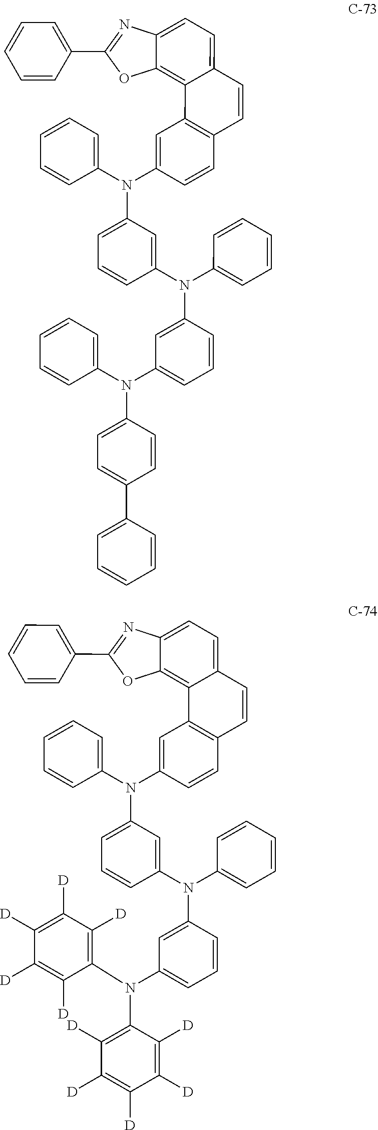

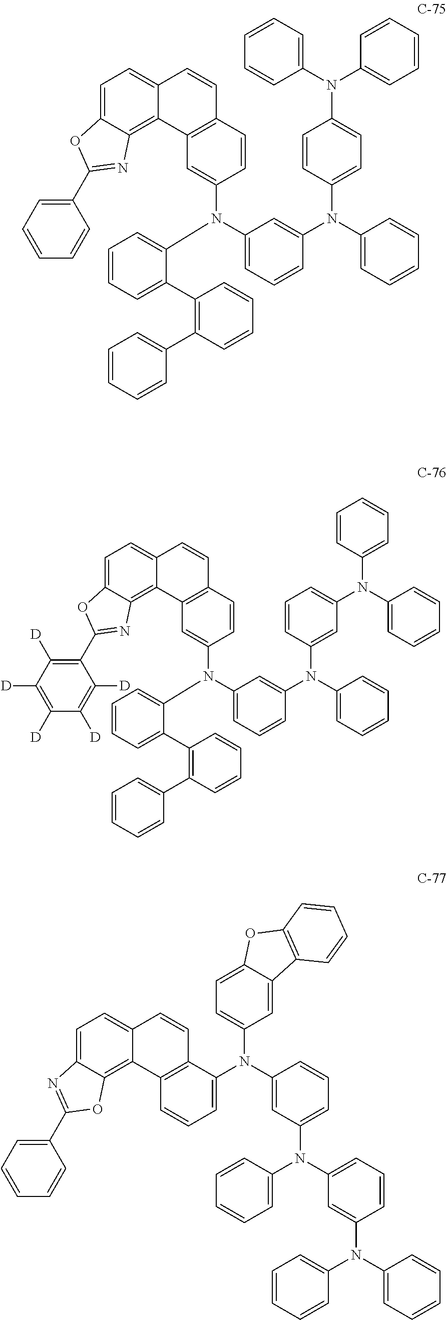 [SMILES: c1ccc(-c2nc3c(ccc4ccc5ccc(N(c6cccc(N(c7ccccc7)c7ccc(N(c8ccccc8)c8ccccc8)cc7)c6)c6ccccc6-c6ccccc6-c6ccccc6)cc5c43)o2)cc1 ; [2H]c1c([2H])c([2H])c(-c2nc3c(ccc4ccc5ccc(N(c6cccc(N(c7ccccc7)c7cccc(N(c8ccccc8)c8ccccc8)c7)c6)c6ccccc6-c6ccccc6-c6ccccc6)cc5c43)o2)c([2H])c1[2H] ; c1ccc(-c2nc3ccc4ccc5c(N(c6cccc(N(c7ccccc7)c7cccc(N(c8ccccc8)c8ccccc8)c7)c6)c6ccc7oc8ccccc8c7c6)cccc5c4c3o2)cc1]

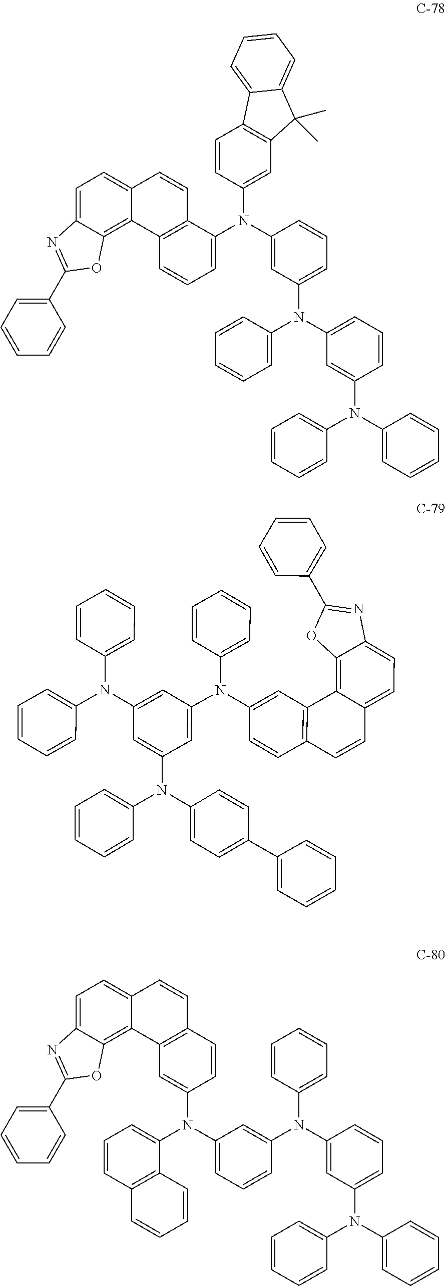

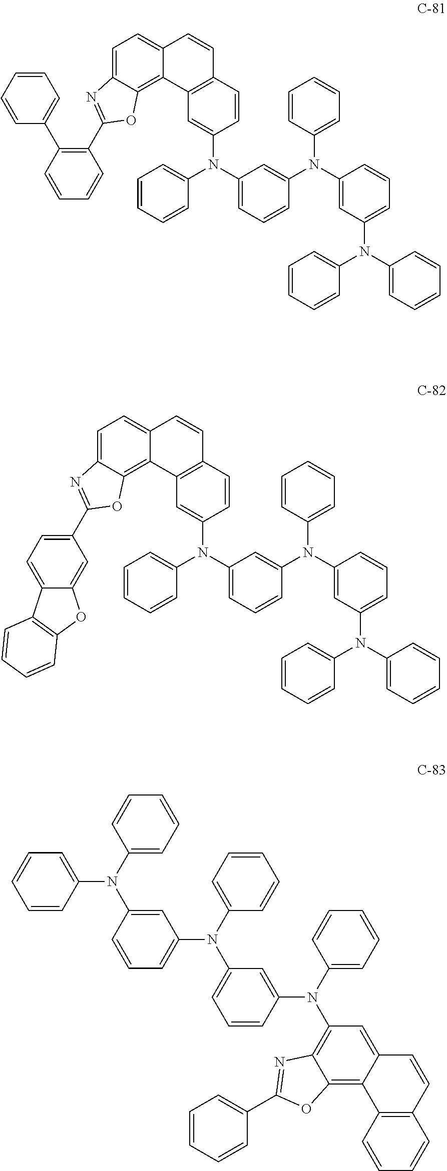 [SMILES: c1ccc(-c2ccccc2-c2nc3ccc4ccc5ccc(N(c6ccccc6)c6cccc(N(c7ccccc7)c7cccc(N(c8ccccc8)c8ccccc8)c7)c6)cc5c4c3o2)cc1 ; c1ccc(N(c2ccccc2)c2cccc(N(c3ccccc3)c3cccc(N(c4ccccc4)c4ccc5ccc6ccc7nc(-c8ccc9c(c8)oc8ccccc89)oc7c6c5c4)c3)c2)cc1 ; c1ccc(-c2nc3c(N(c4ccccc4)c4cccc(N(c5ccccc5)c5cccc(N(c6ccccc6)c6ccccc6)c5)c4)cc4ccc5ccccc5c4c3o2)cc1]

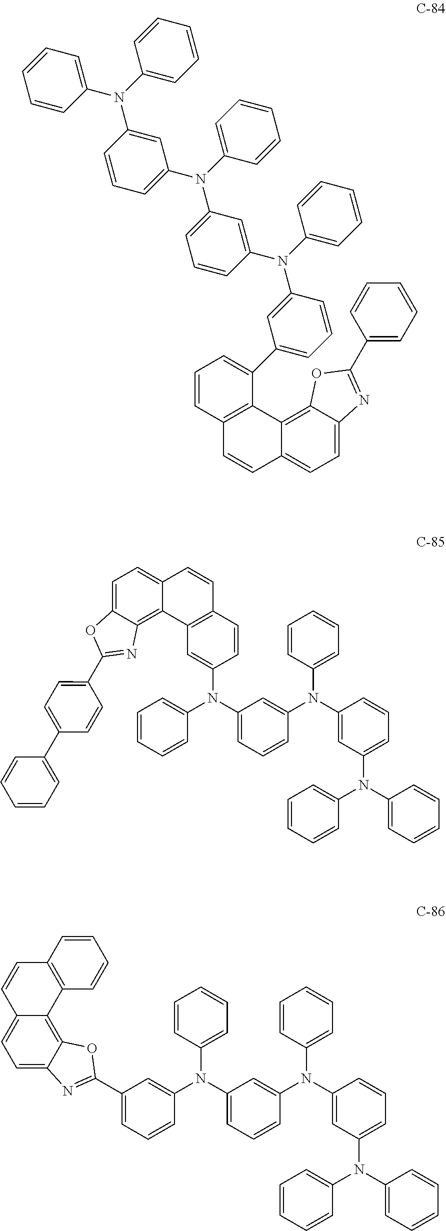 [SMILES: c1ccc(-c2nc3ccc4ccc5cccc(-c6cccc(N(c7ccccc7)c7cccc(N(c8ccccc8)c8cccc(N(c9ccccc9)c9ccccc9)c8)c7)c6)c5c4c3o2)cc1 ; c1ccc(-c2ccc(-c3nc4c(ccc5ccc6ccc(N(c7ccccc7)c7cccc(N(c8ccccc8)c8cccc(N(c9ccccc9)c9ccccc9)c8)c7)cc6c54)o3)cc2)cc1 ; c1ccc(N(c2ccccc2)c2cccc(N(c3ccccc3)c3cccc(N(c4ccccc4)c4cccc(-c5nc6ccc7ccc8ccccc8c7c6o5)c4)c3)c2)cc1]

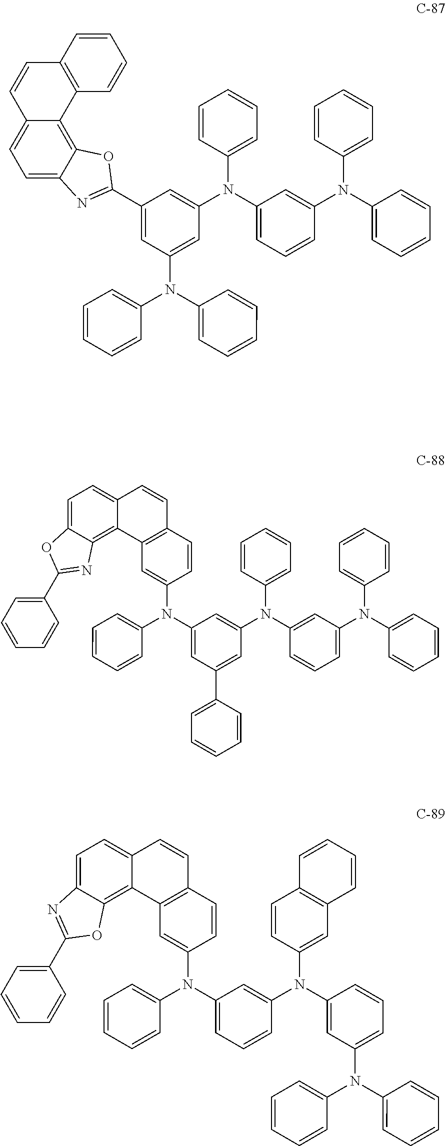 [SMILES: c1ccc(N(c2ccccc2)c2cccc(N(c3ccccc3)c3cc(-c4nc5ccc6ccc7ccccc7c6c5o4)cc(N(c4ccccc4)c4ccccc4)c3)c2)cc1 ; c1ccc(-c2cc(N(c3ccccc3)c3cccc(N(c4ccccc4)c4ccccc4)c3)cc(N(c3ccccc3)c3ccc4ccc5ccc6oc(-c7ccccc7)nc6c5c4c3)c2)cc1 ; c1ccc(-c2nc3ccc4ccc5ccc(N(c6ccccc6)c6cccc(N(c7cccc(N(c8ccccc8)c8ccccc8)c7)c7ccc8ccccc8c7)c6)cc5c4c3o2)cc1]

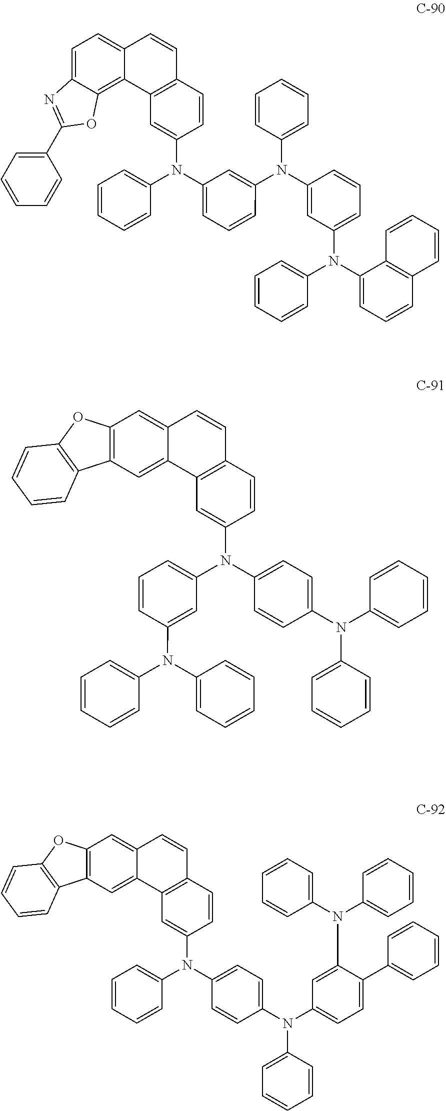 [SMILES: c1ccc(-c2nc3ccc4ccc5ccc(N(c6ccccc6)c6cccc(N(c7ccccc7)c7cccc(N(c8ccccc8)c8cccc9ccccc89)c7)c6)cc5c4c3o2)cc1 ; c1ccc(N(c2ccccc2)c2ccc(N(c3cccc(N(c4ccccc4)c4ccccc4)c3)c3ccc4ccc5cc6oc7ccccc7c6cc5c4c3)cc2)cc1 ; c1ccc(-c2ccc(N(c3ccccc3)c3ccc(N(c4ccccc4)c4ccc5ccc6cc7oc8ccccc8c7cc6c5c4)cc3)cc2N(c2ccccc2)c2ccccc2)cc1]

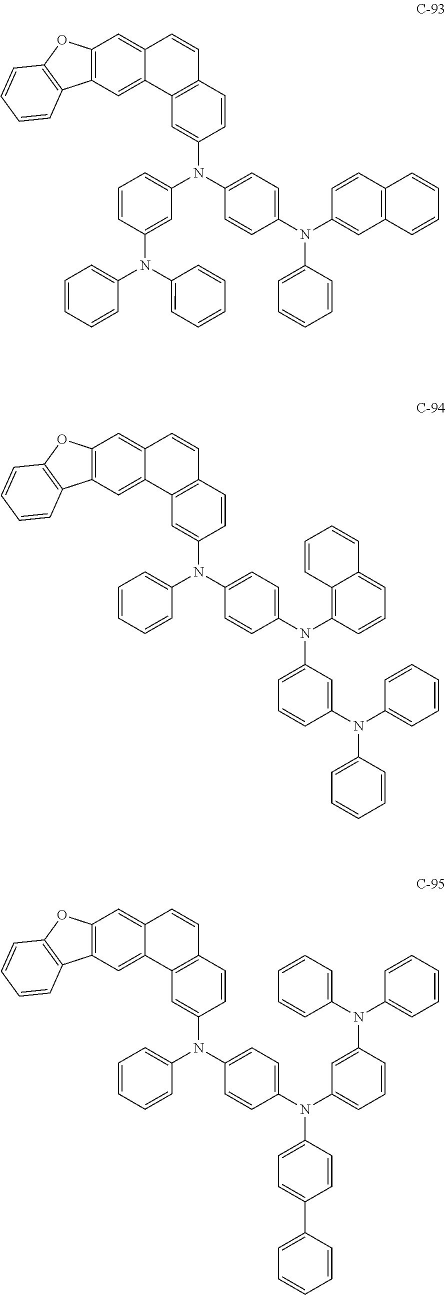 [SMILES: c1ccc(N(c2ccccc2)c2cccc(N(c3ccc(N(c4ccccc4)c4ccc5ccccc5c4)cc3)c3ccc4ccc5cc6oc7ccccc7c6cc5c4c3)c2)cc1 ; c1ccc(N(c2ccccc2)c2cccc(N(c3ccc(N(c4ccccc4)c4ccc5ccc6cc7oc8ccccc8c7cc6c5c4)cc3)c3cccc4ccccc34)c2)cc1 ; c1ccc(-c2ccc(N(c3ccc(N(c4ccccc4)c4ccc5ccc6cc7oc8ccccc8c7cc6c5c4)cc3)c3cccc(N(c4ccccc4)c4ccccc4)c3)cc2)cc1]

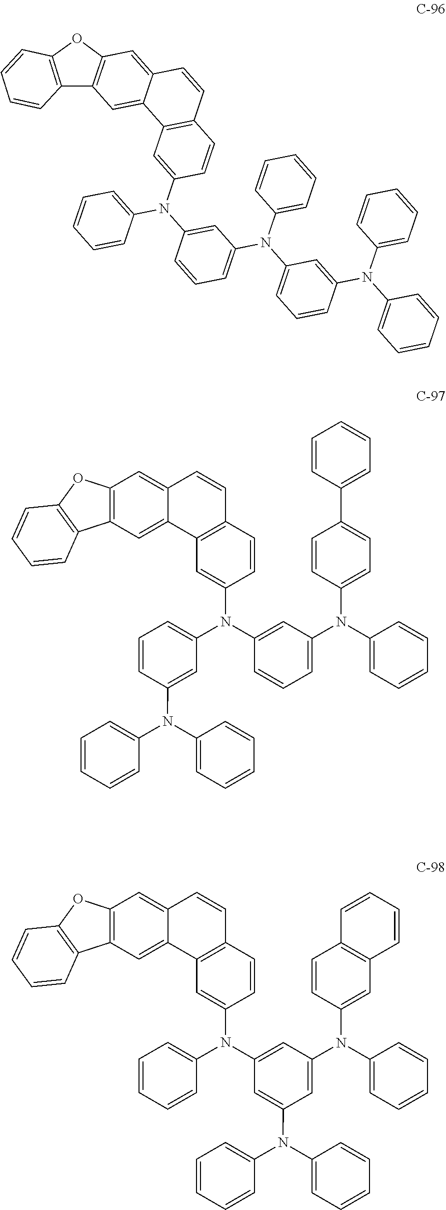 [SMILES: c1ccc(N(c2ccccc2)c2cccc(N(c3ccccc3)c3cccc(N(c4ccccc4)c4ccc5ccc6cc7oc8ccccc8c7cc6c5c4)c3)c2)cc1 ; c1ccc(-c2ccc(N(c3ccccc3)c3cccc(N(c4cccc(N(c5ccccc5)c5ccccc5)c4)c4ccc5ccc6cc7oc8ccccc8c7cc6c5c4)c3)cc2)cc1 ; c1ccc(N(c2ccccc2)c2cc(N(c3ccccc3)c3ccc4ccccc4c3)cc(N(c3ccccc3)c3ccc4ccc5cc6oc7ccccc7c6cc5c4c3)c2)cc1]

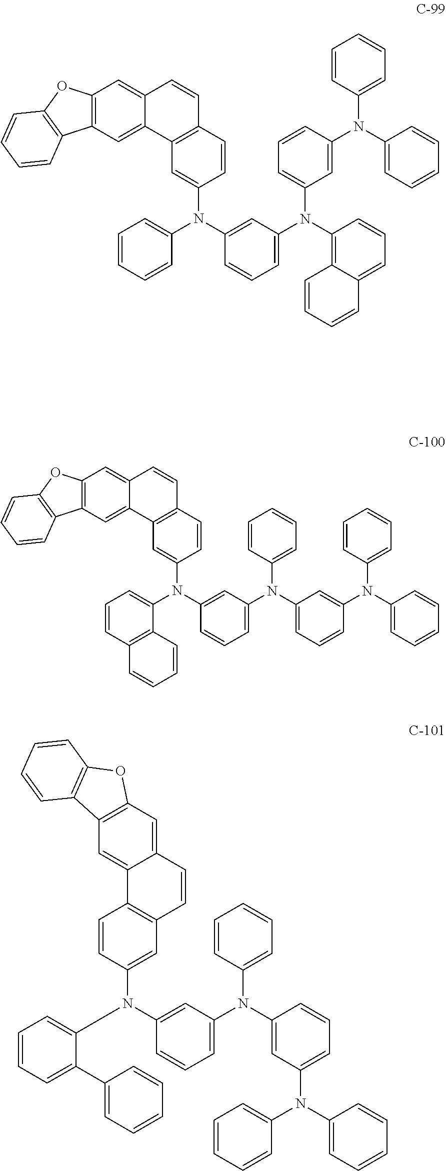 [SMILES: c1ccc(N(c2ccccc2)c2cccc(N(c3cccc(N(c4ccccc4)c4ccc5ccc6cc7oc8ccccc8c7cc6c5c4)c3)c3cccc4ccccc34)c2)cc1 ; c1ccc(N(c2ccccc2)c2cccc(N(c3ccccc3)c3cccc(N(c4ccc5ccc6cc7oc8ccccc8c7cc6c5c4)c4cccc5ccccc45)c3)c2)cc1 ; c1ccc(-c2ccccc2N(c2cccc(N(c3ccccc3)c3cccc(N(c4ccccc4)c4ccccc4)c3)c2)c2ccc3c(ccc4cc5oc6ccccc6c5cc43)c2)cc1]

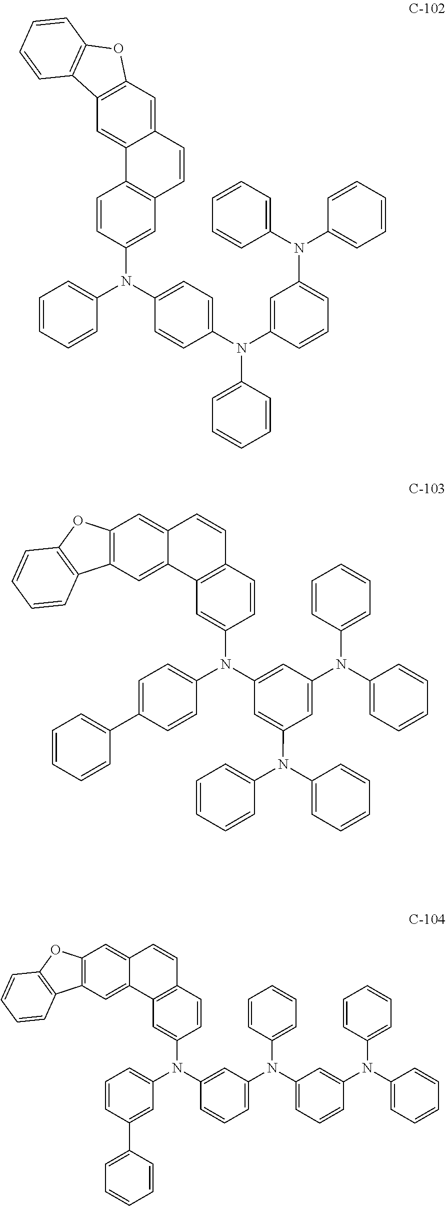 [SMILES: c1ccc(N(c2ccccc2)c2cccc(N(c3ccccc3)c3ccc(N(c4ccccc4)c4ccc5c(ccc6cc7oc8ccccc8c7cc65)c4)cc3)c2)cc1 ; c1ccc(-c2ccc(N(c3cc(N(c4ccccc4)c4ccccc4)cc(N(c4ccccc4)c4ccccc4)c3)c3ccc4ccc5cc6oc7ccccc7c6cc5c4c3)cc2)cc1 ; c1ccc(-c2cccc(N(c3cccc(N(c4ccccc4)c4cccc(N(c5ccccc5)c5ccccc5)c4)c3)c3ccc4ccc5cc6oc7ccccc7c6cc5c4c3)c2)cc1]

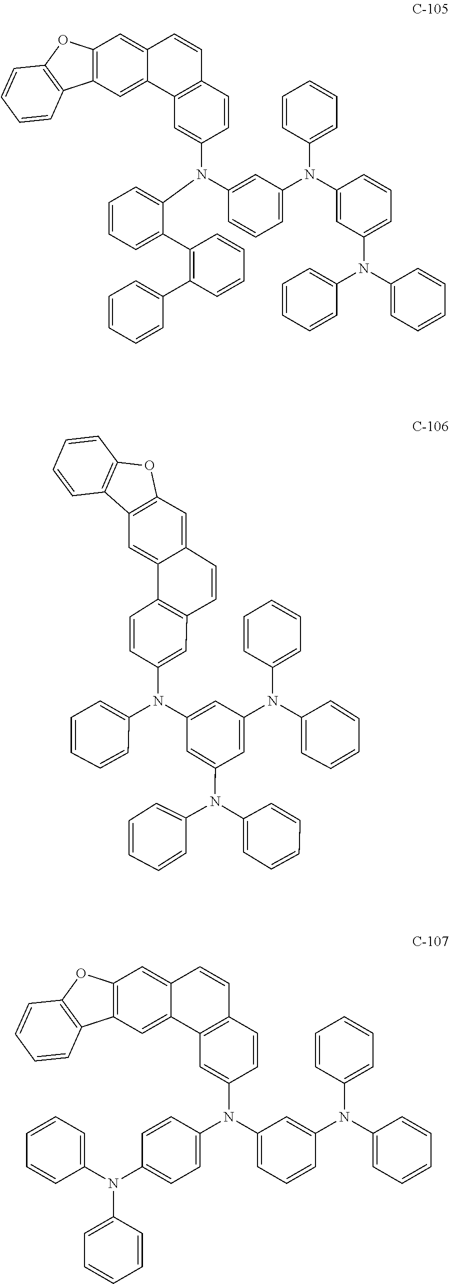 [SMILES: c1ccc(-c2ccccc2-c2ccccc2N(c2cccc(N(c3ccccc3)c3cccc(N(c4ccccc4)c4ccccc4)c3)c2)c2ccc3ccc4cc5oc6ccccc6c5cc4c3c2)cc1 ; c1ccc(N(c2ccccc2)c2cc(N(c3ccccc3)c3ccccc3)cc(N(c3ccccc3)c3ccc4c(ccc5cc6oc7ccccc7c6cc54)c3)c2)cc1 ; c1ccc(N(c2ccccc2)c2ccc(N(c3cccc(N(c4ccccc4)c4ccccc4)c3)c3ccc4ccc5cc6oc7ccccc7c6cc5c4c3)cc2)cc1]

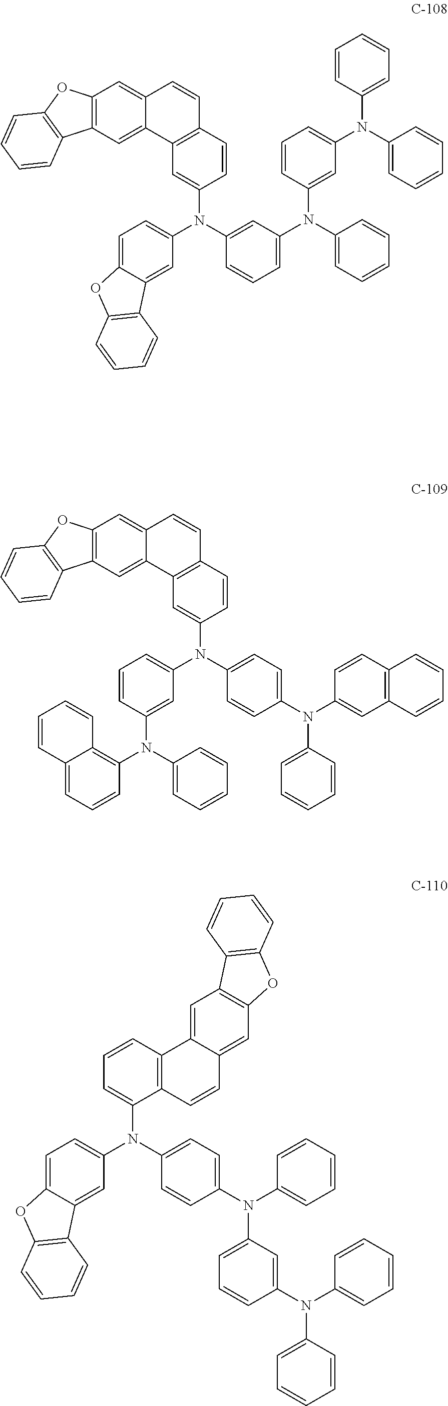 [SMILES: c1ccc(N(c2ccccc2)c2cccc(N(c3ccccc3)c3cccc(N(c4ccc5ccc6cc7oc8ccccc8c7cc6c5c4)c4ccc5oc6ccccc6c5c4)c3)c2)cc1 ; c1ccc(N(c2ccc(N(c3cccc(N(c4ccccc4)c4cccc5ccccc45)c3)c3ccc4ccc5cc6oc7ccccc7c6cc5c4c3)cc2)c2ccc3ccccc3c2)cc1 ; c1ccc(N(c2ccccc2)c2cccc(N(c3ccccc3)c3ccc(N(c4ccc5oc6ccccc6c5c4)c4cccc5c4ccc4cc6oc7ccccc7c6cc45)cc3)c2)cc1]

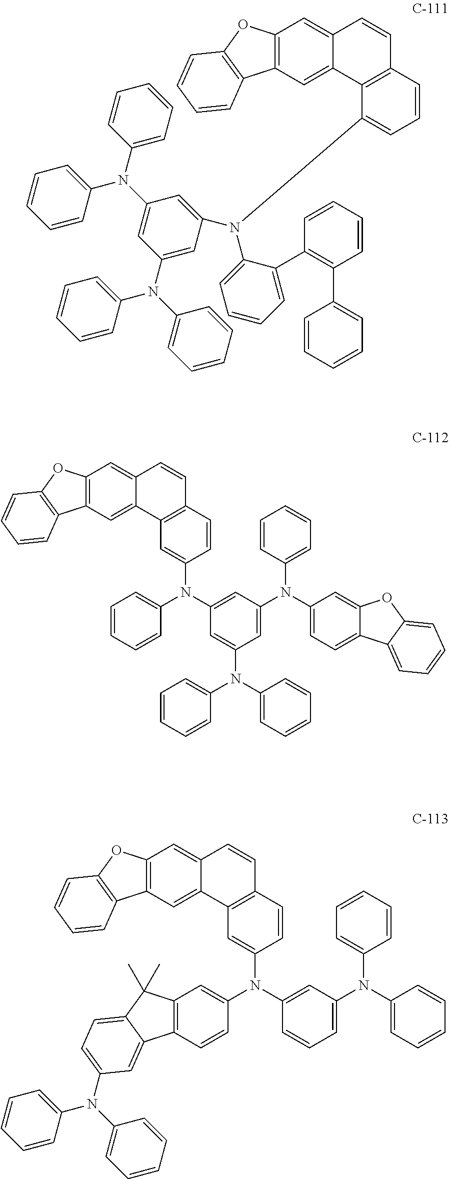 [SMILES: c1ccc(-c2ccccc2-c2ccccc2N(c2cc(N(c3ccccc3)c3ccccc3)cc(N(c3ccccc3)c3ccccc3)c2)c2cccc3ccc4cc5oc6ccccc6c5cc4c23)cc1 ; c1ccc(N(c2ccccc2)c2cc(N(c3ccccc3)c3ccc4c(c3)oc3ccccc34)cc(N(c3ccccc3)c3ccc4ccc5cc6oc7ccccc7c6cc5c4c3)c2)cc1 ; CC1(C)c2ccc(N(c3ccccc3)c3ccccc3)cc2-c2ccc(N(c3cccc(N(c4ccccc4)c4ccccc4)c3)c3ccc4ccc5cc6oc7ccccc7c6cc5c4c3)cc21]

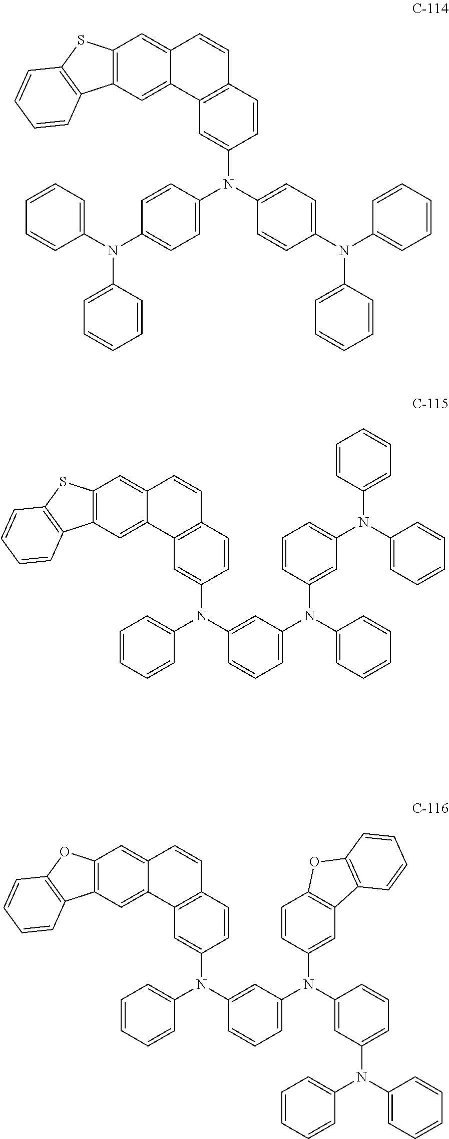 [SMILES: c1ccc(N(c2ccccc2)c2ccc(N(c3ccc(N(c4ccccc4)c4ccccc4)cc3)c3ccc4ccc5cc6sc7ccccc7c6cc5c4c3)cc2)cc1 ; c1ccc(N(c2ccccc2)c2cccc(N(c3ccccc3)c3cccc(N(c4ccccc4)c4ccc5ccc6cc7sc8ccccc8c7cc6c5c4)c3)c2)cc1 ; c1ccc(N(c2ccccc2)c2cccc(N(c3cccc(N(c4ccccc4)c4ccc5ccc6cc7oc8ccccc8c7cc6c5c4)c3)c3ccc4oc5ccccc5c4c3)c2)cc1]

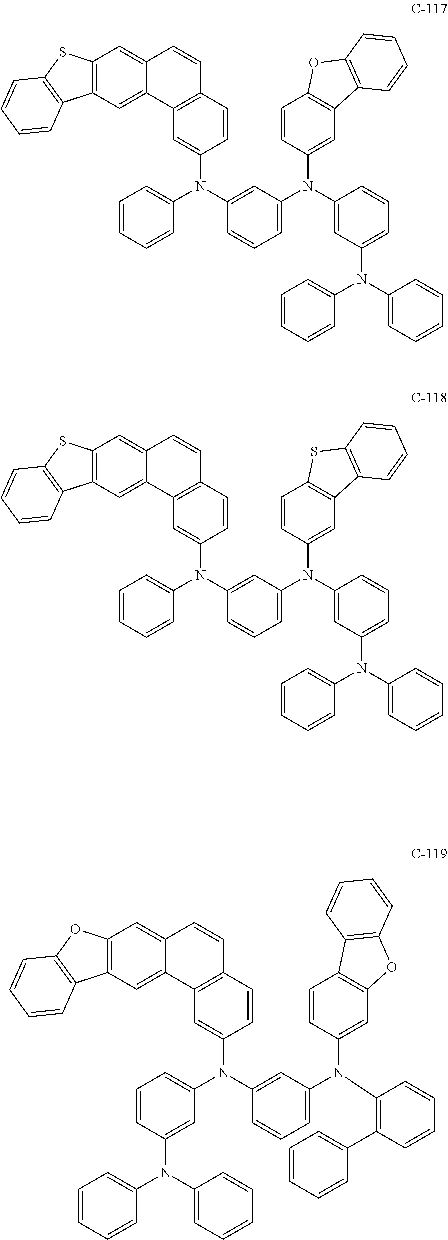 [SMILES: c1ccc(N(c2ccccc2)c2cccc(N(c3cccc(N(c4ccccc4)c4ccc5ccc6cc7sc8ccccc8c7cc6c5c4)c3)c3ccc4oc5ccccc5c4c3)c2)cc1 ; c1ccc(N(c2ccccc2)c2cccc(N(c3cccc(N(c4ccccc4)c4ccc5ccc6cc7sc8ccccc8c7cc6c5c4)c3)c3ccc4sc5ccccc5c4c3)c2)cc1 ; c1ccc(-c2ccccc2N(c2cccc(N(c3cccc(N(c4ccccc4)c4ccccc4)c3)c3ccc4ccc5cc6oc7ccccc7c6cc5c4c3)c2)c2ccc3c(c2)oc2ccccc23)cc1]

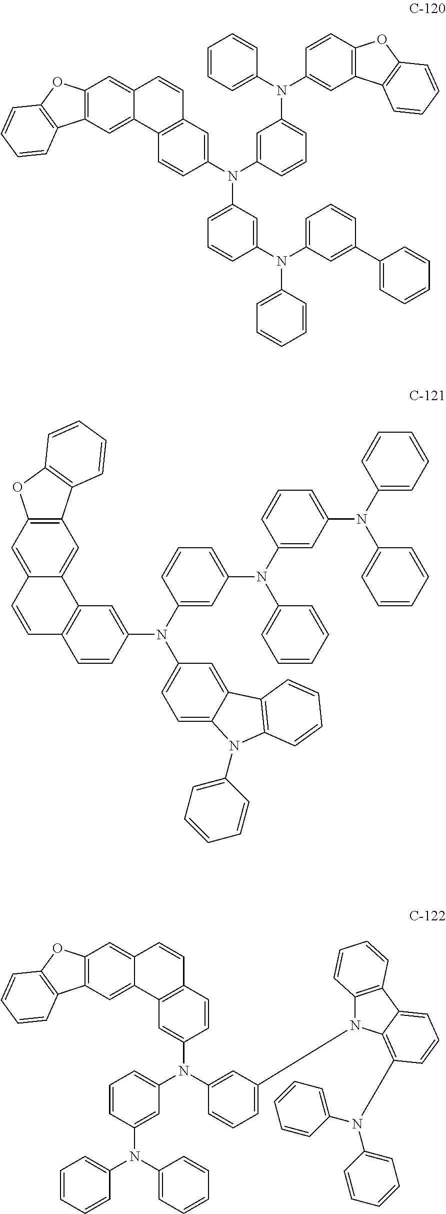 [SMILES: c1ccc(-c2cccc(N(c3ccccc3)c3cccc(N(c4cccc(N(c5ccccc5)c5ccc6oc7ccccc7c6c5)c4)c4ccc5c(ccc6cc7oc8ccccc8c7cc65)c4)c3)c2)cc1 ; c1ccc(N(c2ccccc2)c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3cccc(-n4c5ccccc5c5cccc(N(c6ccccc6)c6ccccc6)c54)c3)c3ccc4ccc5cc6oc7ccccc7c6cc5c4c3)c2)cc1]

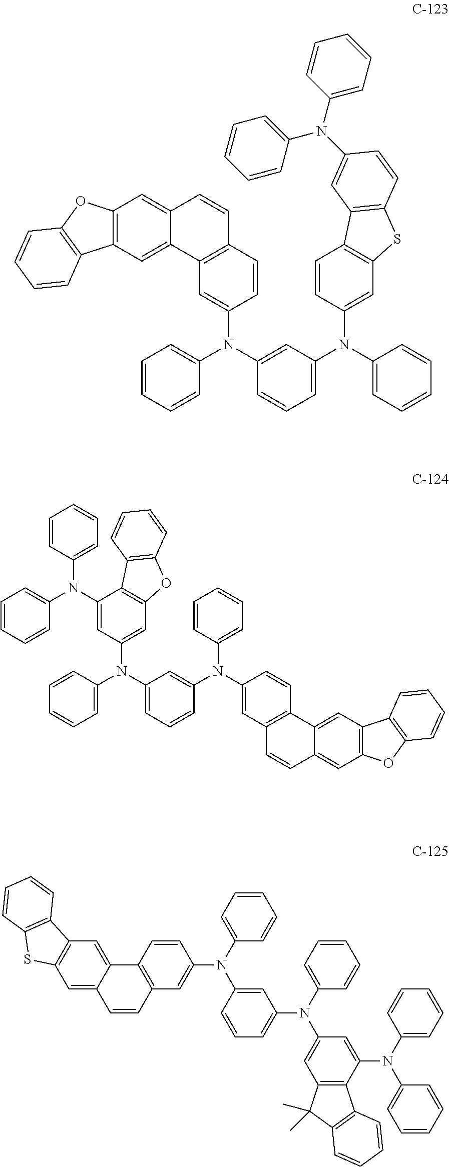 [SMILES: c1ccc(N(c2cccc(N(c3ccccc3)c3ccc4ccc5cc6oc7ccccc7c6cc5c4c3)c2)c2ccc3c(c2)sc2ccc(N(c4ccccc4)c4ccccc4)cc23)cc1 ; c1ccc(N(c2cccc(N(c3ccccc3)c3cc(N(c4ccccc4)c4ccccc4)c4c(c3)oc3ccccc34)c2)c2ccc3c(ccc4cc5oc6ccccc6c5cc43)c2)cc1 ; CC1(C)c2ccccc2-c2c(N(c3ccccc3)c3ccccc3)cc(N(c3ccccc3)c3cccc(N(c4ccccc4)c4ccc5c(ccc6cc7sc8ccccc8c7cc65)c4)c3)cc21]

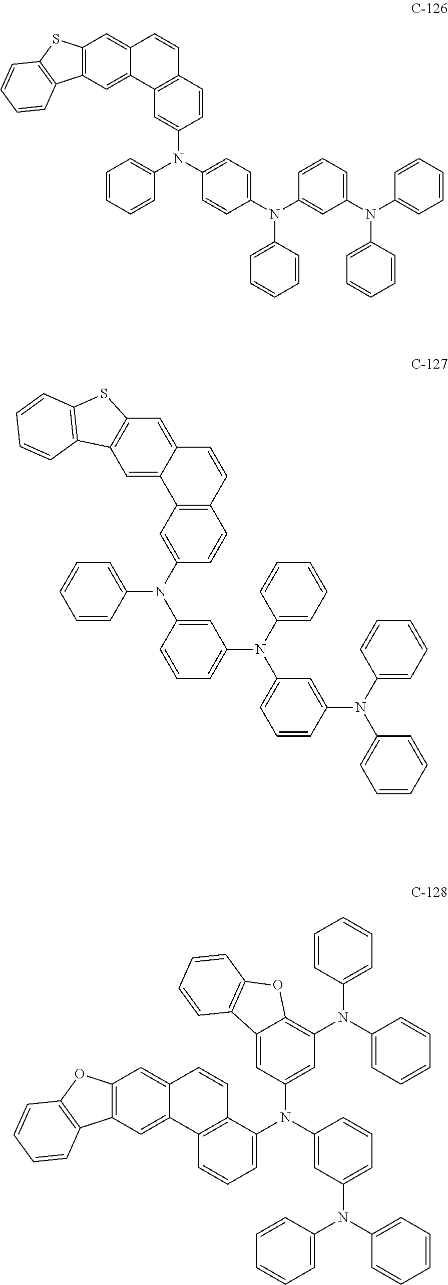 [SMILES: c1ccc(N(c2ccccc2)c2cccc(N(c3ccccc3)c3ccc(N(c4ccccc4)c4ccc5ccc6cc7sc8ccccc8c7cc6c5c4)cc3)c2)cc1 ; c1ccc(N(c2ccccc2)c2cccc(N(c3ccccc3)c3cccc(N(c4ccccc4)c4ccc5ccc6cc7sc8ccccc8c7cc6c5c4)c3)c2)cc1 ; c1ccc(N(c2ccccc2)c2cccc(N(c3cc(N(c4ccccc4)c4ccccc4)c4oc5ccccc5c4c3)c3cccc4c3ccc3cc5oc6ccccc6c5cc34)c2)cc1]

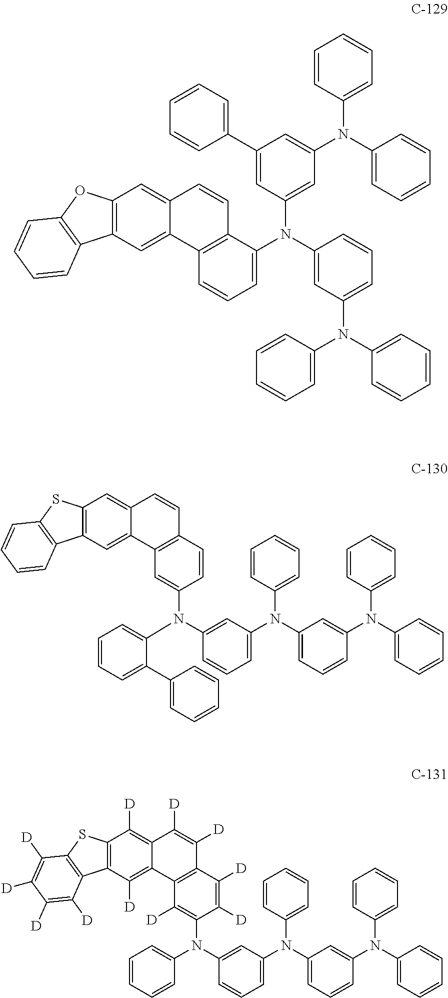 [SMILES: c1ccc(-c2cc(N(c3ccccc3)c3ccccc3)cc(N(c3cccc(N(c4ccccc4)c4ccccc4)c3)c3cccc4c3ccc3cc5oc6ccccc6c5cc34)c2)cc1 ; c1ccc(-c2ccccc2N(c2cccc(N(c3ccccc3)c3cccc(N(c4ccccc4)c4ccccc4)c3)c2)c2ccc3ccc4cc5sc6ccccc6c5cc4c3c2)cc1 ; [2H]c1c([2H])c([2H])c2c(sc3c([2H])c4c([2H])c([2H])c5c([2H])c([2H])c(N(c6ccccc6)c6cccc(N(c7ccccc7)c7cccc(N(c8ccccc8)c8ccccc8)c7)c6)c([2H])c5c4c([2H])c32)c1[2H]]

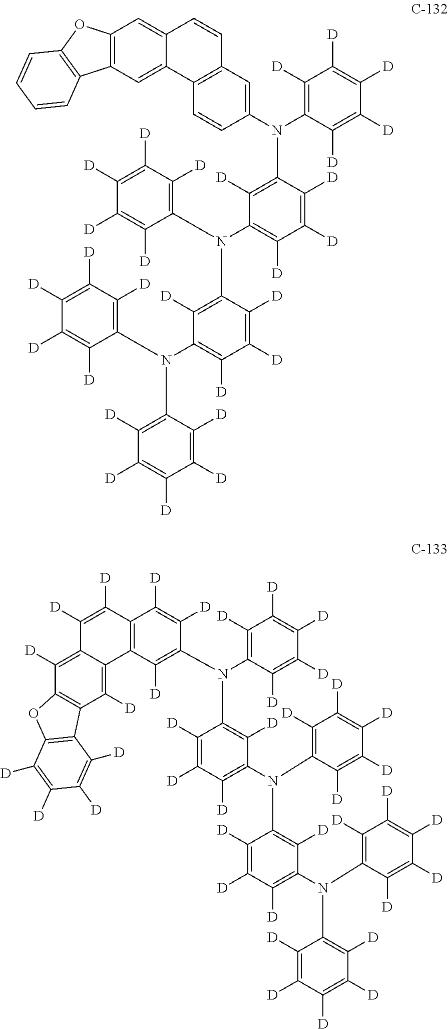

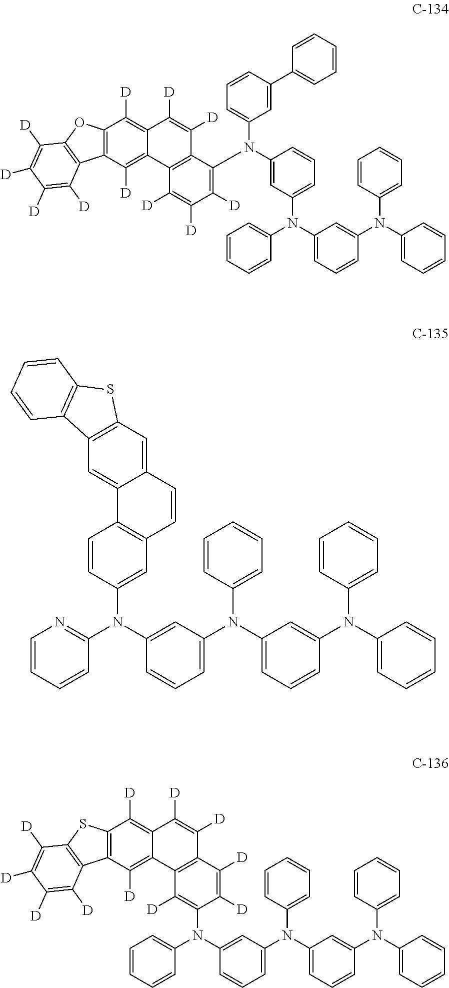 [SMILES: [2H]c1c([2H])c([2H])c2c(oc3c([2H])c4c([2H])c([2H])c5c(N(c6cccc(-c7ccccc7)c6)c6cccc(N(c7ccccc7)c7cccc(N(c8ccccc8)c8ccccc8)c7)c6)c([2H])c([2H])c([2H])c5c4c([2H])c32)c1[2H] ; c1ccc(N(c2ccccc2)c2cccc(N(c3ccccc3)c3cccc(N(c4ccc5c(ccc6cc7sc8ccccc8c7cc65)c4)c4ccccn4)c3)c2)cc1 ; [2H]c1c([2H])c([2H])c2c(sc3c([2H])c4c([2H])c([2H])c5c([2H])c([2H])c(N(c6ccccc6)c6cccc(N(c7ccccc7)c7cccc(N(c8ccccc8)c8ccccc8)c7)c6)c([2H])c5c4c([2H])c32)c1[2H]]

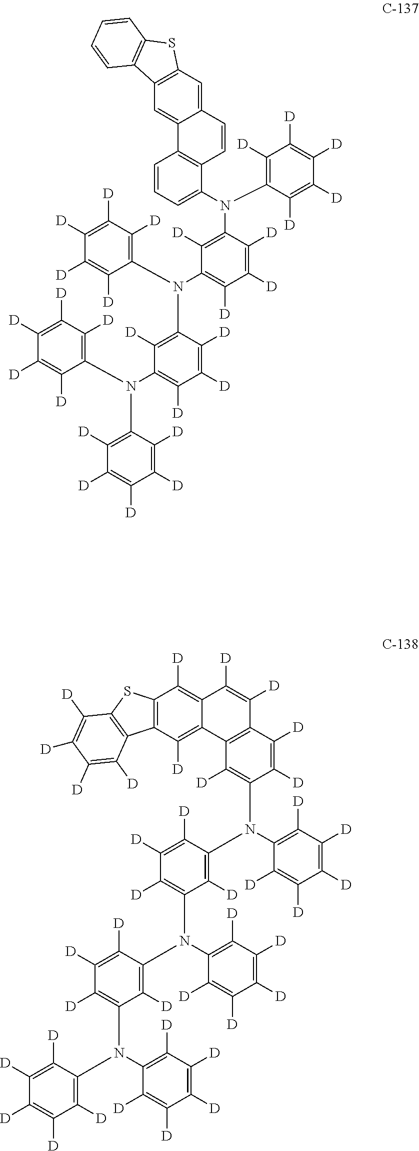

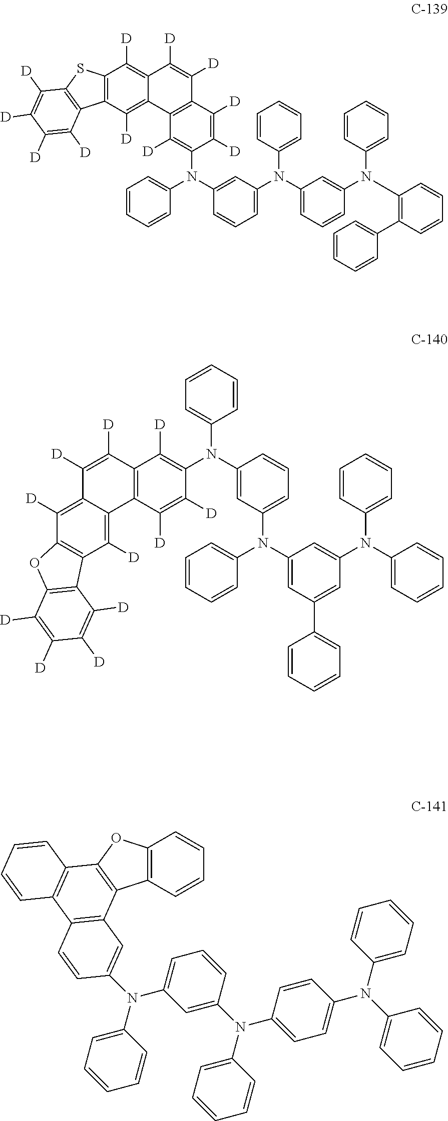 [SMILES: [2H]c1c([2H])c([2H])c2c(sc3c([2H])c4c([2H])c([2H])c5c([2H])c([2H])c(N(c6ccccc6)c6cccc(N(c7ccccc7)c7cccc(N(c8ccccc8)c8ccccc8-c8ccccc8)c7)c6)c([2H])c5c4c([2H])c32)c1[2H] ; [2H]c1c([2H])c([2H])c2c(oc3c([2H])c4c([2H])c([2H])c5c([2H])c(N(c6ccccc6)c6cccc(N(c7ccccc7)c7cc(-c8ccccc8)cc(N(c8ccccc8)c8ccccc8)c7)c6)c([2H])c([2H])c5c4c([2H])c32)c1[2H] ; c1ccc(N(c2ccccc2)c2ccc(N(c3ccccc3)c3cccc(N(c4ccccc4)c4ccc5c6ccccc6c6oc7ccccc7c6c5c4)c3)cc2)cc1]

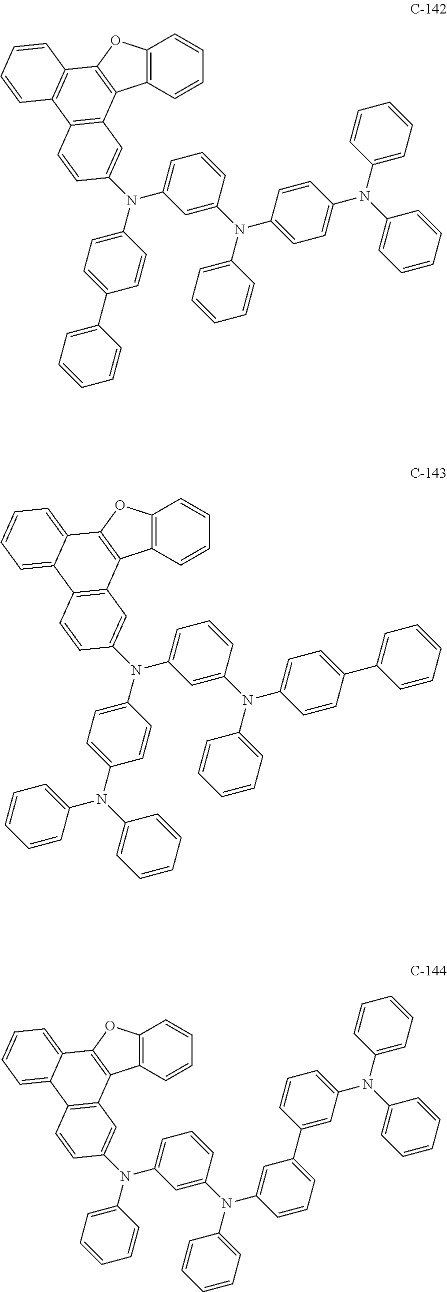 [SMILES: c1ccc(-c2ccc(N(c3cccc(N(c4ccccc4)c4ccc(N(c5ccccc5)c5ccccc5)cc4)c3)c3ccc4c5ccccc5c5oc6ccccc6c5c4c3)cc2)cc1 ; c1ccc(-c2ccc(N(c3ccccc3)c3cccc(N(c4ccc(N(c5ccccc5)c5ccccc5)cc4)c4ccc5c6ccccc6c6oc7ccccc7c6c5c4)c3)cc2)cc1 ; c1ccc(N(c2ccccc2)c2cccc(-c3cccc(N(c4ccccc4)c4cccc(N(c5ccccc5)c5ccc6c7ccccc7c7oc8ccccc8c7c6c5)c4)c3)c2)cc1]

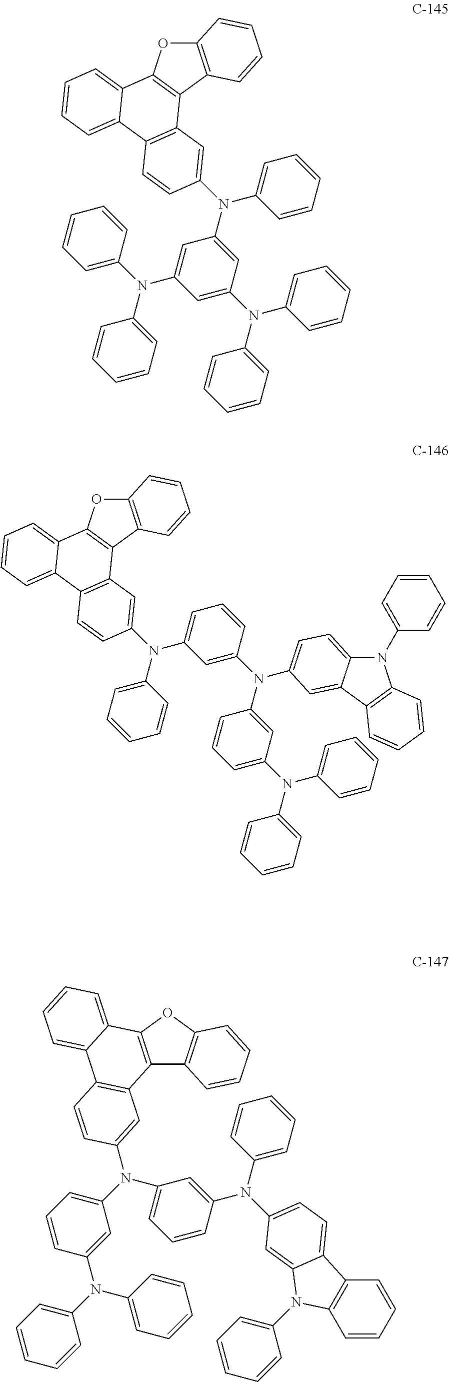 [SMILES: c1ccc(N(c2ccccc2)c2cc(N(c3ccccc3)c3ccccc3)cc(N(c3ccccc3)c3ccc4c5ccccc5c5oc6ccccc6c5c4c3)c2)cc1 ; c1ccc(N(c2ccccc2)c2cccc(N(c3cccc(N(c4ccccc4)c4ccc5c6ccccc6c6oc7ccccc7c6c5c4)c3)c3ccc4c(c3)c3ccccc3n4-c3ccccc3)c2)cc1 ; c1ccc(N(c2ccccc2)c2cccc(N(c3cccc(N(c4ccccc4)c4ccc5c6ccccc6n(-c6ccccc6)c5c4)c3)c3ccc4c5ccccc5c5oc6ccccc6c5c4c3)c2)cc1]

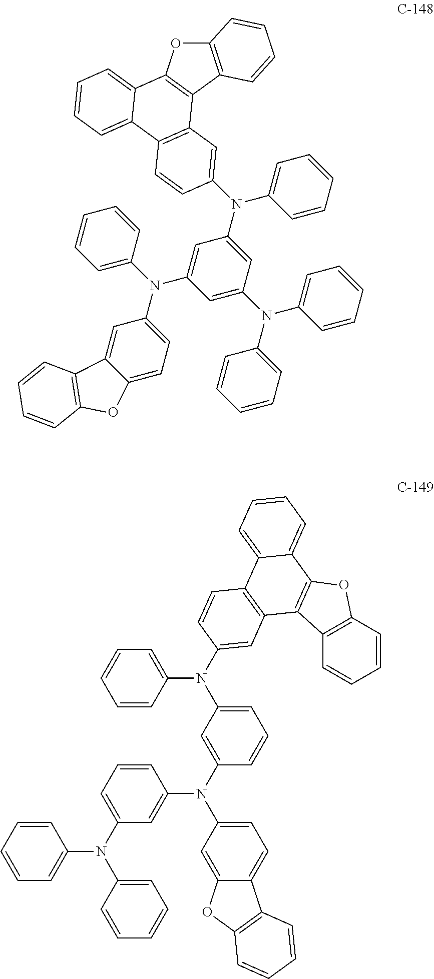 [SMILES: c1ccc(N(c2ccccc2)c2cc(N(c3ccccc3)c3ccc4oc5ccccc5c4c3)cc(N(c3ccccc3)c3ccc4c5ccccc5c5oc6ccccc6c5c4c3)c2)cc1 ; c1ccc(N(c2ccccc2)c2cccc(N(c3cccc(N(c4ccccc4)c4ccc5c6ccccc6c6oc7ccccc7c6c5c4)c3)c3ccc4c(c3)oc3ccccc34)c2)cc1]

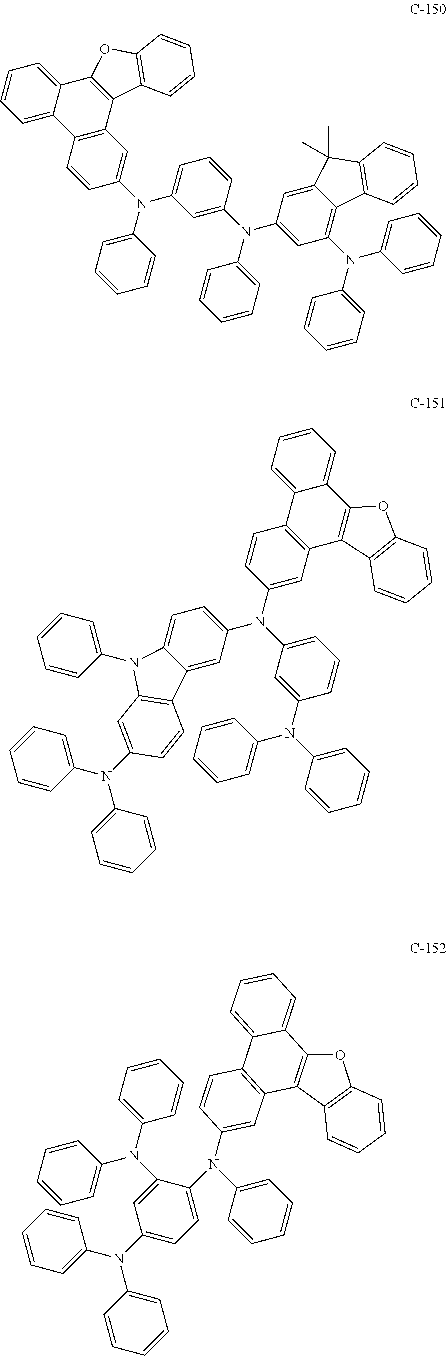 [SMILES: CC1(C)c2ccccc2-c2c(N(c3ccccc3)c3ccccc3)cc(N(c3ccccc3)c3cccc(N(c4ccccc4)c4ccc5c6ccccc6c6oc7ccccc7c6c5c4)c3)cc21 ; c1ccc(N(c2ccccc2)c2cccc(N(c3ccc4c5ccccc5c5oc6ccccc6c5c4c3)c3ccc4c(c3)c3ccc(N(c5ccccc5)c5ccccc5)cc3n4-c3ccccc3)c2)cc1 ; c1ccc(N(c2ccccc2)c2ccc(N(c3ccccc3)c3ccc4c5ccccc5c5oc6ccccc6c5c4c3)c(N(c3ccccc3)c3ccccc3)c2)cc1]

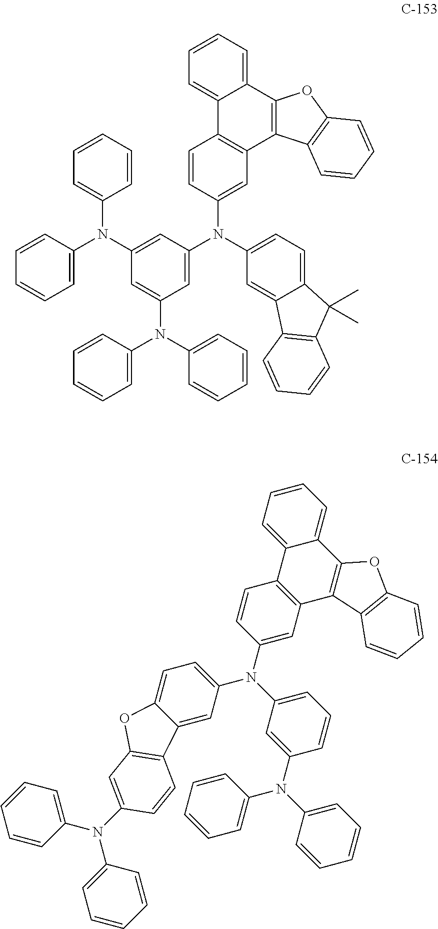 [SMILES: CC1(C)c2ccccc2-c2cc(N(c3cc(N(c4ccccc4)c4ccccc4)cc(N(c4ccccc4)c4ccccc4)c3)c3ccc4c5ccccc5c5oc6ccccc6c5c4c3)ccc21 ; c1ccc(N(c2ccccc2)c2cccc(N(c3ccc4oc5cc(N(c6ccccc6)c6ccccc6)ccc5c4c3)c3ccc4c5ccccc5c5oc6ccccc6c5c4c3)c2)cc1]

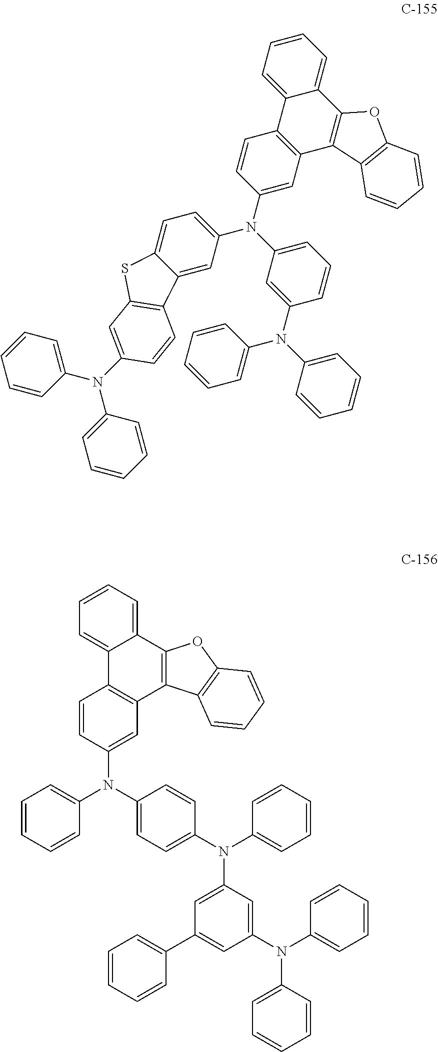 [SMILES: c1ccc(N(c2ccccc2)c2cccc(N(c3ccc4sc5cc(N(c6ccccc6)c6ccccc6)ccc5c4c3)c3ccc4c5ccccc5c5oc6ccccc6c5c4c3)c2)cc1 ; c1ccc(-c2cc(N(c3ccccc3)c3ccccc3)cc(N(c3ccccc3)c3ccc(N(c4ccccc4)c4ccc5c6ccccc6c6oc7ccccc7c6c5c4)cc3)c2)cc1]

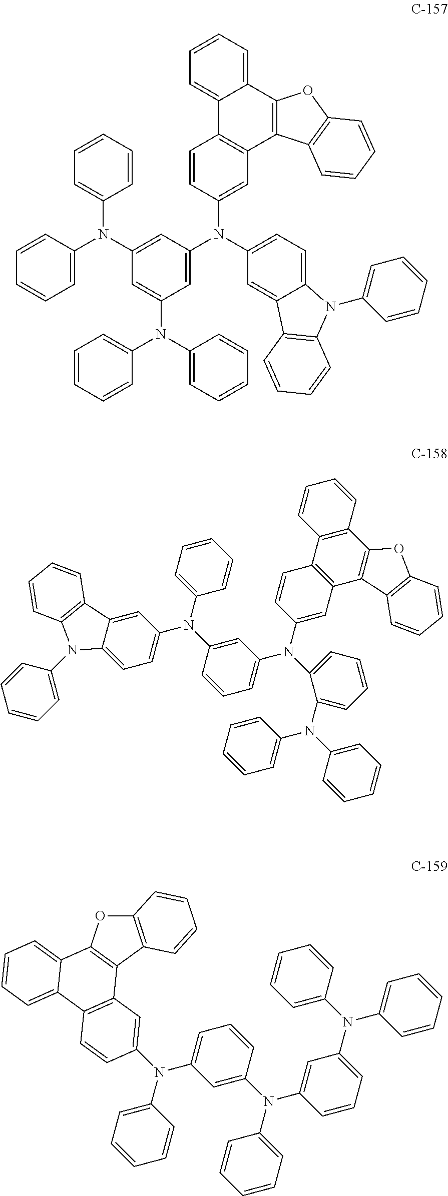 [SMILES: c1ccc(N(c2ccccc2)c2cc(N(c3ccccc3)c3ccccc3)cc(N(c3ccc4c5ccccc5c5oc6ccccc6c5c4c3)c3ccc4c(c3)c3ccccc3n4-c3ccccc3)c2)cc1 ; c1ccc(N(c2cccc(N(c3ccc4c5ccccc5c5oc6ccccc6c5c4c3)c3ccccc3N(c3ccccc3)c3ccccc3)c2)c2ccc3c(c2)c2ccccc2n3-c2ccccc2)cc1 ; c1ccc(N(c2ccccc2)c2cccc(N(c3ccccc3)c3cccc(N(c4ccccc4)c4ccc5c6ccccc6c6oc7ccccc7c6c5c4)c3)c2)cc1]

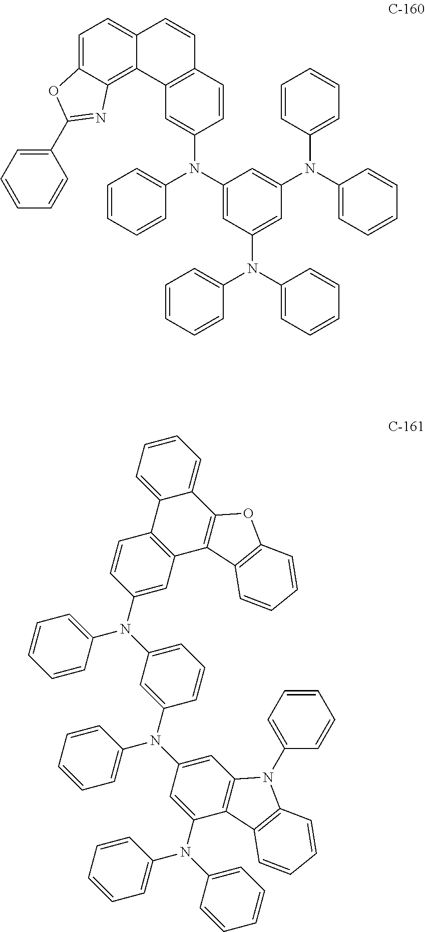 [SMILES: c1ccc(-c2nc3c(ccc4ccc5ccc(N(c6ccccc6)c6cc(N(c7ccccc7)c7ccccc7)cc(N(c7ccccc7)c7ccccc7)c6)cc5c43)o2)cc1 ; c1ccc(N(c2cccc(N(c3ccccc3)c3cc(N(c4ccccc4)c4ccccc4)c4c5ccccc5n(-c5ccccc5)c4c3)c2)c2ccc3c4ccccc4c4oc5ccccc5c4c3c2)cc1]

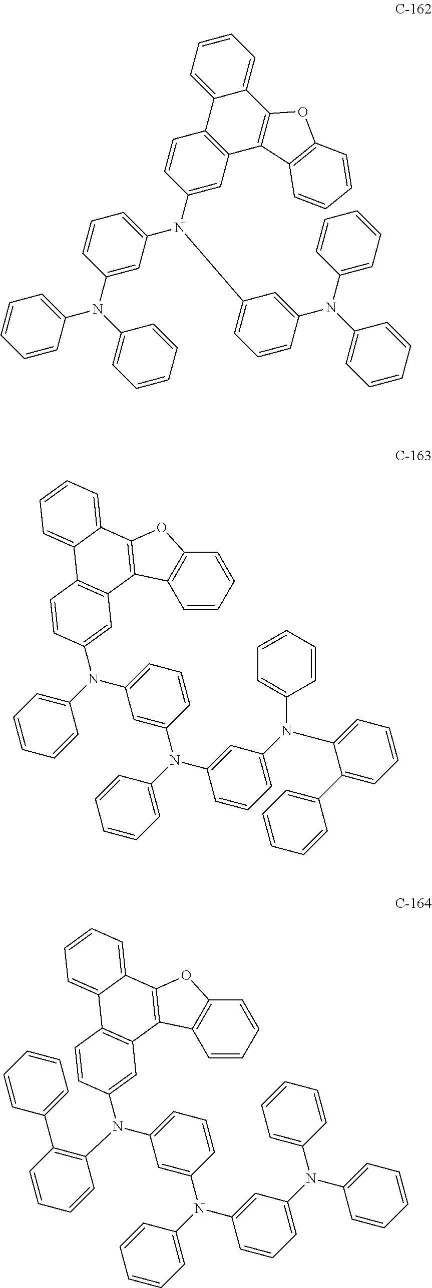 [SMILES: c1ccc(N(c2ccccc2)c2cccc(N(c3cccc(N(c4ccccc4)c4ccccc4)c3)c3ccc4c5ccccc5c5oc6ccccc6c5c4c3)c2)cc1 ; c1ccc(-c2ccccc2N(c2ccccc2)c2cccc(N(c3ccccc3)c3cccc(N(c4ccccc4)c4ccc5c6ccccc6c6oc7ccccc7c6c5c4)c3)c2)cc1 ; c1ccc(-c2ccccc2N(c2cccc(N(c3ccccc3)c3cccc(N(c4ccccc4)c4ccccc4)c3)c2)c2ccc3c4ccccc4c4oc5ccccc5c4c3c2)cc1]

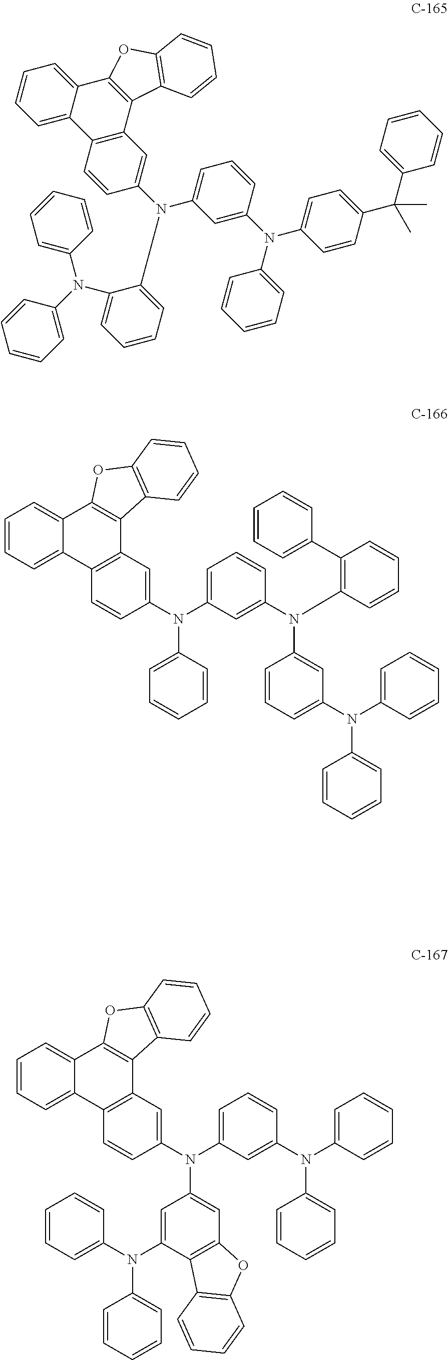 [SMILES: CC(C)(c1ccccc1)c1ccc(N(c2ccccc2)c2cccc(N(c3ccc4c5ccccc5c5oc6ccccc6c5c4c3)c3ccccc3N(c3ccccc3)c3ccccc3)c2)cc1 ; c1ccc(-c2ccccc2N(c2cccc(N(c3ccccc3)c3ccccc3)c2)c2cccc(N(c3ccccc3)c3ccc4c5ccccc5c5oc6ccccc6c5c4c3)c2)cc1 ; c1ccc(N(c2ccccc2)c2cccc(N(c3cc(N(c4ccccc4)c4ccccc4)c4c(c3)oc3ccccc34)c3ccc4c5ccccc5c5oc6ccccc6c5c4c3)c2)cc1]

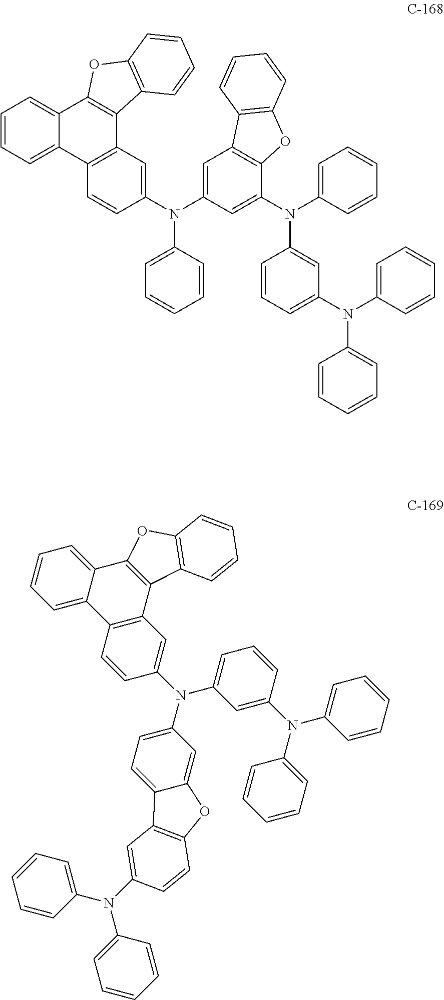 [SMILES: c1ccc(N(c2ccccc2)c2cccc(N(c3ccccc3)c3cc(N(c4ccccc4)c4ccc5c6ccccc6c6oc7ccccc7c6c5c4)cc4c3oc3ccccc34)c2)cc1 ; c1ccc(N(c2ccccc2)c2cccc(N(c3ccc4c(c3)oc3ccc(N(c5ccccc5)c5ccccc5)cc34)c3ccc4c5ccccc5c5oc6ccccc6c5c4c3)c2)cc1]

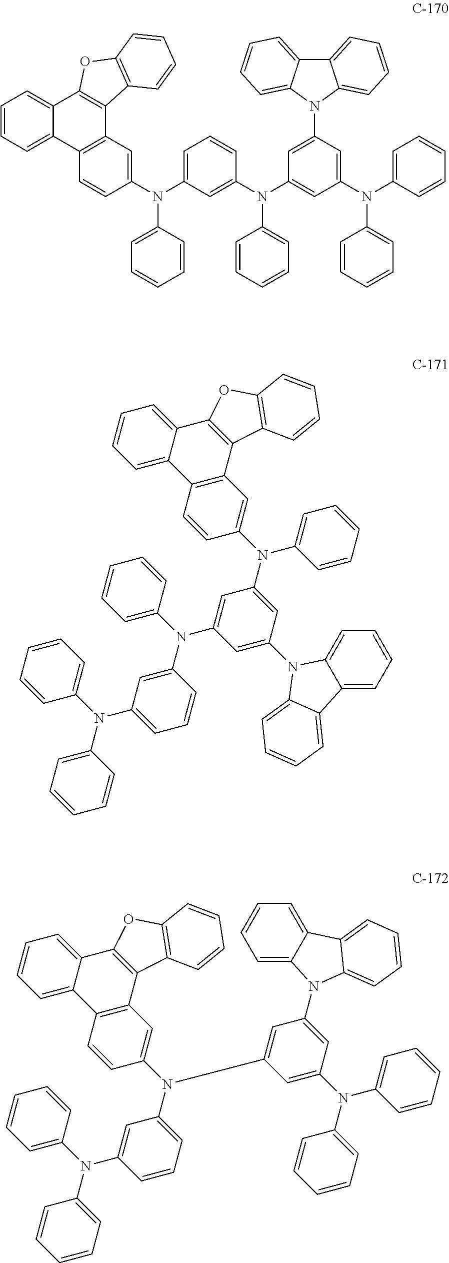 [SMILES: c1ccc(N(c2ccccc2)c2cc(N(c3ccccc3)c3cccc(N(c4ccccc4)c4ccc5c6ccccc6c6oc7ccccc7c6c5c4)c3)cc(-n3c4ccccc4c4ccccc43)c2)cc1 ; c1ccc(N(c2ccccc2)c2cccc(N(c3ccccc3)c3cc(N(c4ccccc4)c4ccc5c6ccccc6c6oc7ccccc7c6c5c4)cc(-n4c5ccccc5c5ccccc54)c3)c2)cc1 ; c1ccc(N(c2ccccc2)c2cccc(N(c3cc(N(c4ccccc4)c4ccccc4)cc(-n4c5ccccc5c5ccccc54)c3)c3ccc4c5ccccc5c5oc6ccccc6c5c4c3)c2)cc1]

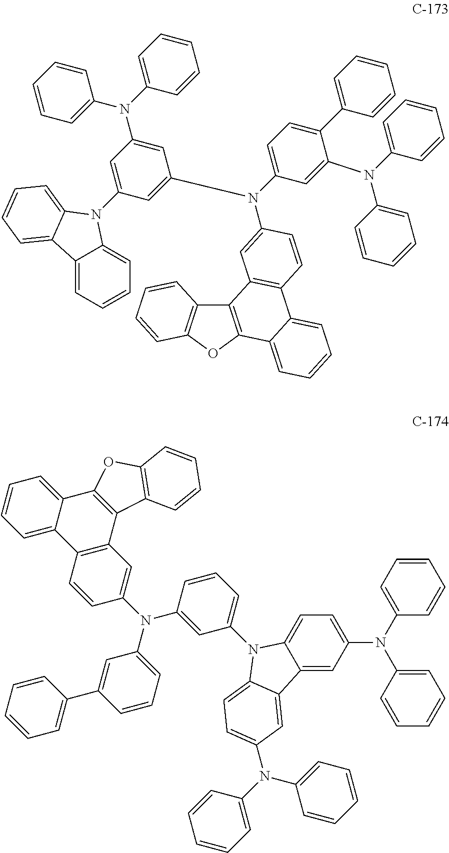 [SMILES: c1ccc(-c2ccc(N(c3cc(N(c4ccccc4)c4ccccc4)cc(-n4c5ccccc5c5ccccc54)c3)c3ccc4c5ccccc5c5oc6ccccc6c5c4c3)cc2N(c2ccccc2)c2ccccc2)cc1 ; c1ccc(-c2cccc(N(c3cccc(-n4c5ccc(N(c6ccccc6)c6ccccc6)cc5c5cc(N(c6ccccc6)c6ccccc6)ccc54)c3)c3ccc4c5ccccc5c5oc6ccccc6c5c4c3)c2)cc1]

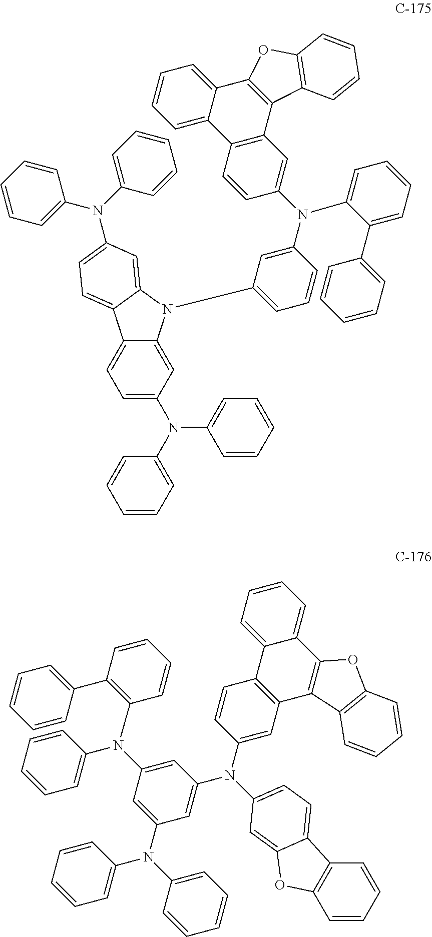 [SMILES: c1ccc(-c2ccccc2N(c2cccc(-n3c4cc(N(c5ccccc5)c5ccccc5)ccc4c4ccc(N(c5ccccc5)c5ccccc5)cc43)c2)c2ccc3c4ccccc4c4oc5ccccc5c4c3c2)cc1 ; c1ccc(-c2ccccc2N(c2ccccc2)c2cc(N(c3ccccc3)c3ccccc3)cc(N(c3ccc4c(c3)oc3ccccc34)c3ccc4c5ccccc5c5oc6ccccc6c5c4c3)c2)cc1]

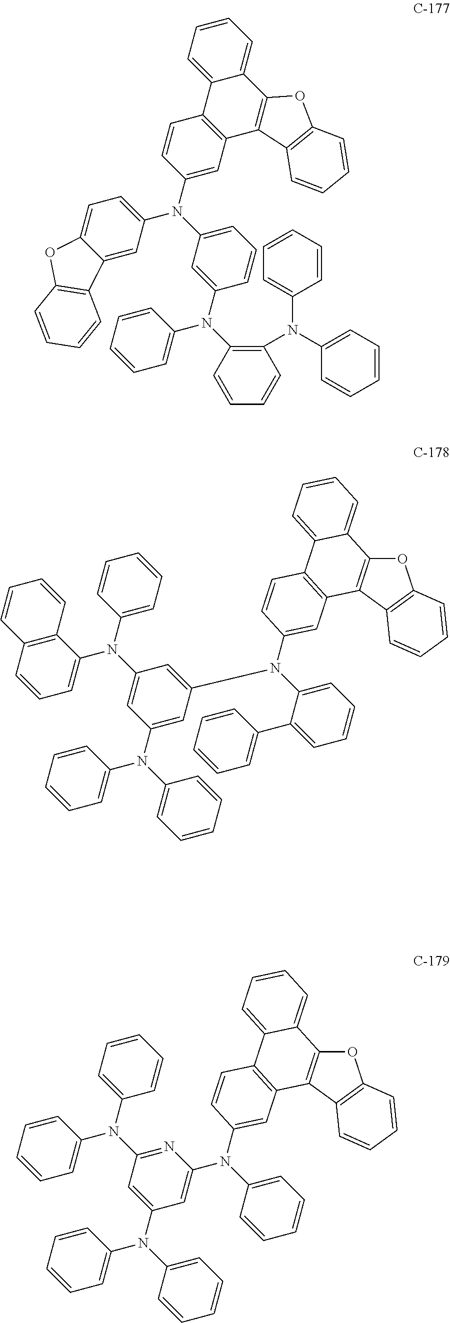 [SMILES: c1ccc(N(c2ccccc2)c2ccccc2N(c2ccccc2)c2cccc(N(c3ccc4oc5ccccc5c4c3)c3ccc4c5ccccc5c5oc6ccccc6c5c4c3)c2)cc1 ; c1ccc(-c2ccccc2N(c2cc(N(c3ccccc3)c3ccccc3)cc(N(c3ccccc3)c3cccc4ccccc34)c2)c2ccc3c4ccccc4c4oc5ccccc5c4c3c2)cc1 ; c1ccc(N(c2ccccc2)c2cc(N(c3ccccc3)c3ccccc3)nc(N(c3ccccc3)c3ccc4c5ccccc5c5oc6ccccc6c5c4c3)c2)cc1]

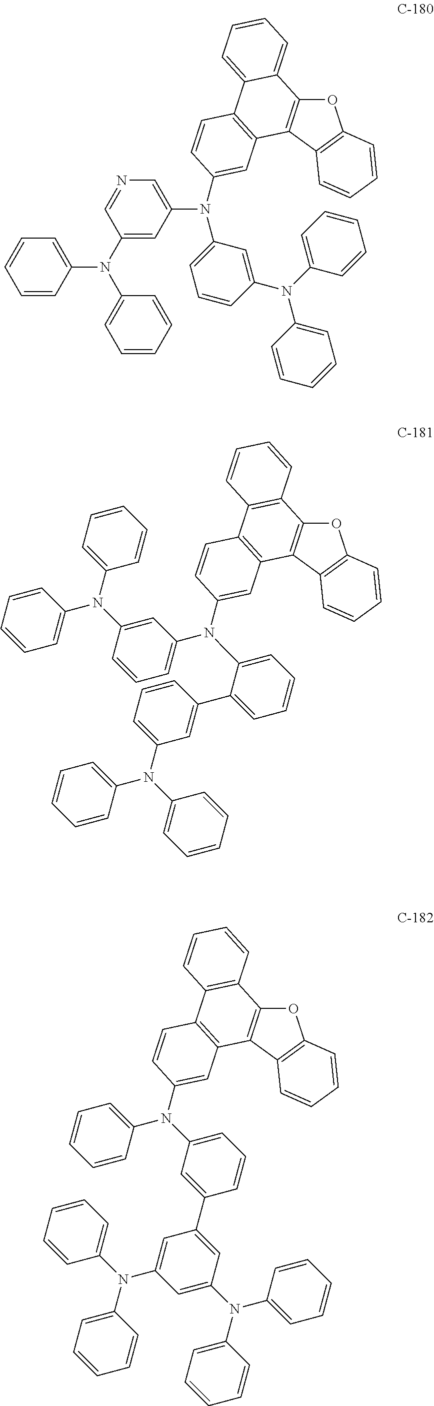 [SMILES: c1ccc(N(c2ccccc2)c2cccc(N(c3cncc(N(c4ccccc4)c4ccccc4)c3)c3ccc4c5ccccc5c5oc6ccccc6c5c4c3)c2)cc1 ; c1ccc(N(c2ccccc2)c2cccc(-c3ccccc3N(c3cccc(N(c4ccccc4)c4ccccc4)c3)c3ccc4c5ccccc5c5oc6ccccc6c5c4c3)c2)cc1 ; c1ccc(N(c2ccccc2)c2cc(-c3cccc(N(c4ccccc4)c4ccc5c6ccccc6c6oc7ccccc7c6c5c4)c3)cc(N(c3ccccc3)c3ccccc3)c2)cc1]

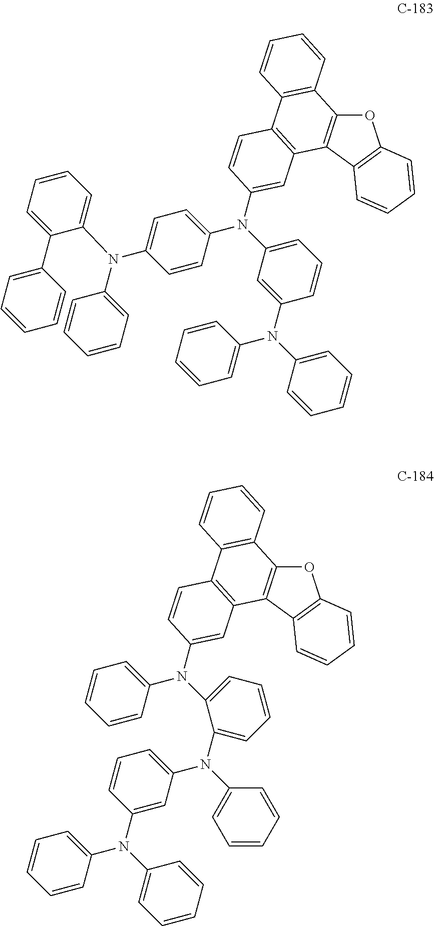 [SMILES: c1ccc(-c2ccccc2N(c2ccccc2)c2ccc(N(c3cccc(N(c4ccccc4)c4ccccc4)c3)c3ccc4c5ccccc5c5oc6ccccc6c5c4c3)cc2)cc1 ; c1ccc(N(c2ccccc2)c2cccc(N(c3ccccc3)c3ccccc3N(c3ccccc3)c3ccc4c5ccccc5c5oc6ccccc6c5c4c3)c2)cc1]

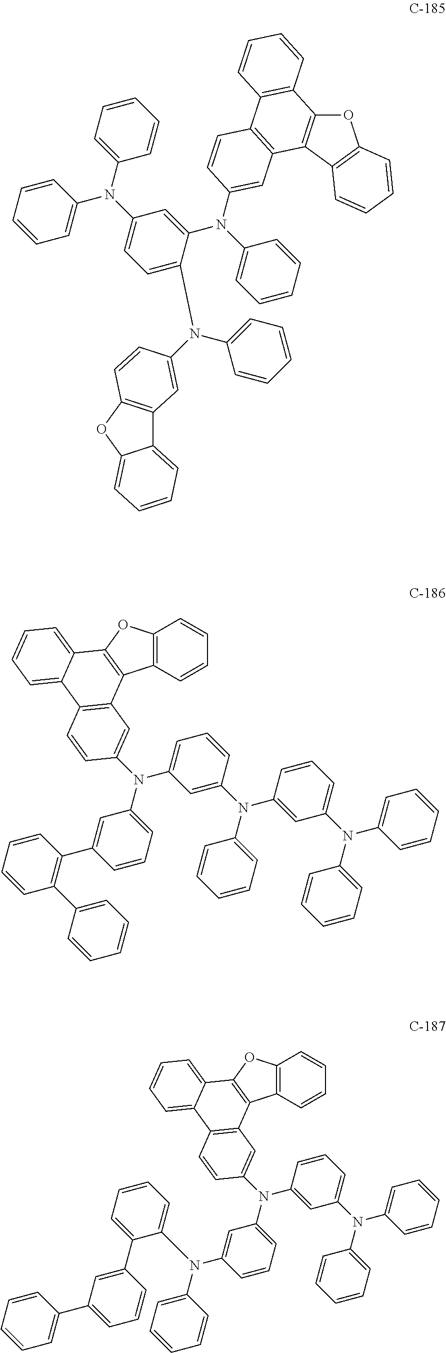 [SMILES: c1ccc(N(c2ccccc2)c2ccc(N(c3ccccc3)c3ccc4oc5ccccc5c4c3)c(N(c3ccccc3)c3ccc4c5ccccc5c5oc6ccccc6c5c4c3)c2)cc1 ; c1ccc(-c2ccccc2-c2cccc(N(c3cccc(N(c4ccccc4)c4cccc(N(c5ccccc5)c5ccccc5)c4)c3)c3ccc4c5ccccc5c5oc6ccccc6c5c4c3)c2)cc1 ; c1ccc(-c2cccc(-c3ccccc3N(c3ccccc3)c3cccc(N(c4cccc(N(c5ccccc5)c5ccccc5)c4)c4ccc5c6ccccc6c6oc7ccccc7c6c5c4)c3)c2)cc1]

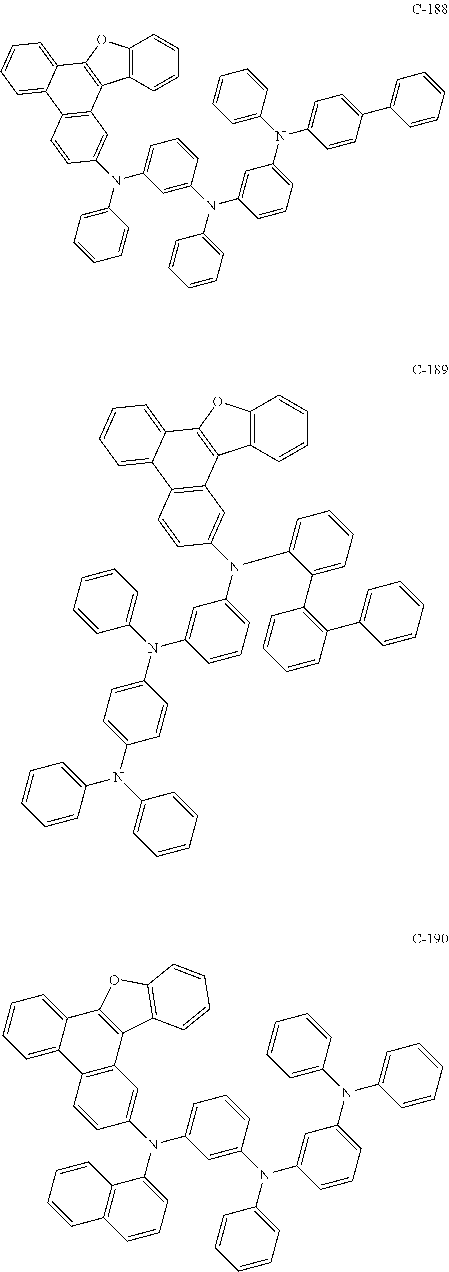 [SMILES: c1ccc(-c2ccc(N(c3ccccc3)c3cccc(N(c4ccccc4)c4cccc(N(c5ccccc5)c5ccc6c7ccccc7c7oc8ccccc8c7c6c5)c4)c3)cc2)cc1 ; c1ccc(-c2ccccc2-c2ccccc2N(c2cccc(N(c3ccccc3)c3ccc(N(c4ccccc4)c4ccccc4)cc3)c2)c2ccc3c4ccccc4c4oc5ccccc5c4c3c2)cc1 ; c1ccc(N(c2ccccc2)c2cccc(N(c3ccccc3)c3cccc(N(c4ccc5c6ccccc6c6oc7ccccc7c6c5c4)c4cccc5ccccc45)c3)c2)cc1]

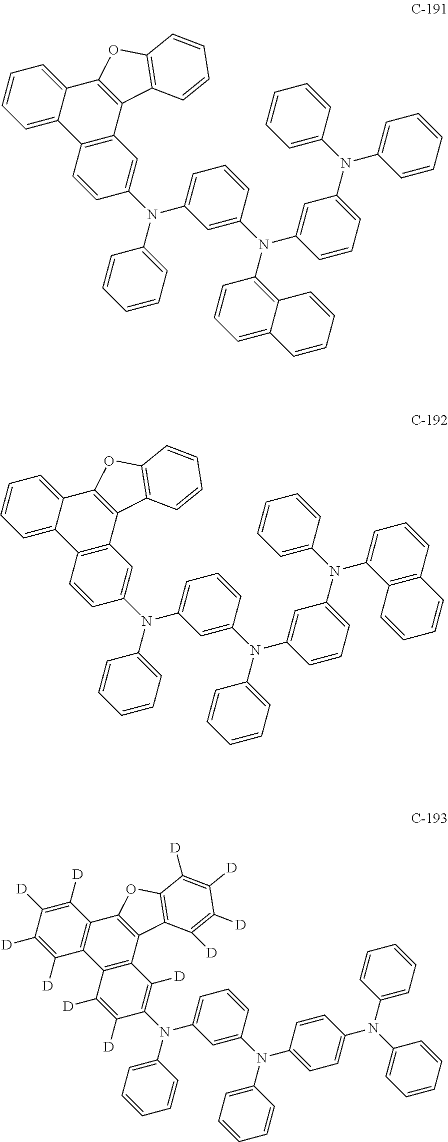 [SMILES: c1ccc(N(c2ccccc2)c2cccc(N(c3cccc(N(c4ccccc4)c4ccc5c6ccccc6c6oc7ccccc7c6c5c4)c3)c3cccc4ccccc34)c2)cc1 ; c1ccc(N(c2cccc(N(c3ccccc3)c3ccc4c5ccccc5c5oc6ccccc6c5c4c3)c2)c2cccc(N(c3ccccc3)c3cccc4ccccc34)c2)cc1 ; [2H]c1c([2H])c([2H])c2c(oc3c4c([2H])c([2H])c([2H])c([2H])c4c4c([2H])c([2H])c(N(c5ccccc5)c5cccc(N(c6ccccc6)c6ccc(N(c7ccccc7)c7ccccc7)cc6)c5)c([2H])c4c32)c1[2H]]

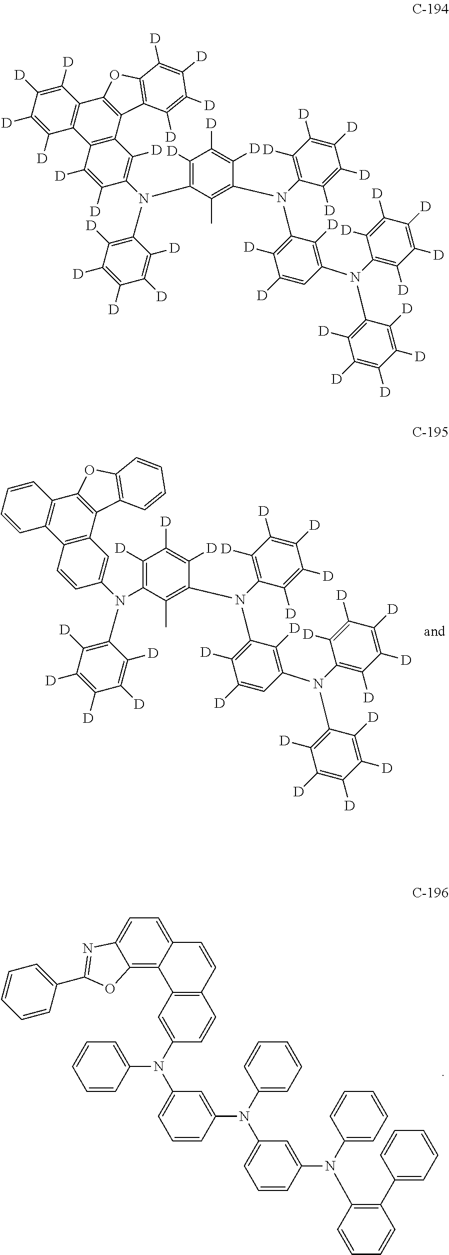

9. A plurality of host materials comprising a first host material comprising the organic electroluminescent compound according to claim 1 and a second host material comprising an organic electroluminescent compound represented by the following formula 2: 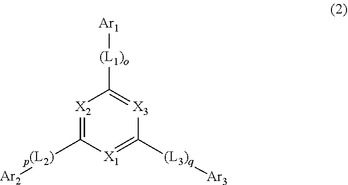
in formula 2,
X₁ to X3, each independently, represent CR′ or N; with the proviso that at least two of X₁ to X₃ represent N;
R′ represents hydrogen or deuterium;
L₁ to L₃, each independently, represent a single bond, a substituted or unsubstituted (C6-C30)arylene, or a substituted or unsubstituted (3- to 30-membered)heteroarylene;
Ar₁ to Ar₃, each independently, represent a substituted or unsubstituted (C6-C30)aryl, or a substituted or unsubstituted (3- to 30-membered)heteroaryl; and
o to q, each independently, represent an integer of 1 or 2; in which if o to q represent an integer of 2 or more, each of Li to each of L 3 may be the same or different.

10. The plurality of host materials according to claim 9, wherein at least one of Ari to Ar 3 in formula 2 is represented by the following formula 2-1: 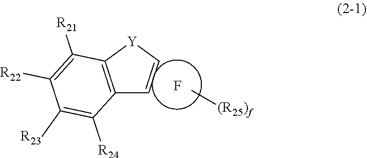
in formula 2-1,
Y represents O, S, N(R₃₁), or C(R₃₂)(R₃₃);
R₃₁ is a position connected to any one of L₁ to L₃, or a substituted or unsubstituted (C6-C30)aryl;
R₃₂ and R₃₃, each independently, are a position connected to any one of L₁ to L₃, or represent a substituted or unsubstituted (C1-C30)alkyl, a substituted or unsubstituted (C6-C30)aryl, or a substituted or unsubstituted (3- to 30-membered)heteroaryl;
F represents a benzene ring or a naphthalene ring;
f represents an integer of 4 to 6; and
R₂₁ to R₂₅, each independently, are a position connected to any one of L₁ to L₃; or represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to an adjacent substituent(s) to form a ring(s).

11. The plurality of host materials according to claim 9, wherein Ar₁ to Ar₃ in formula 2, each independently, represent a (C6-C30)aryl unsubstituted or substituted with at least one of deuterium, a (C1-C30)alkyl(s), and a (C6-C30)aryl(s).

12. The plurality of host materials according to claim 9, wherein the compound represented by formula 2 is at least one selected from the group consisting of the following compounds:

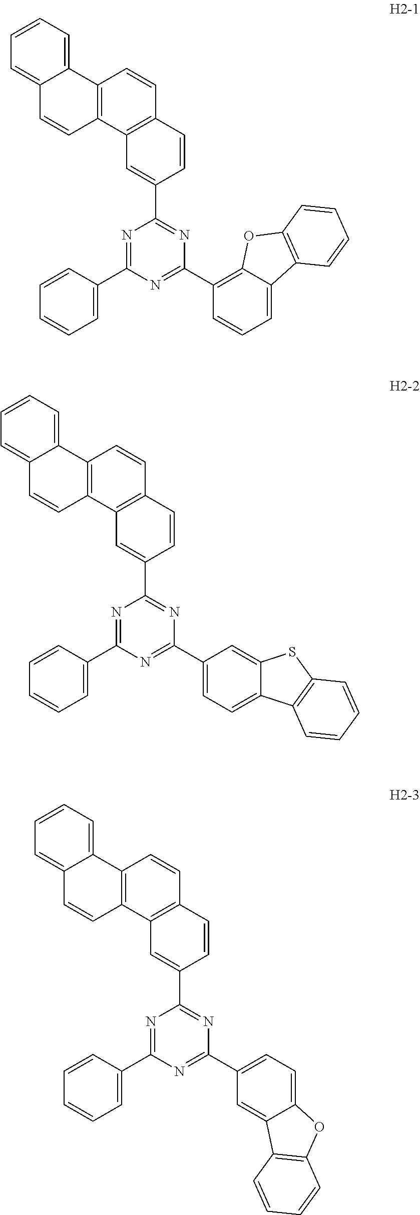 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

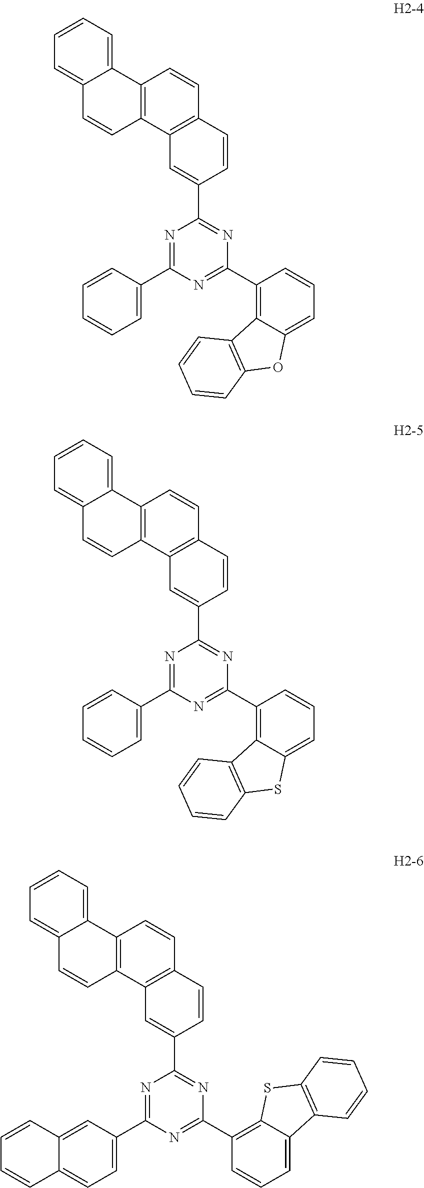 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

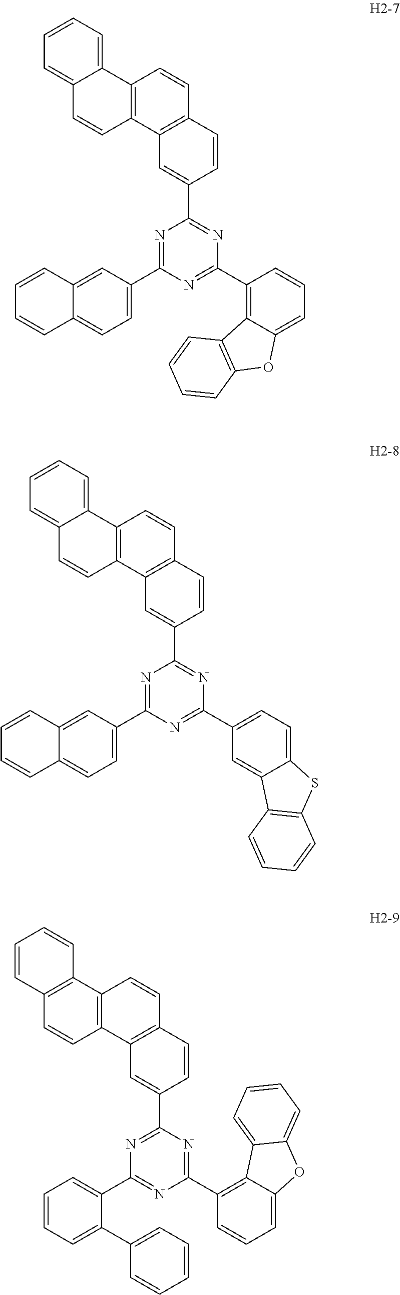 [SMILES: c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1 ; c1ccc(-c2ccccc2-c2nc(-c3ccc4ccc5c6ccccc6ccc5c4c3)nc(-c3cccc4oc5ccccc5c34)n2)cc1]

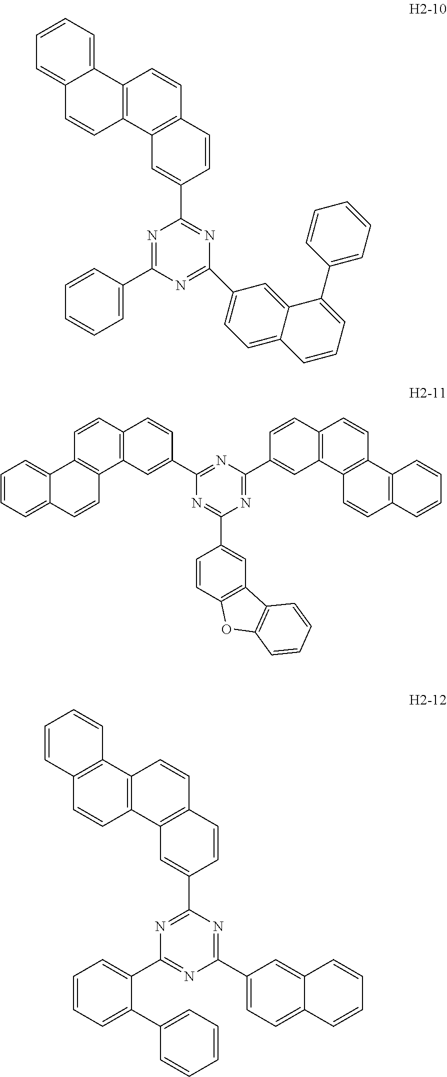 [SMILES: c1ccc(-c2nc(-c3ccc4cccc(-c5ccccc5)c4c3)nc(-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21 ; c1ccc(-c2ccccc2-c2nc(-c3ccc4ccccc4c3)nc(-c3ccc4ccc5c6ccccc6ccc5c4c3)n2)cc1]

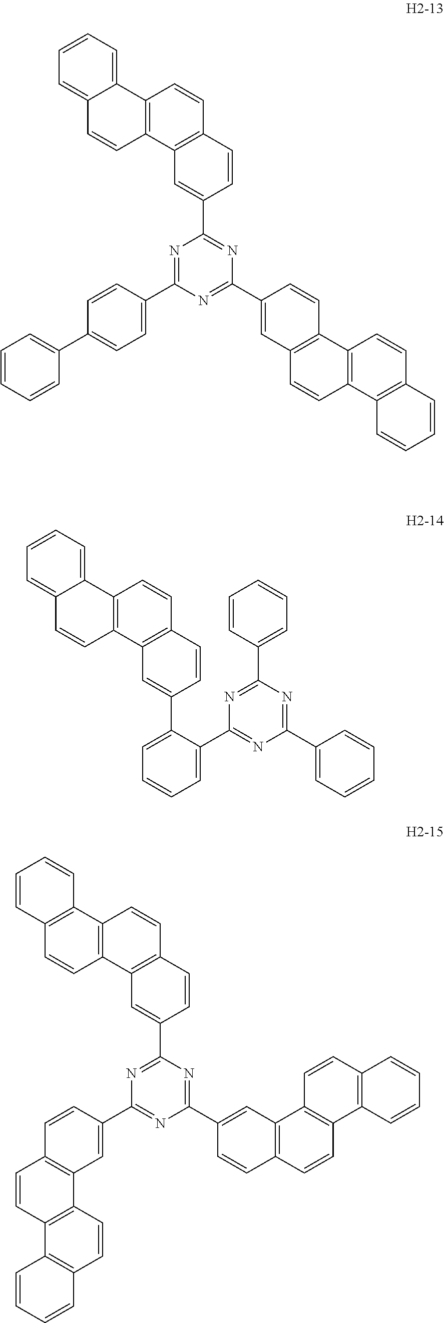 [SMILES: c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21]

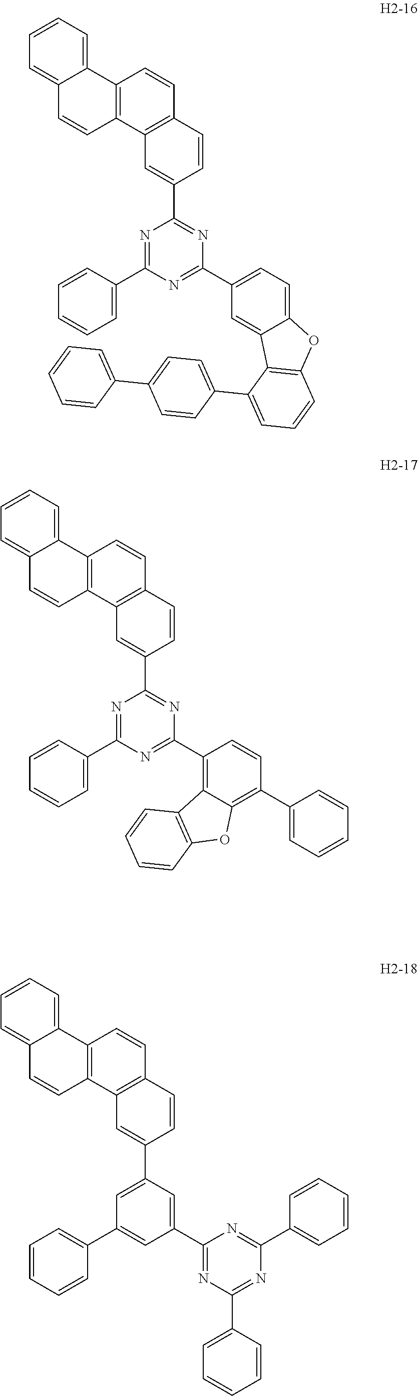 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1]

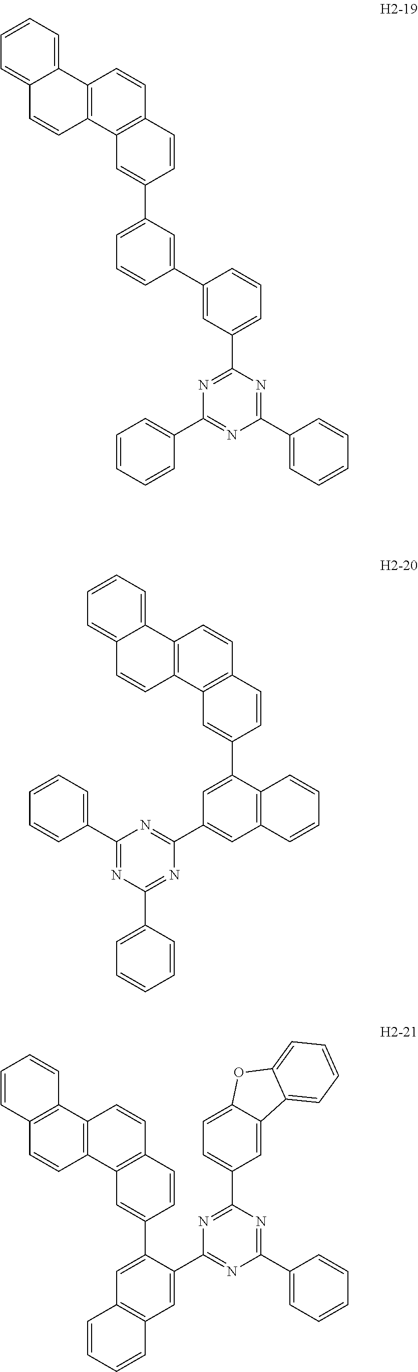 [SMILES: c1ccc(-c2nc(-c3ccccc3)nc(-c3cccc(-c4cccc(-c5ccc6ccc7c8ccccc8ccc7c6c5)c4)c3)n2)cc1 ; c1ccc(-c2nc(-c3ccccc3)nc(-c3cc(-c4ccc5ccc6c7ccccc7ccc6c5c4)c4ccccc4c3)n2)cc1 ; c1ccc(-c2nc(-c3ccc4oc5ccccc5c4c3)nc(-c3cc4ccccc4cc3-c3ccc4ccc5c6ccccc6ccc5c4c3)n2)cc1]

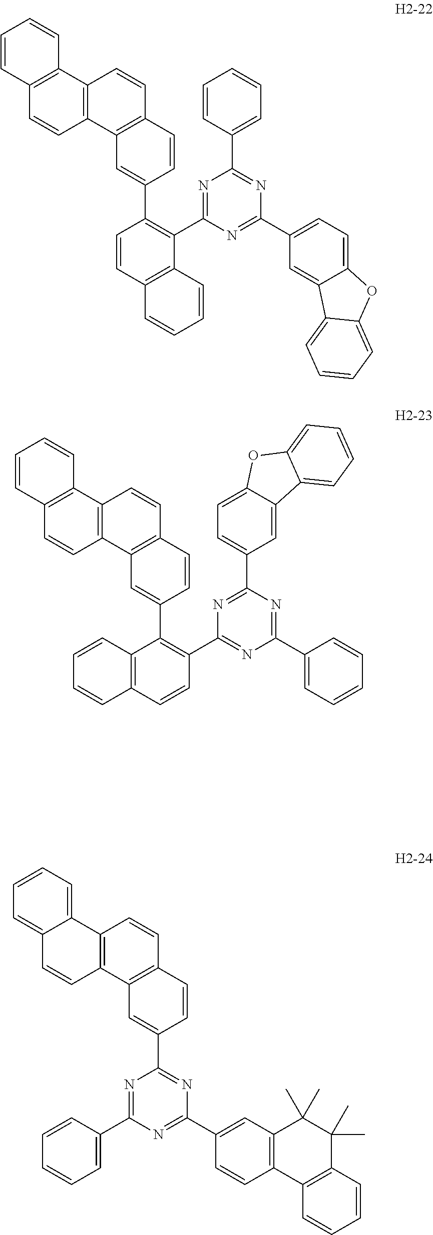 [SMILES: c1ccc(-c2nc(-c3ccc4oc5ccccc5c4c3)nc(-c3c(-c4ccc5ccc6c7ccccc7ccc6c5c4)ccc4ccccc34)n2)cc1 ; c1ccc(-c2nc(-c3ccc4oc5ccccc5c4c3)nc(-c3ccc4ccccc4c3-c3ccc4ccc5c6ccccc6ccc5c4c3)n2)cc1 ; CC1(C)c2ccccc2-c2ccc(-c3nc(-c4ccccc4)nc(-c4ccc5ccc6c7ccccc7ccc6c5c4)n3)cc2C1(C)C]

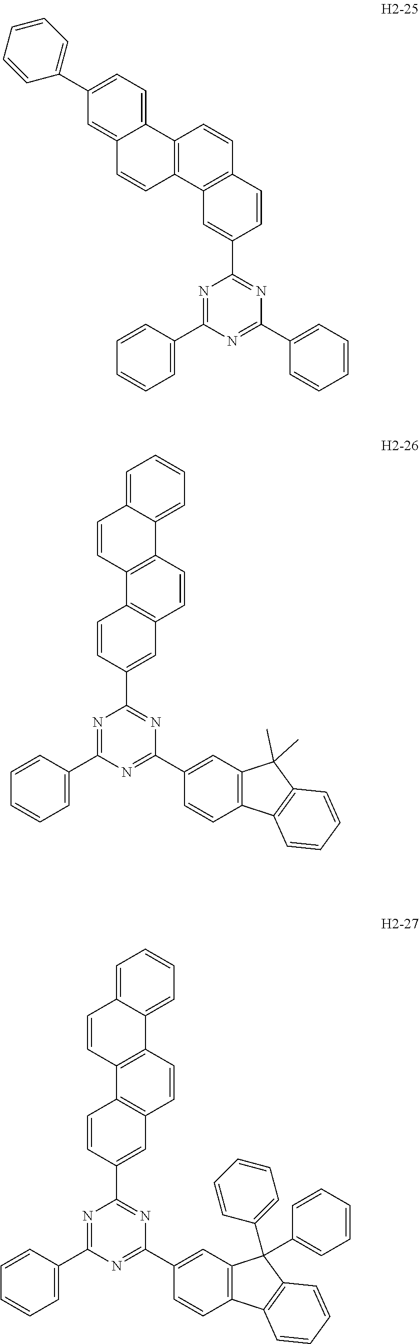 [SMILES: c1ccc(-c2ccc3c(ccc4c5cc(-c6nc(-c7ccccc7)nc(-c7ccccc7)n6)ccc5ccc34)c2)cc1 ; CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1]

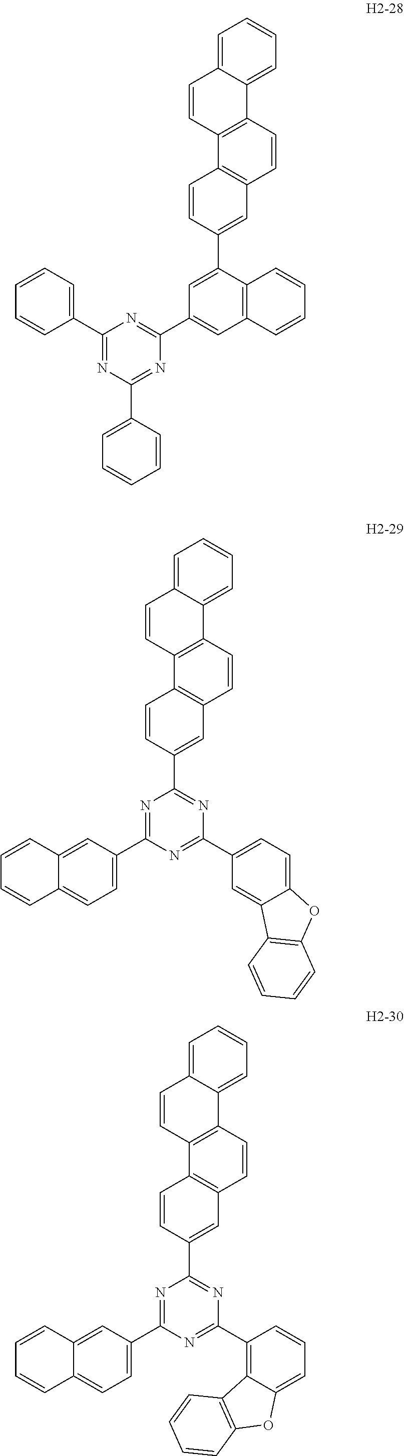 [SMILES: c1ccc(-c2nc(-c3ccccc3)nc(-c3cc(-c4ccc5c(ccc6c7ccccc7ccc56)c4)c4ccccc4c3)n2)cc1 ; c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1]

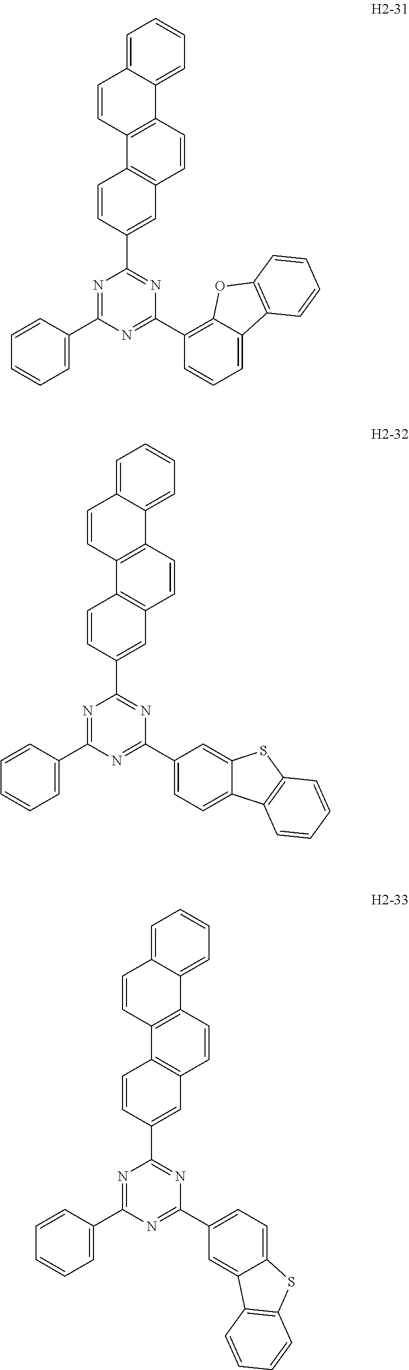 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

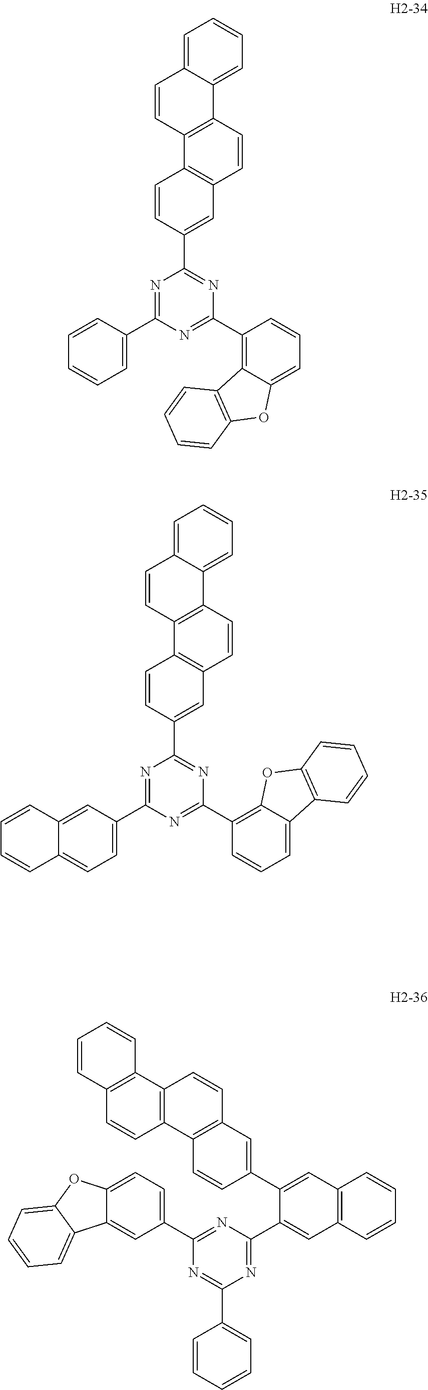 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1 ; c1ccc(-c2nc(-c3ccc4oc5ccccc5c4c3)nc(-c3cc4ccccc4cc3-c3ccc4c(ccc5c6ccccc6ccc45)c3)n2)cc1]

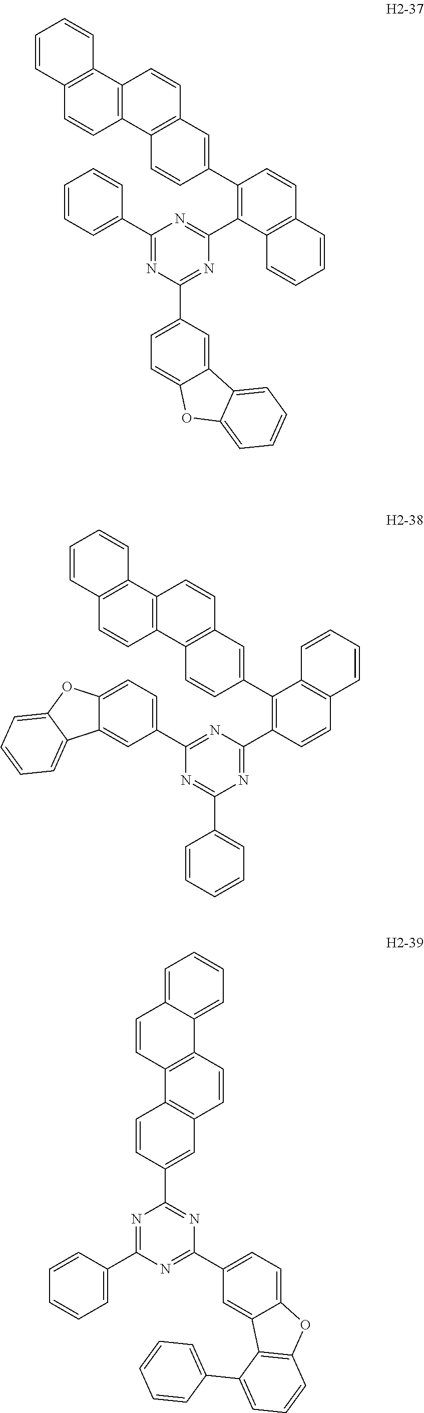 [SMILES: c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1]

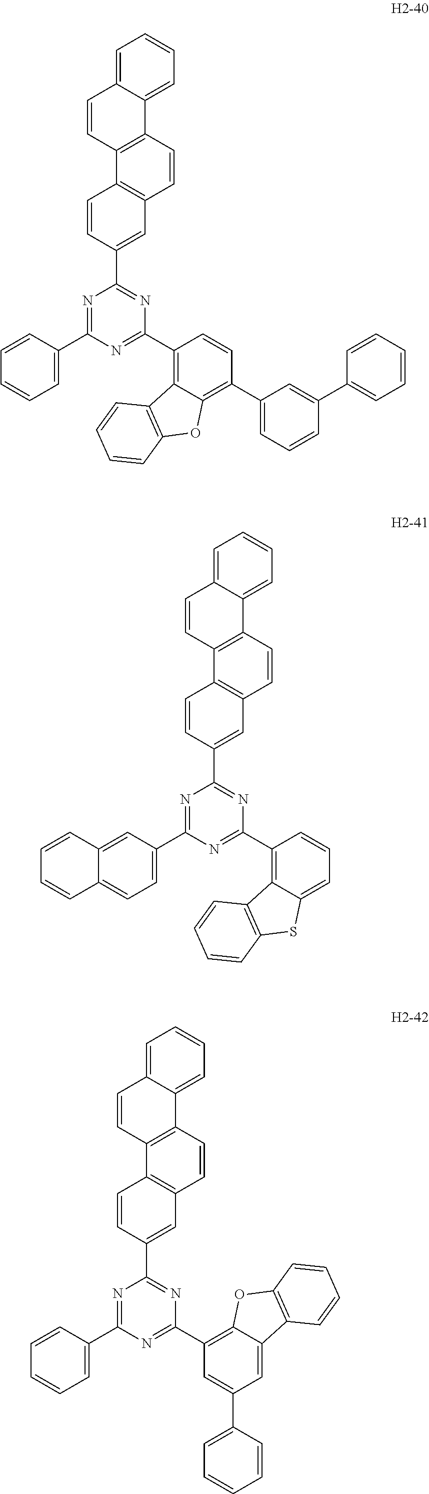 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1]

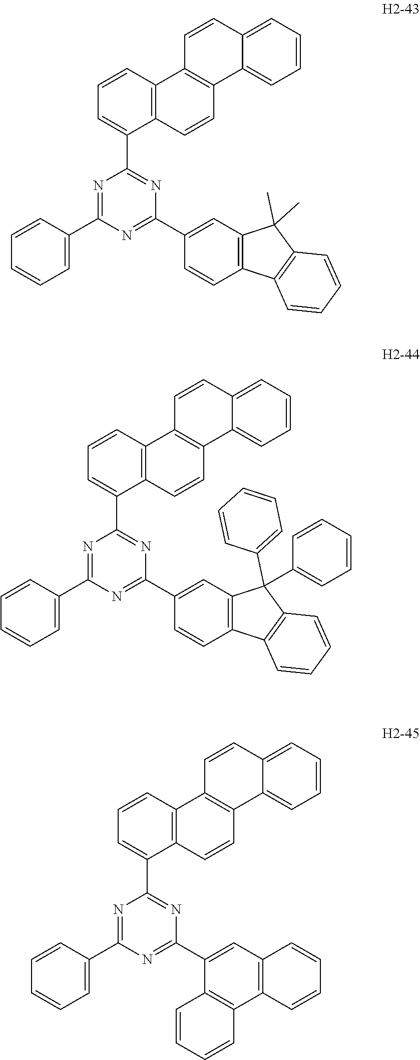 [SMILES: CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1 ; c1ccc(-c2nc(-c3cc4ccccc4c4ccccc34)nc(-c3cccc4c3ccc3c5ccccc5ccc43)n2)cc1]

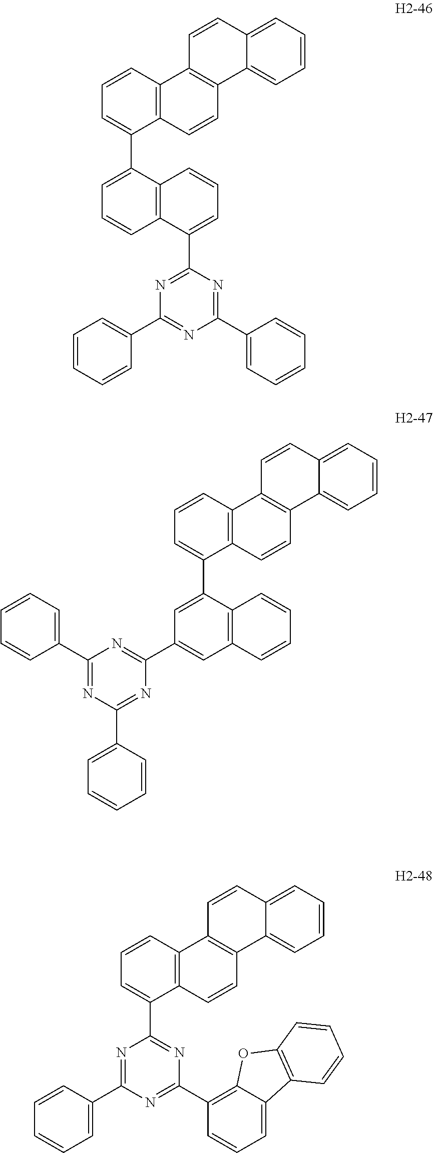 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1]

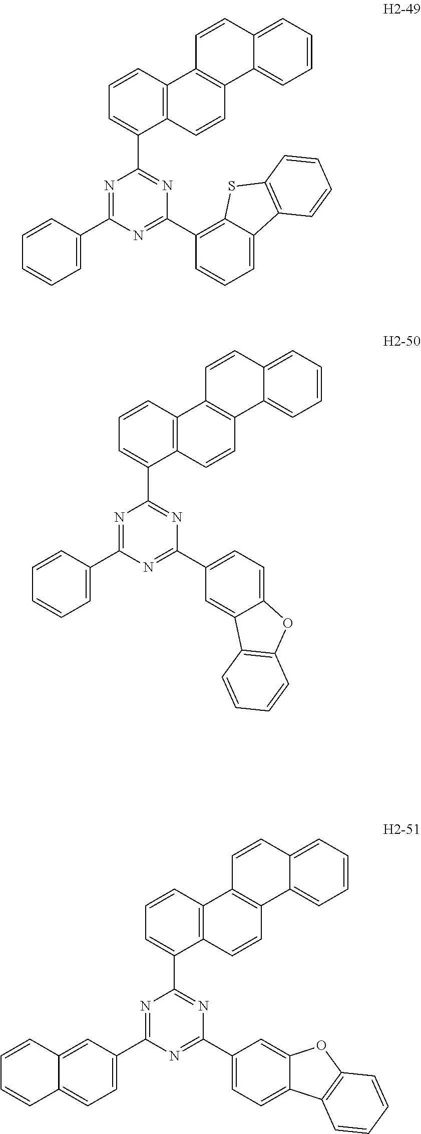 [SMILES: c1ccc(-c2nc(-c3cccc4c3ccc3c5ccccc5ccc43)nc(-c3cccc4c3sc3ccccc34)n2)cc1 ; c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1]

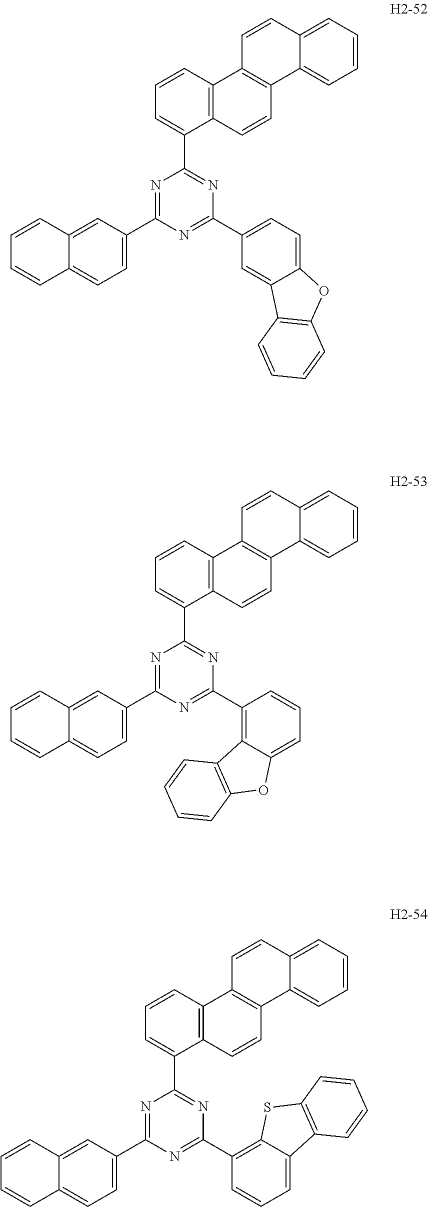 [SMILES: c1ccc2cc(-c3nc(-c4ccc5oc6ccccc6c5c4)nc(-c4cccc5c4ccc4c6ccccc6ccc54)n3)ccc2c1 ; c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1]

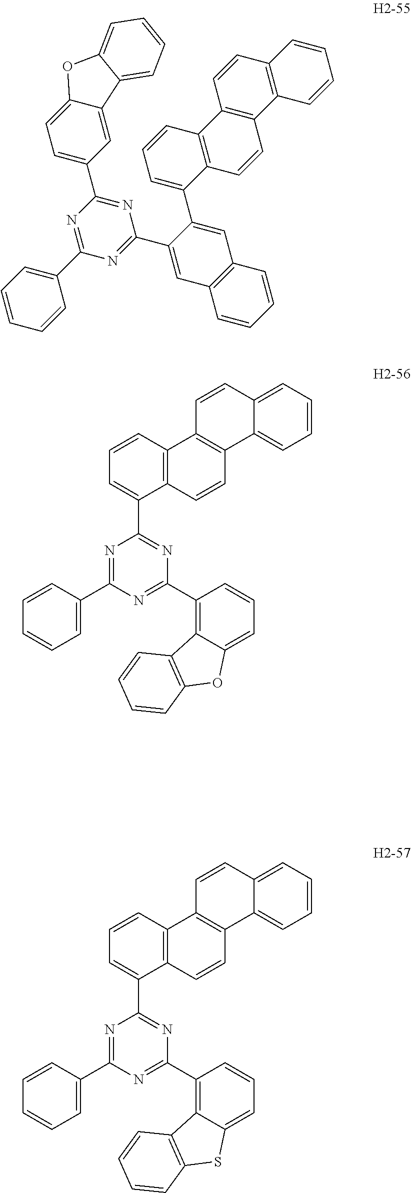 [SMILES: c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1]

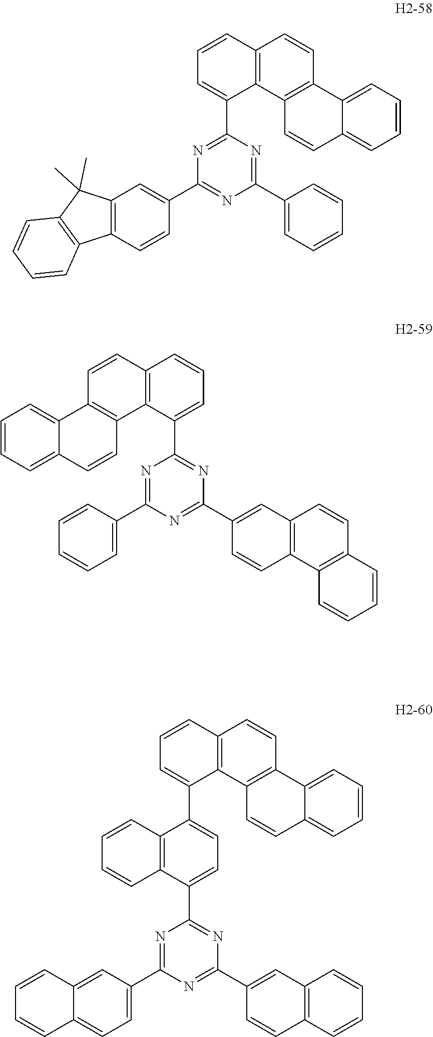 [SMILES: CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1 ; c1ccc2cc(-c3nc(-c4ccc5ccccc5c4)nc(-c4ccc(-c5cccc6ccc7c8ccccc8ccc7c56)c5ccccc45)n3)ccc2c1]

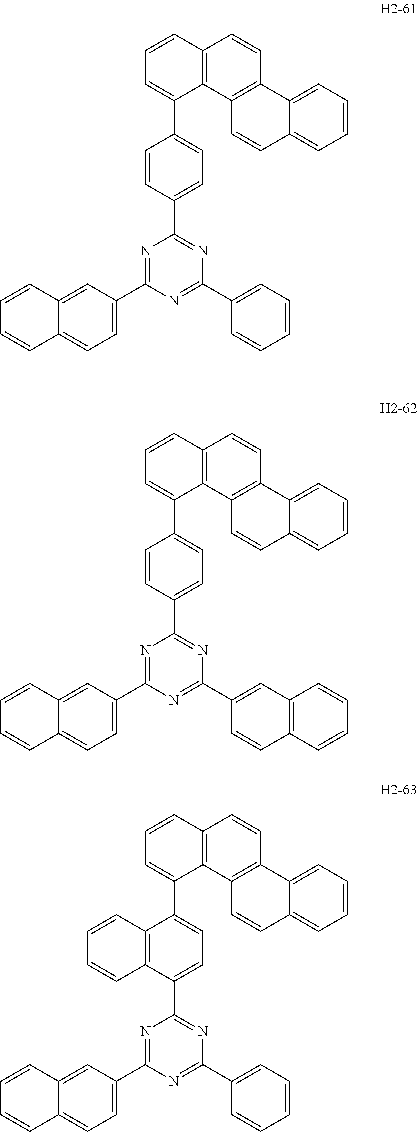 [SMILES: c1ccc(-c2nc(-c3ccc(-c4cccc5ccc6c7ccccc7ccc6c45)cc3)nc(-c3ccc4ccccc4c3)n2)cc1 ; c1ccc2cc(-c3nc(-c4ccc(-c5cccc6ccc7c8ccccc8ccc7c56)cc4)nc(-c4ccc5ccccc5c4)n3)ccc2c1 ; c1ccc(-c2nc(-c3ccc4ccccc4c3)nc(-c3ccc(-c4cccc5ccc6c7ccccc7ccc6c45)c4ccccc34)n2)cc1]

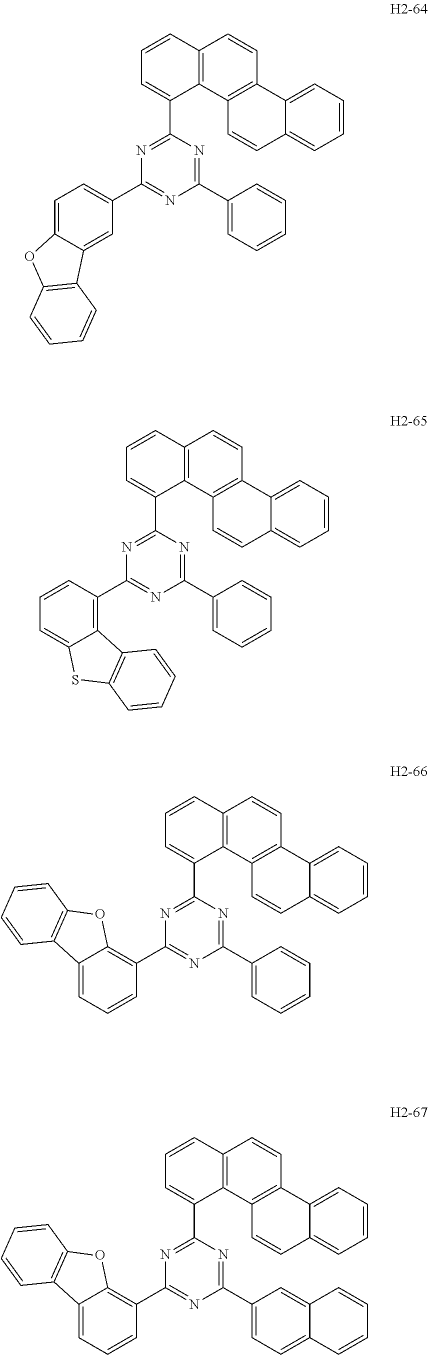 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1]

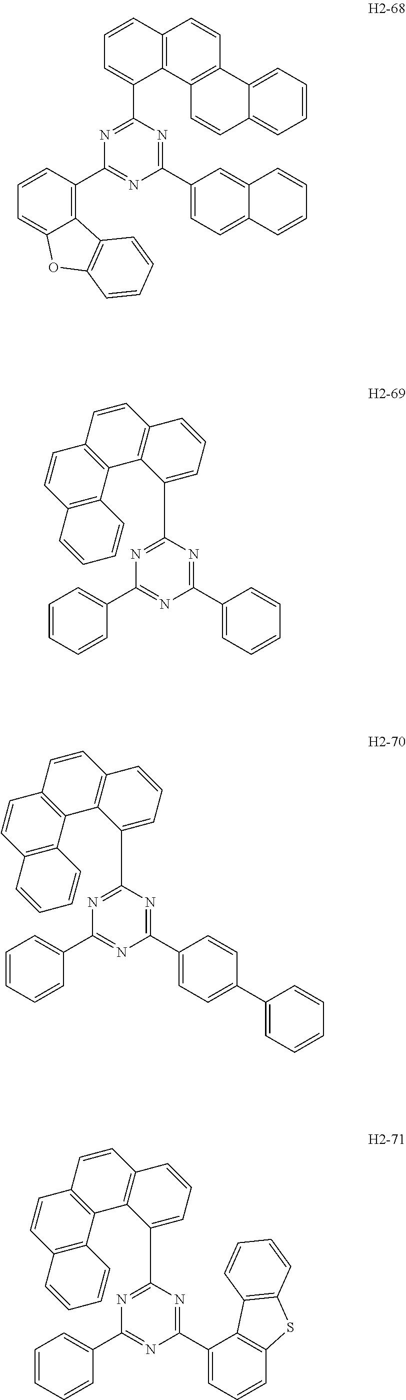 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3cccc4sc5ccccc5c34)nc(-c3cccc4ccc5ccc6ccccc6c5c34)n2)cc1]

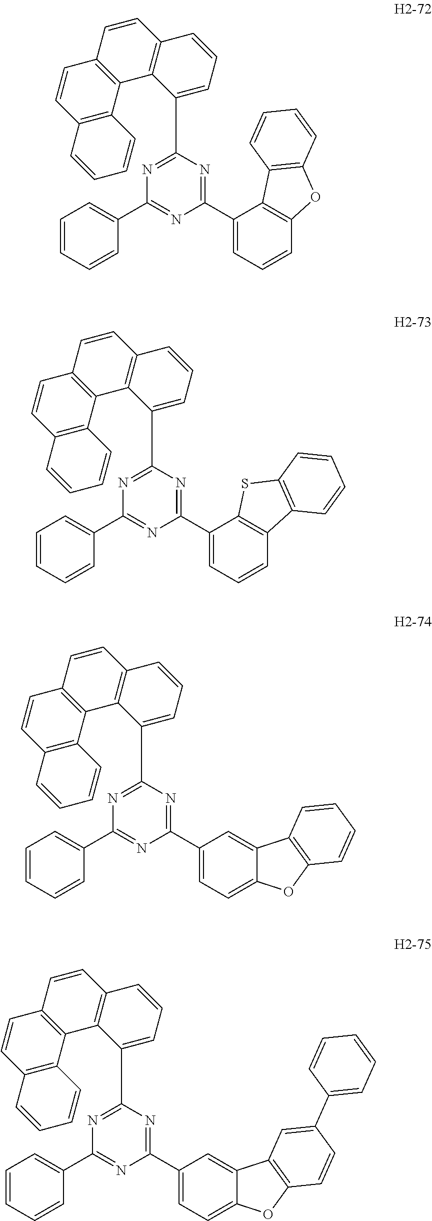 [SMILES: c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1 ; c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1]

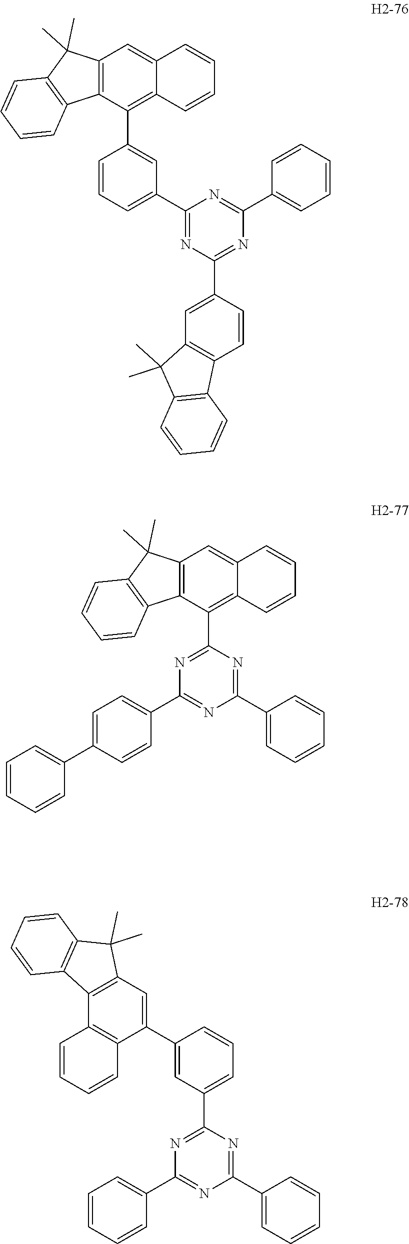 [SMILES: CC1(C)c2ccccc2-c2ccc(-c3nc(-c4ccccc4)nc(-c4cccc(-c5c6c(cc7ccccc57)C(C)(C)c5ccccc5-6)c4)n3)cc21 ; CC1(C)c2ccccc2-c2c1cc1ccccc1c2-c1nc(-c2ccccc2)nc(-c2ccc(-c3ccccc3)cc2)n1 ; CC1(C)c2ccccc2-c2c1cc(-c1cccc(-c3nc(-c4ccccc4)nc(-c4ccccc4)n3)c1)c1ccccc21]

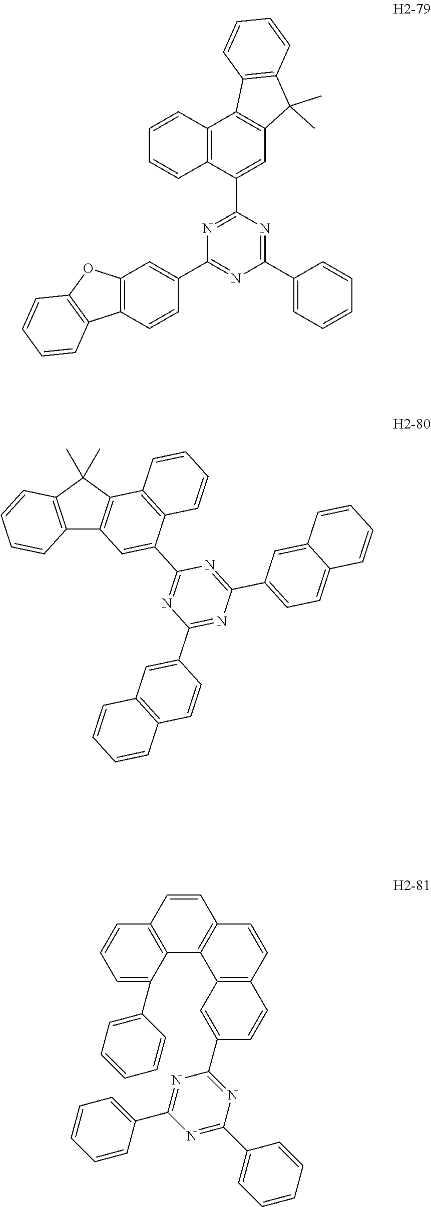 [SMILES: CC1(C)c2ccccc2-c2c1cc(-c1nc(-c3ccccc3)nc(-c3ccc4c(c3)oc3ccccc34)n1)c1ccccc21 ; CC1(C)c2ccccc2-c2cc(-c3nc(-c4ccc5ccccc5c4)nc(-c4ccc5ccccc5c4)n3)c3ccccc3c21 ; c1ccc(-c2nc(-c3ccccc3)nc(-c3ccc4ccc5ccc6cccc(-c7ccccc7)c6c5c4c3)n2)cc1]

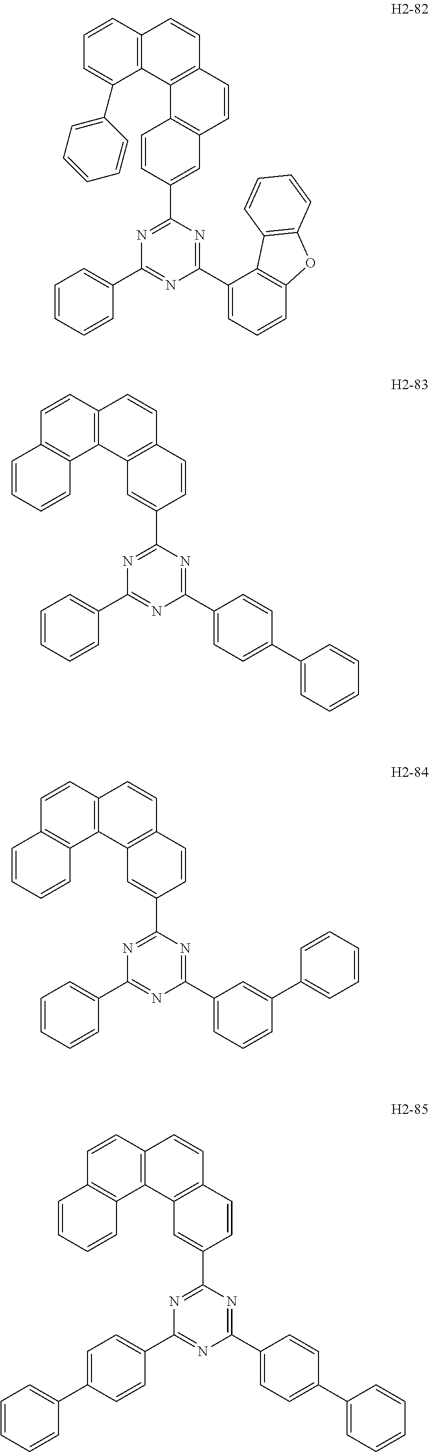 [SMILES: c1ccc(-c2nc(-c3ccc4c(ccc5ccc6cccc(-c7ccccc7)c6c54)c3)nc(-c3cccc4oc5ccccc5c34)n2)cc1 ; c1ccc(-c2ccc(-c3nc(-c4ccccc4)nc(-c4ccc5ccc6ccc7ccccc7c6c5c4)n3)cc2)cc1 ; c1ccc(-c2cccc(-c3nc(-c4ccccc4)nc(-c4ccc5ccc6ccc7ccccc7c6c5c4)n3)c2)cc1 ; c1ccc(-c2ccc(-c3nc(-c4ccc(-c5ccccc5)cc4)nc(-c4ccc5ccc6ccc7ccccc7c6c5c4)n3)cc2)cc1]

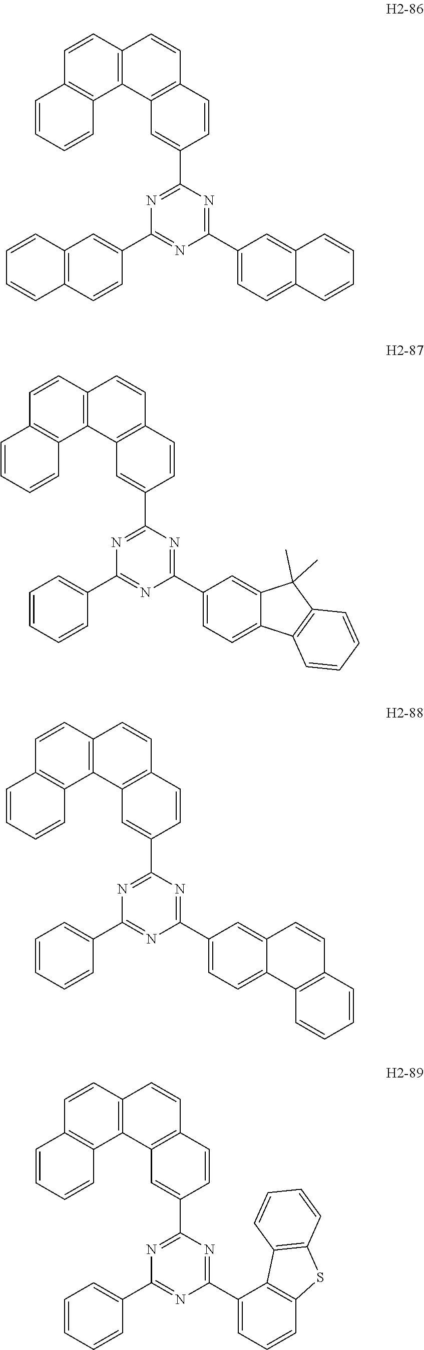 [SMILES: c1ccc2cc(-c3nc(-c4ccc5ccccc5c4)nc(-c4ccc5ccc6ccc7ccccc7c6c5c4)n3)ccc2c1 ; CC1(C)c2ccccc2-c2ccc(-c3nc(-c4ccccc4)nc(-c4ccc5ccc6ccc7ccccc7c6c5c4)n3)cc21 ; c1ccc(-c2nc(-c3ccc4c(ccc5ccccc54)c3)nc(-c3ccc4ccc5ccc6ccccc6c5c4c3)n2)cc1 ; c1ccc(-c2nc(-c3ccc4ccc5ccc6ccccc6c5c4c3)nc(-c3cccc4sc5ccccc5c34)n2)cc1]

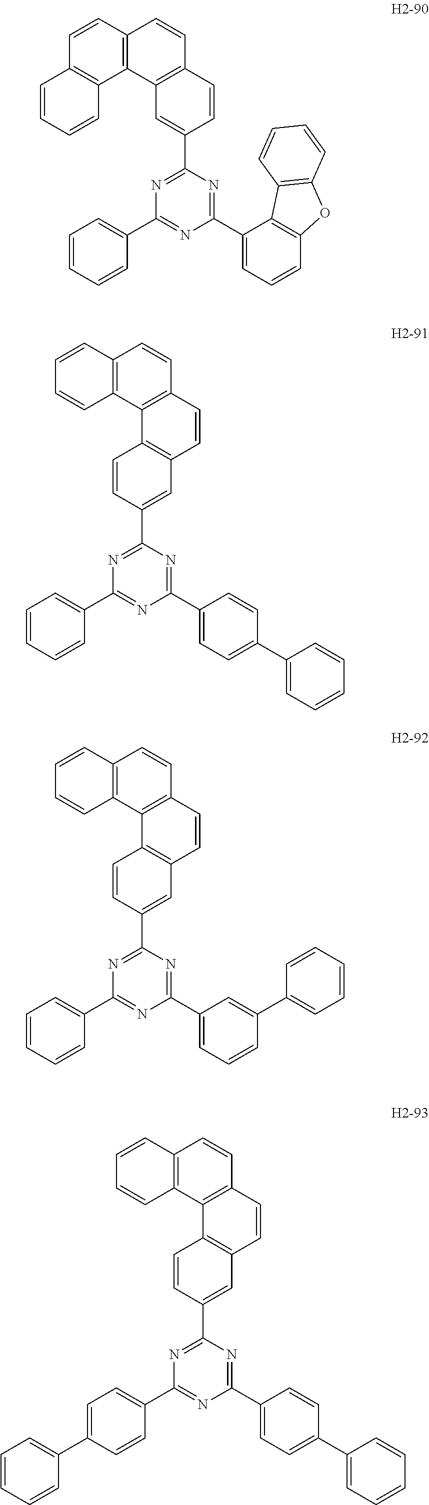 [SMILES: c1ccc(-c2nc(-c3ccc4ccc5ccc6ccccc6c5c4c3)nc(-c3cccc4oc5ccccc5c34)n2)cc1 ; c1ccc(-c2ccc(-c3nc(-c4ccccc4)nc(-c4ccc5c(ccc6ccc7ccccc7c65)c4)n3)cc2)cc1 ; c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1]

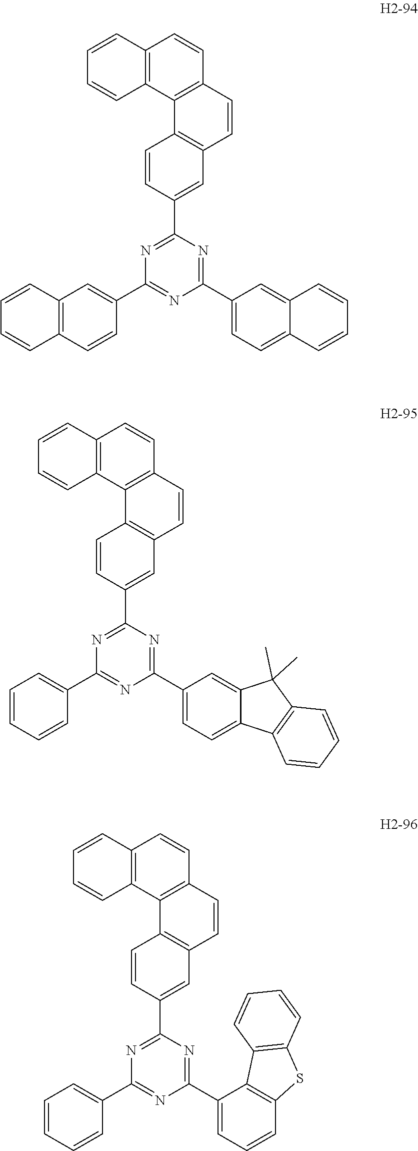 [SMILES: c1ccc2cc(-c3nc(-c4ccc5ccccc5c4)nc(-c4ccc5c(ccc6ccc7ccccc7c65)c4)n3)ccc2c1 ; CC1(C)c2ccccc2-c2ccc(-c3nc(-c4ccccc4)nc(-c4ccc5c(ccc6ccc7ccccc7c65)c4)n3)cc21 ; c1ccc(-c2nc(-c3ccc4c(ccc5ccc6ccccc6c54)c3)nc(-c3cccc4sc5ccccc5c34)n2)cc1]

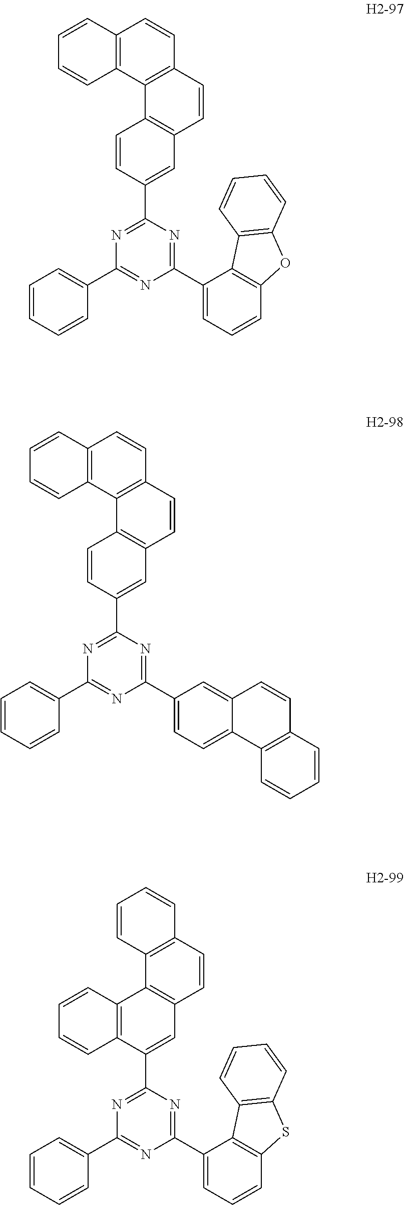 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3ccc4c(ccc5ccccc54)c3)nc(-c3ccc4c(ccc5ccc6ccccc6c54)c3)n2)cc1 ; c1ccc(-c2nc(-c3cc4ccc5ccccc5c4c4ccccc34)nc(-c3cccc4sc5ccccc5c34)n2)cc1]

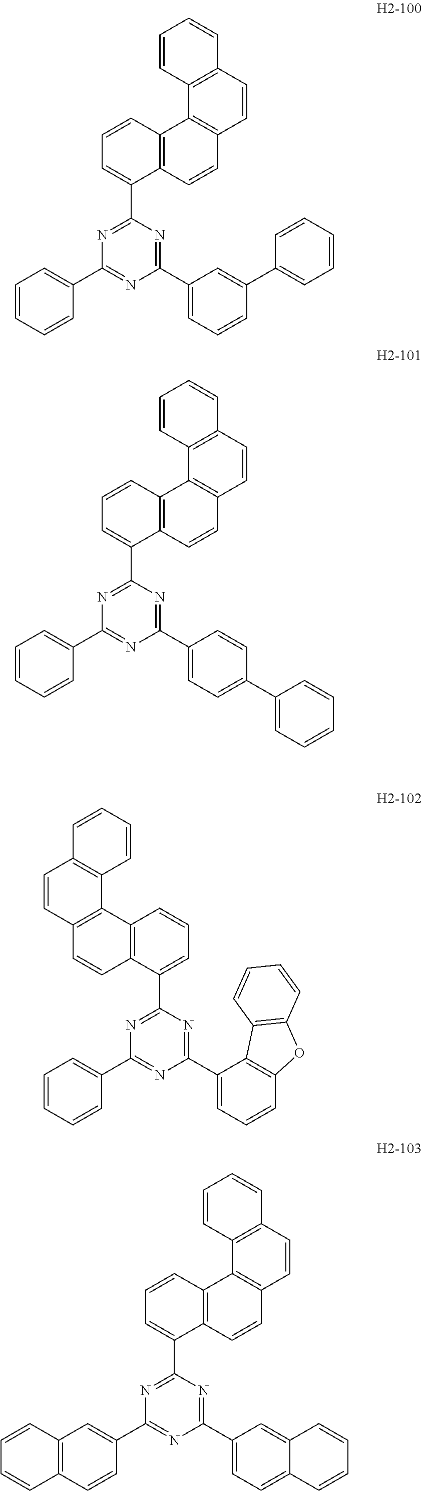 [SMILES: c1ccc(-c2cccc(-c3nc(-c4ccccc4)nc(-c4cccc5c4ccc4ccc6ccccc6c45)n3)c2)cc1 ; c1ccc(-c2ccc(-c3nc(-c4ccccc4)nc(-c4cccc5c4ccc4ccc6ccccc6c45)n3)cc2)cc1 ; c1ccc(-c2nc(-c3cccc4c3ccc3ccc5ccccc5c34)nc(-c3cccc4oc5ccccc5c34)n2)cc1 ; c1ccc2cc(-c3nc(-c4ccc5ccccc5c4)nc(-c4cccc5c4ccc4ccc6ccccc6c45)n3)ccc2c1]

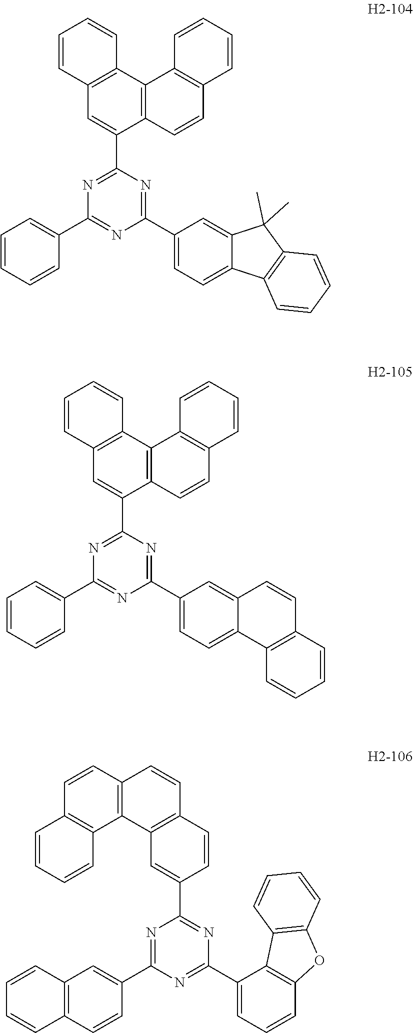 [SMILES: CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1 ; c1ccc2cc(-c3nc(-c4ccc5ccc6ccc7ccccc7c6c5c4)nc(-c4cccc5oc6ccccc6c45)n3)ccc2c1]

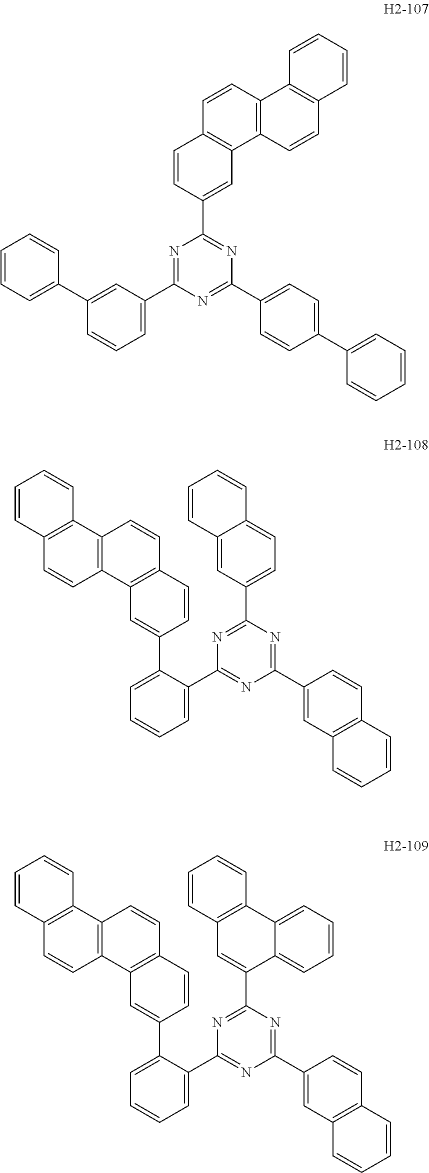 [SMILES: c1ccc(-c2ccc(-c3nc(-c4cccc(-c5ccccc5)c4)nc(-c4ccc5ccc6c7ccccc7ccc6c5c4)n3)cc2)cc1 ; c1ccc(-c2nc(-c3ccc4ccccc4c3)nc(-c3ccc4ccccc4c3)n2)c(-c2ccc3ccc4c5ccccc5ccc4c3c2)c1 ; c1ccc(-c2nc(-c3ccc4ccccc4c3)nc(-c3cc4ccccc4c4ccccc34)n2)c(-c2ccc3ccc4c5ccccc5ccc4c3c2)c1]

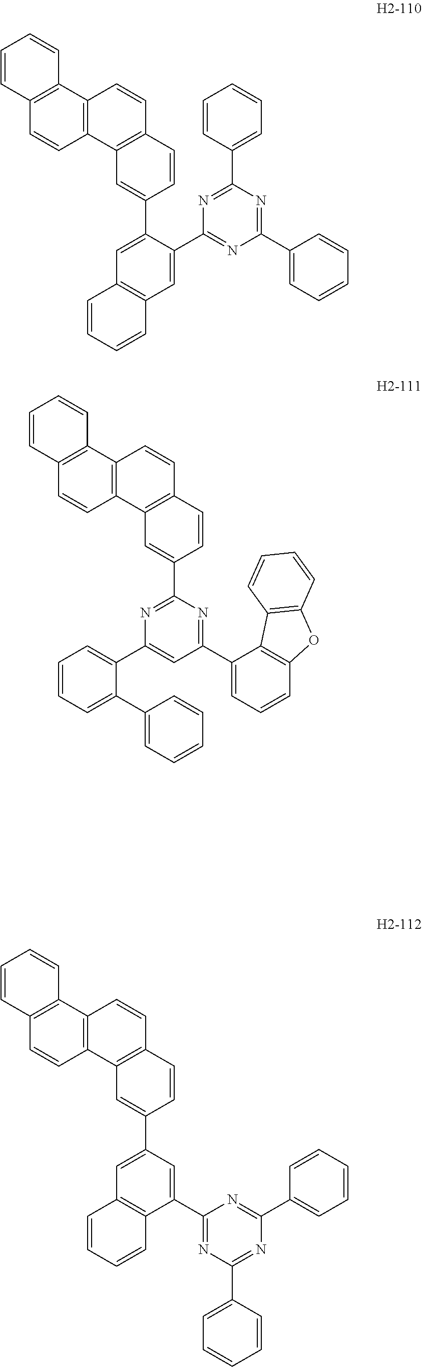 [SMILES: c1ccc(-c2nc(-c3ccccc3)nc(-c3cc4ccccc4cc3-c3ccc4ccc5c6ccccc6ccc5c4c3)n2)cc1 ; c1ccc(-c2ccccc2-c2cc(-c3cccc4oc5ccccc5c34)nc(-c3ccc4ccc5c6ccccc6ccc5c4c3)n2)cc1 ; c1ccc(-c2nc(-c3ccccc3)nc(-c3cc(-c4ccc5ccc6c7ccccc7ccc6c5c4)cc4ccccc34)n2)cc1]

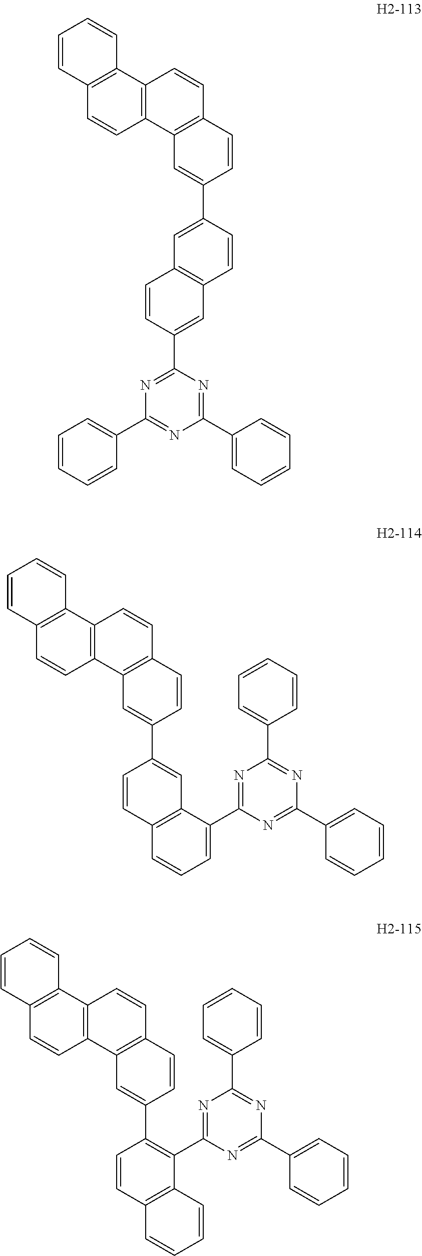 [SMILES: c1ccc(-c2nc(-c3ccccc3)nc(-c3ccc4cc(-c5ccc6ccc7c8ccccc8ccc7c6c5)ccc4c3)n2)cc1 ; c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1]

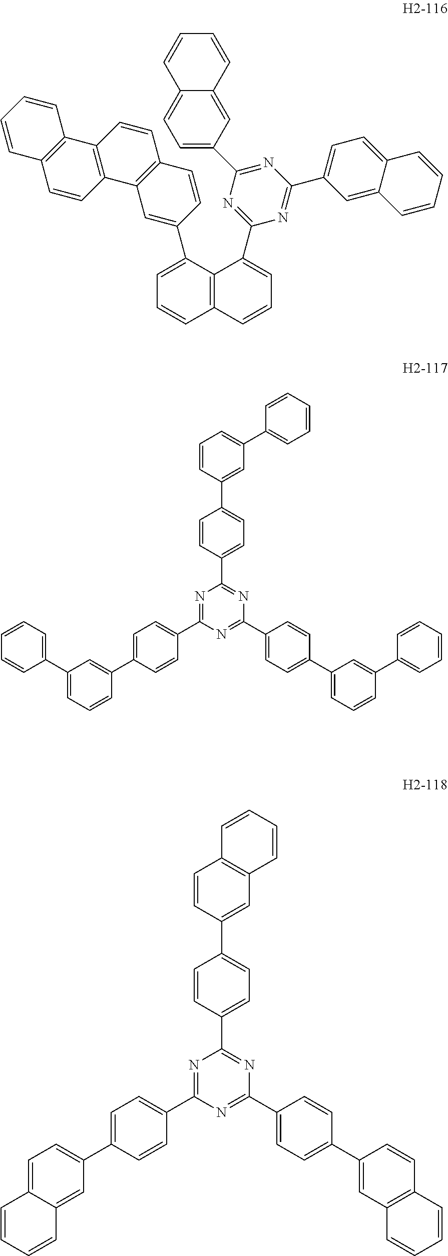 [SMILES: c1ccc2cc(-c3nc(-c4ccc5ccccc5c4)nc(-c4cccc5cccc(-c6ccc7ccc8c9ccccc9ccc8c7c6)c45)n3)ccc2c1 ; c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4nc(-c5ccc(-c6ccc7ccccc7c6)cc5)nc(-c5ccc(-c6ccc7ccccc7c6)cc5)n4)cc3)ccc2c1]

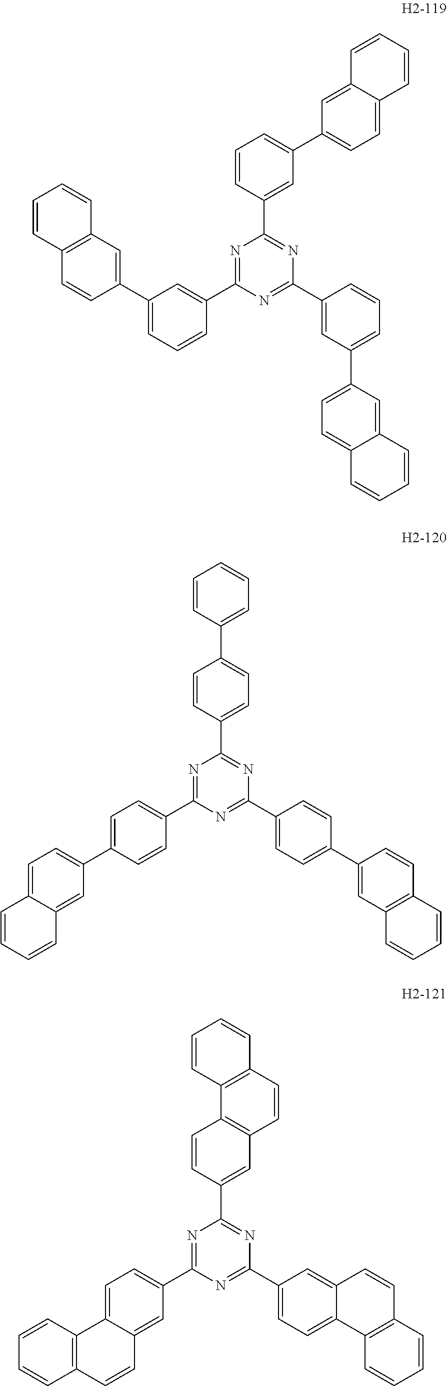 [SMILES: c1cc(-c2ccc3ccccc3c2)cc(-c2nc(-c3cccc(-c4ccc5ccccc5c4)c3)nc(-c3cccc(-c4ccc5ccccc5c4)c3)n2)c1 ; c1ccc(-c2ccc(-c3nc(-c4ccc(-c5ccc6ccccc6c5)cc4)nc(-c4ccc(-c5ccc6ccccc6c5)cc4)n3)cc2)cc1 ; c1ccc2c(c1)ccc1cc(-c3nc(-c4ccc5c(ccc6ccccc65)c4)nc(-c4ccc5c(ccc6ccccc65)c4)n3)ccc12]

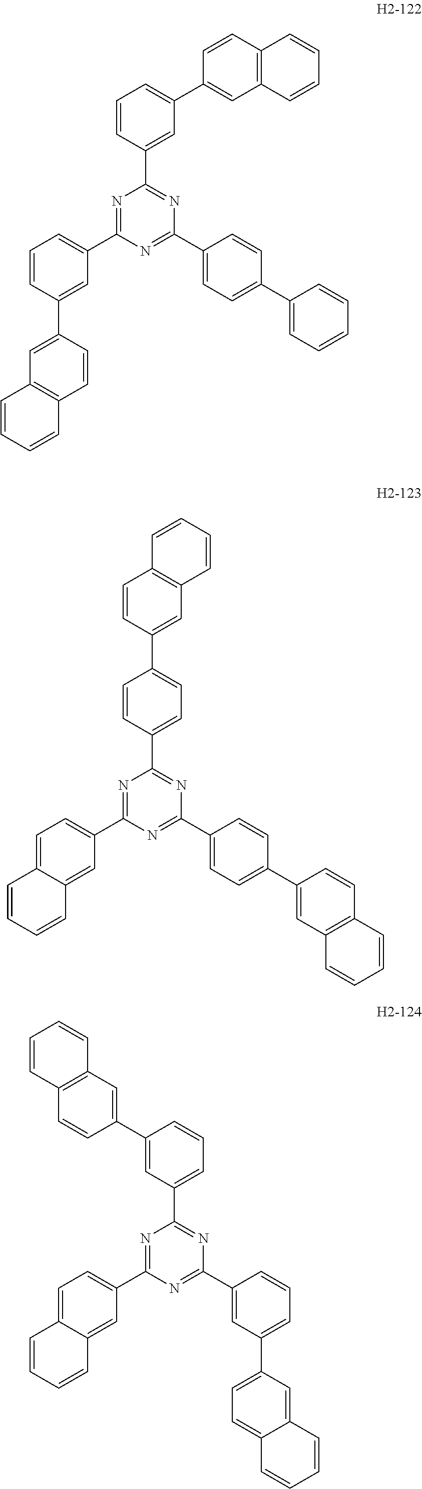 [SMILES: c1ccc(-c2ccc(-c3nc(-c4cccc(-c5ccc6ccccc6c5)c4)nc(-c4cccc(-c5ccc6ccccc6c5)c4)n3)cc2)cc1 ; c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1]

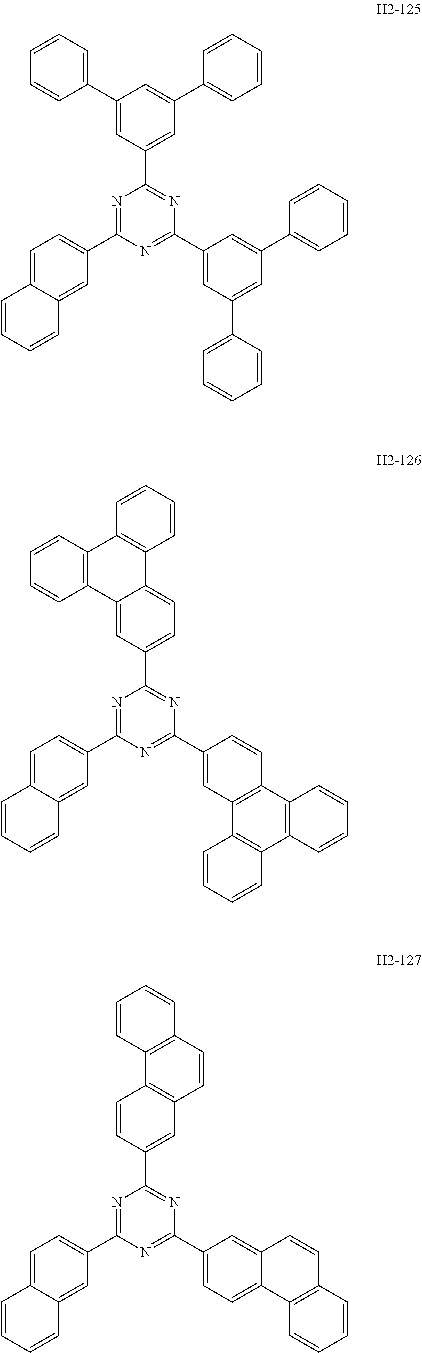 [SMILES: c1ccc(-c2cc(-c3ccccc3)cc(-c3nc(-c4cc(-c5ccccc5)cc(-c5ccccc5)c4)nc(-c4ccc5ccccc5c4)n3)c2)cc1 ; c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1]

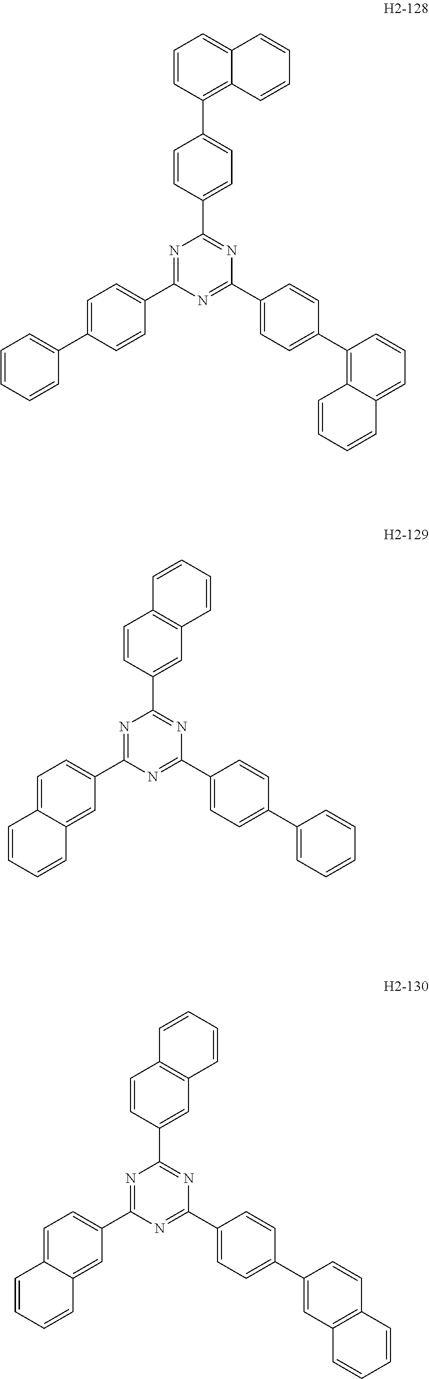 [SMILES: c1ccc(-c2ccc(-c3nc(-c4ccc(-c5cccc6ccccc56)cc4)nc(-c4ccc(-c5cccc6ccccc56)cc4)n3)cc2)cc1 ; c1ccc(-c2ccc(-c3nc(-c4ccc5ccccc5c4)nc(-c4ccc5ccccc5c4)n3)cc2)cc1 ; c1ccc2cc(-c3ccc(-c4nc(-c5ccc6ccccc6c5)nc(-c5ccc6ccccc6c5)n4)cc3)ccc2c1]

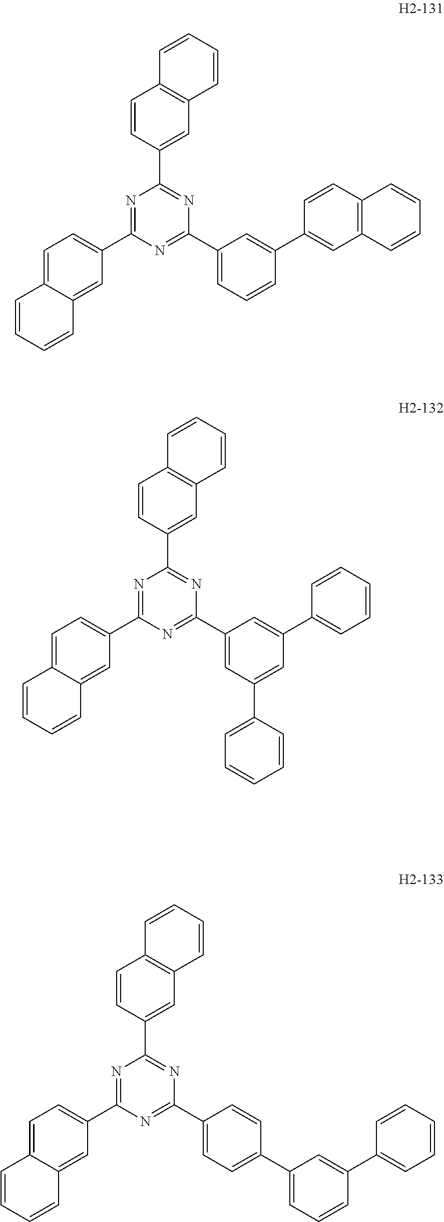 [SMILES: c1cc(-c2ccc3ccccc3c2)cc(-c2nc(-c3ccc4ccccc4c3)nc(-c3ccc4ccccc4c3)n2)c1 ; c1ccc(-c2cc(-c3ccccc3)cc(-c3nc(-c4ccc5ccccc5c4)nc(-c4ccc5ccccc5c4)n3)c2)cc1 ; c1ccc(-c2cccc(-c3ccc(-c4nc(-c5ccc6ccccc6c5)nc(-c5ccc6ccccc6c5)n4)cc3)c2)cc1]

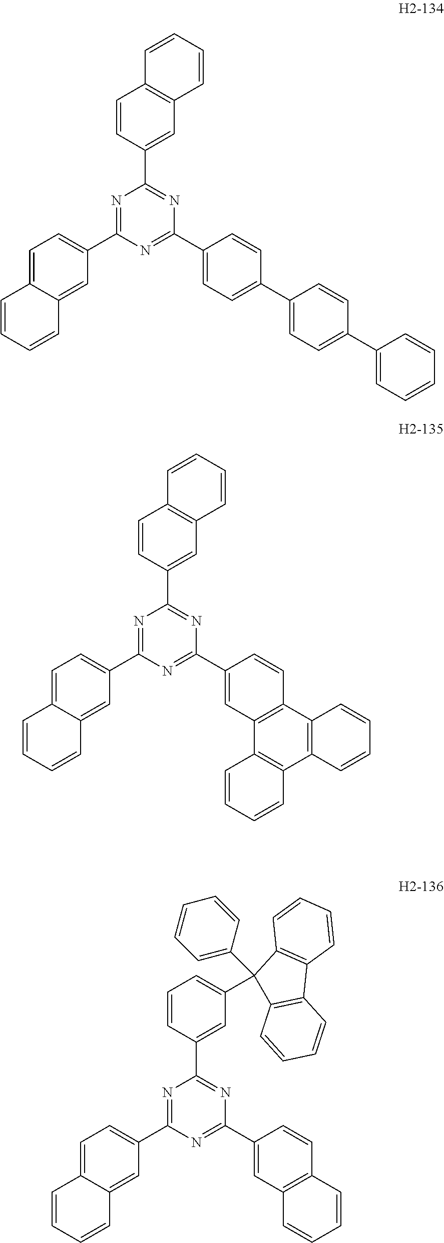 [SMILES: c1ccc(-c2ccc(-c3ccc(-c4nc(-c5ccc6ccccc6c5)nc(-c5ccc6ccccc6c5)n4)cc3)cc2)cc1 ; c1ccc2cc(-c3nc(-c4ccc5ccccc5c4)nc(-c4ccc5c6ccccc6c6ccccc6c5c4)n3)ccc2c1 ; c1ccc(C2(c3cccc(-c4nc(-c5ccc6ccccc6c5)nc(-c5ccc6ccccc6c5)n4)c3)c3ccccc3-c3ccccc32)cc1]

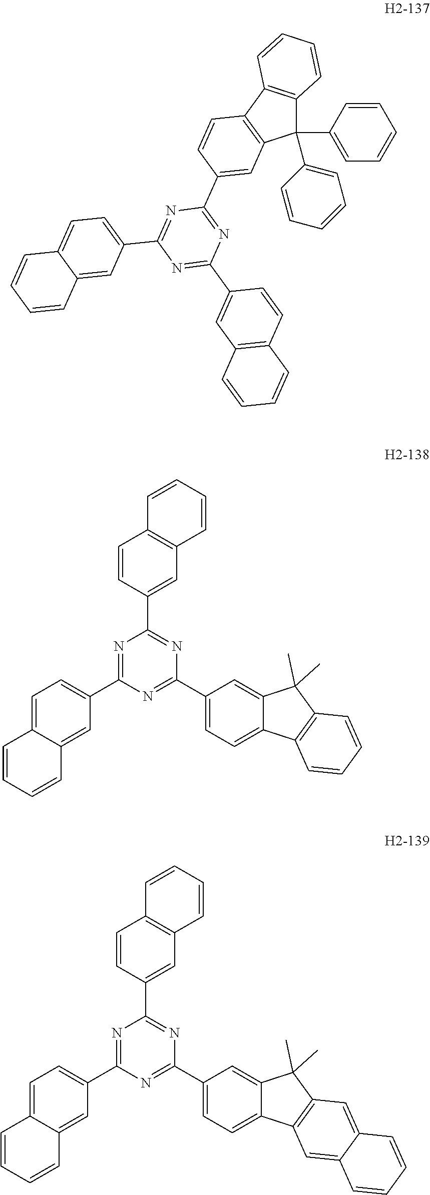 [SMILES: c1ccc(C2(c3ccccc3)c3ccccc3-c3ccc(-c4nc(-c5ccc6ccccc6c5)nc(-c5ccc6ccccc6c5)n4)cc32)cc1 ; CC1(C)c2ccccc2-c2ccc(-c3nc(-c4ccc5ccccc5c4)nc(-c4ccc5ccccc5c4)n3)cc21 ; CC1(C)c2cc(-c3nc(-c4ccc5ccccc5c4)nc(-c4ccc5ccccc5c4)n3)ccc2-c2cc3ccccc3cc21]

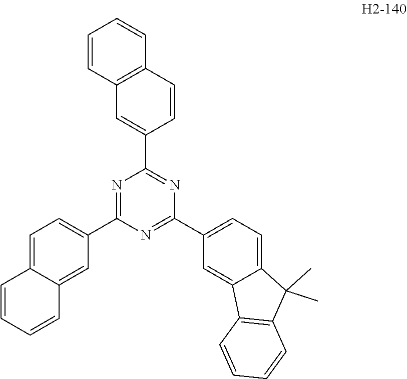 [SMILES: CC1(C)c2ccccc2-c2cc(-c3nc(-c4ccc5ccccc5c4)nc(-c4ccc5ccccc5c4)n3)ccc21]

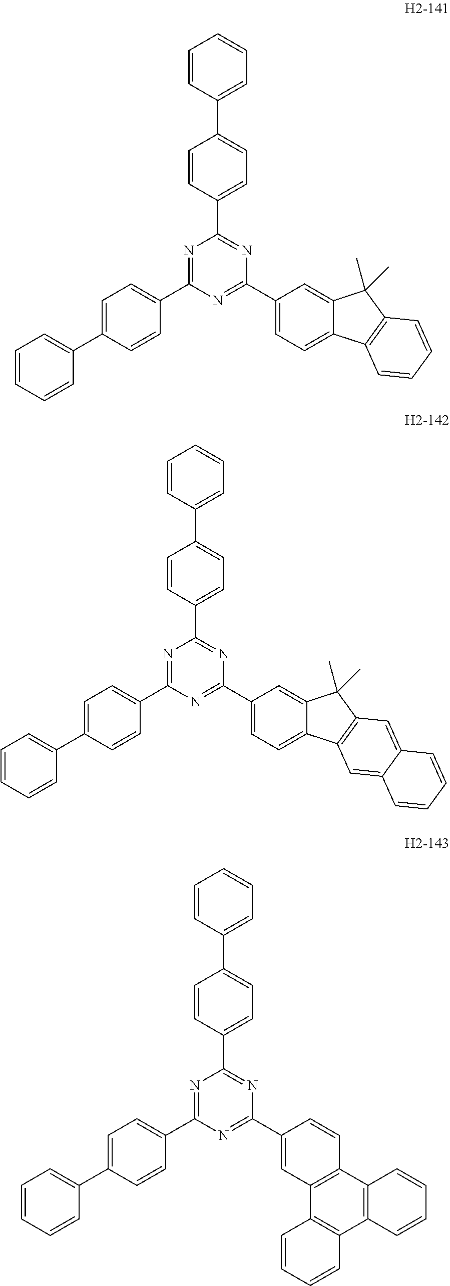 [SMILES: CC1(C)c2ccccc2-c2ccc(-c3nc(-c4ccc(-c5ccccc5)cc4)nc(-c4ccc(-c5ccccc5)cc4)n3)cc21 ; CC1(C)c2cc(-c3nc(-c4ccc(-c5ccccc5)cc4)nc(-c4ccc(-c5ccccc5)cc4)n3)ccc2-c2cc3ccccc3cc21 ; c1ccc(-c2ccc(-c3nc(-c4ccc(-c5ccccc5)cc4)nc(-c4ccc5c6ccccc6c6ccccc6c5c4)n3)cc2)cc1]

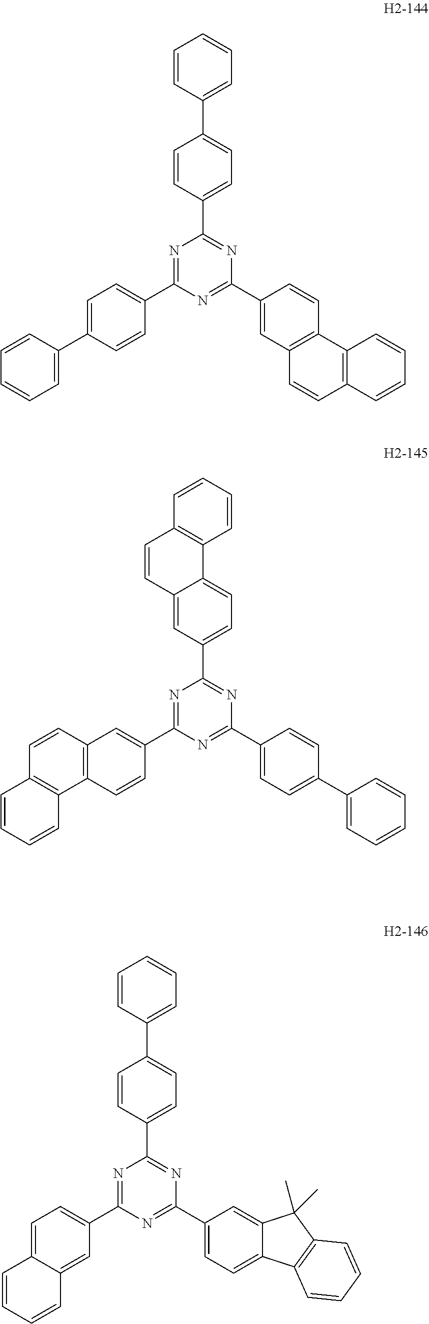 [SMILES: c1ccc(-c2ccc(-c3nc(-c4ccc(-c5ccccc5)cc4)nc(-c4ccc5c(ccc6ccccc65)c4)n3)cc2)cc1 ; c1ccc(-c2ccc(-c3nc(-c4ccc5c(ccc6ccccc65)c4)nc(-c4ccc5c(ccc6ccccc65)c4)n3)cc2)cc1 ; CC1(C)c2ccccc2-c2ccc(-c3nc(-c4ccc(-c5ccccc5)cc4)nc(-c4ccc5ccccc5c4)n3)cc21]

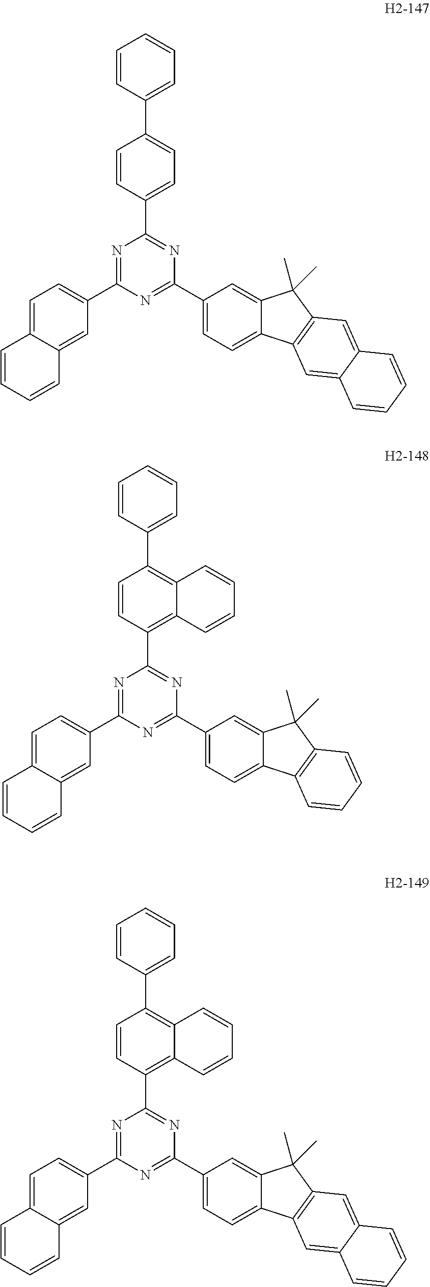 [SMILES: CC1(C)c2cc(-c3nc(-c4ccc(-c5ccccc5)cc4)nc(-c4ccc5ccccc5c4)n3)ccc2-c2cc3ccccc3cc21 ; CC1(C)c2ccccc2-c2ccc(-c3nc(-c4ccc5ccccc5c4)nc(-c4ccc(-c5ccccc5)c5ccccc45)n3)cc21 ; CC1(C)c2cc(-c3nc(-c4ccc5ccccc5c4)nc(-c4ccc(-c5ccccc5)c5ccccc45)n3)ccc2-c2cc3ccccc3cc21]

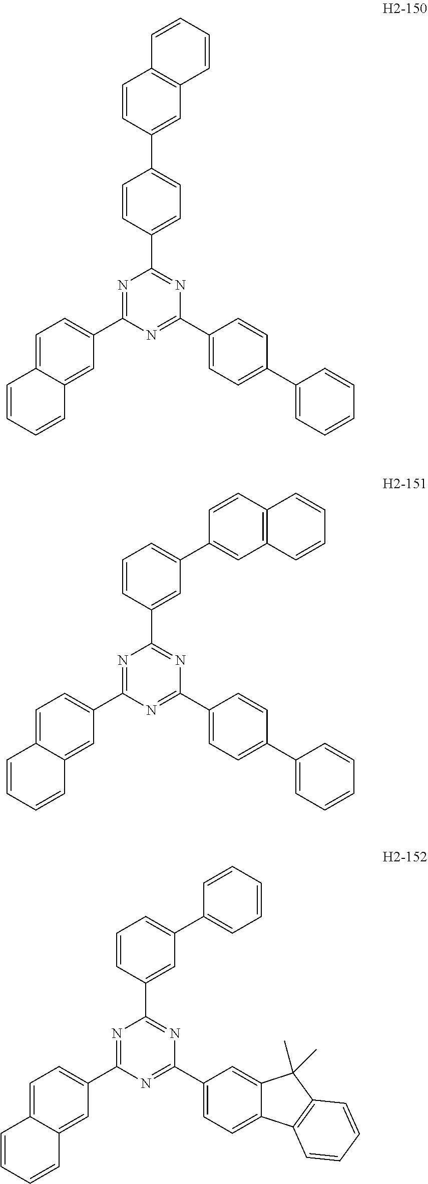 [SMILES: c1ccc(-c2ccc(-c3nc(-c4ccc(-c5ccc6ccccc6c5)cc4)nc(-c4ccc5ccccc5c4)n3)cc2)cc1 ; c1ccc(-c2ccc(-c3nc(-c4cccc(-c5ccc6ccccc6c5)c4)nc(-c4ccc5ccccc5c4)n3)cc2)cc1 ; CC1(C)c2ccccc2-c2ccc(-c3nc(-c4cccc(-c5ccccc5)c4)nc(-c4ccc5ccccc5c4)n3)cc21]

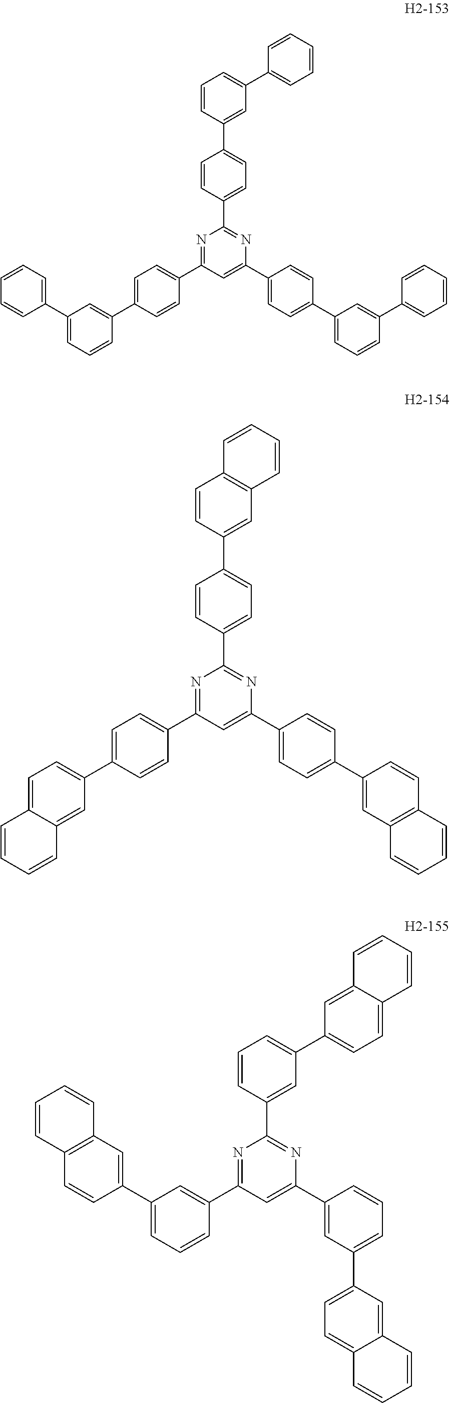 [SMILES: c1ccc(-c2cccc(-c3ccc(-c4cc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4cc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2cc(-c3cccc(-c4ccc5ccccc5c4)c3)nc(-c3cccc(-c4ccc5ccccc5c4)c3)n2)c1]

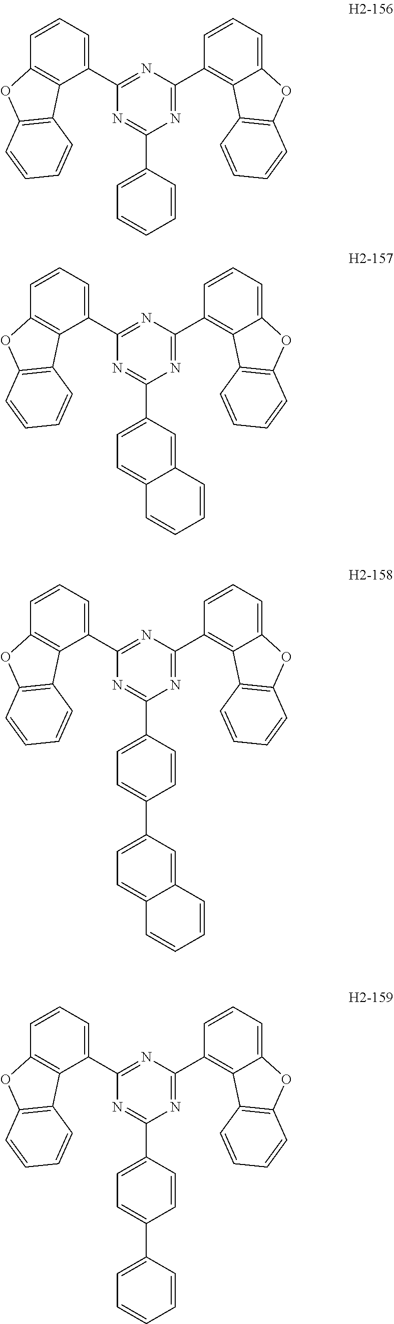 [SMILES: c1ccc(-c2nc(-c3cccc4oc5ccccc5c34)nc(-c3cccc4oc5ccccc5c34)n2)cc1 ; c1ccc2cc(-c3nc(-c4cccc5oc6ccccc6c45)nc(-c4cccc5oc6ccccc6c45)n3)ccc2c1 ; c1ccc2cc(-c3ccc(-c4nc(-c5cccc6oc7ccccc7c56)nc(-c5cccc6oc7ccccc7c56)n4)cc3)ccc2c1 ; c1ccc(-c2ccc(-c3nc(-c4cccc5oc6ccccc6c45)nc(-c4cccc5oc6ccccc6c45)n3)cc2)cc1]

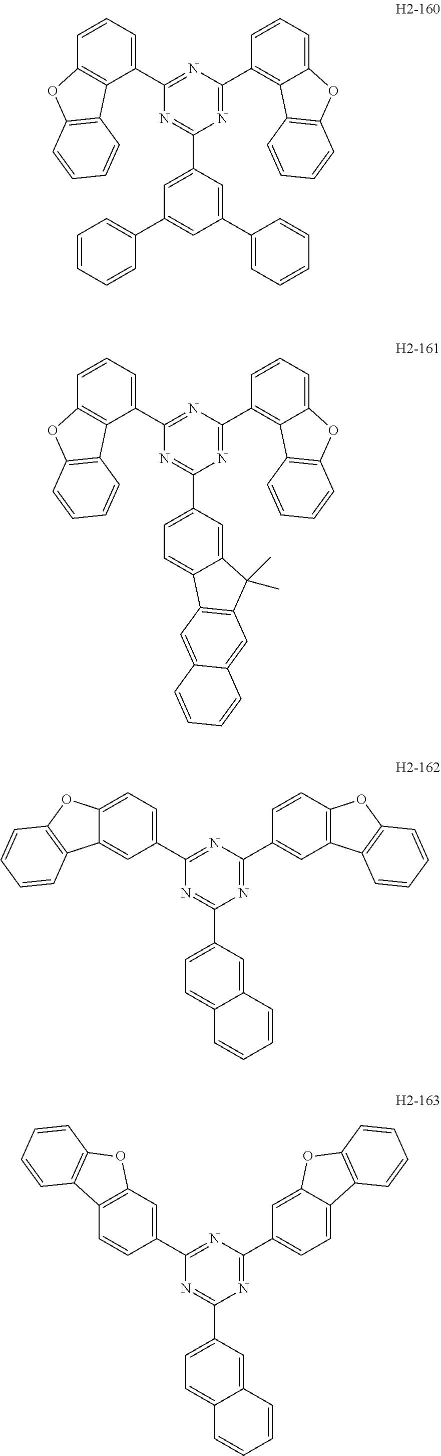

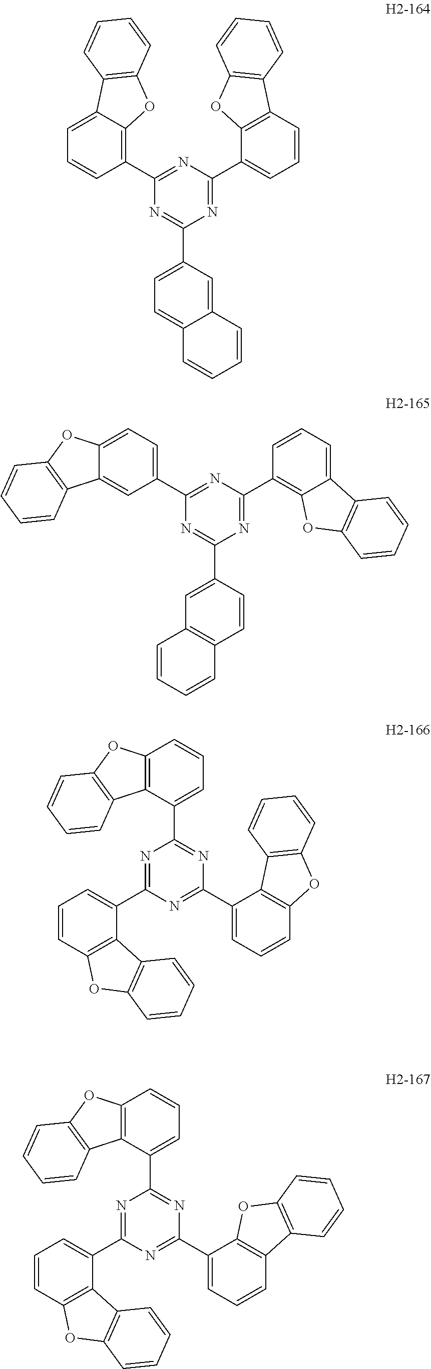

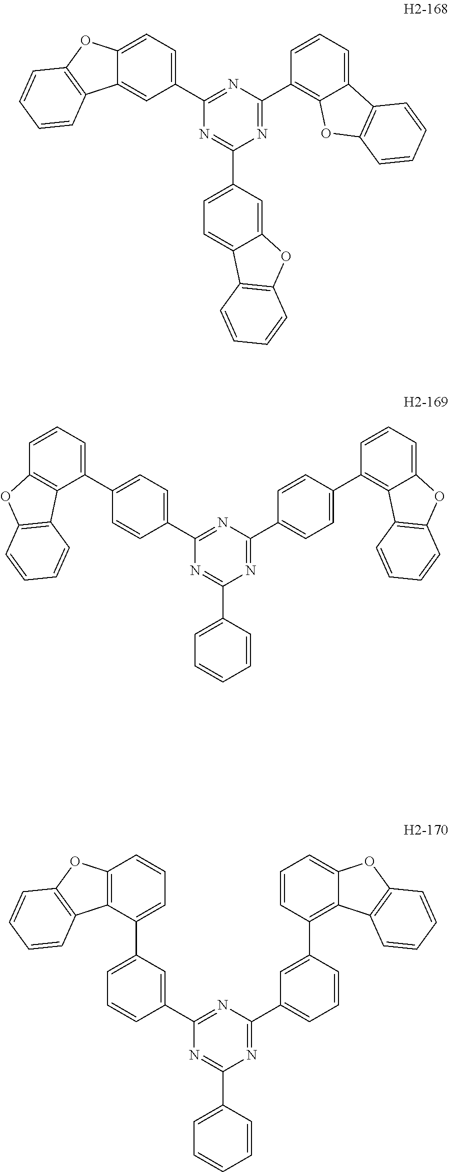 [SMILES: c1ccc2c(c1)oc1cc(-c3nc(-c4ccc5oc6ccccc6c5c4)nc(-c4cccc5c4oc4ccccc45)n3)ccc12 ; c1ccc(-c2nc(-c3ccc(-c4cccc5oc6ccccc6c45)cc3)nc(-c3ccc(-c4cccc5oc6ccccc6c45)cc3)n2)cc1 ; c1ccc(-c2nc(-c3cccc(-c4cccc5oc6ccccc6c45)c3)nc(-c3cccc(-c4cccc5oc6ccccc6c45)c3)n2)cc1]

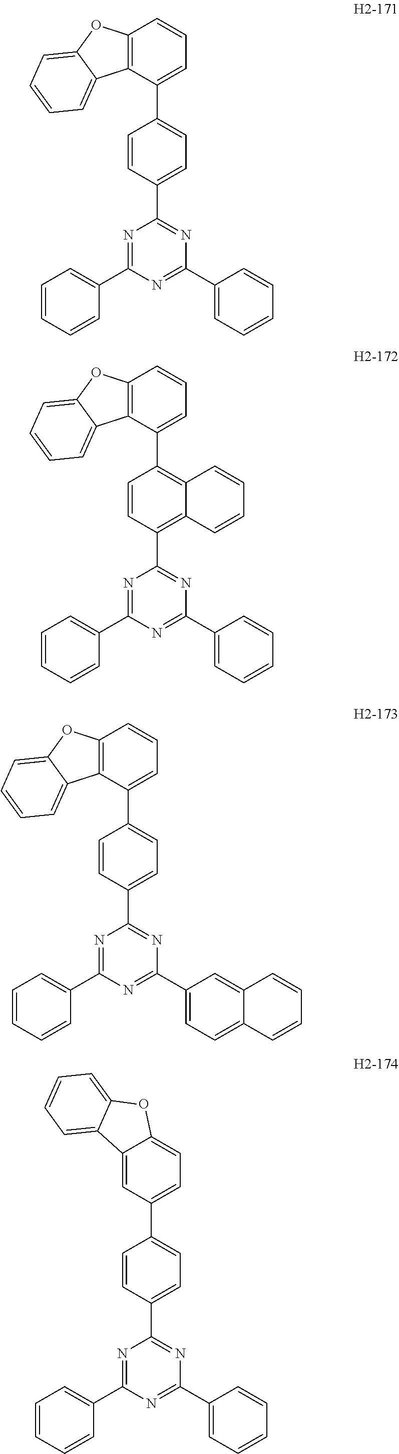

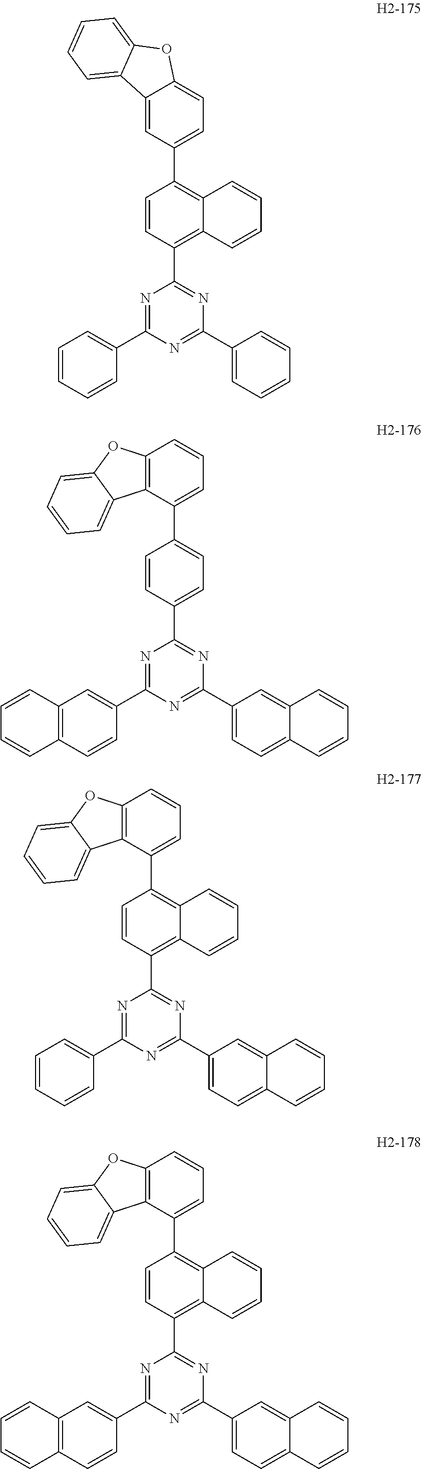

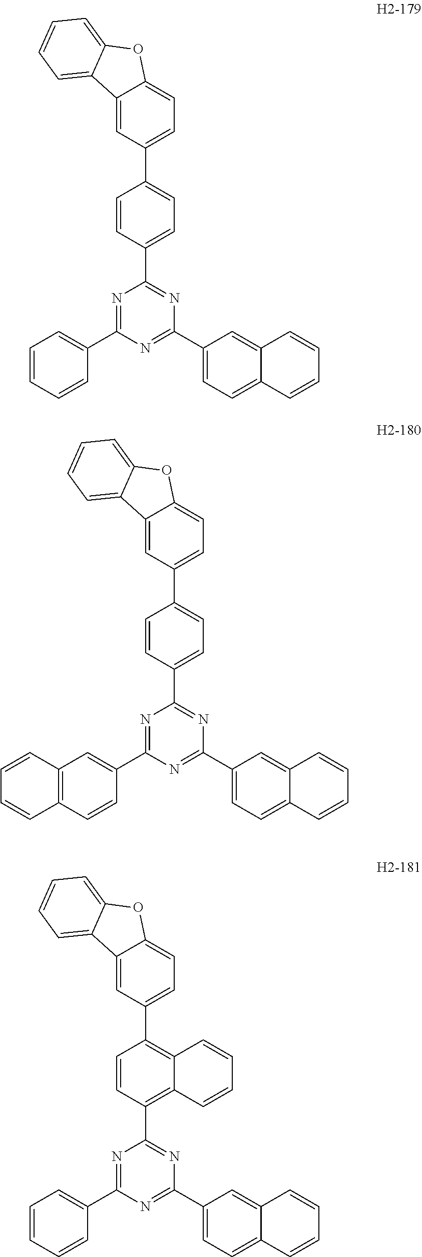 [SMILES: c1ccc(-c2nc(-c3ccc(-c4ccc5oc6ccccc6c5c4)cc3)nc(-c3ccc4ccccc4c3)n2)cc1 ; c1ccc2cc(-c3nc(-c4ccc(-c5ccc6oc7ccccc7c6c5)cc4)nc(-c4ccc5ccccc5c4)n3)ccc2c1 ; c1ccc(-c2nc(-c3ccc4ccccc4c3)nc(-c3ccc(-c4ccc5oc6ccccc6c5c4)c4ccccc34)n2)cc1]

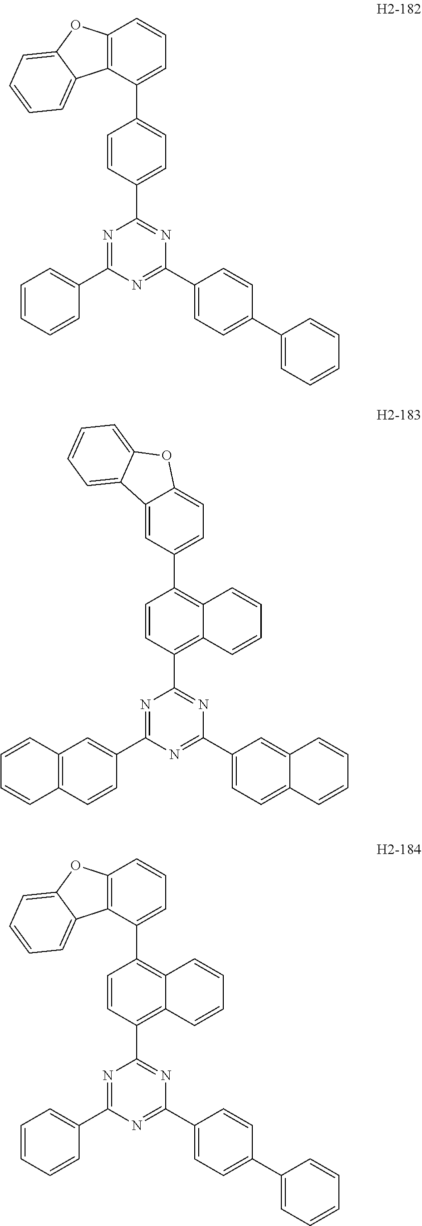 [SMILES: c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5cccc6oc7ccccc7c56)c5ccccc45)n3)cc2)cc1]

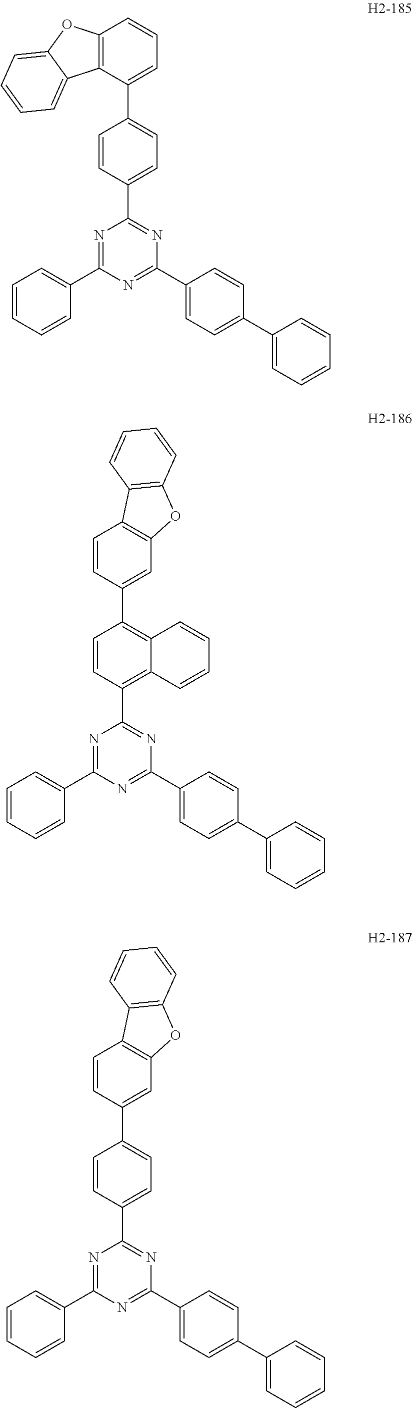 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1]

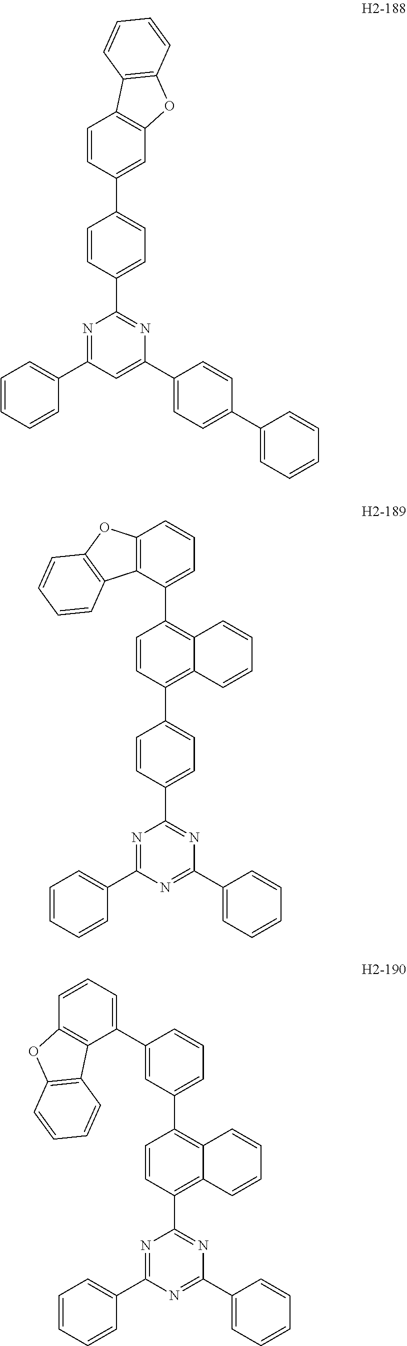

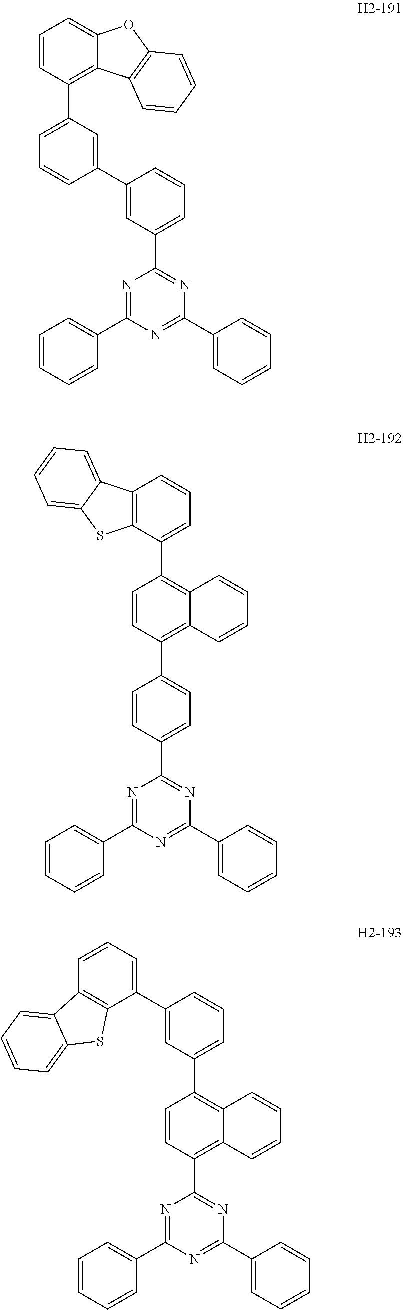 [SMILES: c1ccc(-c2nc(-c3ccccc3)nc(-c3cccc(-c4cccc(-c5cccc6oc7ccccc7c56)c4)c3)n2)cc1 ; c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1]

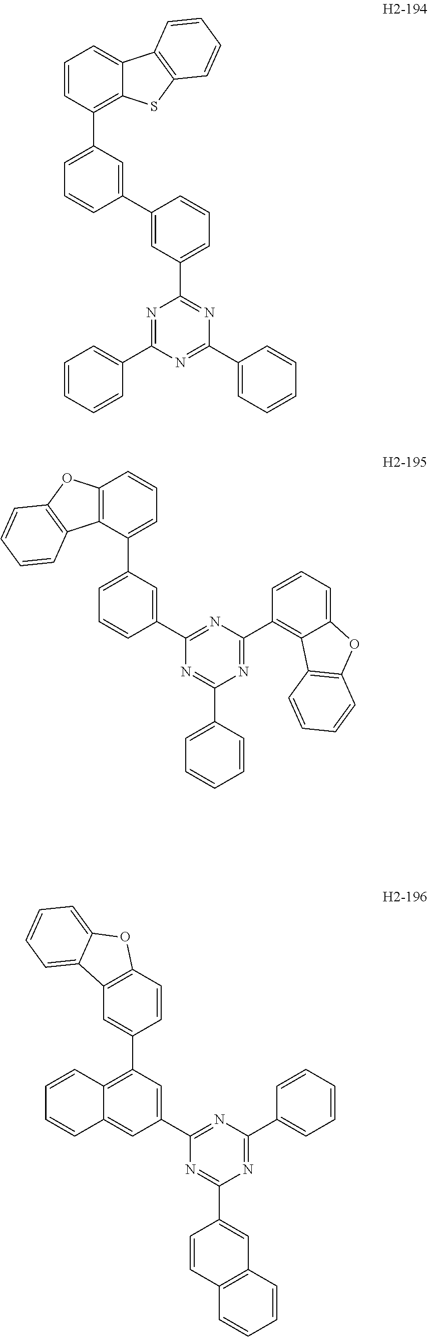 [SMILES: c1ccc(-c2nc(-c3ccccc3)nc(-c3cccc(-c4cccc(-c5cccc6c5sc5ccccc56)c4)c3)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3ccc4ccccc4c3)nc(-c3cc(-c4ccc5oc6ccccc6c5c4)c4ccccc4c3)n2)cc1]

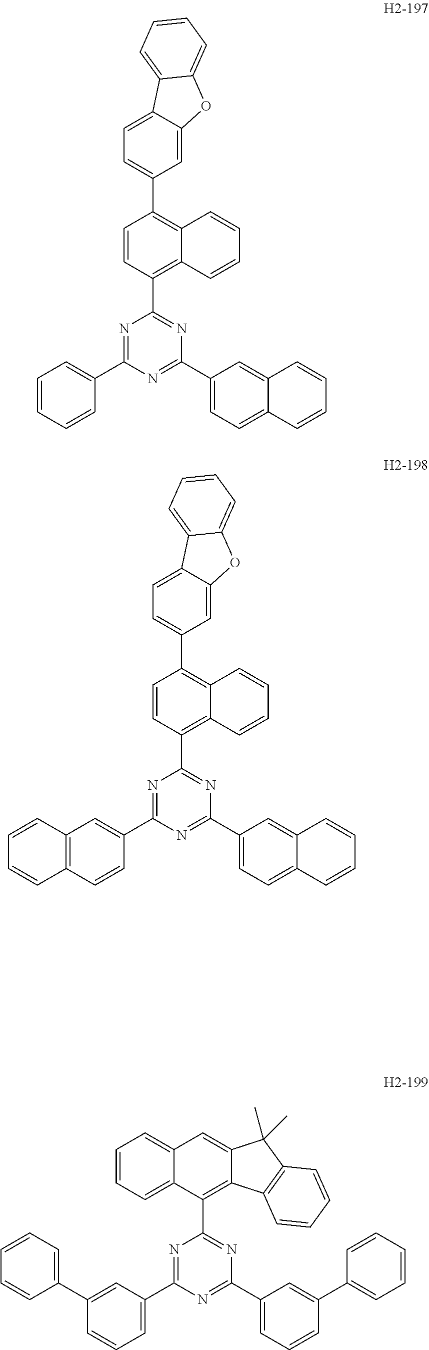 [SMILES: c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1 ; CC1(C)c2ccccc2-c2c1cc1ccccc1c2-c1nc(-c2cccc(-c3ccccc3)c2)nc(-c2cccc(-c3ccccc3)c2)n1]

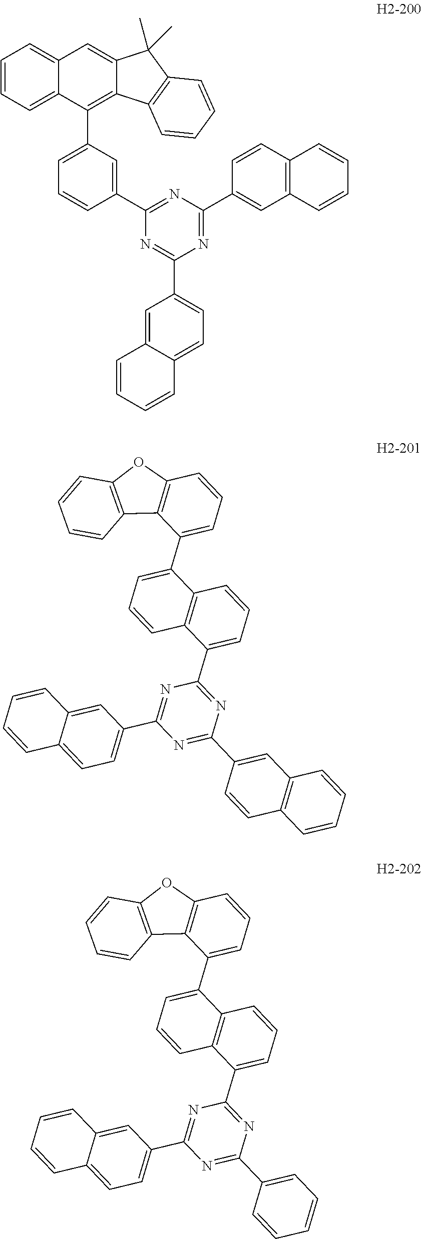 [SMILES: CC1(C)c2ccccc2-c2c1cc1ccccc1c2-c1cccc(-c2nc(-c3ccc4ccccc4c3)nc(-c3ccc4ccccc4c3)n2)c1 ; c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1]

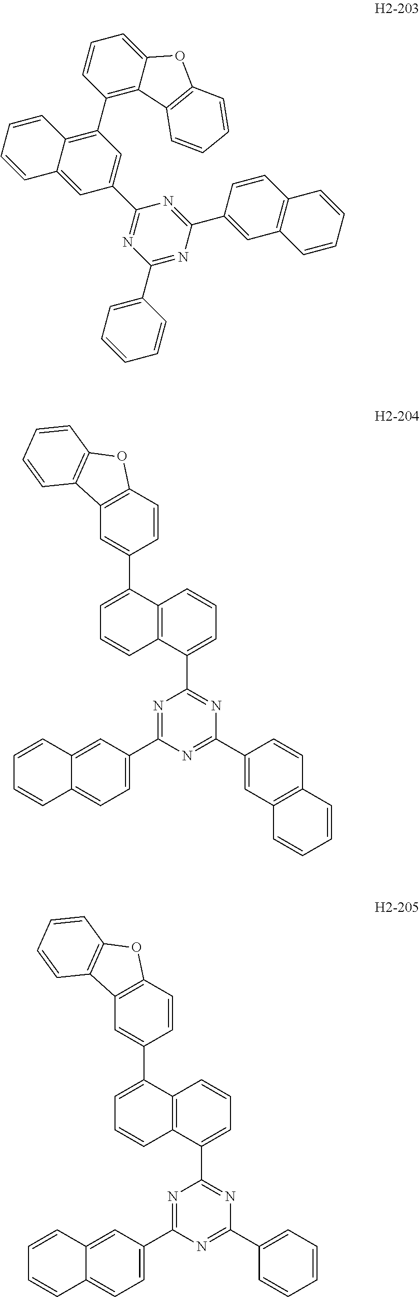 [SMILES: c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1 ; c1ccc(-c2nc(-c3ccc4ccccc4c3)nc(-c3cccc4c(-c5ccc6oc7ccccc7c6c5)cccc34)n2)cc1]

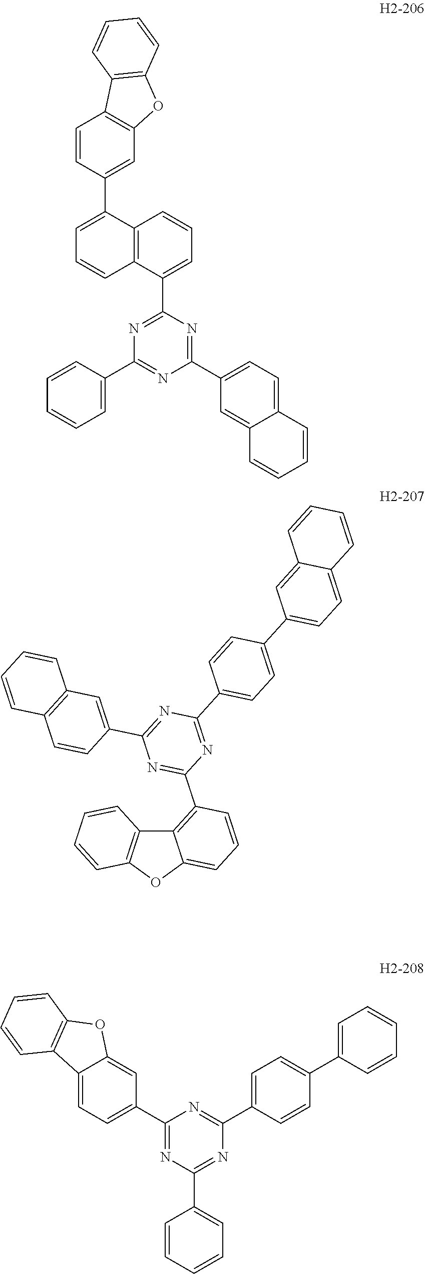

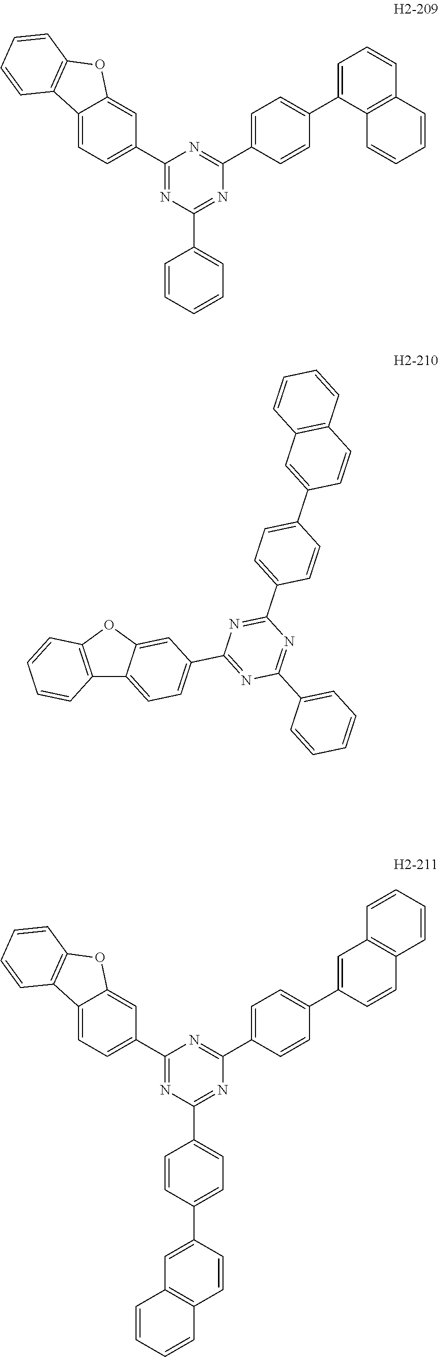 [SMILES: c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1 ; c1ccc2cc(-c3ccc(-c4nc(-c5ccc(-c6ccc7ccccc7c6)cc5)nc(-c5ccc6c(c5)oc5ccccc56)n4)cc3)ccc2c1]

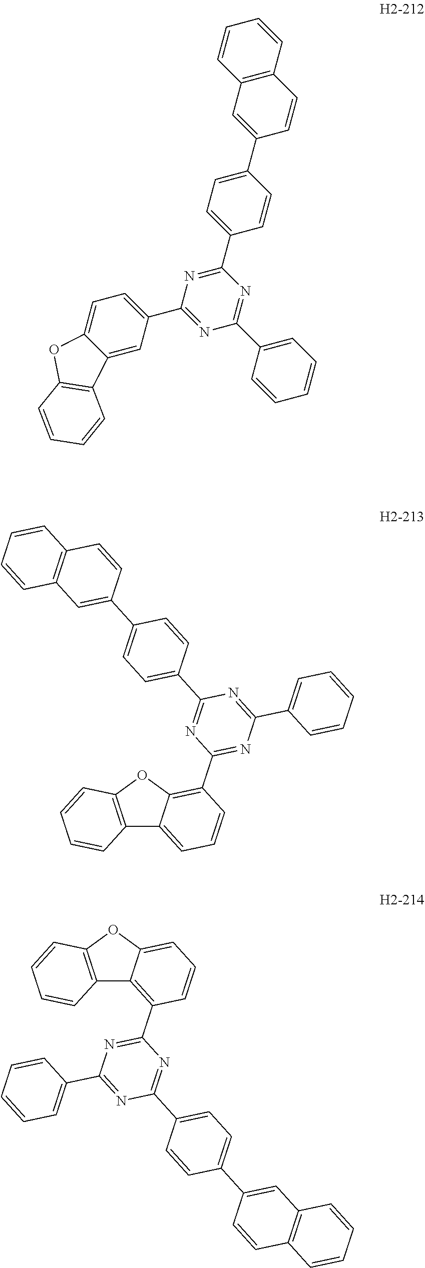 [SMILES: c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1 ; c1ccc(-c2nc(-c3ccc(-c4ccc5ccccc5c4)cc3)nc(-c3cccc4oc5ccccc5c34)n2)cc1]

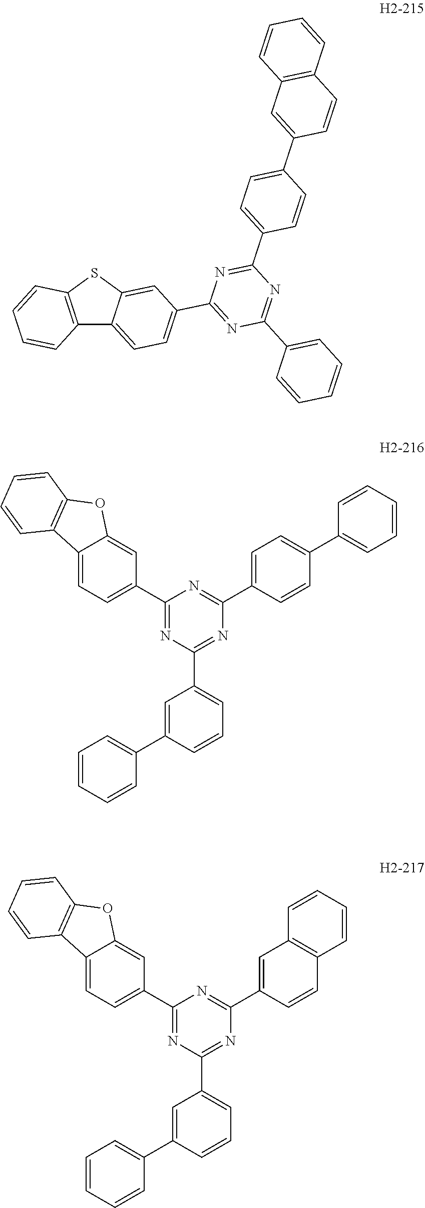 [SMILES: c1ccc(-c2nc(-c3ccc(-c4ccc5ccccc5c4)cc3)nc(-c3ccc4c(c3)sc3ccccc34)n2)cc1 ; c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1]

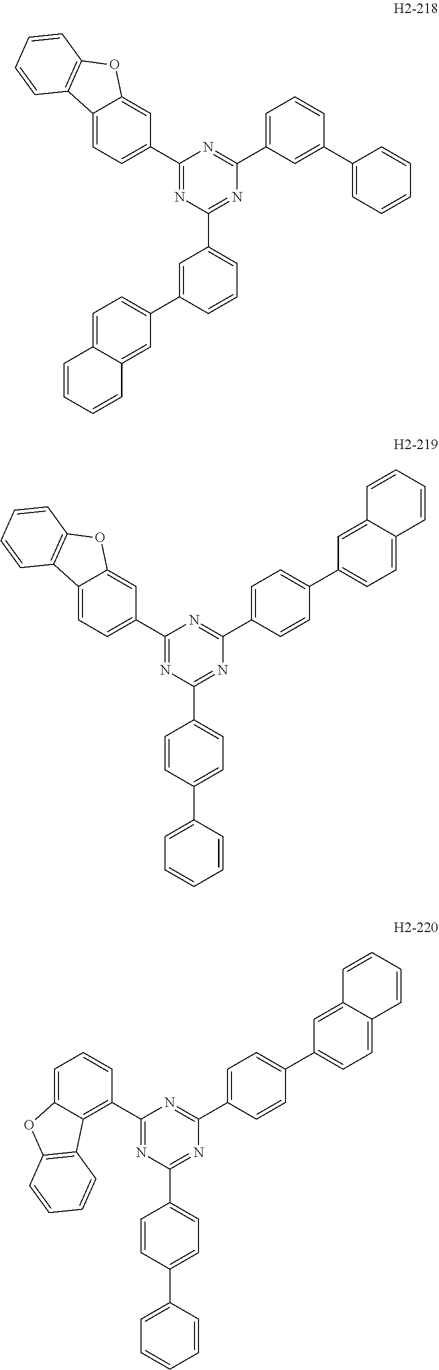 [SMILES: c1ccc(-c2cccc(-c3nc(-c4cccc(-c5ccc6ccccc6c5)c4)nc(-c4ccc5c(c4)oc4ccccc45)n3)c2)cc1 ; c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

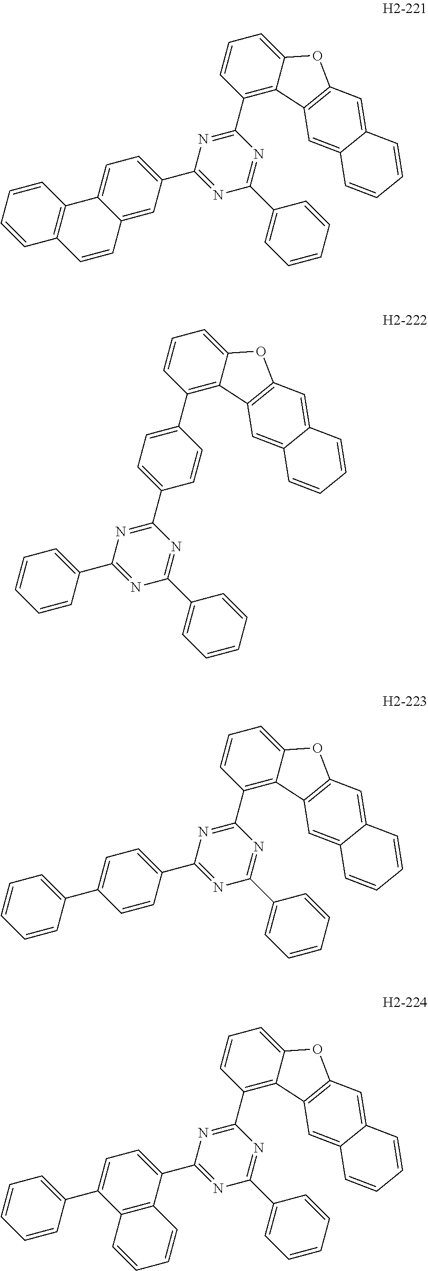 [SMILES: c1ccc(-c2nc(-c3ccc4c(ccc5ccccc54)c3)nc(-c3cccc4oc5cc6ccccc6cc5c34)n2)cc1 ; c1ccc(-c2nc(-c3ccccc3)nc(-c3ccc(-c4cccc5oc6cc7ccccc7cc6c45)cc3)n2)cc1 ; c1ccc(-c2ccc(-c3nc(-c4ccccc4)nc(-c4cccc5oc6cc7ccccc7cc6c45)n3)cc2)cc1 ; c1ccc(-c2nc(-c3ccc(-c4ccccc4)c4ccccc34)nc(-c3cccc4oc5cc6ccccc6cc5c34)n2)cc1]

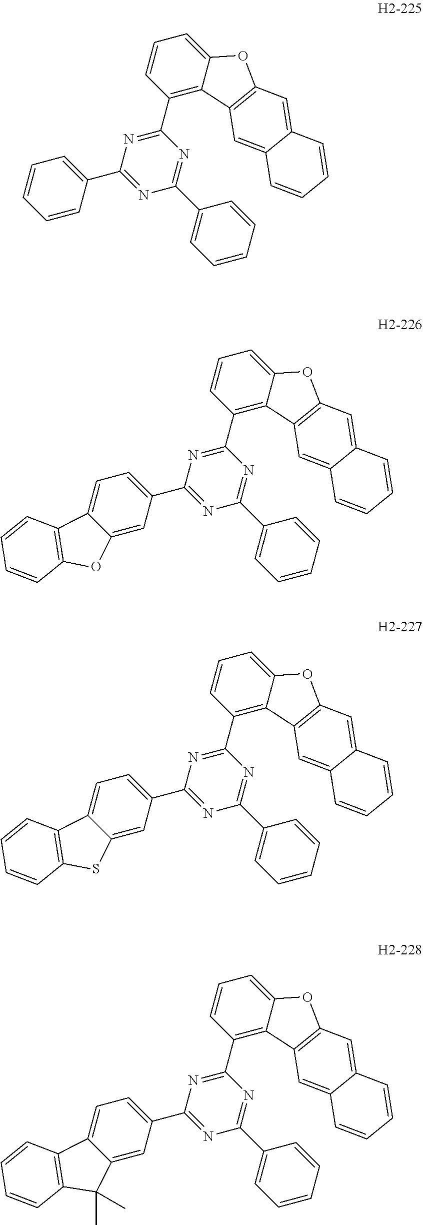 [SMILES: c1ccc(-c2nc(-c3ccccc3)nc(-c3cccc4oc5cc6ccccc6cc5c34)n2)cc1 ; c1ccc(-c2nc(-c3ccc4c(c3)oc3ccccc34)nc(-c3cccc4oc5cc6ccccc6cc5c34)n2)cc1 ; c1ccc(-c2nc(-c3ccc4c(c3)sc3ccccc34)nc(-c3cccc4oc5cc6ccccc6cc5c34)n2)cc1 ; CC1(C)c2ccccc2-c2ccc(-c3nc(-c4ccccc4)nc(-c4cccc5oc6cc7ccccc7cc6c45)n3)cc21]

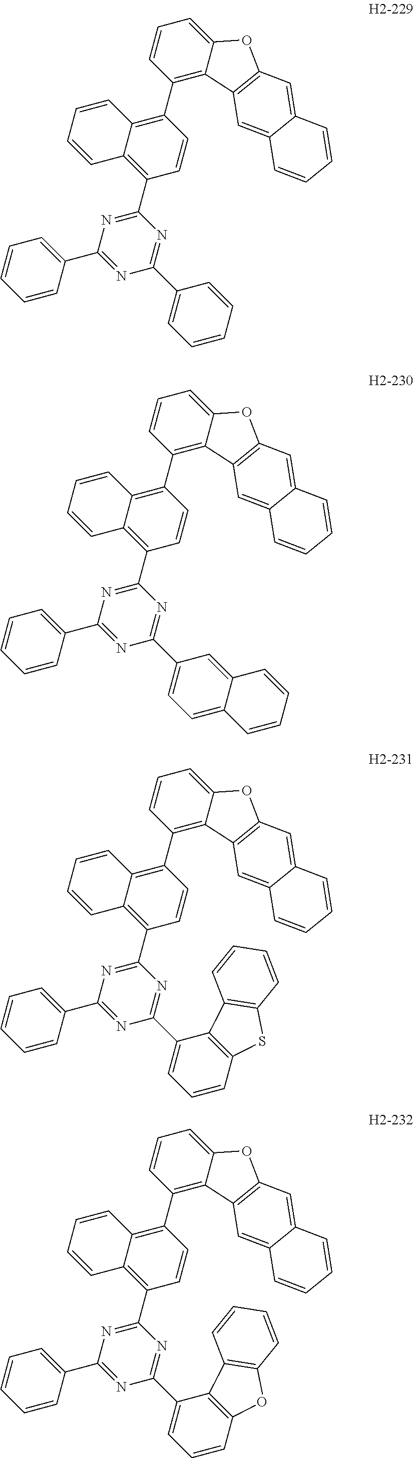

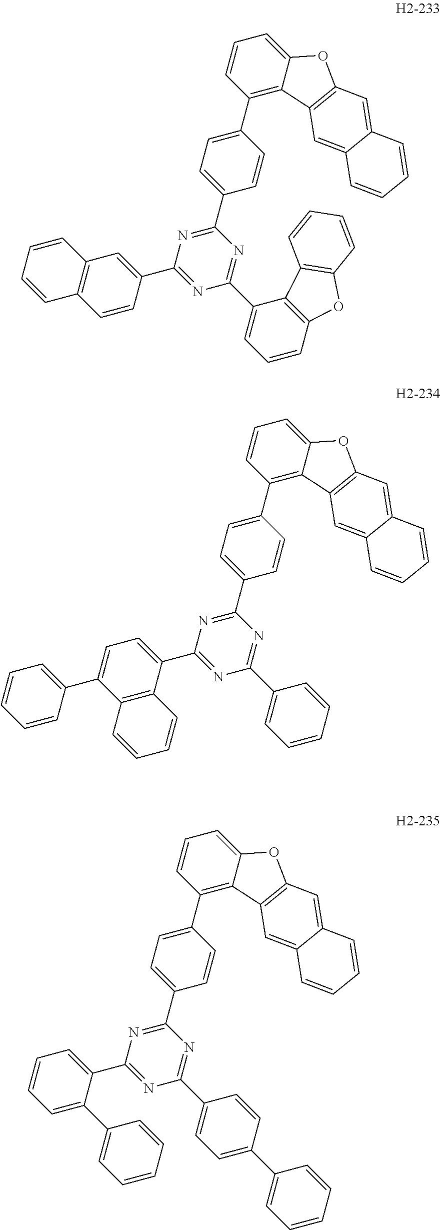 [SMILES: c1ccc2cc(-c3nc(-c4ccc(-c5cccc6oc7cc8ccccc8cc7c56)cc4)nc(-c4cccc5oc6ccccc6c45)n3)ccc2c1 ; c1ccc(-c2nc(-c3ccc(-c4cccc5oc6cc7ccccc7cc6c45)cc3)nc(-c3ccc(-c4ccccc4)c4ccccc34)n2)cc1 ; c1ccc(-c2ccc(-c3nc(-c4ccc(-c5cccc6oc7cc8ccccc8cc7c56)cc4)nc(-c4ccccc4-c4ccccc4)n3)cc2)cc1]

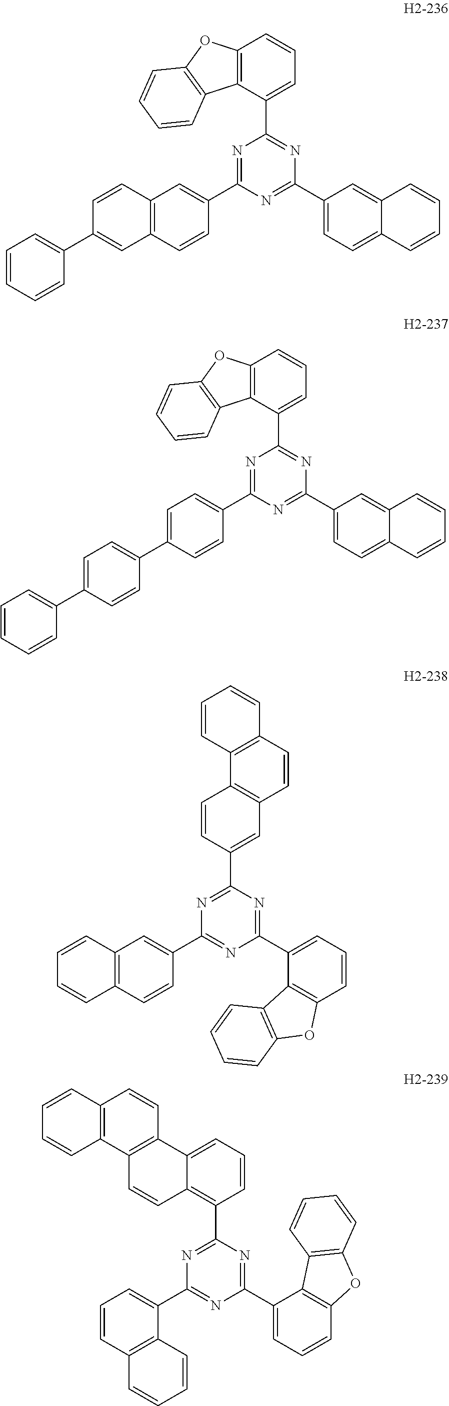 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc2cc(-c3nc(-c4ccc5c(ccc6ccccc65)c4)nc(-c4cccc5oc6ccccc6c45)n3)ccc2c1 ; c1ccc2c(-c3nc(-c4cccc5c4ccc4c6ccccc6ccc54)nc(-c4cccc5oc6ccccc6c45)n3)cccc2c1]

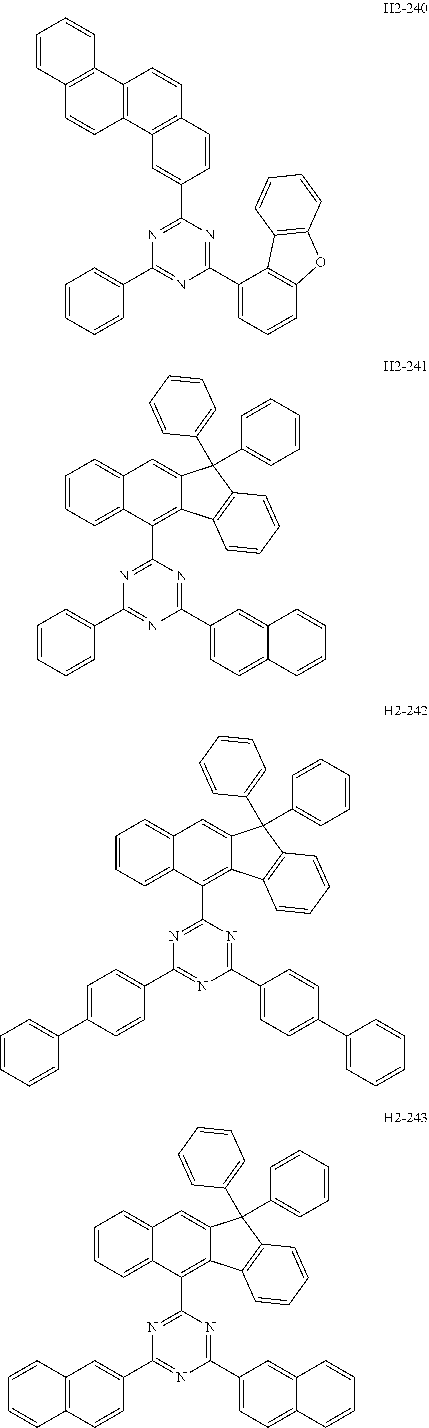 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccccc4c3)nc(-c3c4c(cc5ccccc35)C(c3ccccc3)(c3ccccc3)c3ccccc3-4)n2)cc1 ; c1ccc(-c2ccc(-c3nc(-c4ccc(-c5ccccc5)cc4)nc(-c4c5c(cc6ccccc46)C(c4ccccc4)(c4ccccc4)c4ccccc4-5)n3)cc2)cc1 ; c1ccc(C2(c3ccccc3)c3ccccc3-c3c2cc2ccccc2c3-c2nc(-c3ccc4ccccc4c3)nc(-c3ccc4ccccc4c3)n2)cc1]

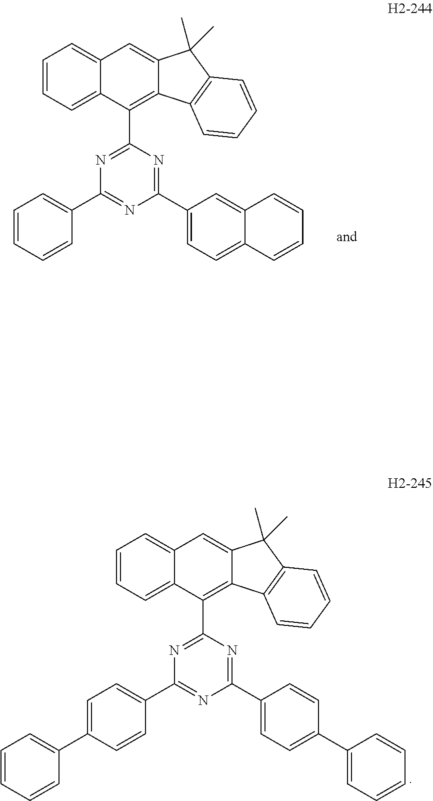 [SMILES: CC1(C)c2ccccc2-c2c1cc1ccccc1c2-c1nc(-c2ccccc2)nc(-c2ccc3ccccc3c2)n1 ; CC1(C)c2ccccc2-c2c1cc1ccccc1c2-c1nc(-c2ccc(-c3ccccc3)cc2)nc(-c2ccc(-c3ccccc3)cc2)n1]

13. An organic electroluminescent device comprising the organic electroluminescent compound according to claim 1.

14. An organic electroluminescent device comprising an anode, a cathode, and at least one light-emitting layer between the anode and the cathode, wherein the at least one light-emitting layer comprises the plurality of host materials according to claim 9.
